(19) United States
(12) Patent Application Publication
(10) Pub. No.: US 2025/0042877 A1
(43) Pub. Date: Feb. 6, 2025

(54) ORGANIC COMPOUND, ORGANIC DEVICE, LIGHT-EMITTING APPARATUS, AND ELECTRONIC APPLIANCE

(71) Applicant: SEMICONDUCTOR ENERGY LABORATORY CO., LTD., Atsugi-shi, Kanagawa-ken (JP)

(72) Inventors: Yuki HAYASHI, Atsugi, Kanagawa (JP); Sachiko KAWAKAMI, Atsugi, Kanagawa (JP); Naoaki HASHIMOTO, Sagamihara, Kanagawa (JP)

(21) Appl. No.: 18/710,010

(22) PCT Filed: Nov. 18, 2022
(86) PCT No.: PCT/IB2022/061119

§ 371 Date: May 14, 2024

(30) Foreign Application Priority Data
Nov. 30, 2021 (JP) 2021-194181

(51) Int. Cl. C07D 403/10; H10K 50/16; H10K 85/60

(52) U.S. Cl. CPC C07D 403/10; H10K 85/6572; H10K 50/16

(57) ABSTRACT

A novel compound, a synthesis method thereof, and an organic device including the novel compound are provided. A compound represented by General Formula (G1) is provided. In General Formula (G1), each of R1, R2, and R5 to R7 independently represents any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms. R3 represents a condensed ring composed of 4 to 10 rings containing nitrogen as an element forming the ring, and R4 represents any of a substituted or unsubstituted condensed ring having 1 to 60 carbon atoms, a substituted or unsubstituted aryl group with a molecular weight of 78 or more, and a substituted or unsubstituted heteroaryl group with a molecular weight of 80 or more. At least one of A1 to A3 represents nitrogen, and each of the others represents substituted carbon. Each of substituents of A1 to A3 independently represents any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms.

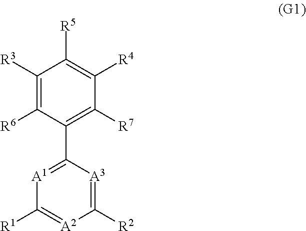

### TECHNICAL FIELD

One embodiment of the present invention relates to an organic compound, an organic device, a light-emitting device, a light-emitting apparatus, a light-emitting and light-receiving device, a display apparatus, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Accordingly, more specific examples of the technical field of one embodiment of the present invention disclosed in this specification include a semiconductor device, a display apparatus, a liquid crystal display apparatus, a light-emitting apparatus, a lighting device, a power storage device, a memory device, an imaging device, a driving method thereof, and a manufacturing method thereof.

### BACKGROUND ART

Light-emitting devices (organic EL elements or light-emitting elements) including organic compounds and utilizing electroluminescence (EL) have been put into practical use. In the basic structure of such light-emitting devices, an organic compound layer containing a light-emitting material (an EL layer) is interposed between a pair of electrodes. Carriers are injected by application of voltage to the device, and recombination energy of the carriers is used, whereby light emission can be obtained from the light-emitting material.

Such light-emitting devices are of self-luminous type; thus, they have higher visibility than liquid crystal displays and are suitable for pixels of a display. Displays using such light-emitting devices are also highly advantageous in that they require no backlight and can be fabricated to be thin and lightweight. Another feature is an extremely high response speed.

Since light-emitting layers of such light-emitting devices can be successively formed two-dimensionally, planar light emission can be obtained. This feature is difficult to realize with point light sources typified by incandescent lamps and LEDs or linear light sources typified by fluorescent lamps; thus, the light-emitting devices also have great potential as planar light sources, which can be used for lighting and the like.

Displays or lighting devices using light-emitting devices can be suitably used for a variety of electronic devices as described above, and research and development of light-emitting devices have progressed for higher efficiency and a longer lifetime.

Examples of an electron-transport material with a long lifetime for an organic electroluminescent device include an azine compound disclosed in Patent Document 1. However, further improvement has been required from a variety of aspects such as improvement in element lifetime and high-temperature resistance.

### REFERENCE

### Patent Document

[Patent Document 1] Japanese Published Patent Application No. 2011-063584

### SUMMARY OF THE INVENTION

### Problems to be Solved by the Invention

An object of one embodiment of the present invention is to provide a novel organic compound. Another object of one embodiment of the present invention is to provide a synthesis method of a novel organic compound. Another object of one embodiment of the present invention is to provide a novel electron-transport material. Another object of one embodiment of the present invention is to provide a novel organic device. Another object of one embodiment of the present invention is to provide a light-emitting element having favorable emission efficiency.

Another object of one embodiment of the present invention is to provide a compound with a high glass transition temperature Tg.

Another object of one embodiment of the present invention is to provide a compound with high stability.

Another object of one embodiment of the present invention is to provide an organic device with high heat resistance.

Another object of one embodiment of the present invention is to provide an organic device that has a long driving lifetime or less luminance degradation in high-temperature driving.

Another object of one embodiment of the present invention is to provide an organic device with a small variation in voltage in high-temperature driving.

In this specification, an organic device (also referred to as organic element) refers to any device including an organic compound (excluding a human). Examples of an organic device include a light-emitting device (also referred to as light-emitting element) and a light-receiving device (also referred to as light-receiving element). An organic device refers to, for example, a device including an organic compound layer (a thin-film layer with a thickness of several micrometers or smaller) between a pair of electrodes. Not only a device including an electrode, but also a device including an organic compound layer (e.g., a film such as an evaporation film or a coating film) over a substrate, a device including a solvent that includes an organic compound, or the like is sometimes referred to as an organic device. Alternatively, a device including a layer (e.g., a cap layer, a partition wall, a sealing layer, or a color filter) provided around an electrode is sometimes referred to as an organic device.

Note that the description of these objects does not preclude the existence of other objects. In one embodiment of the present invention, there is no need to achieve all of these objects. Other objects will be apparent from the description of the specification, the drawings, the claims, and the like, and other objects can be derived from the description of the specification, the drawings, the claims, and the like.

### Means for Solving the Problems

One embodiment of the present invention is a novel azine compound and a method for fabricating the azine compound. Another embodiment of the present invention is a novel organic device including the azine compound.

Another embodiment of the present invention is an organic compound represented by General Formula (G1) below.

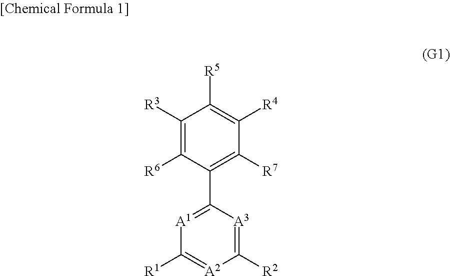

In General Formula (G1), each of R¹, R², and R⁵ to R⁷ independently represents any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms. R³ represents a condensed ring composed of 4 to 10 rings containing nitrogen as an element forming the ring, and R⁴ represents any of a substituted or unsubstituted condensed ring having 1 to 60 carbon atoms, a substituted or unsubstituted aryl group with a molecular weight of 78 or more, and a substituted or unsubstituted heteroaryl group with a molecular weight of 80 or more. Any one of A¹ to A³ represents nitrogen, each of the others represents nitrogen or carbon, and in the case where each of the others represents carbon, each of A¹ to A³ is independently bonded to any one of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms.

Another embodiment of the present invention is the organic compound in which in General Formula (G1), the R³ represents General Formula (G1-1), and the R⁴ represents any one of General Formula (G1-2) to General Formula (G1-6).

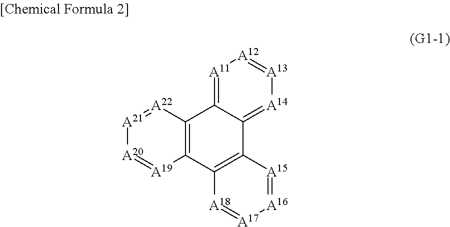

In General Formula (G1-1), any one of A¹¹ to A²² represents nitrogen, another one represents carbon, and each of the others independently represents nitrogen or carbon. Any one of the carbons of A¹¹ to A²² is bonded to General Formula (G1), and each of the others is bonded to any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A¹¹ to A²² may be bonded to form a ring.

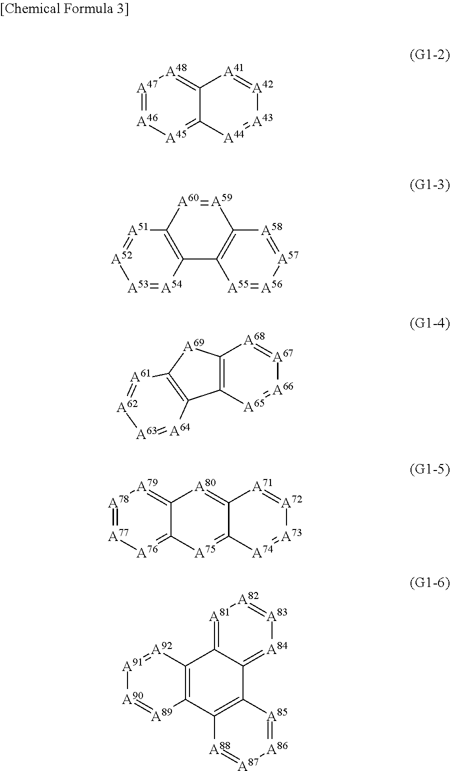

In General Formula (G1-2), any one of A⁴¹ to A⁴⁸ represents carbon, and each of the others independently represents nitrogen or carbon. Any one of the carbons of A⁴¹ to A⁴⁸ is bonded to General Formula (G1), and each of the others is independently bonded to any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁴¹ to A⁴⁸ may be bonded to each other to form a ring. In General Formula (G1-3), any one of A⁵¹ to A⁶⁰ represents carbon, and each of the others independently represents nitrogen or carbon. Any one of the carbons of A⁵¹ to A⁶⁰ is bonded to General Formula (G1), and each of the others is independently bonded to any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁵¹ to A⁶⁰ may be bonded to each other to form a ring. In General Formula (G1-4), any one of A⁶¹ to A⁶⁸ represents carbon, and each of the others independently represents nitrogen or carbon. A⁶⁹ represents nitrogen, carbon, sulfur, or oxygen. Any one of the carbons of A⁶¹ to A⁶⁸ and the nitrogen of A⁶⁹ is bonded to General Formula (G1), and each of the others independently represents any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁶¹ to A⁶⁸ may be bonded to each other to form a ring. In General Formula (G1-5), any one of A⁷¹ to A⁸⁰ represents carbon, and each of the others independently represents nitrogen or carbon. Any one of the carbons of A⁷¹ to A⁸⁰ is bonded to General Formula (G1), and each of the others independently represents any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁷¹ to A⁸⁰ may be bonded to each other to form a ring. In General Formula (G1-6), any one of A⁸¹ to A⁹² represents carbon, and each of the others independently represents nitrogen or carbon. Any one of the carbons of A⁸¹ to A⁹² is bonded to General Formula (G1), and each of the others independently represents any of hydrogen (including deuterium), an alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁸¹ to A⁹² may be bonded to each other to form a ring.

Another embodiment of the present invention is an organic compound represented by Structural Formula (100).

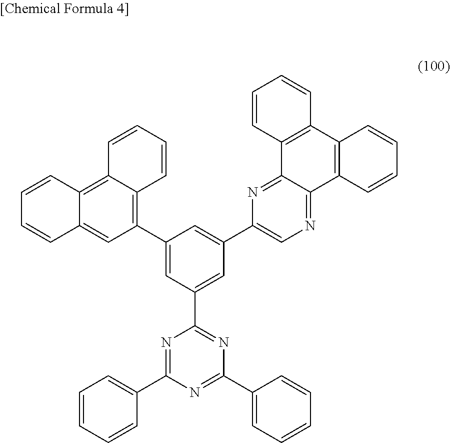 [SMILES: c1ccc(-c2nc(-c3ccccc3)nc(-c3cc(-c4cnc5c6ccccc6c6ccccc6c5n4)cc(-c4cc5ccccc5c5ccccc45)c3)n2)cc1]

Another embodiment of the present invention is the organic compound in which the R¹ and the R² in General Formula (G1) are each a substituted or unsubstituted phenyl group.

Another embodiment of the present invention is the organic compound being represented by General Formula (G1) and having a glass transition temperature higher than or equal to 150° C.

Another embodiment of the present invention is the organic compound in which the R³ and the R⁴ in General Formula (G1) each have a molecular weight of 150 or more.

Another embodiment of the present invention is an organic device including any of the above organic compounds. Another embodiment of the present invention is a light-emitting apparatus including the above device, and a transistor or a substrate.

Another embodiment of the present invention is electronic appliance including any of the above organic compounds. Another embodiment of the present invention is an electronic appliance including the above light-emitting apparatus and a sensor, an operation button, a speaker, or a microphone.

Another embodiment of the present invention is a light-emitting device including an anode, a light-emitting layer, an electron-transport layer including an azine compound, a cathode, and a cap layer. The refractive index of the azine compound is lower than the refractive index of a material contained in the cap layer and the refractive index of a host material in the light-emitting layer.

Another embodiment of the present invention is a light-emitting apparatus including an anode, a hole-transport layer, a light-emitting layer, an electron-transport layer including an azine compound, and a cathode. The glass transition temperature of the azine compound is higher than the glass transition temperature of a material contained in the hole-transport layer and the glass transition temperature of a host material in the light-emitting layer.

Another embodiment of the present invention is a light-emitting apparatus including an anode, a hole-transport layer, a light-emitting layer, an electron-transport layer including an azine compound, a cathode, and a partition wall. The electron-transport layer includes a region overlapping with the partition wall. The glass transition temperature of the azine compound is higher than the glass transition temperature of a material contained in the hole-transport layer and the glass transition temperature of a host material in the light-emitting layer.

Note that these solving means do not preclude the existence of other solving means. Note that one embodiment of the present invention does not need to have all of these solving means. Other solving means will be apparent from the description of the specification, the drawings, the claims, and the like, and other solving means can be derived from the description of the specification, the drawings, the claims, and the like.

### Effect of the Invention

According to one embodiment of the present invention, a novel organic compound can be provided. According to another embodiment of the present invention, a novel carrier-transport material can be provided. According to another embodiment of the present invention, a novel host material can be provided. According to another embodiment of the present invention, a compound with a high glass transition temperature can be provided. According to another embodiment of the present invention, a compound with high stability can be provided. According to another embodiment of the present invention, a novel organic device can be provided. According to another embodiment of the present invention, an organic device with high emission efficiency can be fabricated. According to another embodiment of the present invention, an organic device with low power consumption can be provided. According to another embodiment of the present invention, an organic device with high reliability can be provided. According to another embodiment of the present invention, a thin film including a novel organic compound (also referred to as an organic compound layer) can be provided.

According to another embodiment of the present invention, a light-emitting apparatus, an electronic appliance, and a display apparatus each having high emission efficiency can be provided. According to another embodiment of the present invention, a light-emitting apparatus, an electronic appliance, and a display apparatus each having low power consumption can be provided. According to another embodiment of the present invention, a light-emitting apparatus, an electronic appliance, and a display apparatus each having high reliability can be provided.

Note that the description of these effects does not preclude the existence of other effects. Note that one embodiment of the present invention does not need to have all of these effects. Other effects will be apparent from the description of the specification, the drawings, the claims, and the like, and other effects can be derived from the description of the specification, the drawings, the claims, and the like.

### BRIEF DESCRIPTION OF THE DRAWINGS

FIG. 1A to FIG. 1E are diagrams illustrating structures of a light-emitting device of an embodiment.

FIG. 2A to FIG. 2D are diagrams illustrating a light-emitting apparatus of an embodiment.

FIG. 3A to FIG. 3C are diagrams illustrating a method for manufacturing a light-emitting apparatus of an embodiment.

FIG. 4A to FIG. 4C are diagrams illustrating the method for manufacturing a light-emitting apparatus of an embodiment.

FIG. 5A to FIG. 5C are diagrams illustrating the method for manufacturing a light-emitting apparatus of an embodiment.

FIG. 6A to FIG. 6D are diagrams illustrating the method for manufacturing a light-emitting apparatus of an embodiment.

FIG. 7A to FIG. 7E are diagrams illustrating a light-emitting apparatus of an embodiment.

FIG. 8A to FIG. 8F are diagrams illustrating an apparatus and pixel arrangements of an embodiment.

FIG. 9A to FIG. 9C are diagrams illustrating pixel circuits of an embodiment.

FIG. 10 is a diagram illustrating a light-emitting apparatus of an embodiment.

FIG. 11A to FIG. 11E are diagrams illustrating electronic appliances of an embodiment.

FIG. 12A to FIG. 12E are diagrams illustrating electronic appliances of an embodiment.

FIG. 13A and FIG. 13B are diagrams illustrating electronic appliances of an embodiment.

FIG. 14A and FIG. 14B are diagrams illustrating a lighting device of an embodiment.

FIG. 15 is a diagram illustrating lighting devices of an embodiment.

FIG. 16A to FIG. 16C are diagrams each illustrating a light-emitting device and a light-receiving device of an embodiment.

FIG. 17A and FIG. 17B are diagrams each illustrating a light-emitting device and a light-receiving device of an embodiment.

FIG. 18A and FIG. 18B show ¹H-NMR spectra of Pn-mDBqPTzn.

FIG. 19 shows an absorption spectrum and an emission spectrum of Pn-mDBqPTzn in a dichloromethane solution.

FIG. 20 shows an absorption spectrum and an emission spectrum of a thin film of Pn-mDBqPTzn.

FIG. 21 shows emission spectra of a light-emitting device 1 and a light-emitting device 2.

FIG. 22 shows external quantum efficiency-luminance characteristics of the light-emitting device 1 and the light-emitting device 2.

FIG. 23 shows current-voltage characteristics of the light-emitting device 1 and the light-emitting device 2.

FIG. 24 shows luminance changes over driving time of the light-emitting device 1 and the light-emitting device 2 at room temperature.

FIG. 25 shows luminance changes over driving time of the light-emitting device 1 and the light-emitting device 2 at 85° C.

### MODE FOR CARRYING OUT THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the description in the following embodiments.

### Embodiment 1

This embodiment describes an organic compound of one embodiment of the present invention (also referred to as a compound of the present application). The compound of one embodiment of the present invention is an organic compound including an azine skeleton (also referred to as an azine compound). An azine skeleton refers to a skeleton where one or more nitrogen atoms substitute for one or more carbon atoms of a benzene ring; for example, a pyridine skeleton, a pyrazine skeleton, a pyrimidine skeleton, a pyridazine skeleton, and a triazine skeleton can be given. A compound including an azine skeleton is an organic compound represented by General Formula (G1) below, for example.

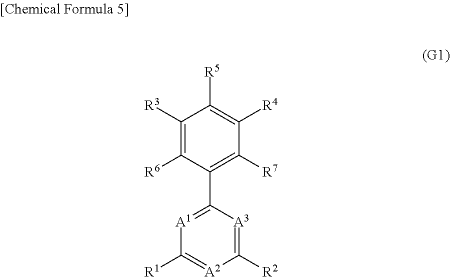

In General Formula (G1), it is preferable that each of R¹ to R⁷ independently represent any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms.

One of R³ and R⁴ preferably represents a condensed ring composed of 4 to 10 rings containing nitrogen as an element forming the ring. The other of R³ and R⁴ preferably represents any of a substituted or unsubstituted condensed ring having 1 to 60 carbon atoms, a substituted or unsubstituted aryl group with a molecular weight of 78 or more, and a heteroaryl group with a molecular weight of 80 or more.

When R³ or R⁴ includes a condensed ring, a carrier-transport property can be increased. Particularly when a condensed ring composed of three or more, preferably four or more rings is included, a carrier-transport property can be further increased. The use of a condensed ring having a helicene structure (a spiral condensed ring), such as phenanthrene, triphenylene, or dibenzoquinoxaline, can widen the band gap and increase the T1 level and the S1 level.

Any one of A¹ to A³ represents nitrogen and each of the others represents nitrogen or carbon, and in the case where each of the others represents carbon, it is preferable that each of A¹ to A³ be independently bonded to any one of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms.

It is preferable that each of R¹ and R² independently include an aryl group with a molecular weight of 70 or more or a heteroaryl group with a molecular weight of 70 or more. It is also preferable that each of R¹ and R² independently include an aryl group with a molecular weight of 150 or more or a heteroaryl group with a molecular weight of 150 or more.

Increasing the molecular weight of a compound and a substituent can improve the resistance (e.g., heat resistance) and the amorphous property of the compound. That is, the glass transition temperature Tg can be increased. Increasing the molecular weight can decrease the solubility of the compound in a solvent and can improve the yield by a purification step such as recrystallization. Since too large a molecular weight hinders sublimation, the molecular weights of R¹ to R⁴ are each preferably less than or equal to 1000, further preferably less than or equal to 500. Specifically, the molecular weights of R¹ to R⁴ are each preferably greater than or equal to 70 and less than or equal to 1000, further preferably greater than or equal to 70 and less than or equal to 500.

In addition, when a substituent includes a condensed ring or the molecular weight is increased, the resistance (e.g., stability) of the compound can be increased. Thus, as R³ or R⁴, it is preferable to use a condensed ring such as a naphthyl group rather than a phenyl group or to use a substituent with a larger molecular weight than a phenyl group.

Note that in this specification, the molecular weight and the atomic weight are rounded off to the nearest whole number for simplification. For example, in the case where the substituent R¹ is a phenyl group, the molecular weight of R¹ is C₆H₅=77. In the case of a naphthyl group, the molecular weight is C₁₀H₇=127. The same applies to the other substituents.

In General Formula (G1), it is preferable that one of R³ and R⁴ represent General Formula (G1-1) and the other of R³ and R⁴ represent any one of General Formula (G1-2) to General Formula (G1-6).

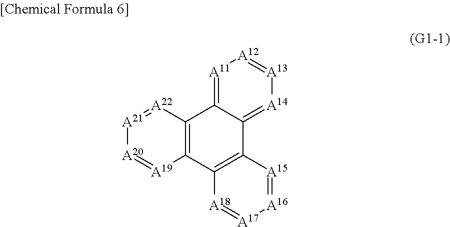

In General Formula (G1-1), it is preferable that any one of A¹¹ to A²² represent nitrogen, another one represent carbon, and each of the others independently represent nitrogen or carbon. It is preferable that any one of A¹¹ to A²² be bonded to General Formula (G1), and each of the others be bonded to any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A¹¹ to A²² may be bonded to each other to form a ring. A structure forming a ring includes, for example, a structure where one or more benzene rings including A¹² and A¹³ are added, and in this case, General Formula (G1-1) includes 5 or more six-membered rings. In the case where a benzene ring is formed in such a manner, a substituted or unsubstituted vinyl group is preferably included as the substituent bonded to A¹¹ to A²². The same applies to General Formula (G1-2) to General Formula (G1-6) to be described later. Note that the structure forming a ring can be simply referred to as a structure further including a ring that includes two of A¹¹ to A²².

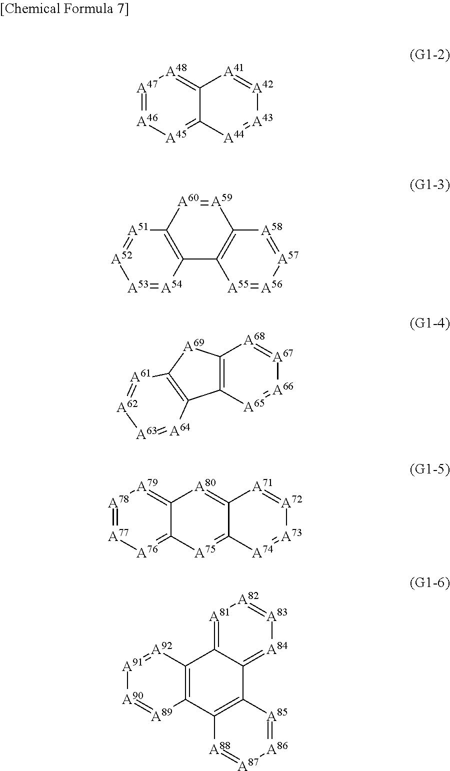

In General Formula (G1-2), it is preferable that any one of A⁴¹ to A⁴⁸ represent carbon, and each of the others independently represent nitrogen or carbon. It is preferable that any one of the carbons of A⁴¹ to A⁴⁸ be bonded to General Formula (G1), and each of the others be independently bonded to any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁴¹ to A⁴⁸ may be bonded to each other to form a ring.

In General Formula (G1-3), it is preferable that any one of A⁵¹ to A⁶⁰ represent carbon, and each of the others independently represent nitrogen or carbon. It is preferable that any one of the carbons of A⁵¹ to A⁶⁰ be bonded to General Formula (G1), and each of the others be independently bonded to any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁵¹ to A⁶⁰ may be bonded to each other to form a ring.

In General Formula (G1-4), it is preferable that any one of A⁶¹ to A⁶⁸ represent carbon, and each of the others independently represent nitrogen or carbon. It is preferable that A⁶⁹ represent nitrogen, carbon, sulfur, or oxygen. It is preferable that any one of the carbons of A⁶¹ to A⁶⁸ and the nitrogen of A⁶⁹ be bonded to General Formula (G1), and each of the others independently represent any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁶¹ to A⁶⁸ may be bonded to each other to form a ring.

In General Formula (G1-5), it is preferable that any one of A⁷¹ to A⁸⁰ represent carbon, and each of the others independently represent nitrogen or carbon. It is preferable that any one of the carbons of A⁷¹ to A⁸⁰ be bonded to General Formula (G1), and each of the others independently represent any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁷¹ to A⁸⁰ may be bonded to each other to form a ring.

In General Formula (G1-6), it is preferable that any one of A⁸¹ to A⁹² represent carbon, and each of the others independently represent nitrogen or carbon. It is preferable that any one of the carbons of A⁸¹ to A⁹² be bonded to General Formula (G1), and each of the others independently represent any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent substituents among substituents bonded to A⁸¹ to A⁹² may be bonded to each other to form a ring.

In addition, the organic compound of one embodiment of the present invention is an organic compound represented by General Formula (G2) below.

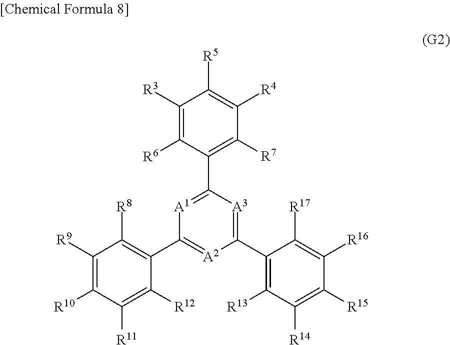

In General Formula (G2), R³ to R⁷ and A¹ to A³ can employ the above structures.

It is preferable that each of R⁸ to R¹⁷ independently represent any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent groups among R⁸ to R¹⁷ may be bonded to each other to form a ring.

Alternatively, the organic compound of one embodiment of the present invention is an organic compound represented by General Formula (G3) below.

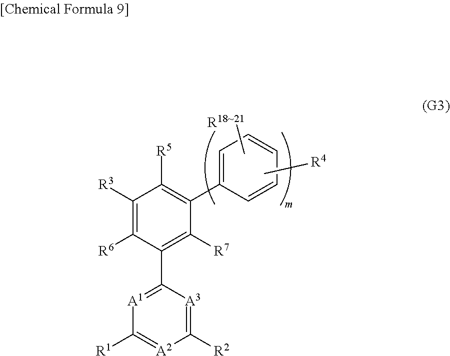

In General Formula (G3), R¹ to R⁷ and A¹ to A³ can employ the above structures.

It is preferable that each of R¹⁸ to R²¹ independently represent any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent groups among R¹⁸ to R²¹ may be bonded to each other to form a ring. Furthermore, m represents 1 or 2. The bonding position of R⁴ may be any of a meta-position, a para-position, and an ortho-position; the sublimation temperature can be decreased in the case of the meta-position, and Tg can be increased in the case of the para-position.

Alternatively, the organic compound of one embodiment of the present invention is an organic compound represented by General Formula (G4) below.

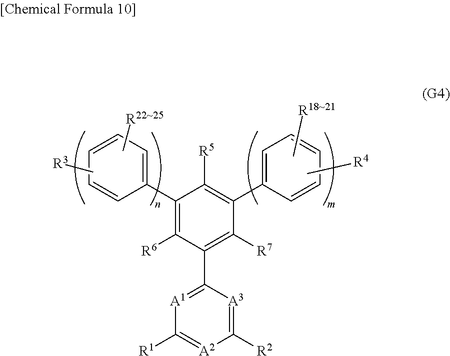

In General Formula (G4), R¹ to R⁷ and A¹ to A³ can employ the above structures.

It is preferable that each of R¹⁸ to R²⁵ independently represent any of hydrogen (including deuterium), a substituted or unsubstituted alkyl group having 1 to 10 carbon atoms, a substituted or unsubstituted aryl group having 1 to 60 carbon atoms, and a substituted or unsubstituted heteroaryl group having 1 to 60 carbon atoms. Adjacent groups among R¹⁸ to R²⁵ may be bonded to each other to form a ring. Furthermore, m represents 1 or 2. Furthermore, n represents 1 or 2. The bonding positions of R³ and R⁴ may each be any of a meta-position, a para-position, and an ortho-position; the sublimation temperature can be decreased in the case of the meta-position, and Tg can be increased in the case of the para-position.

The glass transition temperature Tg of the compound of one embodiment of the present invention is preferably higher than or equal to 100° C. The glass transition temperature Tg of the compound of one embodiment of the present invention is higher than or equal to 120° C., preferably higher than or equal to 130° C., further preferably higher than or equal to 140° C., still further preferably higher than or equal to 150° C., particularly desirably higher than or equal to 160° C.

The solubility of the compound of one embodiment of the present invention is preferably higher than or equal to 0% and lower than 5%. In this specification, the solubility (weight %) is evaluated as follows. When a solute A (g) and a solvent B (g) are put into a glass container and no precipitate is visually observed, the solubility is determined to be higher than or equal to (A/(A+B)×100) %, and when a precipitate is visually observed, the solubility is determined to be lower than (A/(A+B)×100) %.

Note that for A¹ to A⁹² or R¹ to R²⁵ in General Formulae (G1) to (G4), a combination of the above structures (e.g., the substituent structure and the molecular weight) may be employed. For example, it is preferable to combine a structure where “R³ and R⁴ each include a condensed ring composed of three or more rings and R⁴ contains nitrogen” and a structure where “A¹ to A³ are all nitrogen and R¹ and R² each have a molecular weight of 70 or more”. An appropriate combination of a plurality of structures, without limited to the above examples, can bring a synergistic effect enabling a higher glass transition temperature Tg, higher stability, and more stable film quality in a thin film state, for example.

In the above General Formulae (G1) to (G4), specific examples of the substituted or unsubstituted alkyl group having 1 to 10 carbon atoms include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, and a 2,3-dimethylbutyl group.

In General Formulae (G1) to (G4), specific examples of a substituted or unsubstituted aryl group having 6 to 60 carbon atoms and a substituted or unsubstituted heteroaryl group having 2 to 50 carbon atoms include a substituted or unsubstituted phenyl group, a substituted or unsubstituted biphenyl group, a substituted or unsubstituted terphenyl group, a substituted or unsubstituted fluorenyl group, a substituted or unsubstituted spirobifluorenyl group, a substituted or unsubstituted naphthyl group, a substituted or unsubstituted anthracenyl group, a substituted or unsubstituted phenanthryl group, a substituted or unsubstituted fluoranthenyl group, a substituted or unsubstituted pyrenyl group, a substituted or unsubstituted chrysenyl group, a substituted or unsubstituted triphenylenyl group, a substituted or unsubstituted perylenyl group, a substituted or unsubstituted indenyl group, a substituted or unsubstituted banzindenyl group, a substituted or unsubstituted pyrrolyl group, a substituted or unsubstituted indolyl group, a substituted or unsubstituted carbazolyl group, a substituted or unsubstituted furanyl group, a substituted or unsubstituted benzofuranyl group, a substituted or unsubstituted dibenzofuranyl group, a substituted or unsubstituted thiophenyl group, a substituted or unsubstituted benzothiophenyl group, a substituted or unsubstituted dibenzothiophenyl group, a substituted or unsubstituted imidazolyl group, a substituted or unsubstituted benzimidazolyl group, a substituted or unsubstituted triazolyl group, a substituted or unsubstituted oxazolyl group, a substituted or unsubstituted oxadiazolyl group, a substituted or unsubstituted thiazolyl group, a substituted or unsubstituted thiaziazolyl group, a substituted or unsubstituted pyrazolyl group, a substituted or unsubstituted pyridyl group, a substituted or unsubstituted pyrimidyl group, a substituted or unsubstituted pyridazyl group, a substituted or unsubstituted triazinyl group, a substituted or unsubstituted quinolinyl group, a substituted or unsubstituted indolocarbazolyl group, a substituted or unsubstituted benzocarbazolyl group, a substituted or unsubstituted quinoxalinyl group, and a substituted or unsubstituted dibenzoquinoxalinyl group. Note that the bonding position can be any possible one. More Specifically, a substituent represented by any of (R-1) to (R-112) is preferable. The substituent represented by any of (R-1) to (R-112) may further include a substituent.

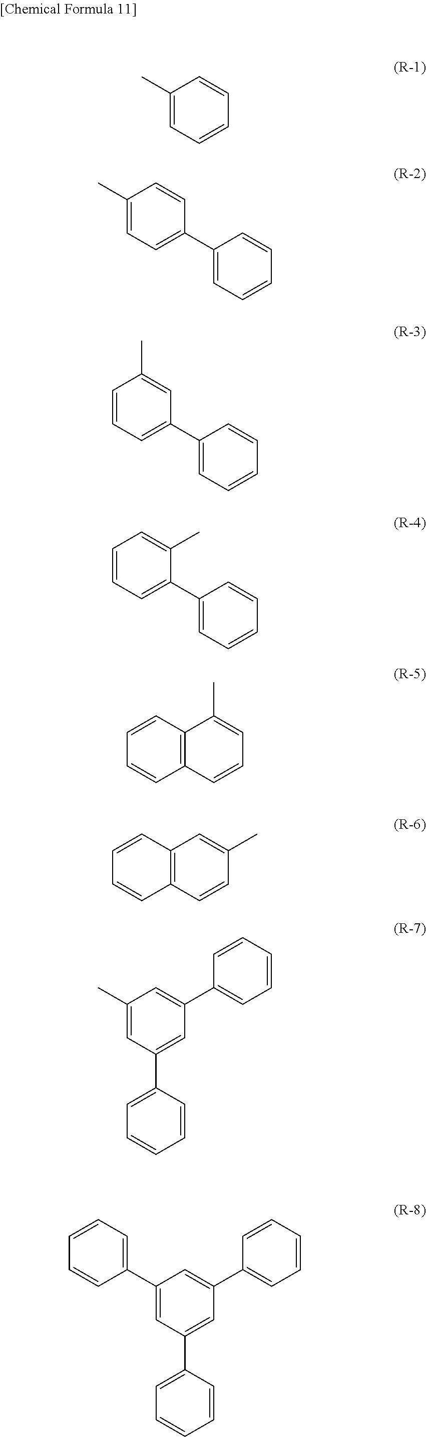

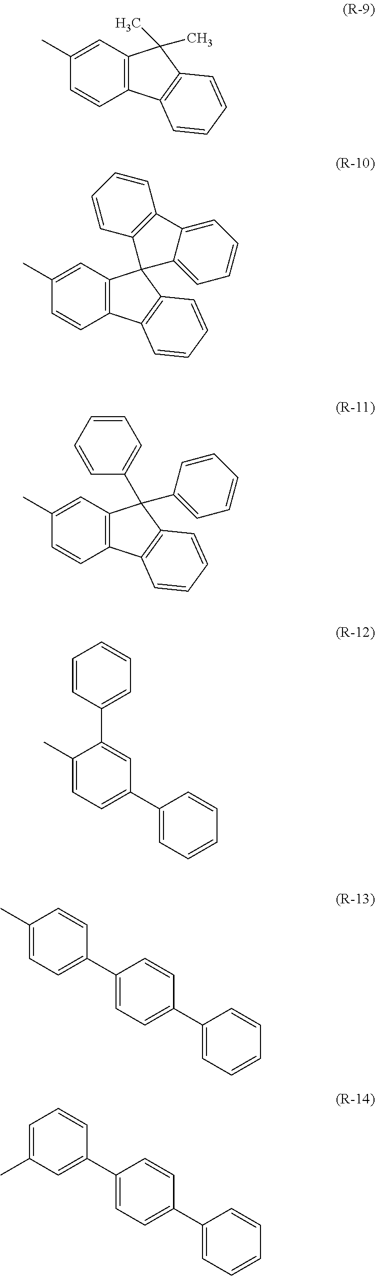

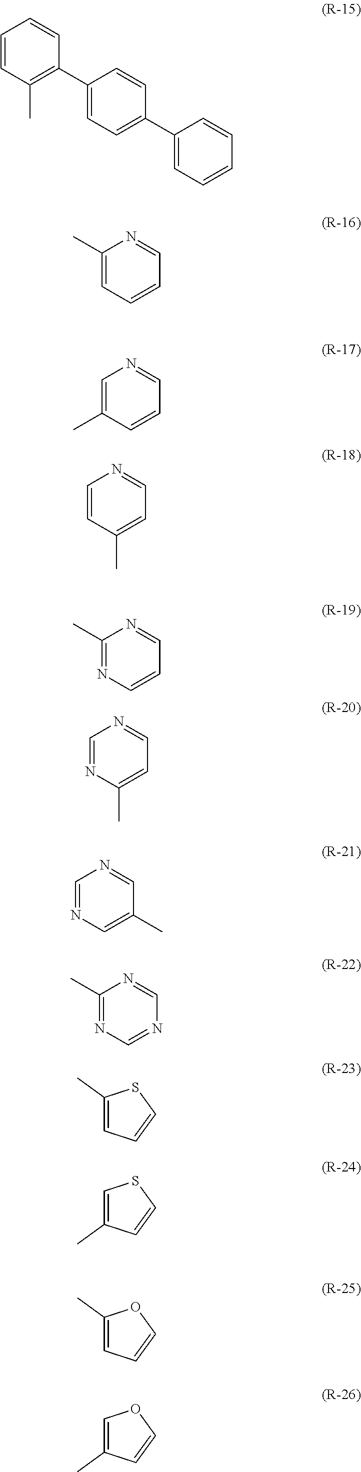

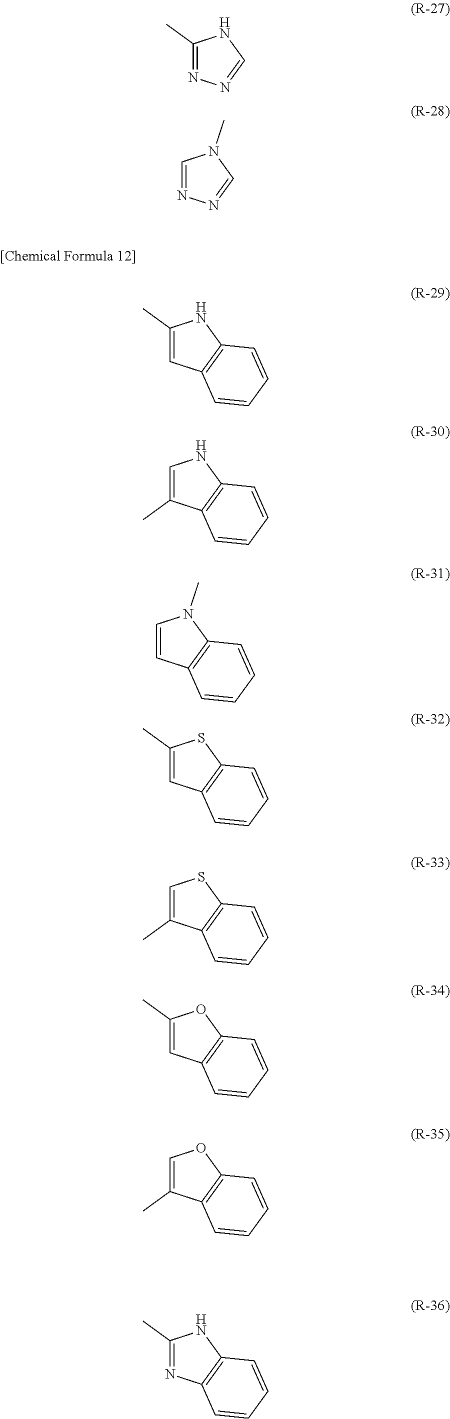

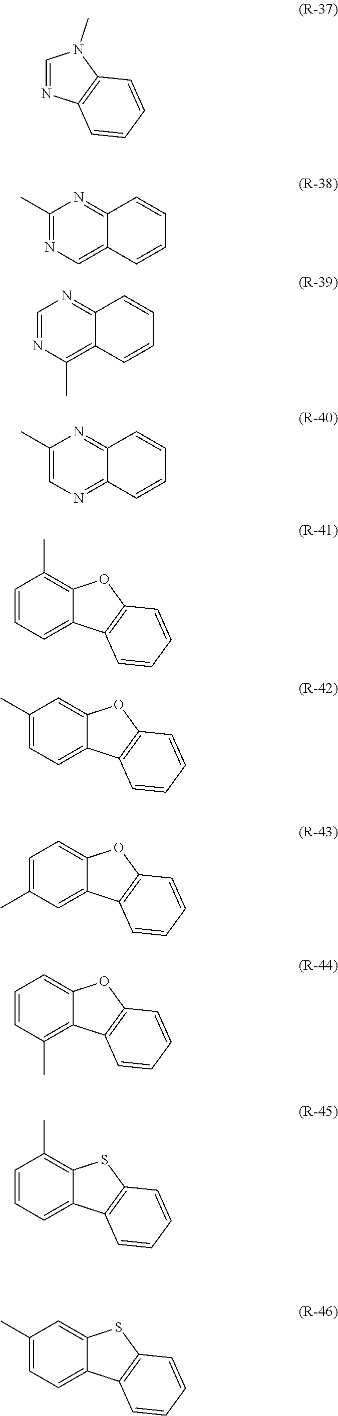

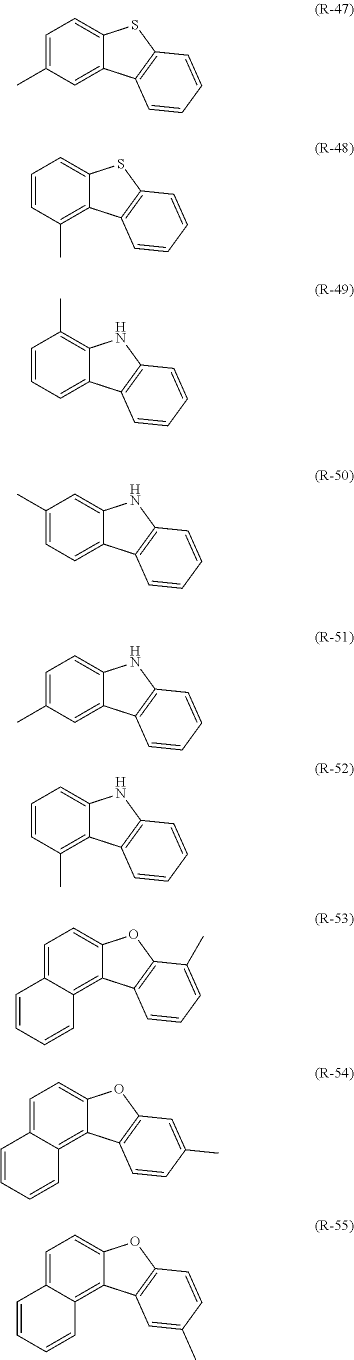

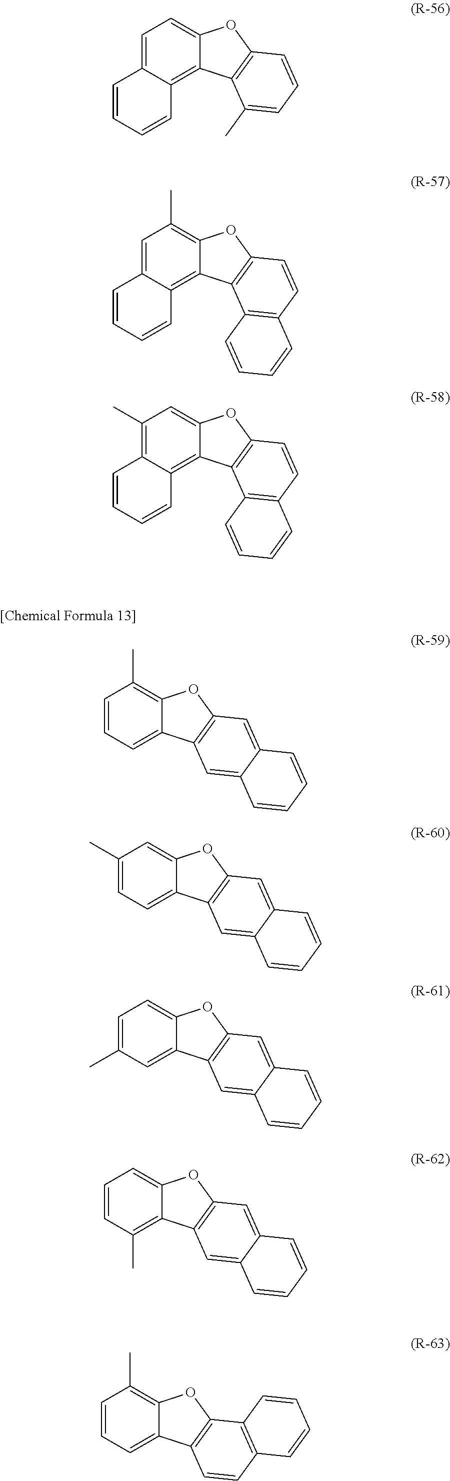

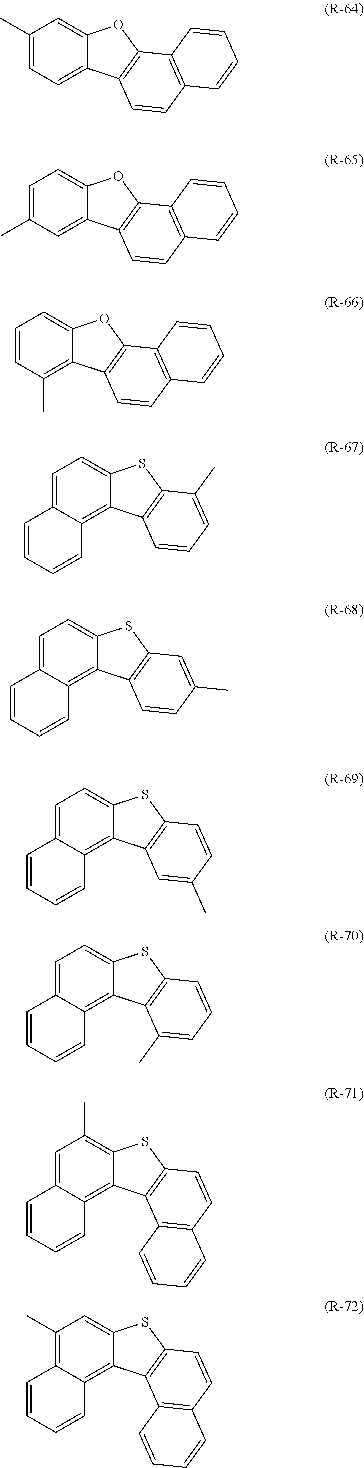

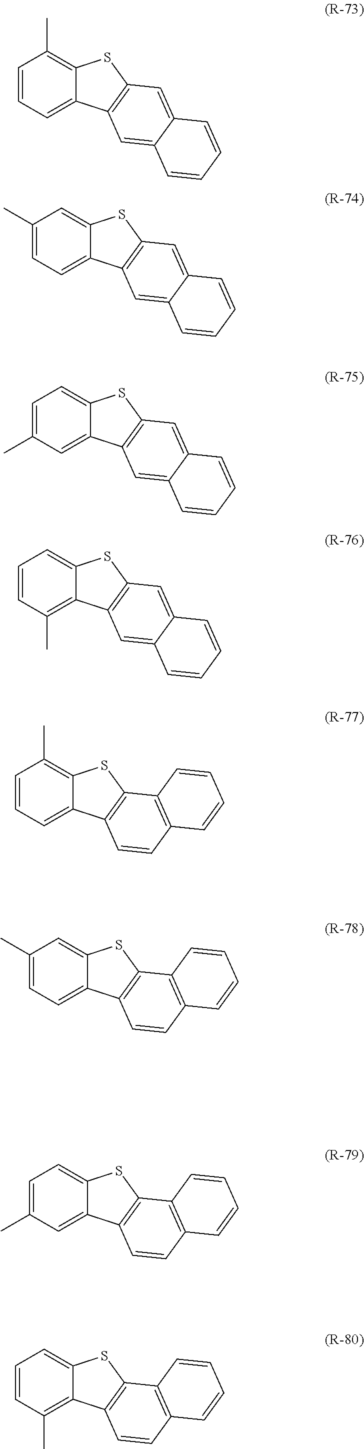

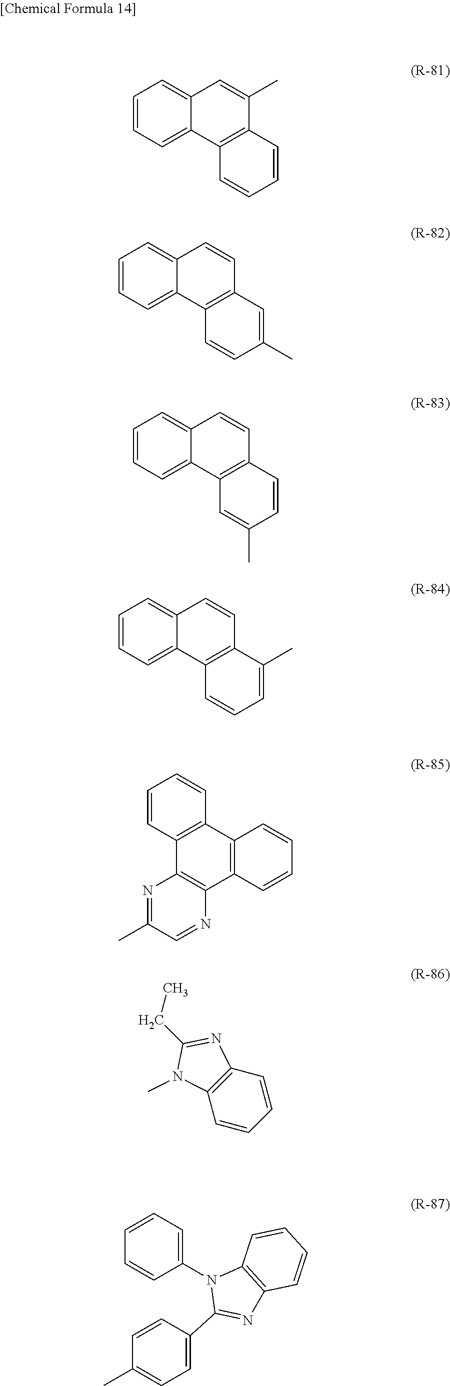

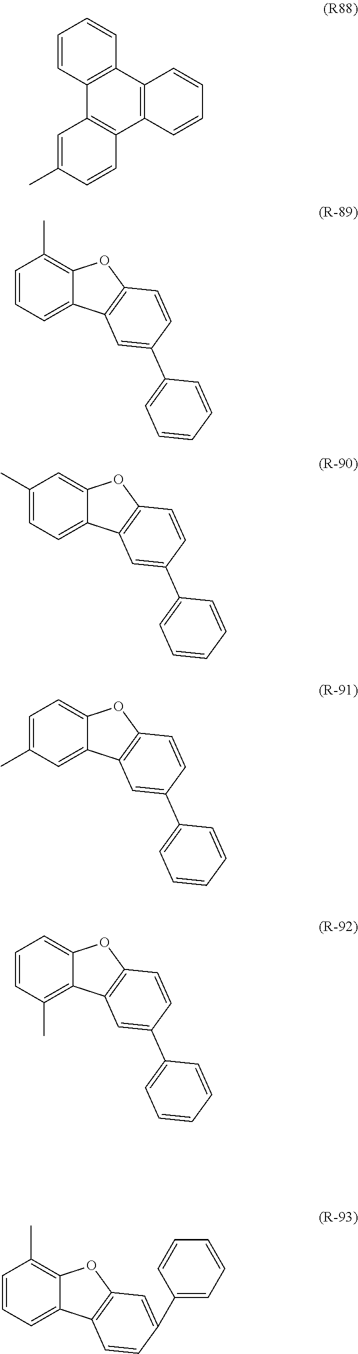

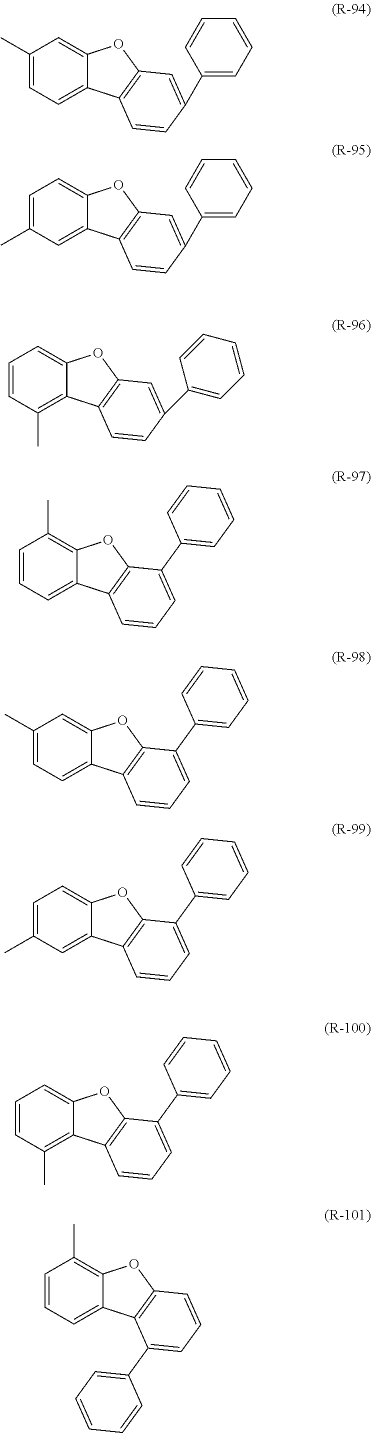

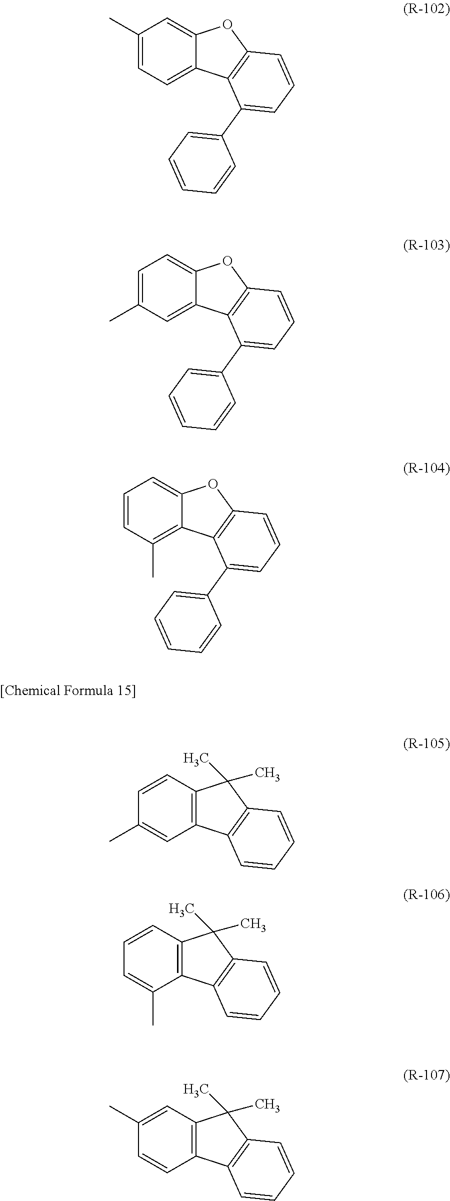

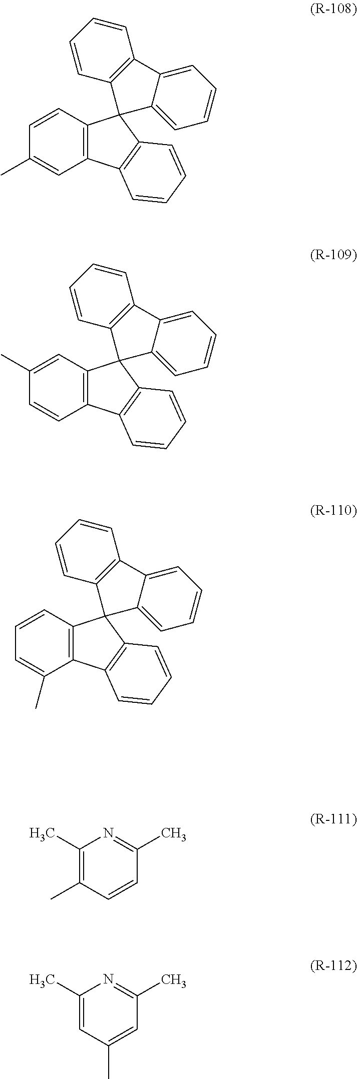

When used for a light-emitting device, the compound of one embodiment of the present invention is preferably used in a thin film (also referred to as an organic compound layer). A thin film including the organic compound of one embodiment of the present invention can be suitably used for a charge-adjustment layer, a charge-generation layer, a light-emitting layer, a hole-injection layer, a hole-transport layer, an electron-transport layer, an electron-injection layer, or a cap layer in the light-emitting device. In addition, the organic compound of one embodiment of the present invention can be used also for a non-light-emitting device. As the non-light-emitting device, a device such as a light-receiving device can be given, for example.

Note that the detailed structure where the organic compound of one embodiment of the present invention is used for a light-emitting device or a light-receiving device will be described in detail in Embodiment 2 or the like to be described later.

Next, specific examples of an organic compound having a structure represented by any of General Formulae (G1) to (G4) and an organic compound that can be used for a light-emitting device are described below.

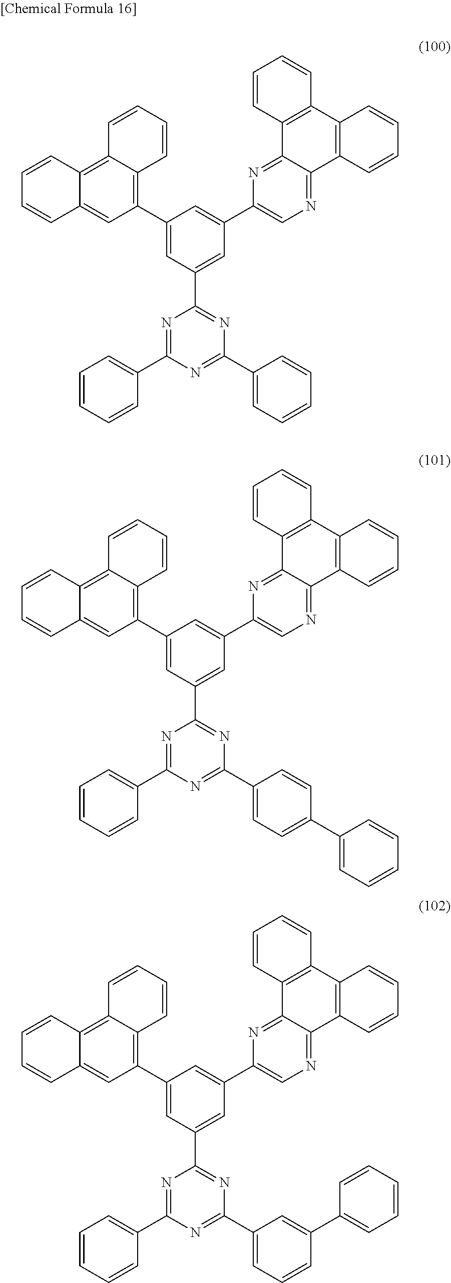

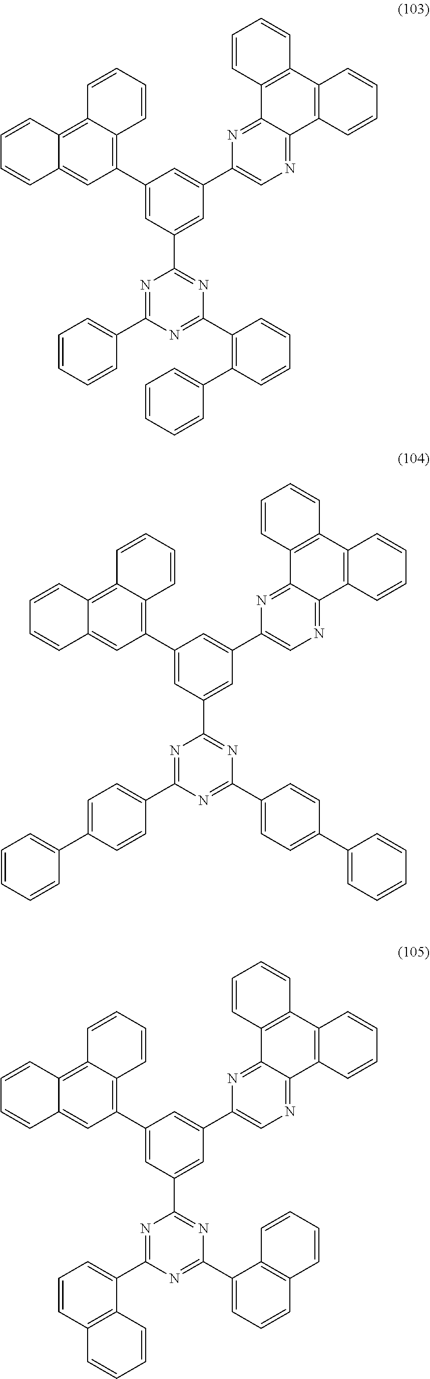

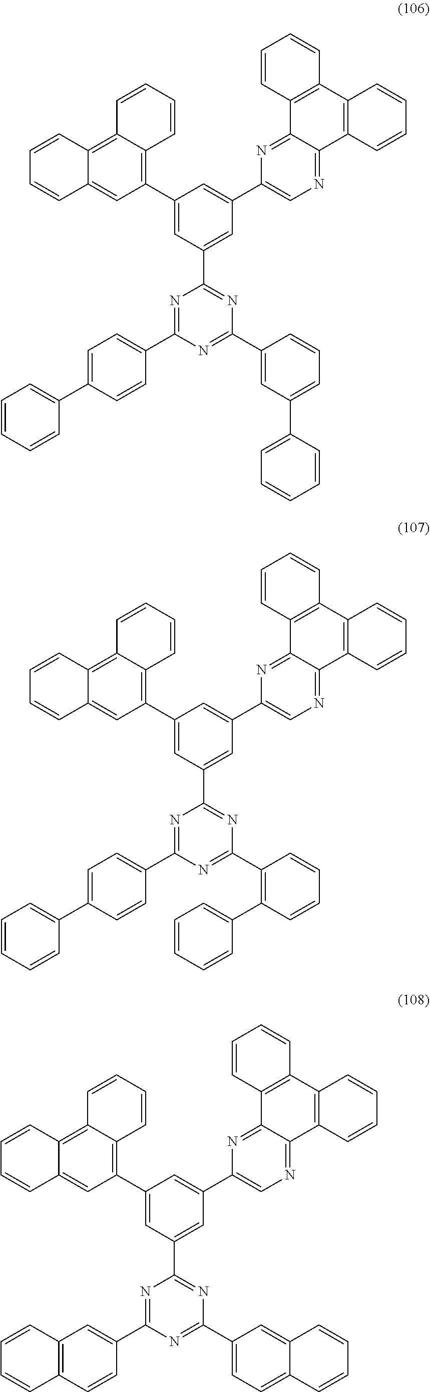

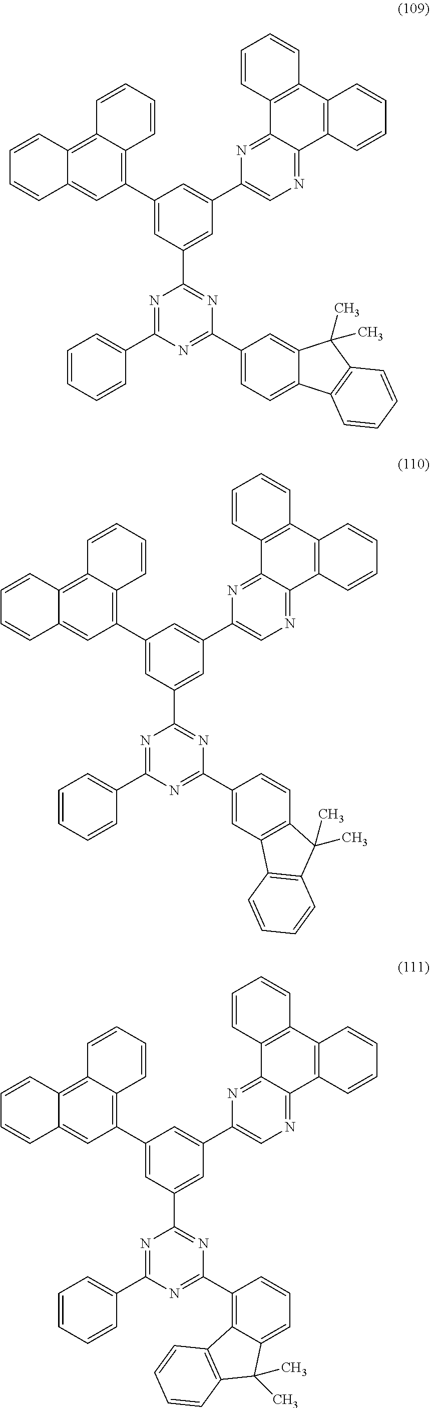

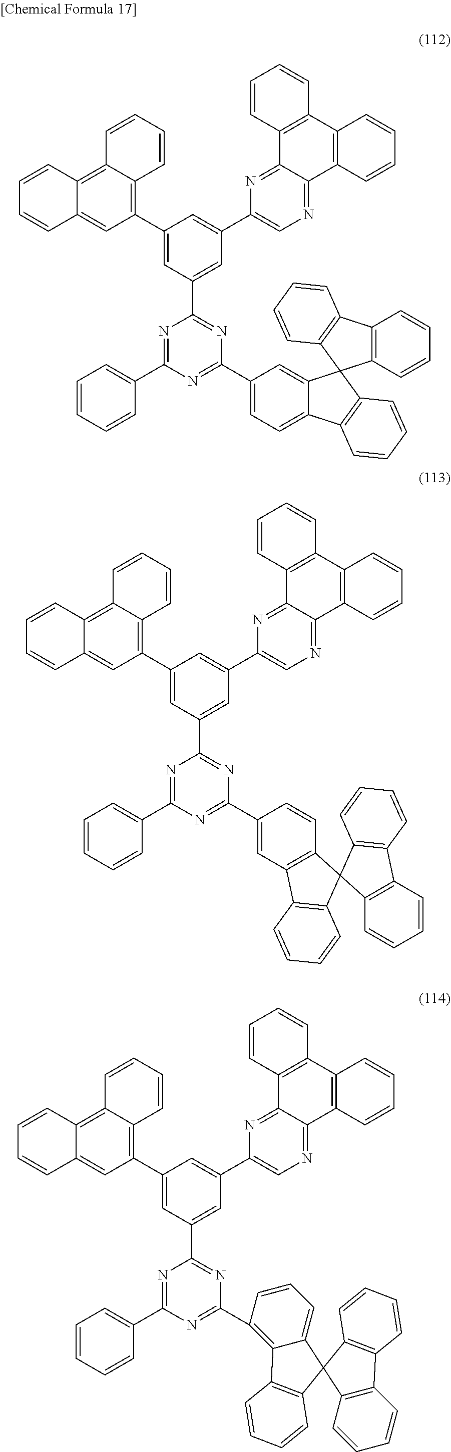

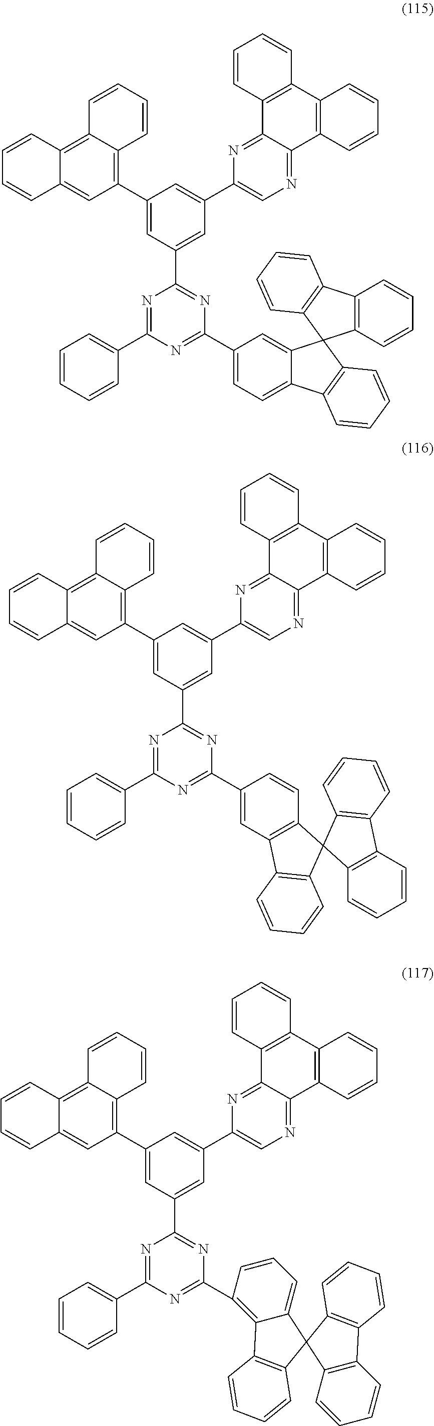

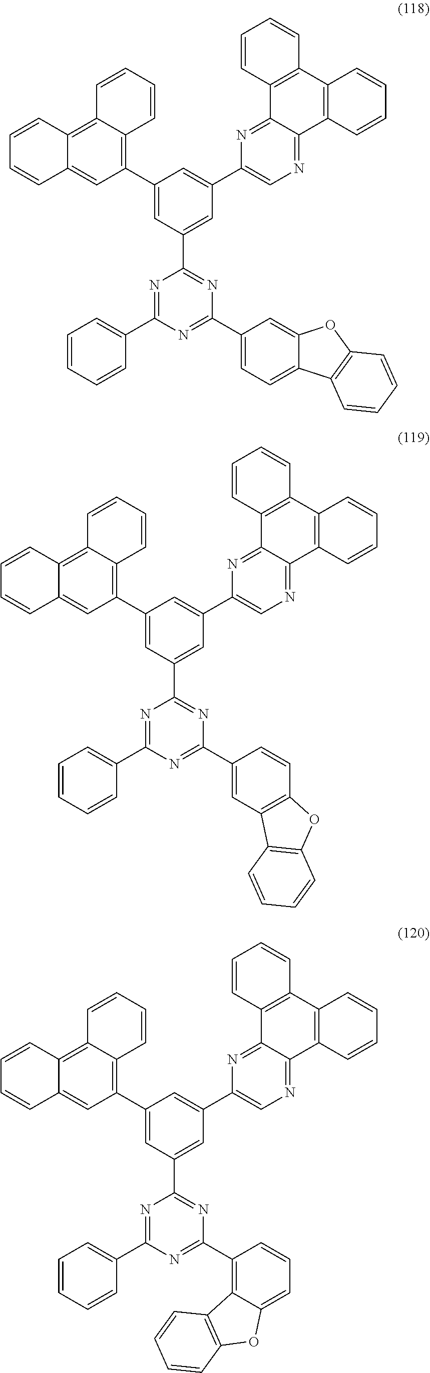

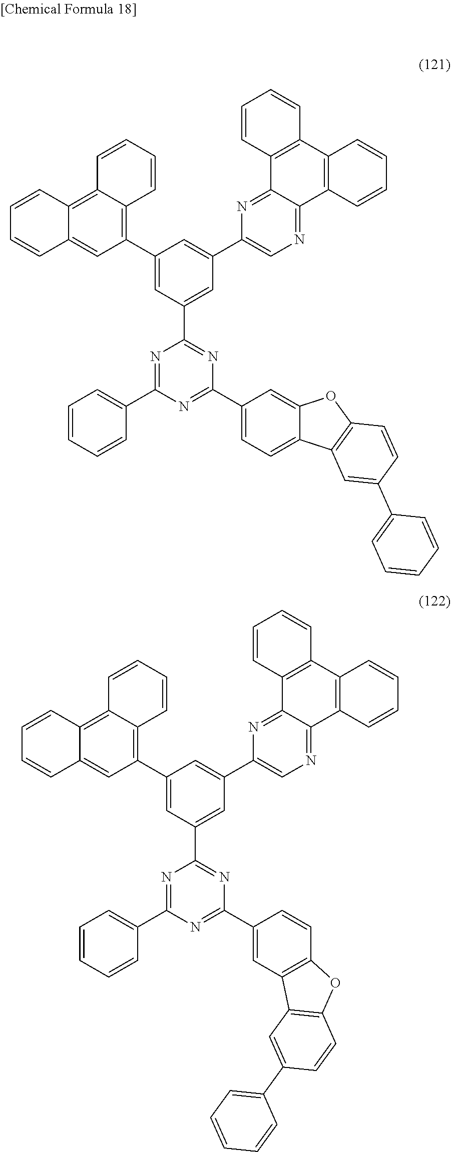

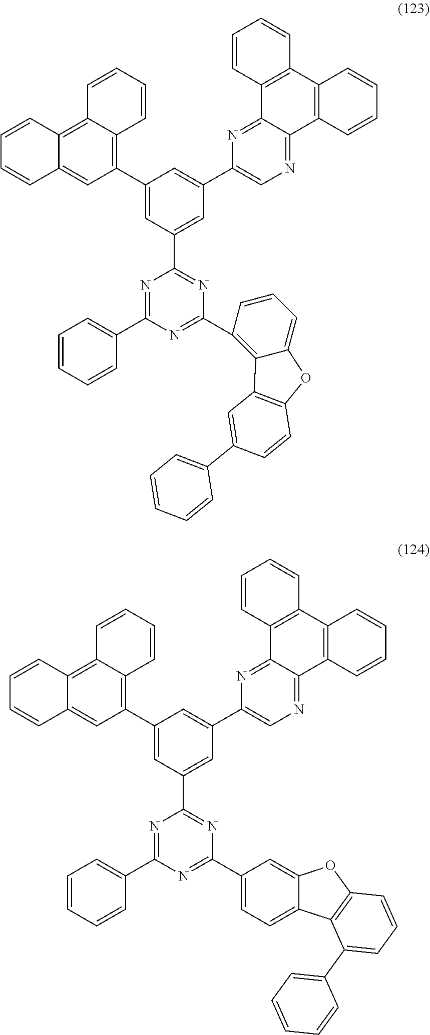

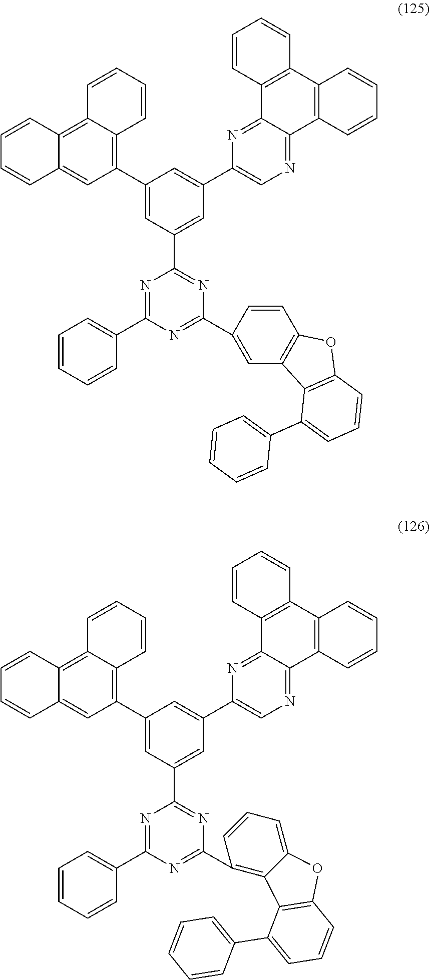

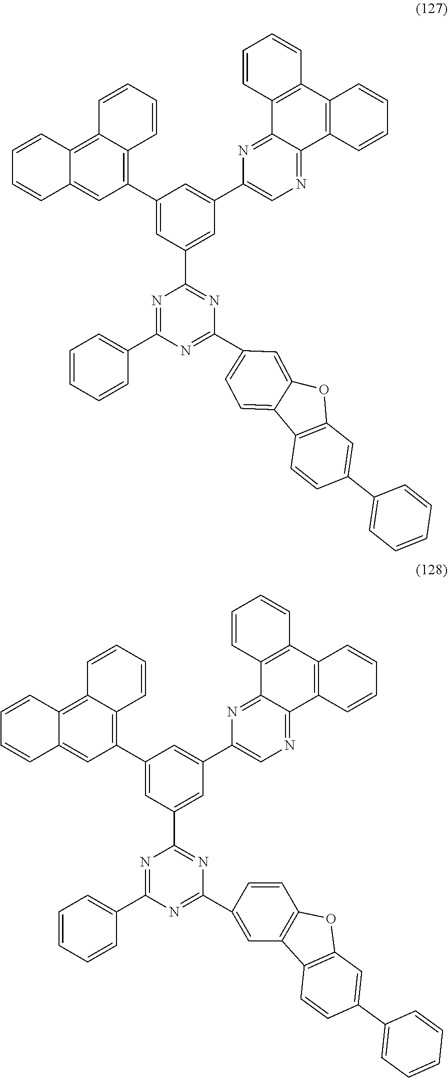

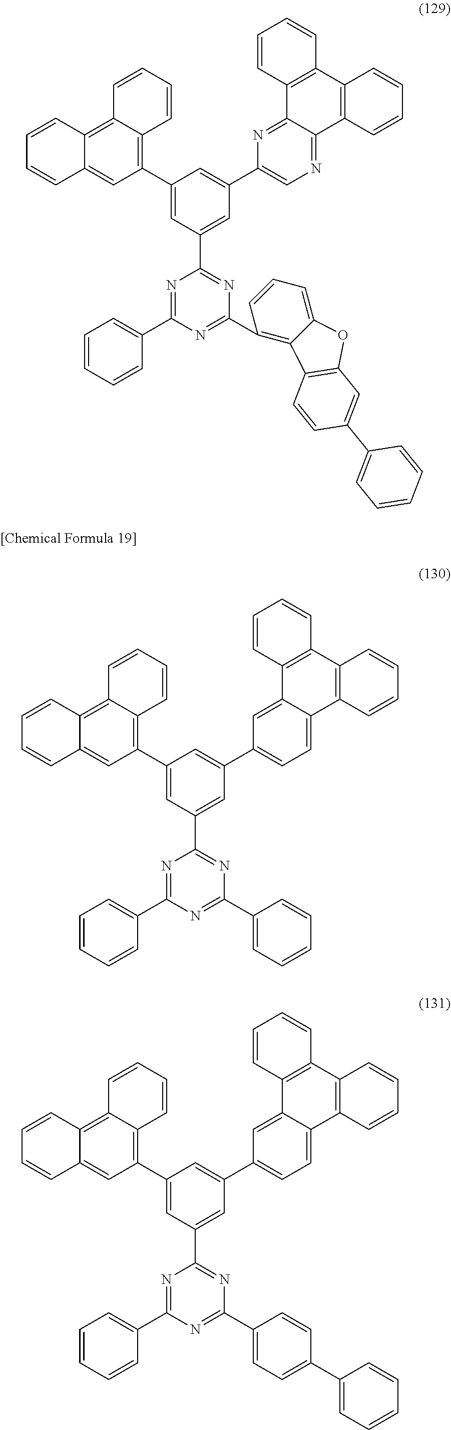

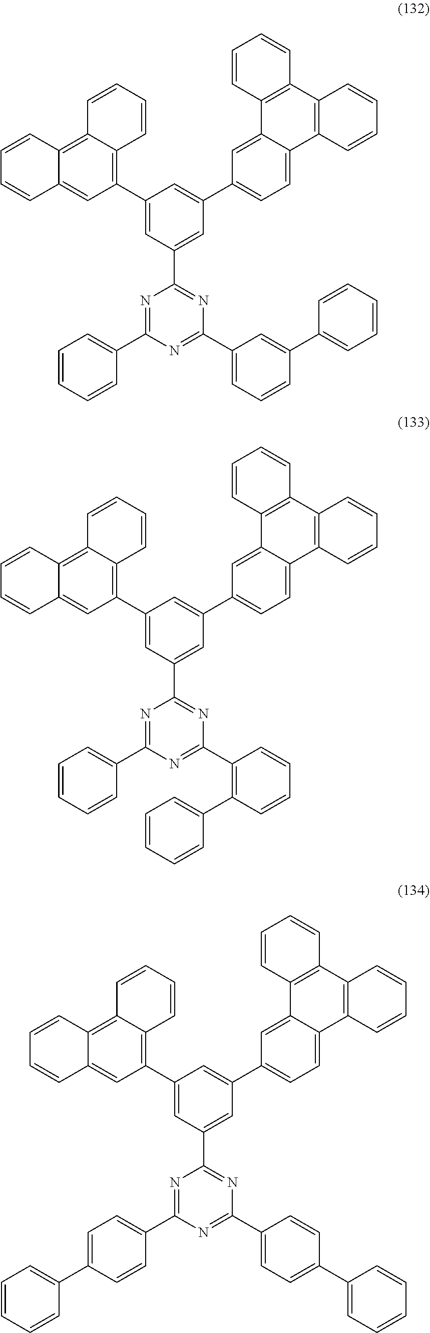

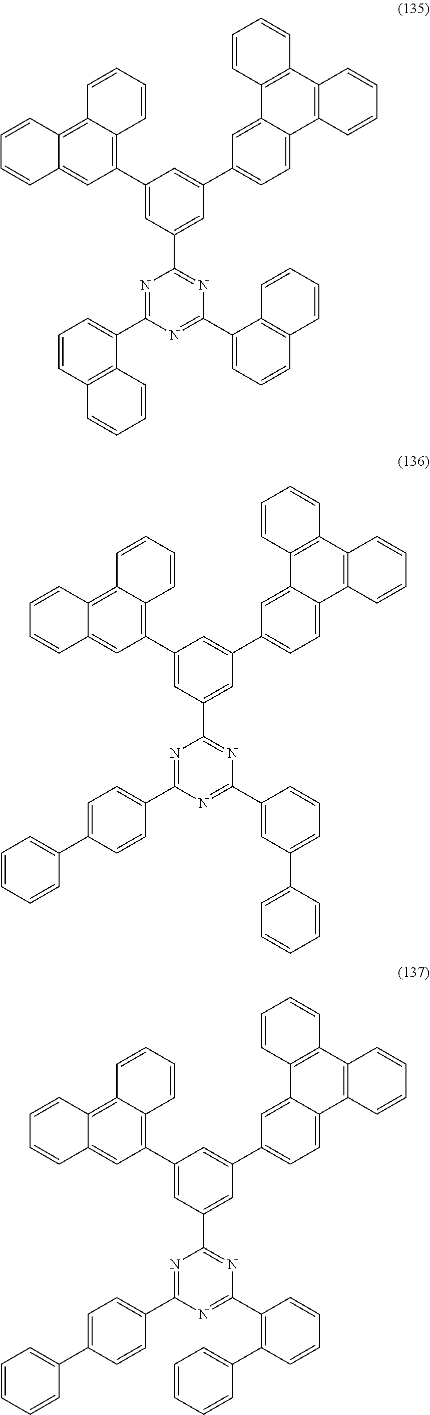

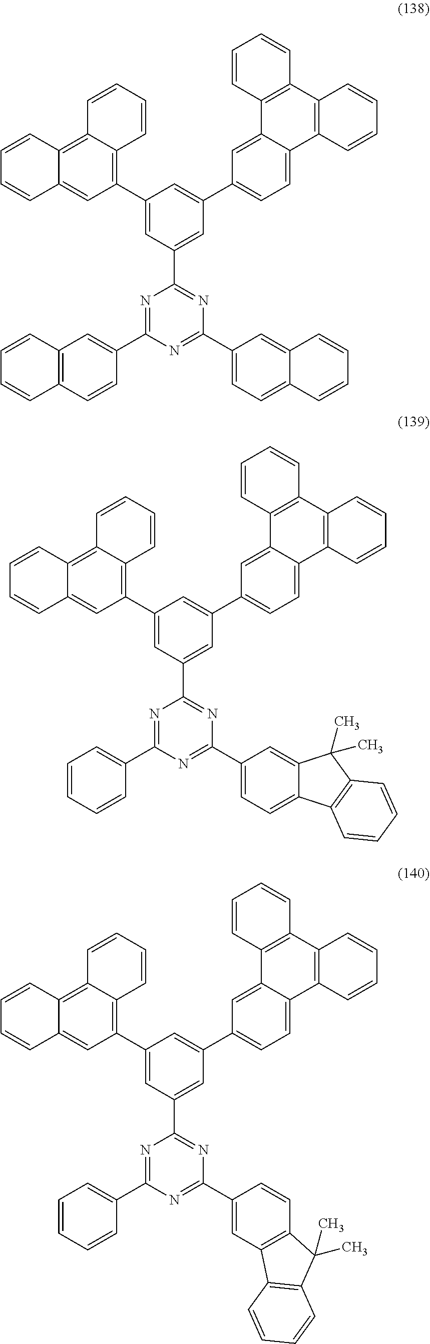

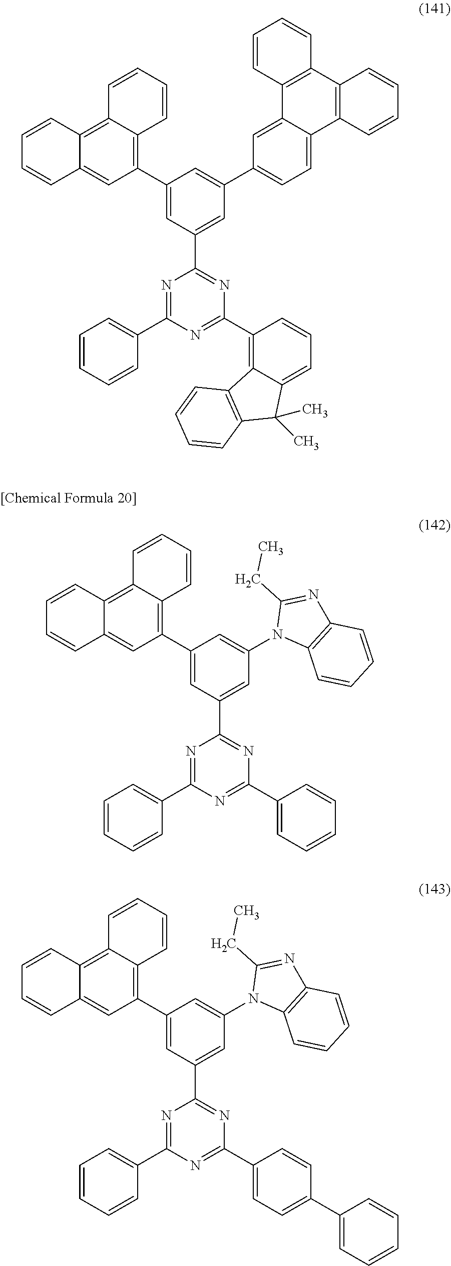

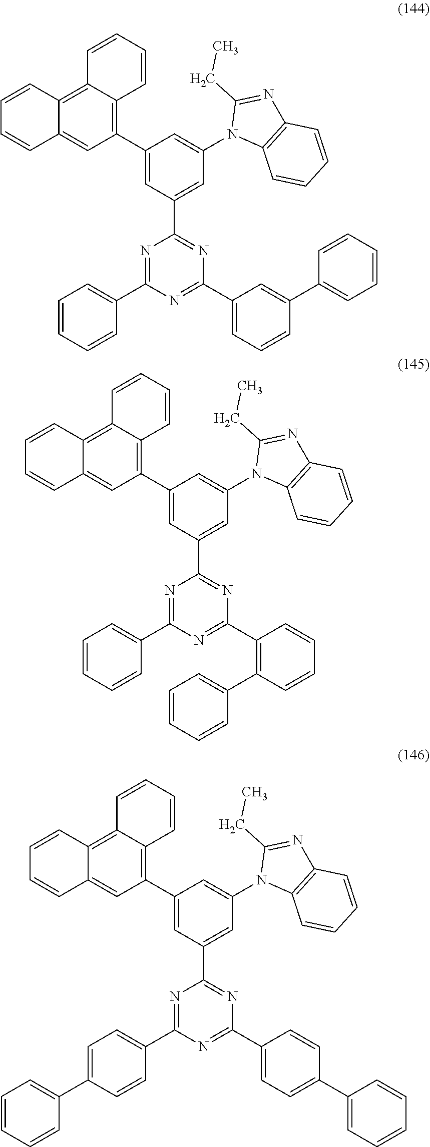

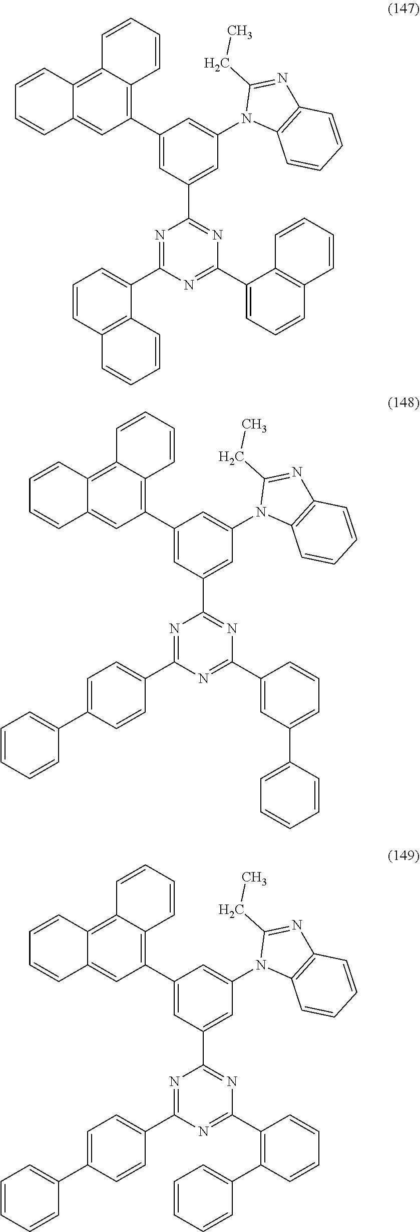

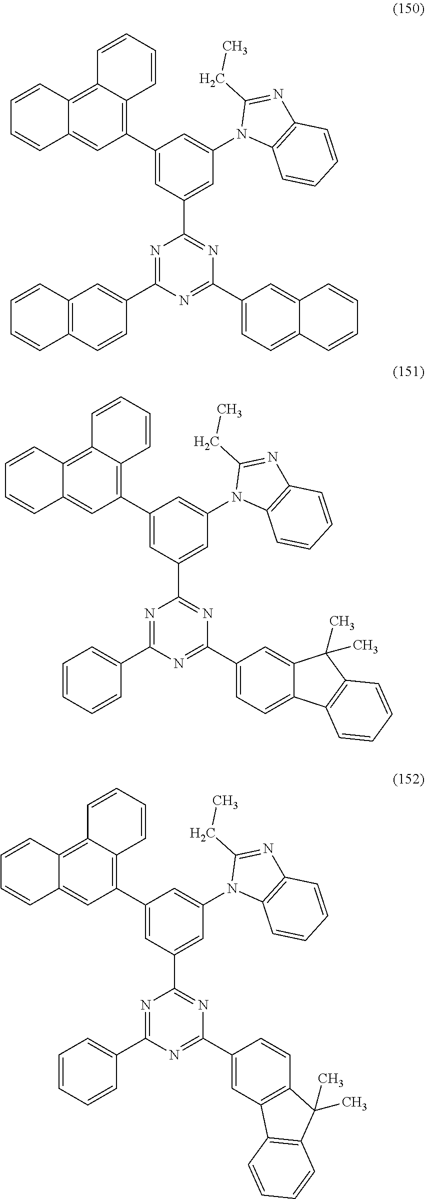

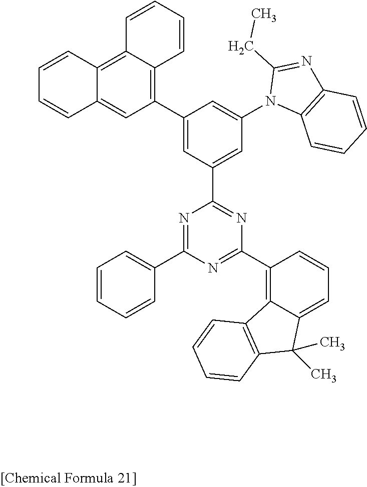

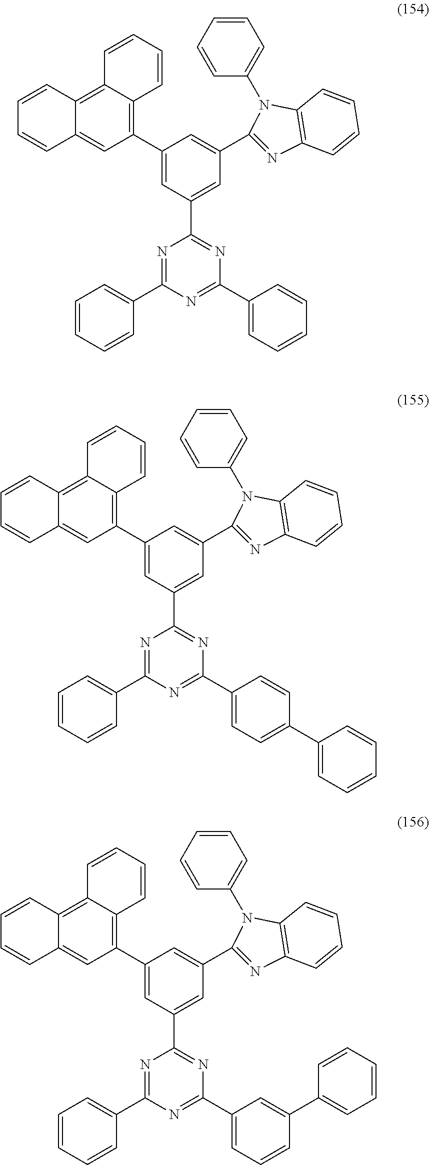

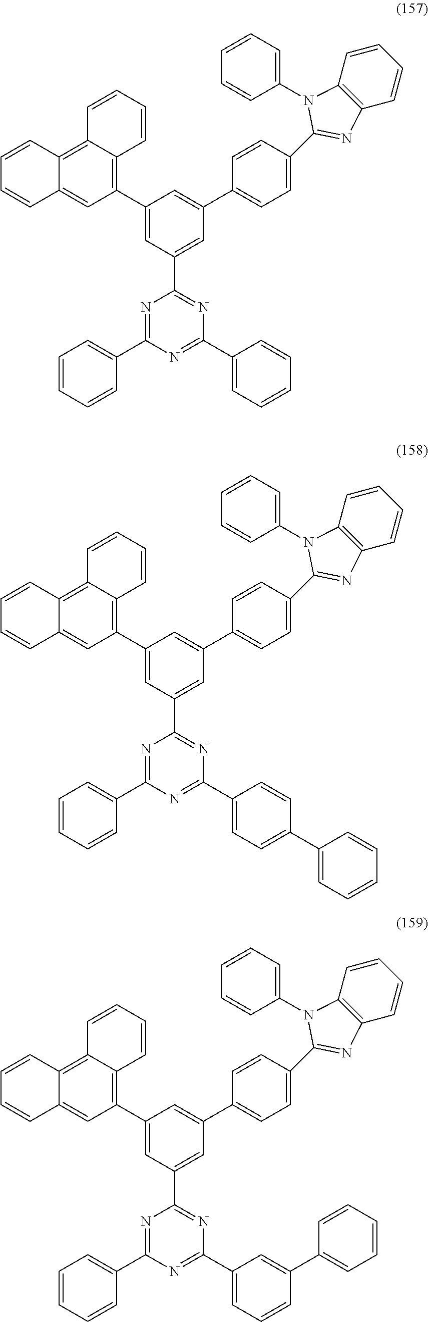

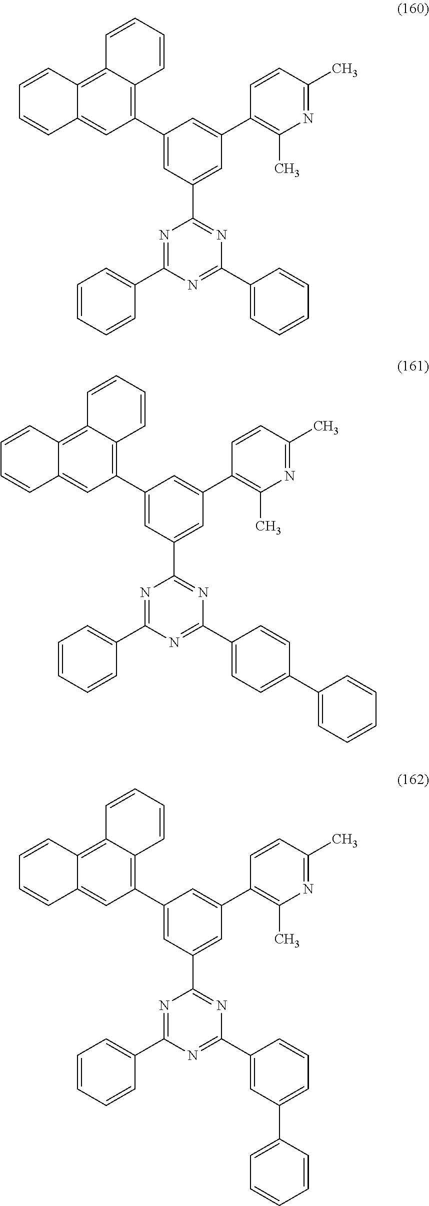

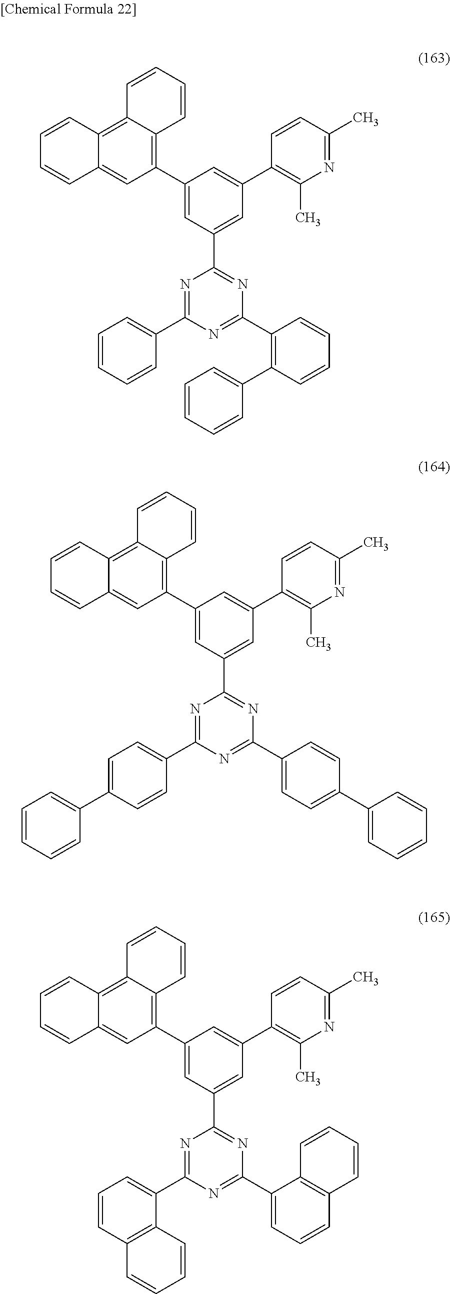

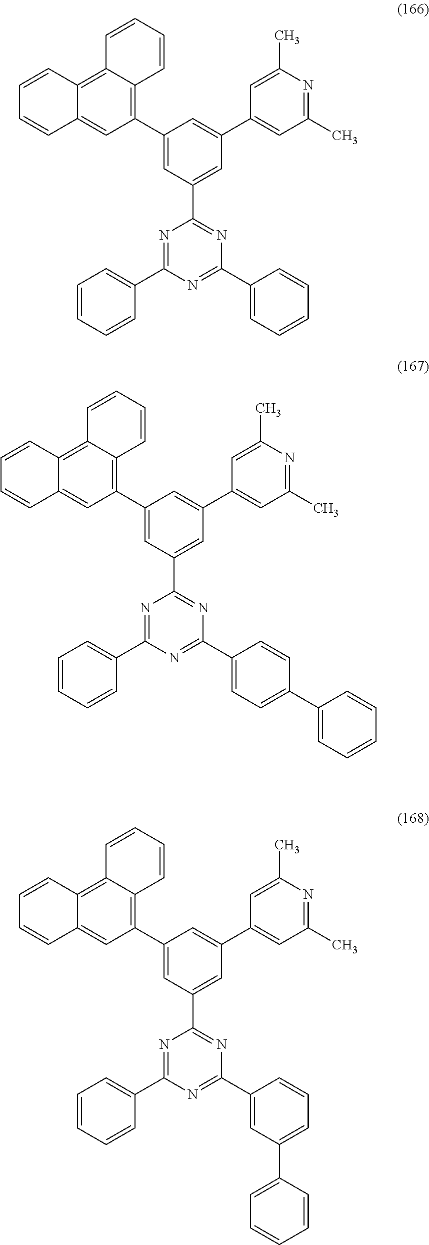

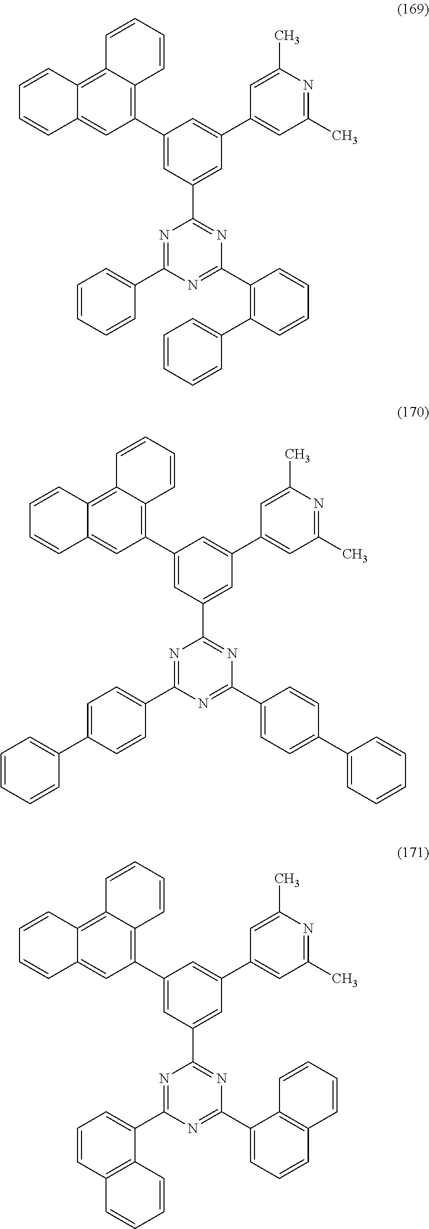

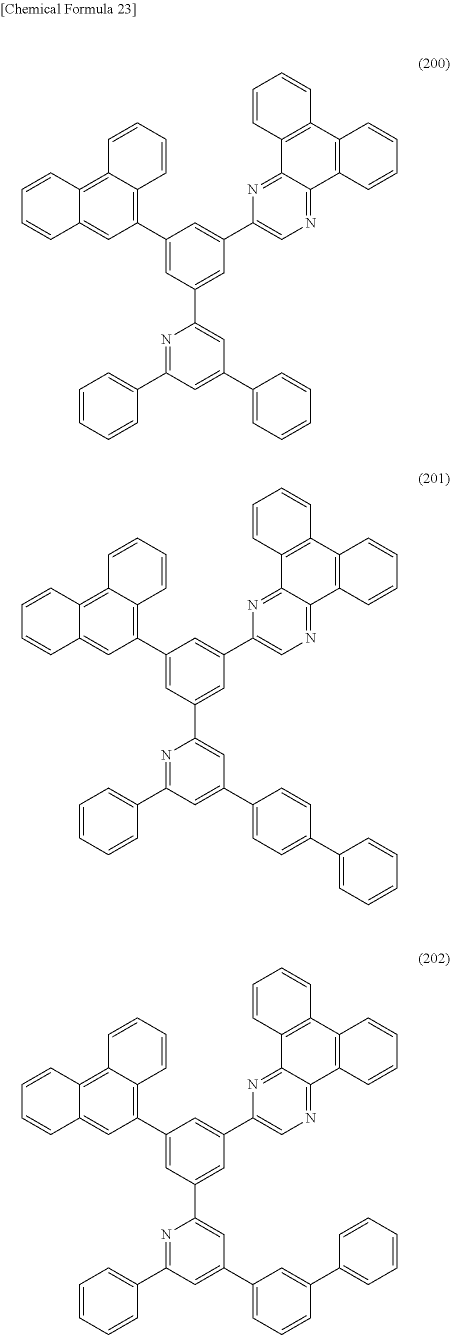

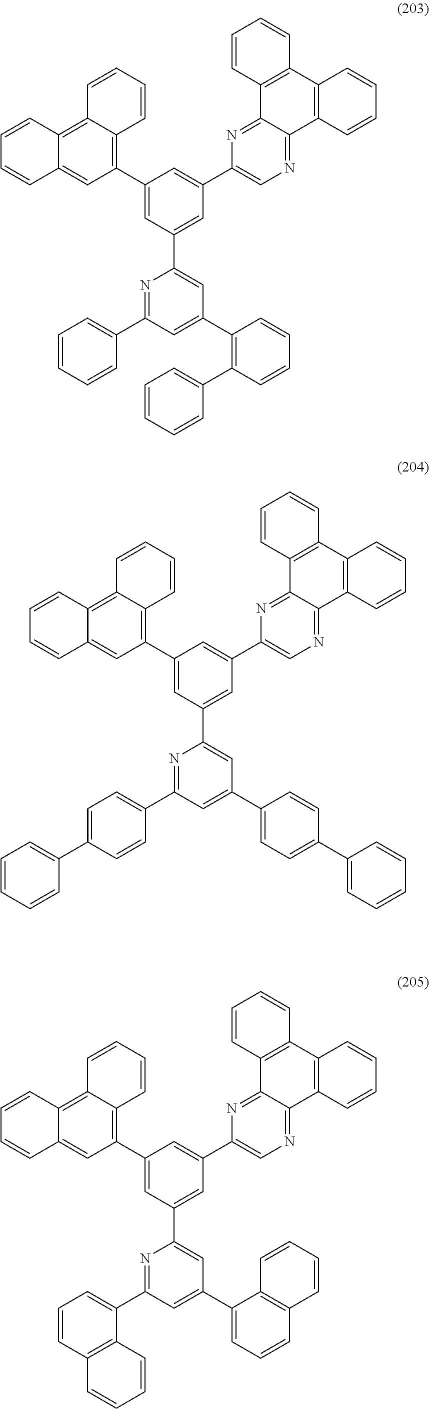

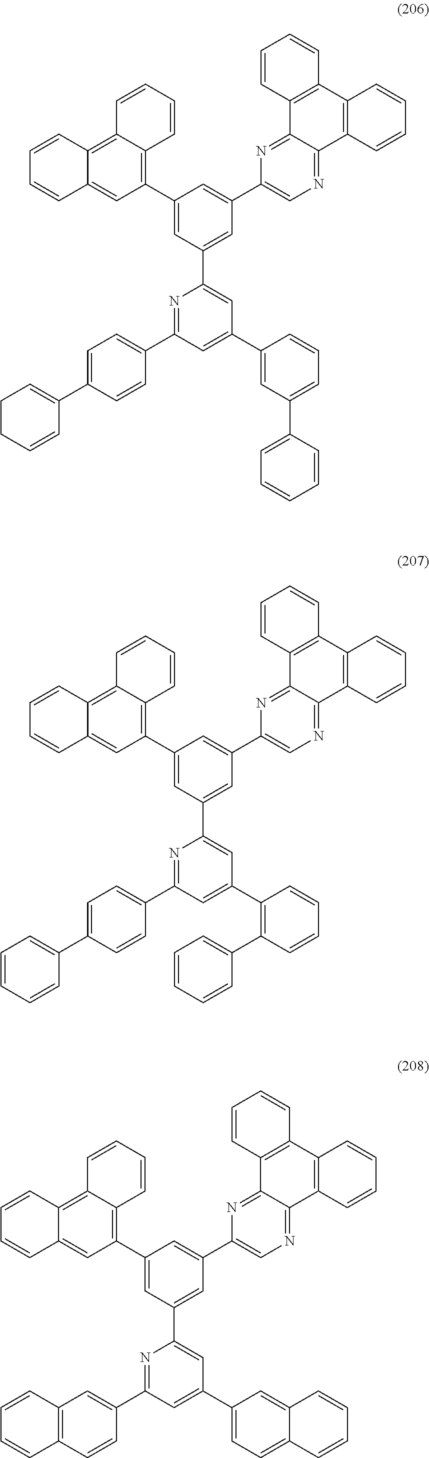

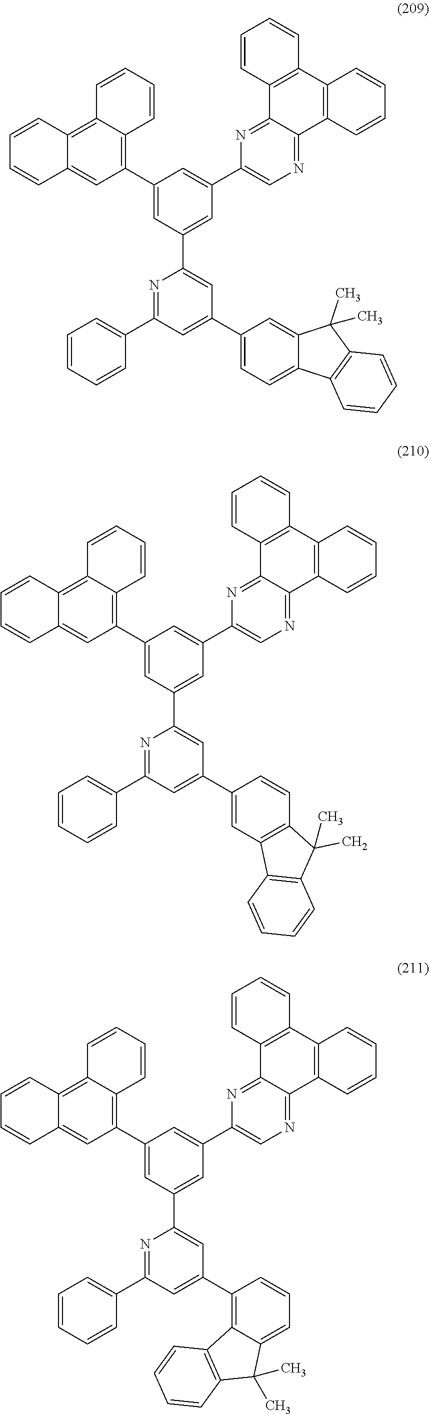

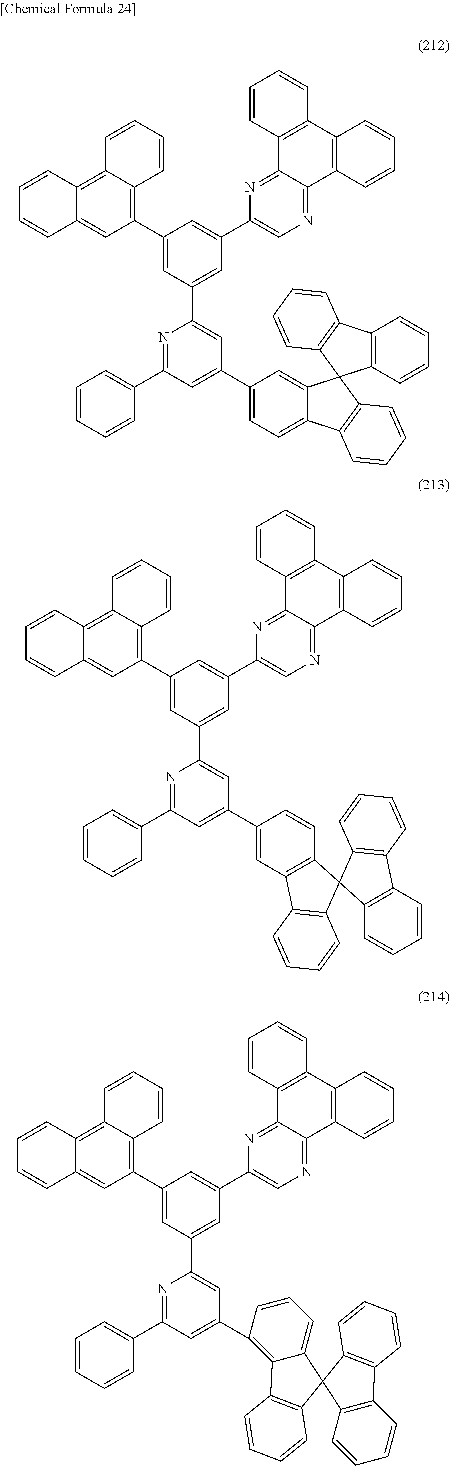

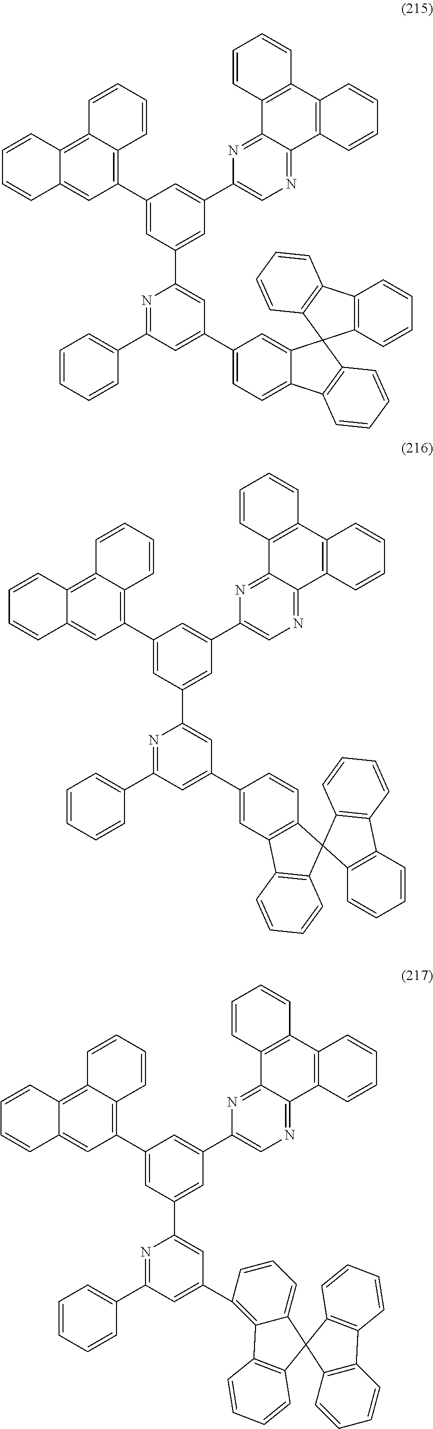

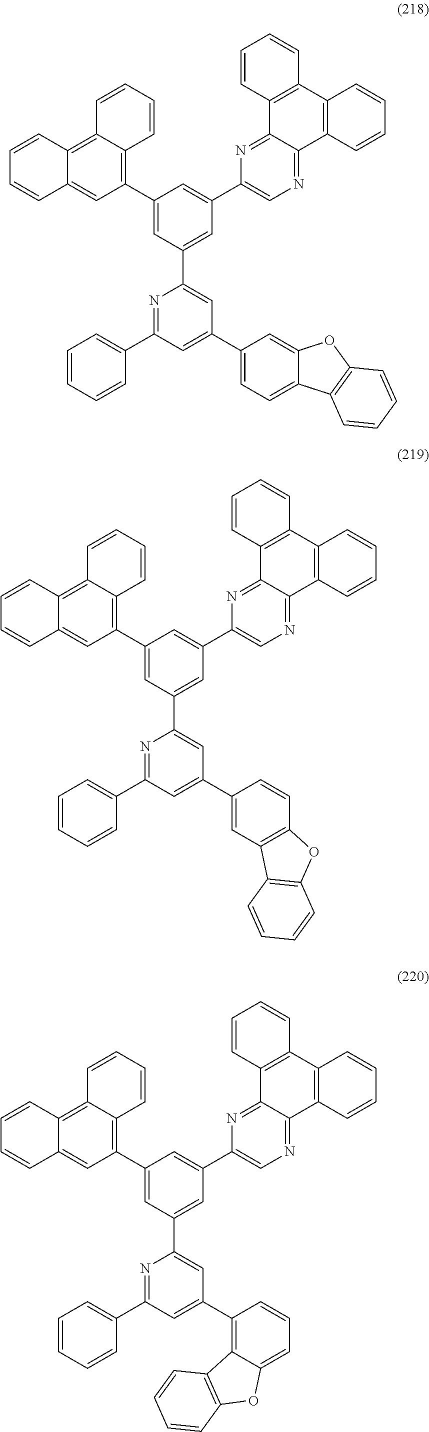

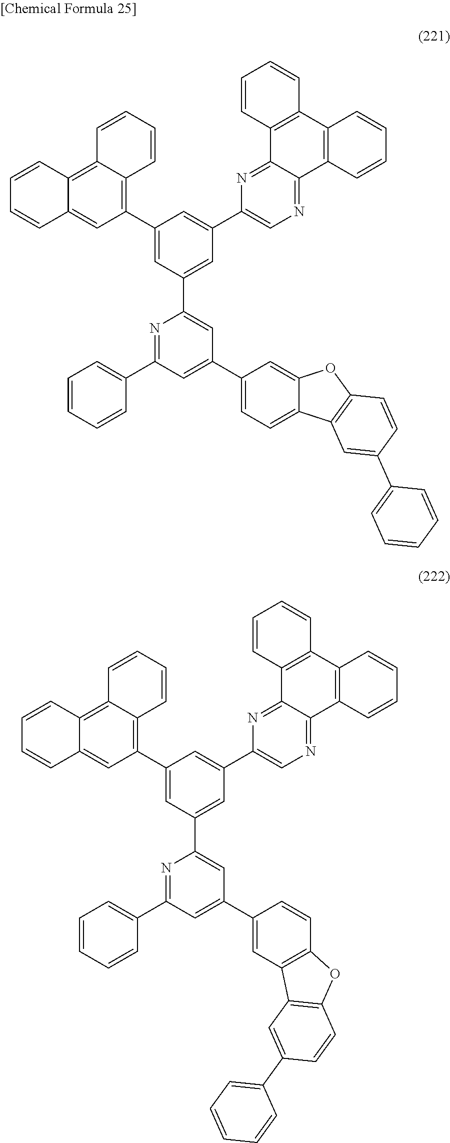

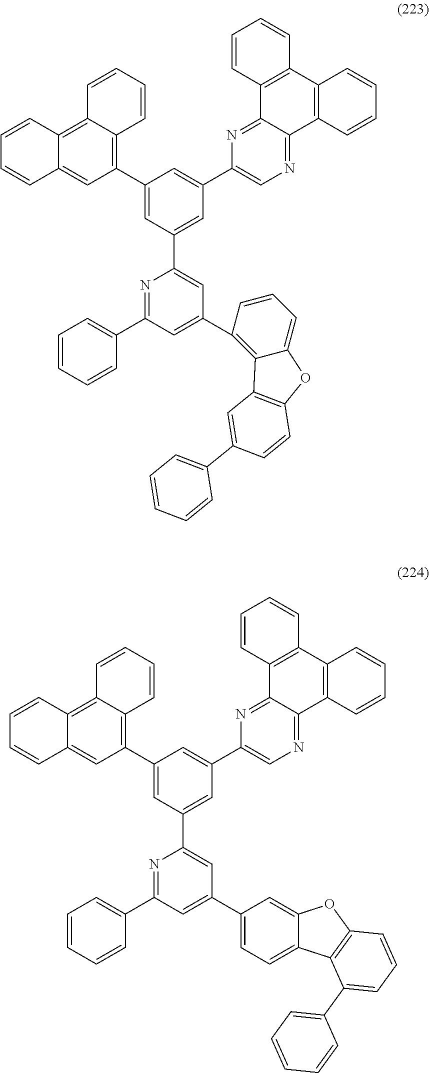

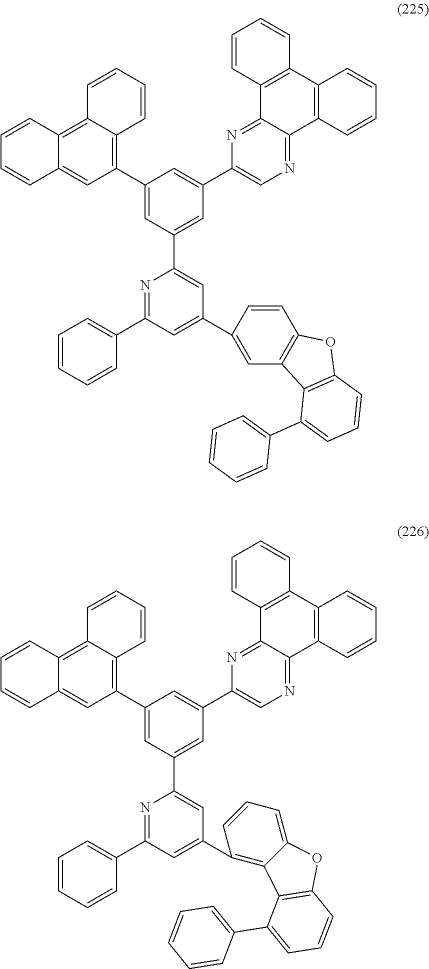

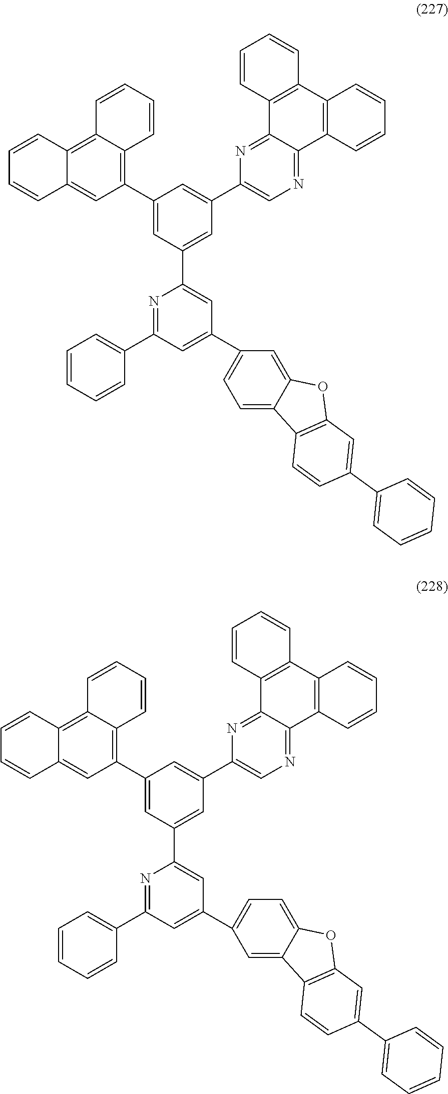

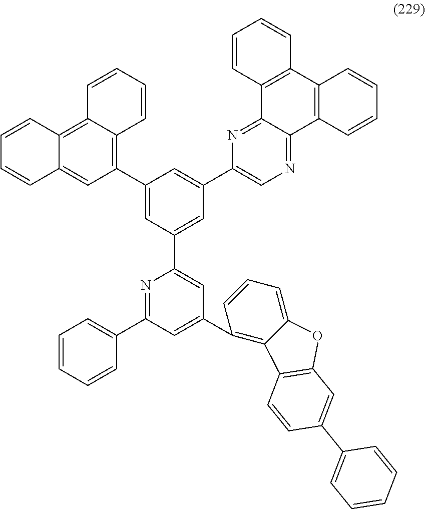

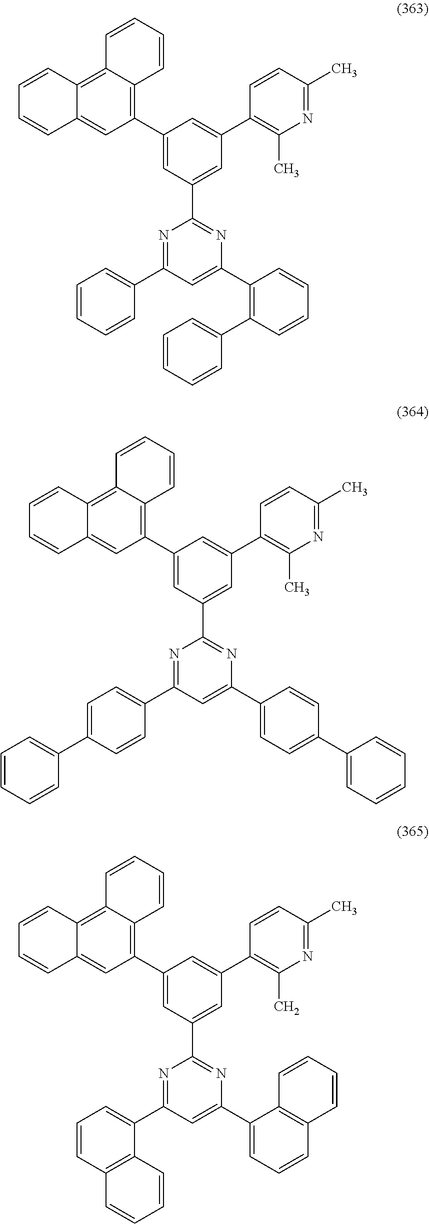

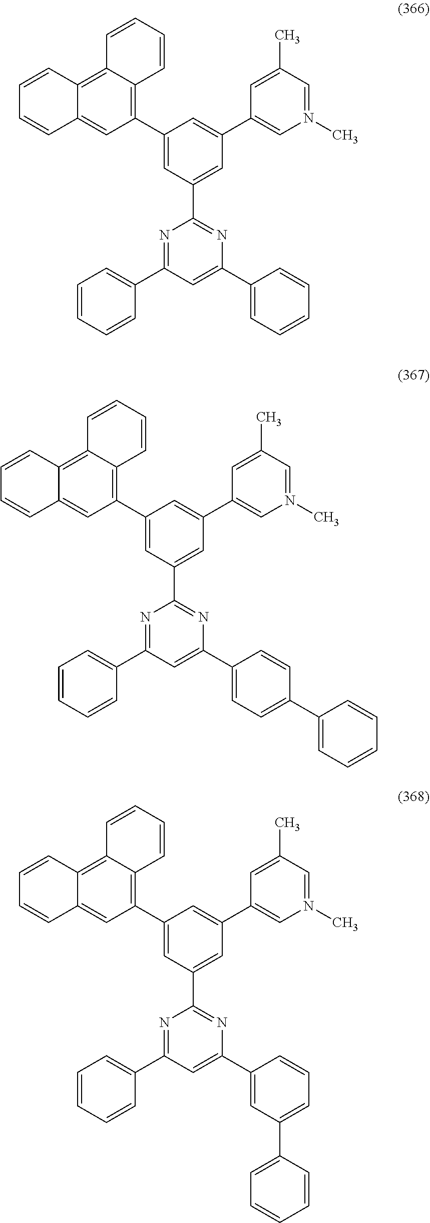

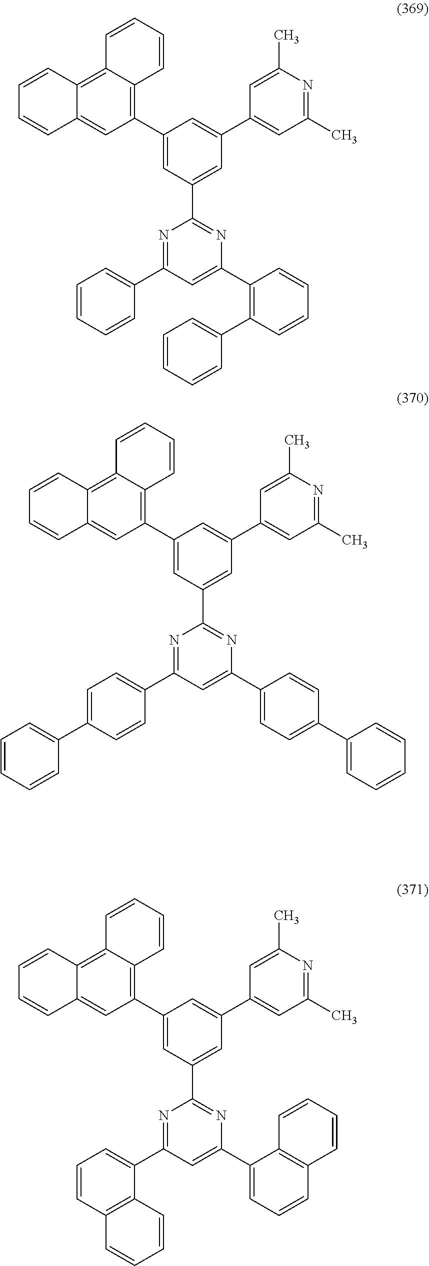

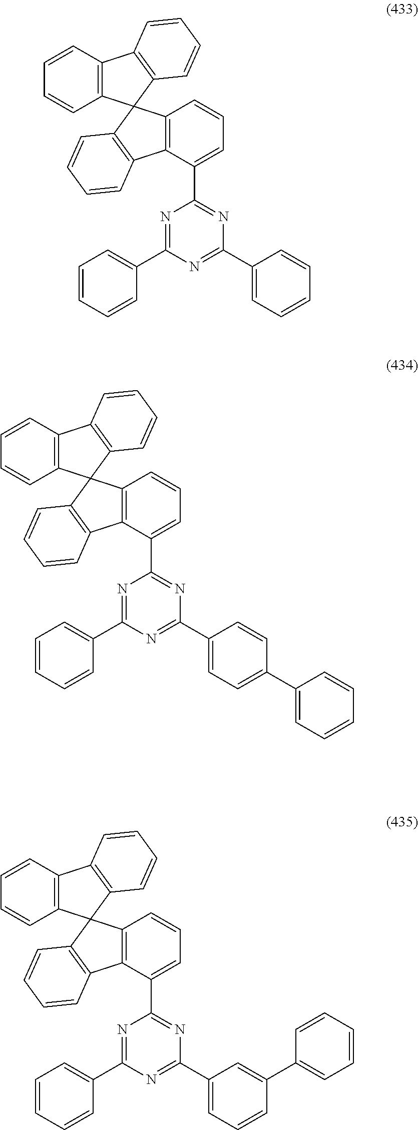

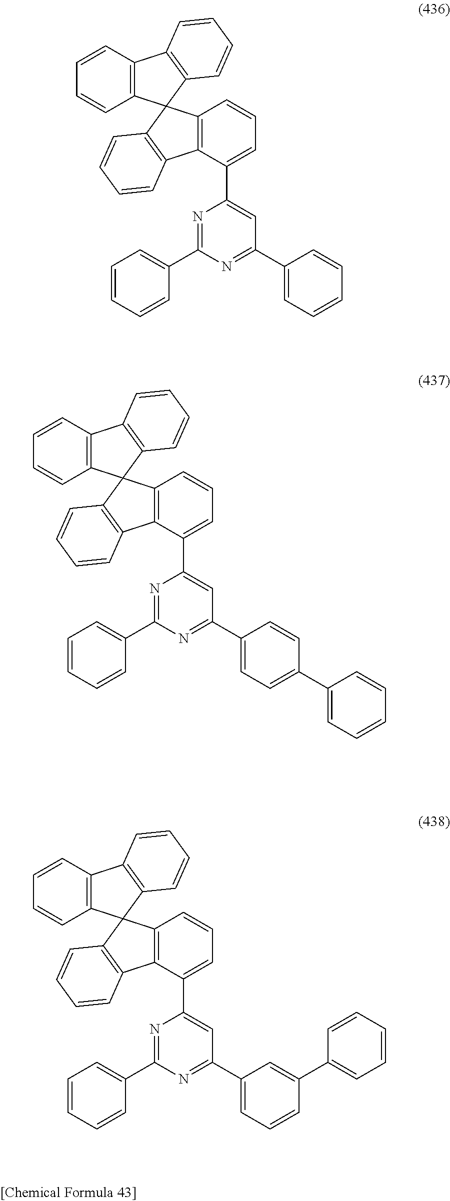

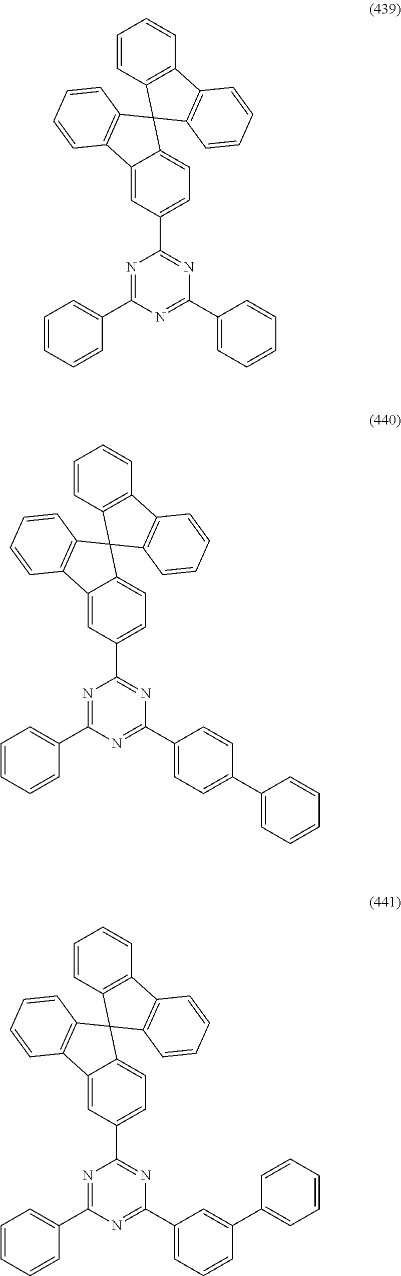

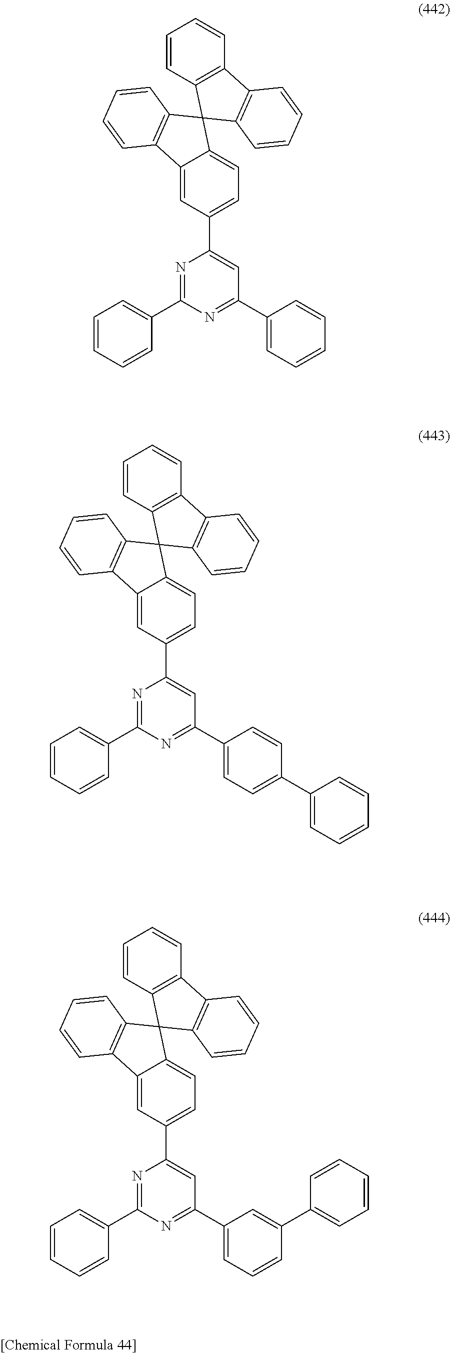

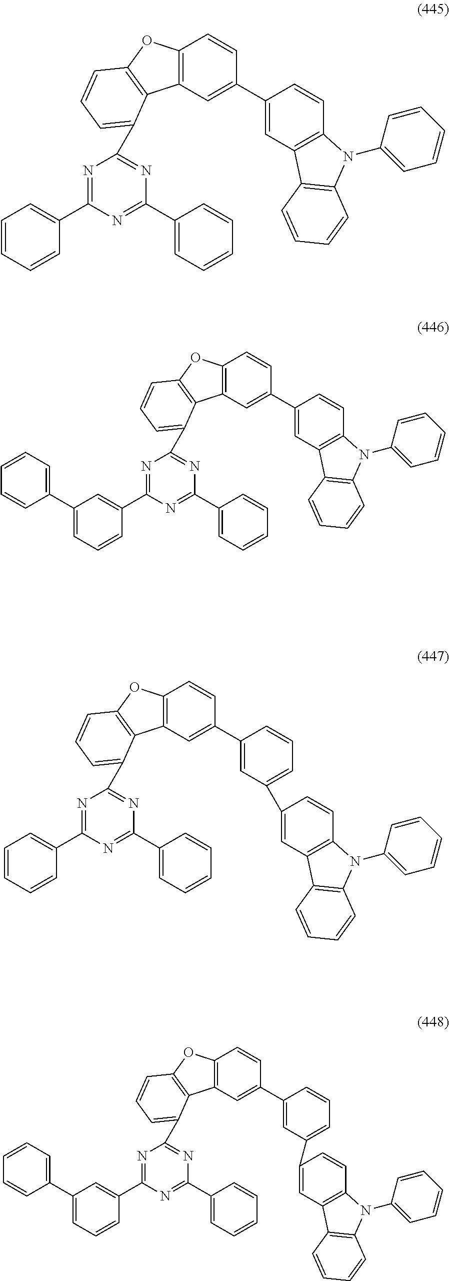

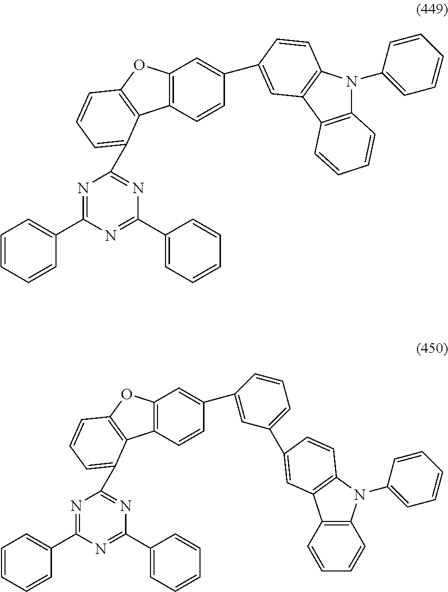

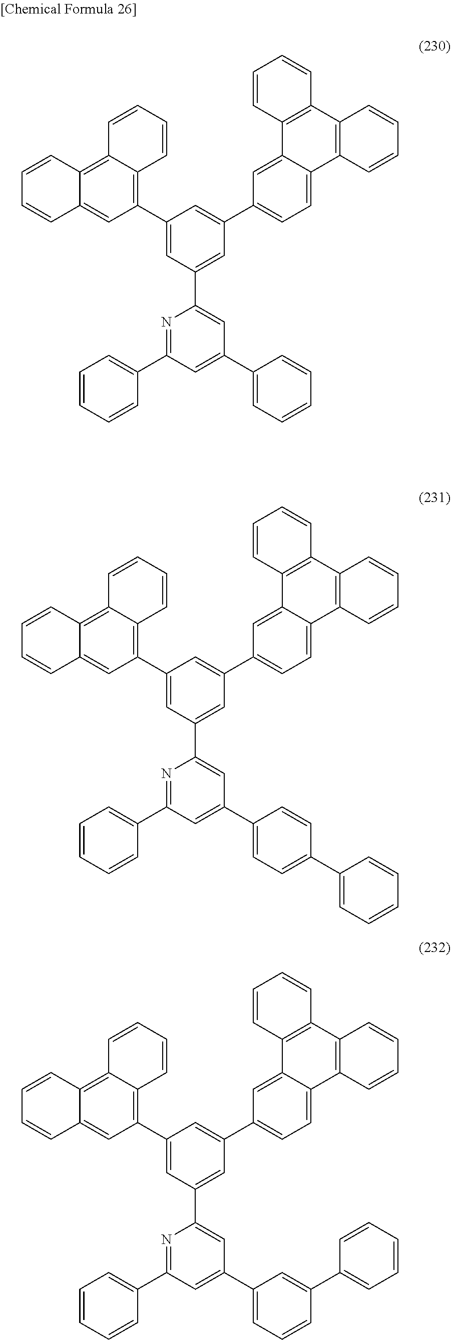

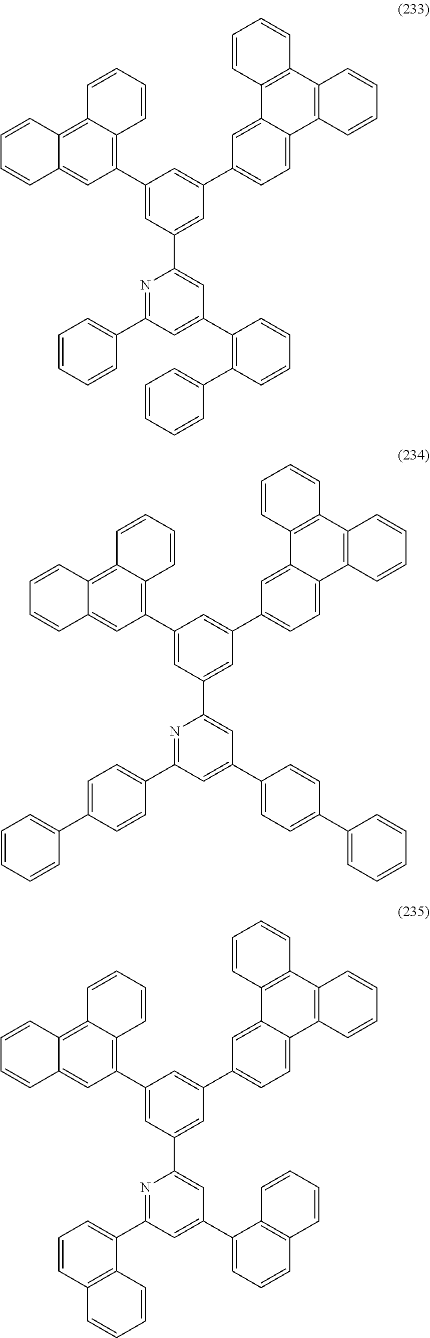

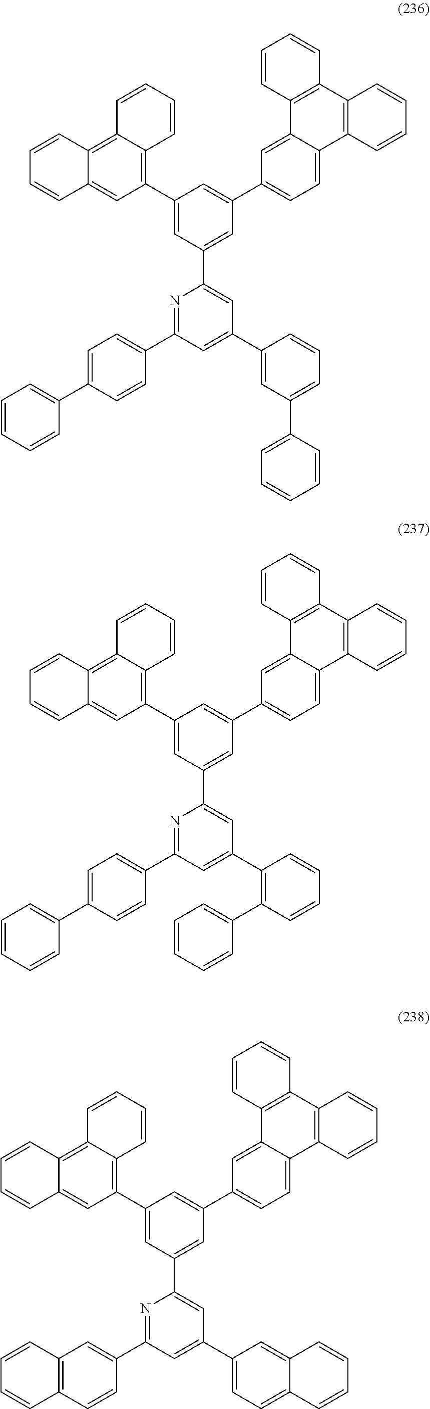

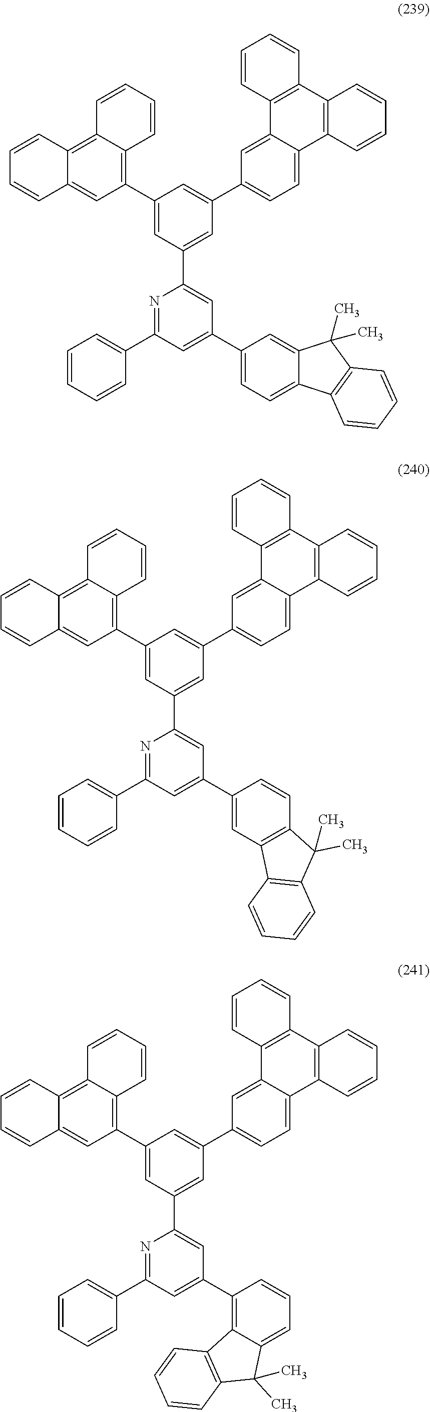

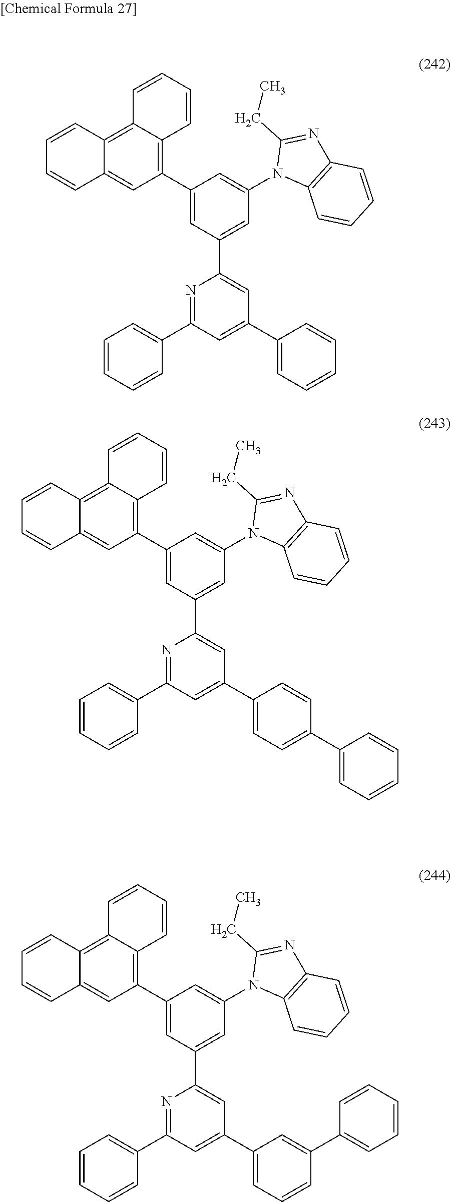

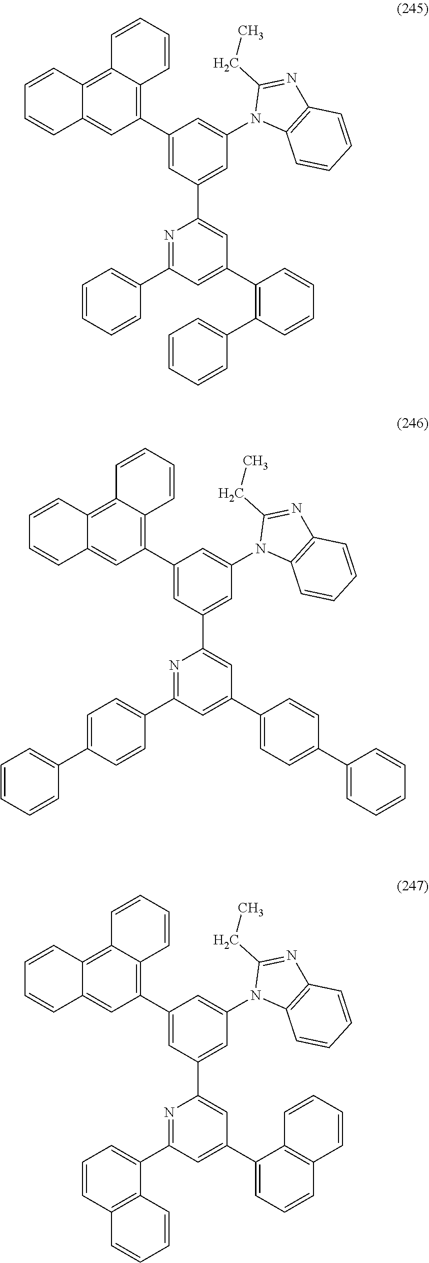

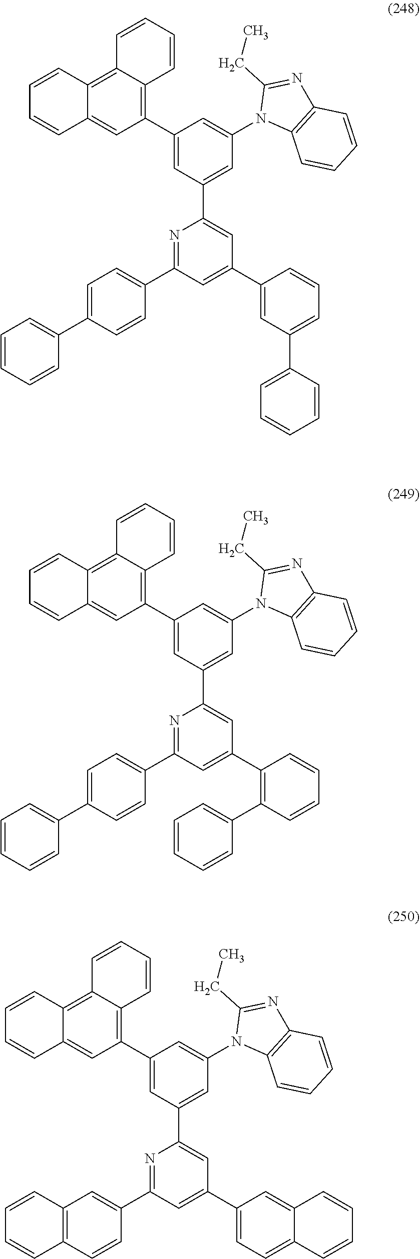

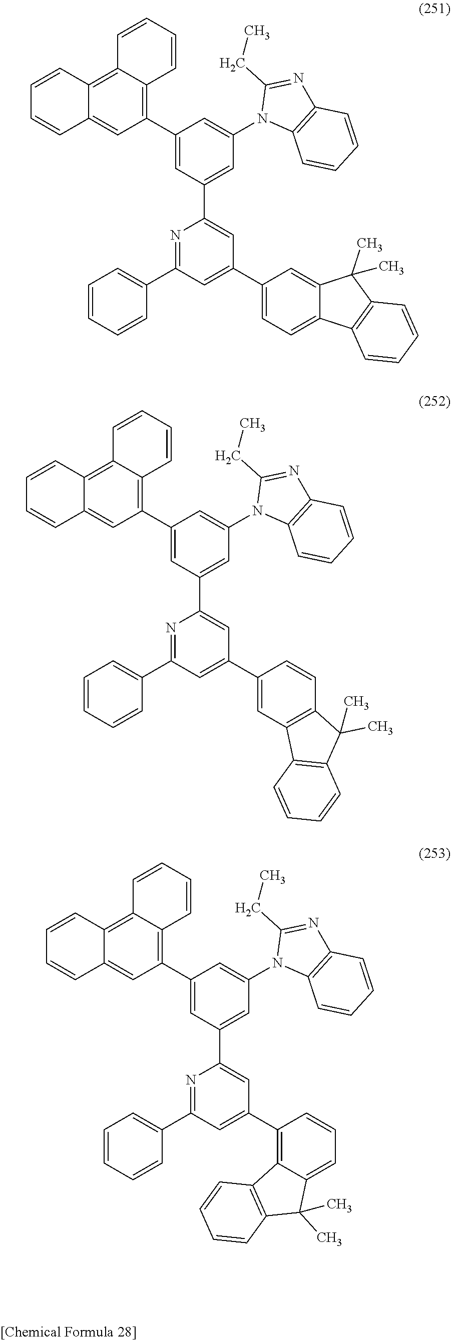

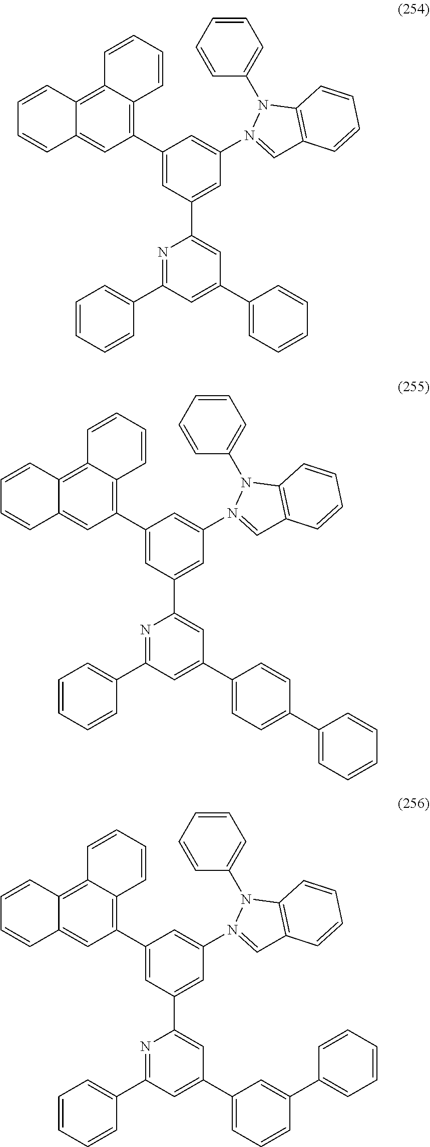

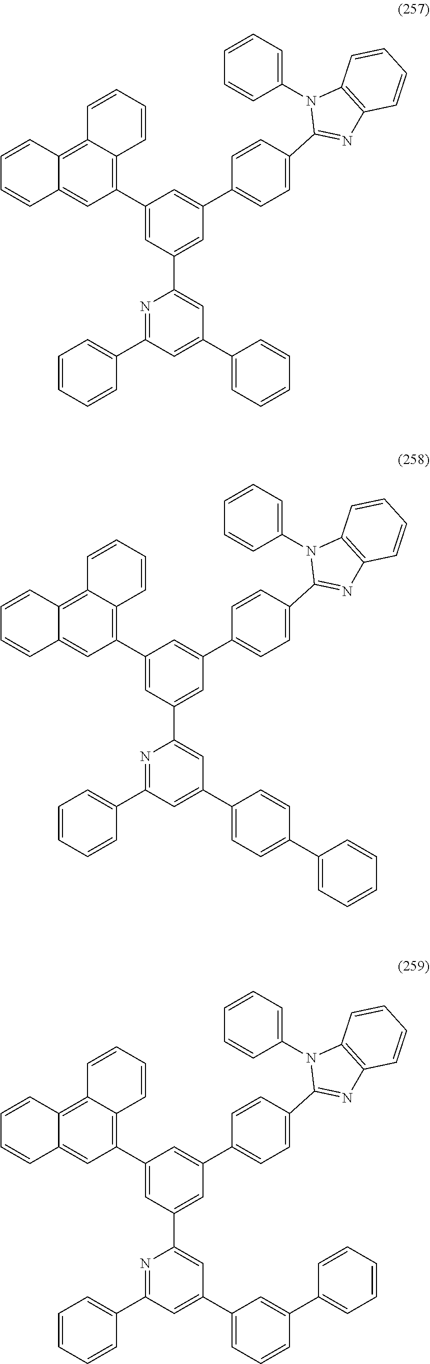

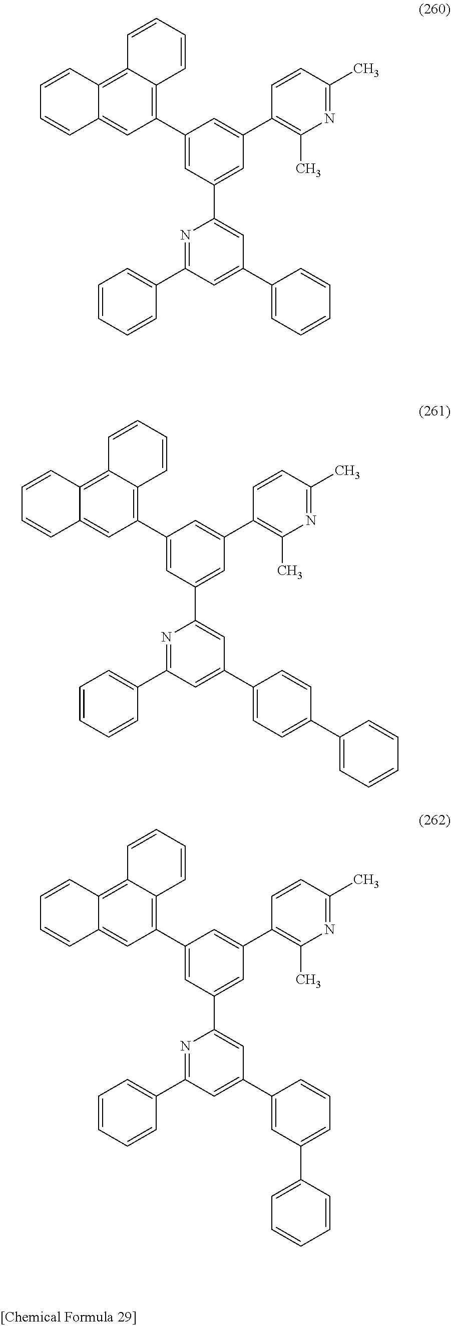

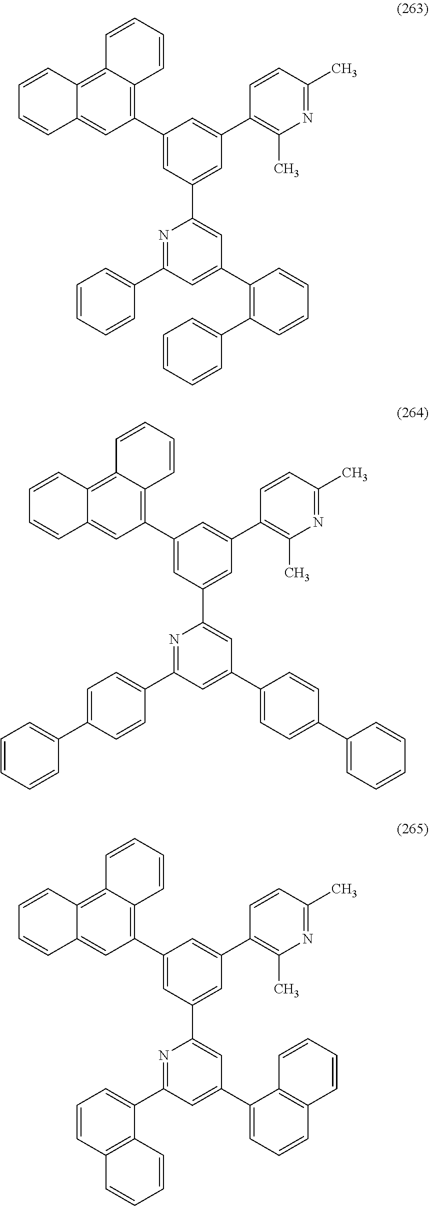

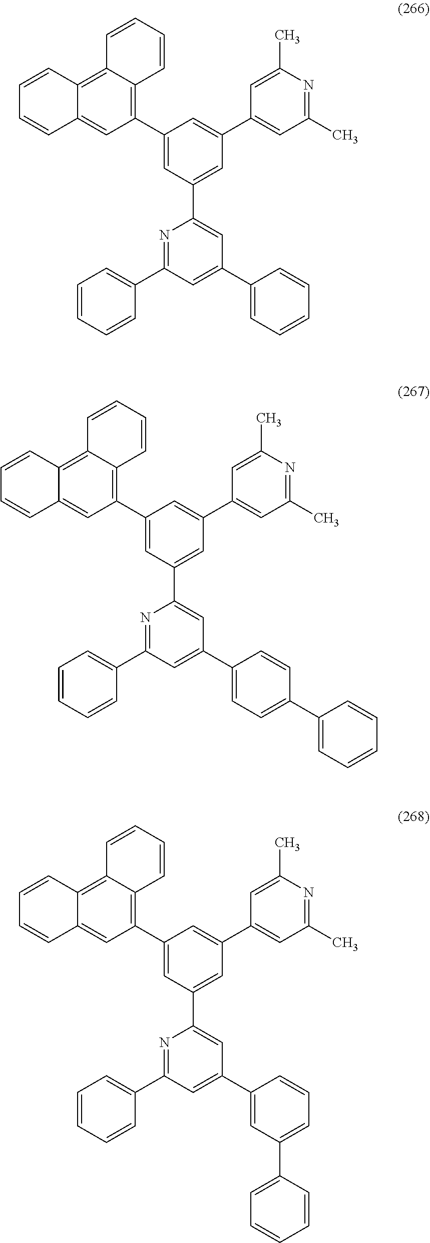

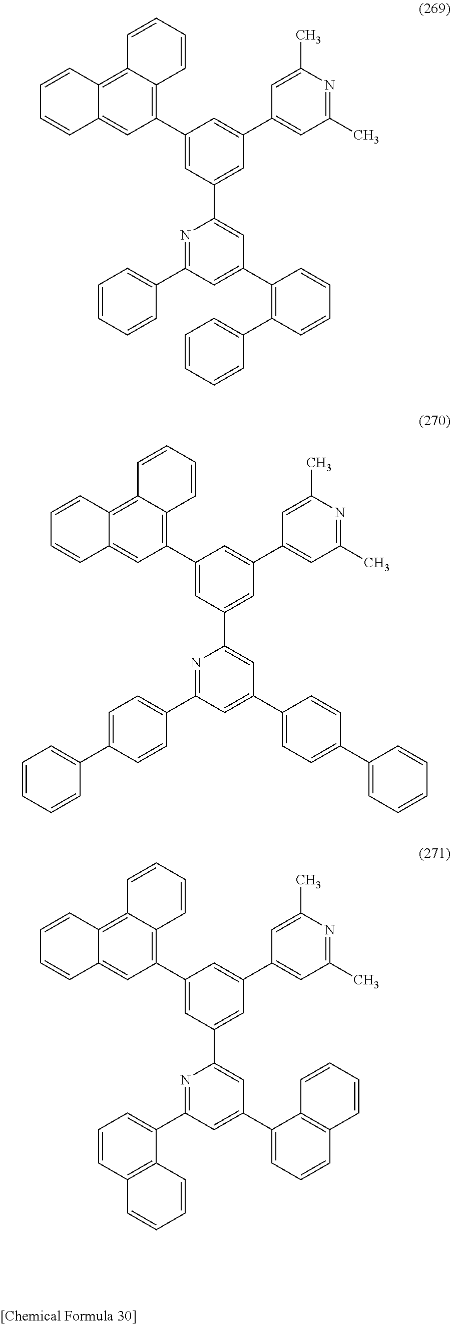

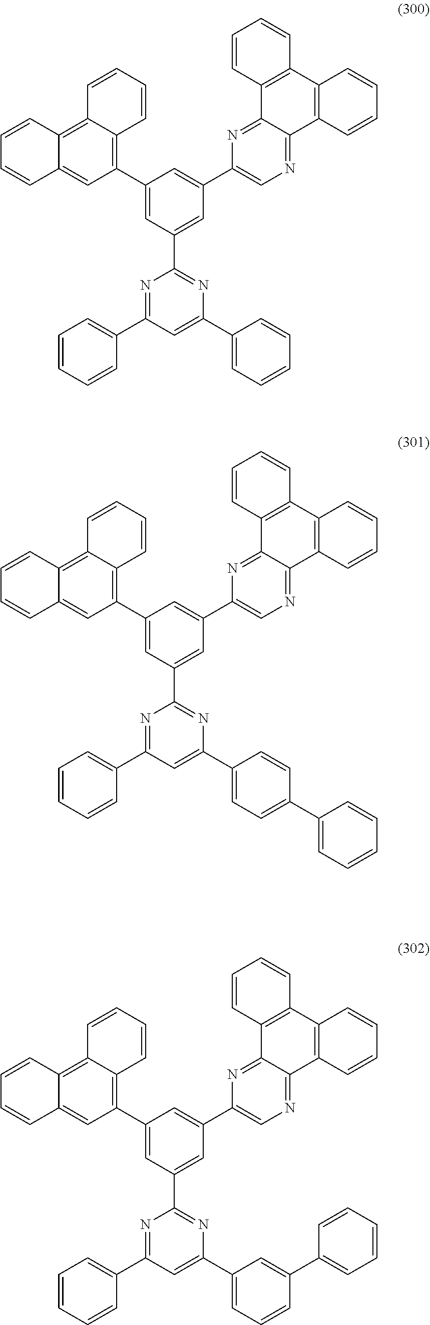

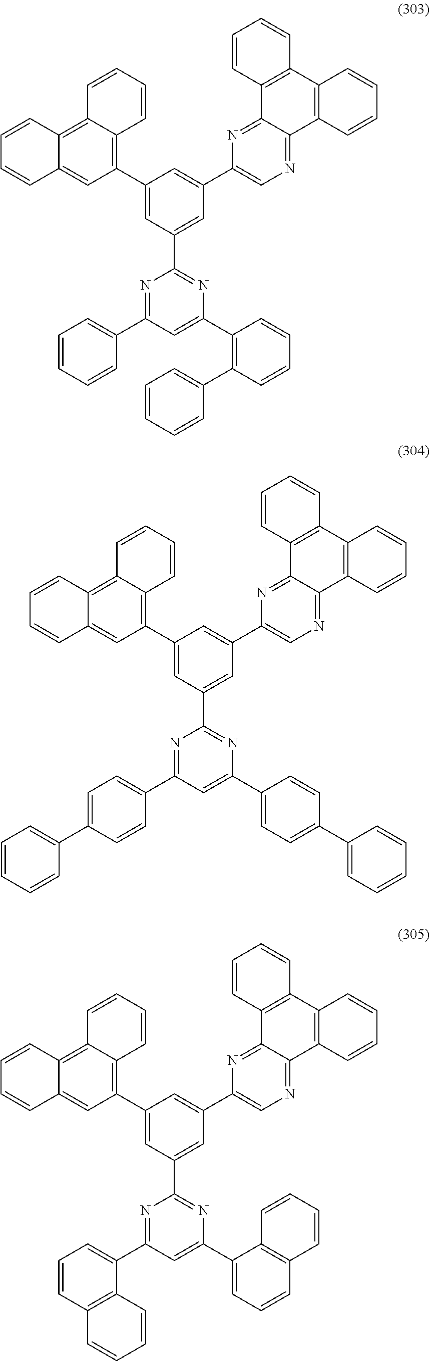

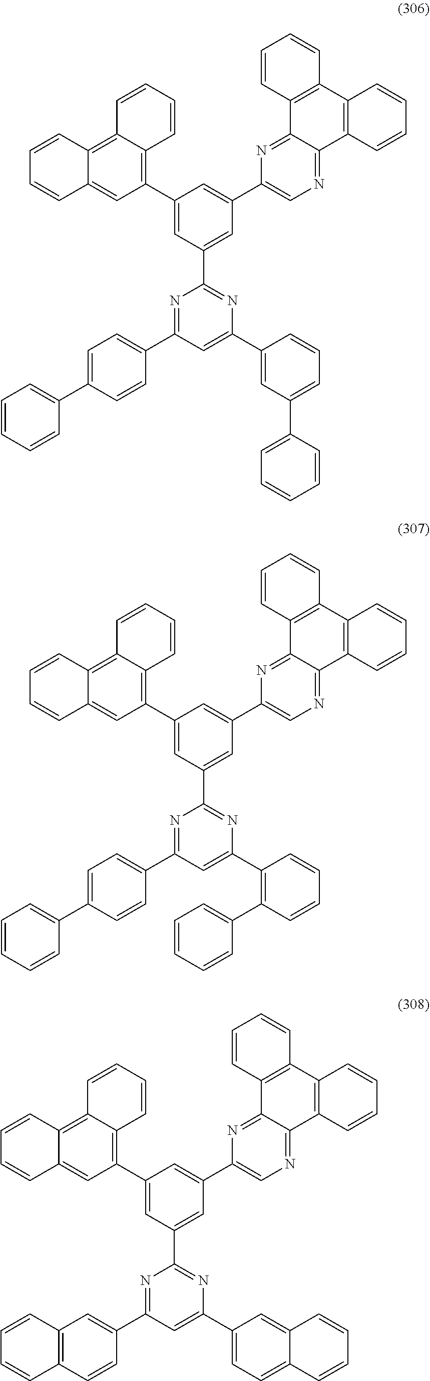

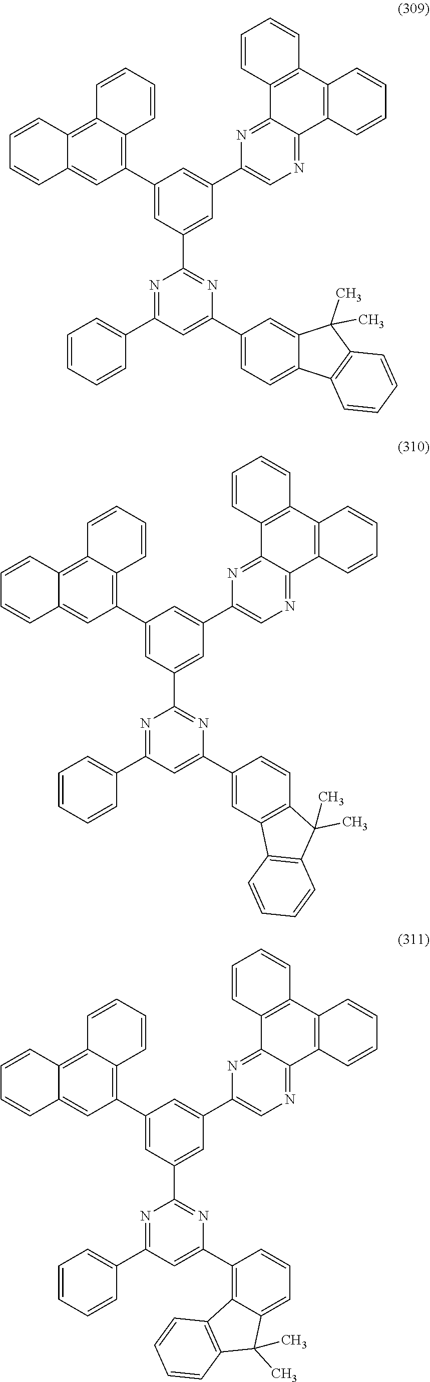

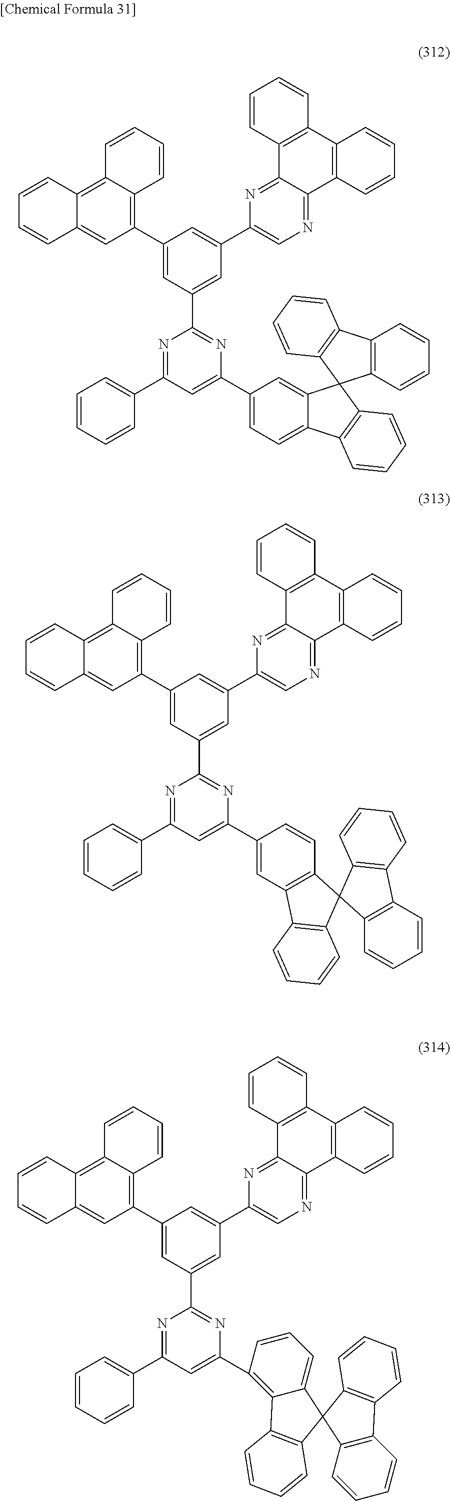

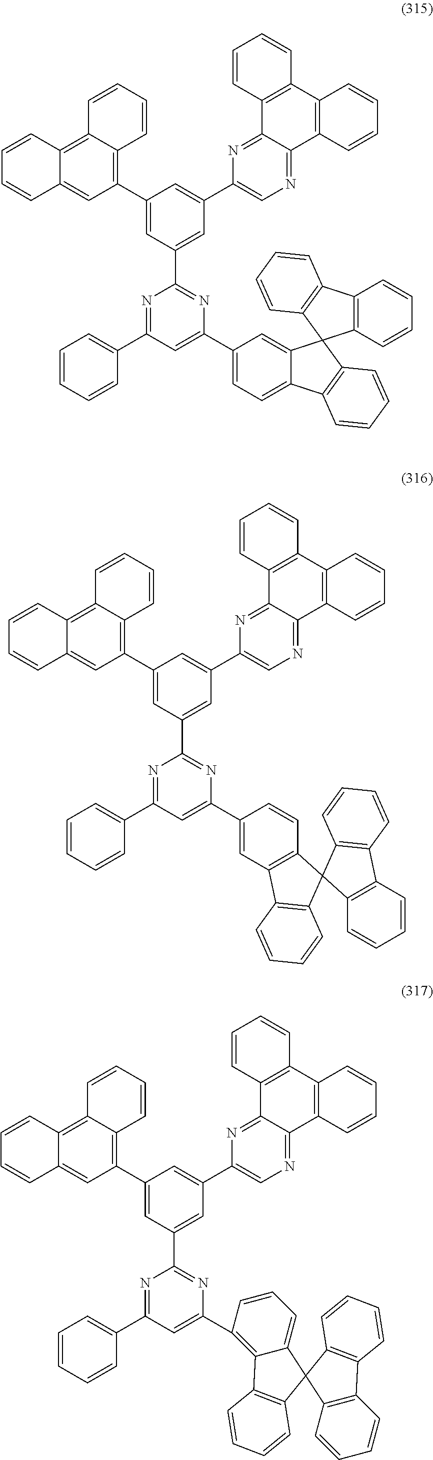

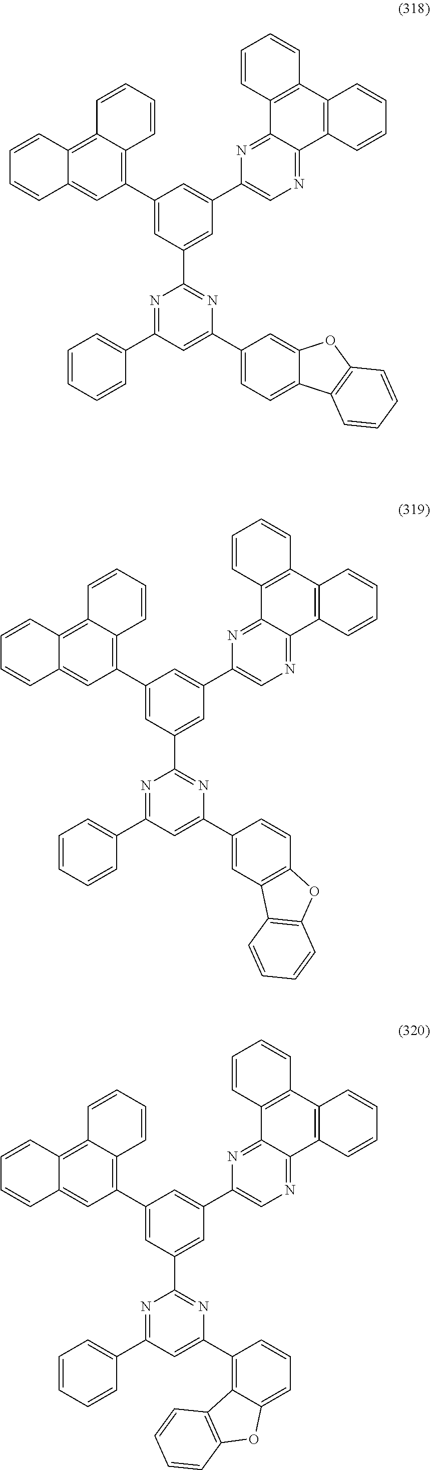

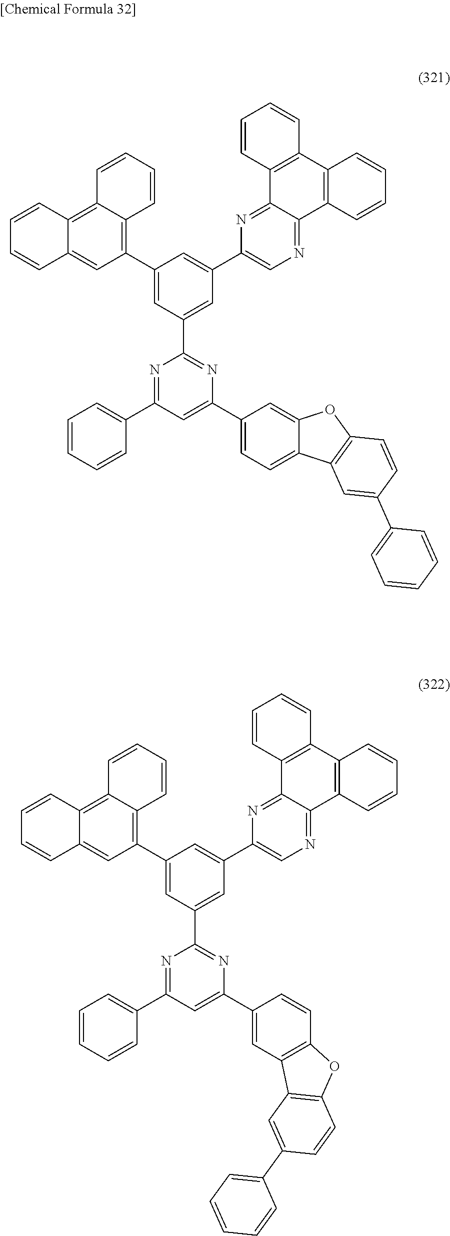

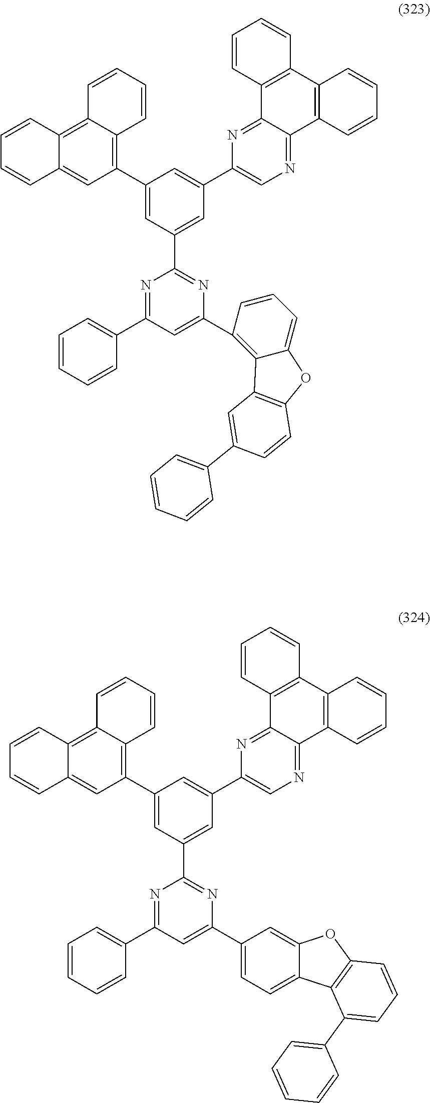

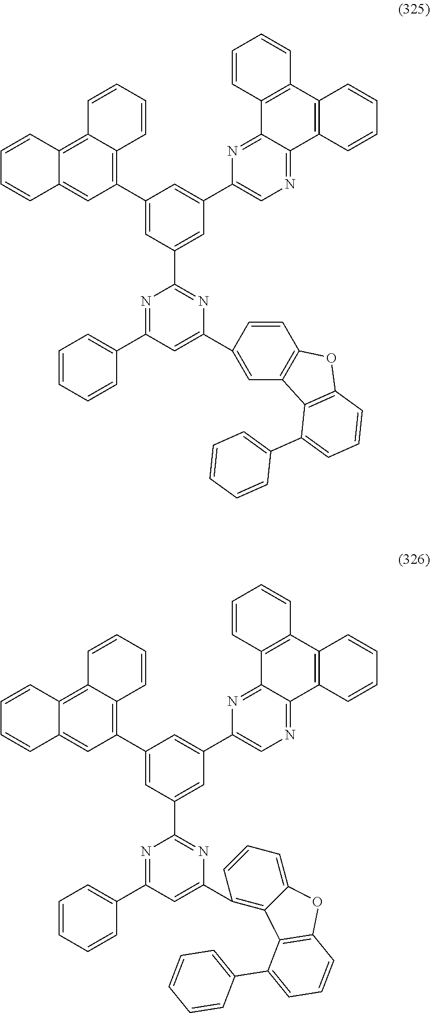

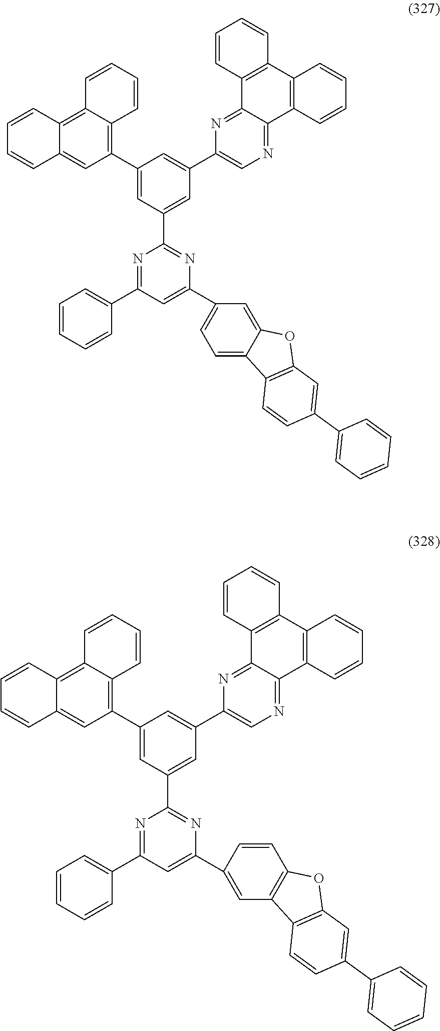

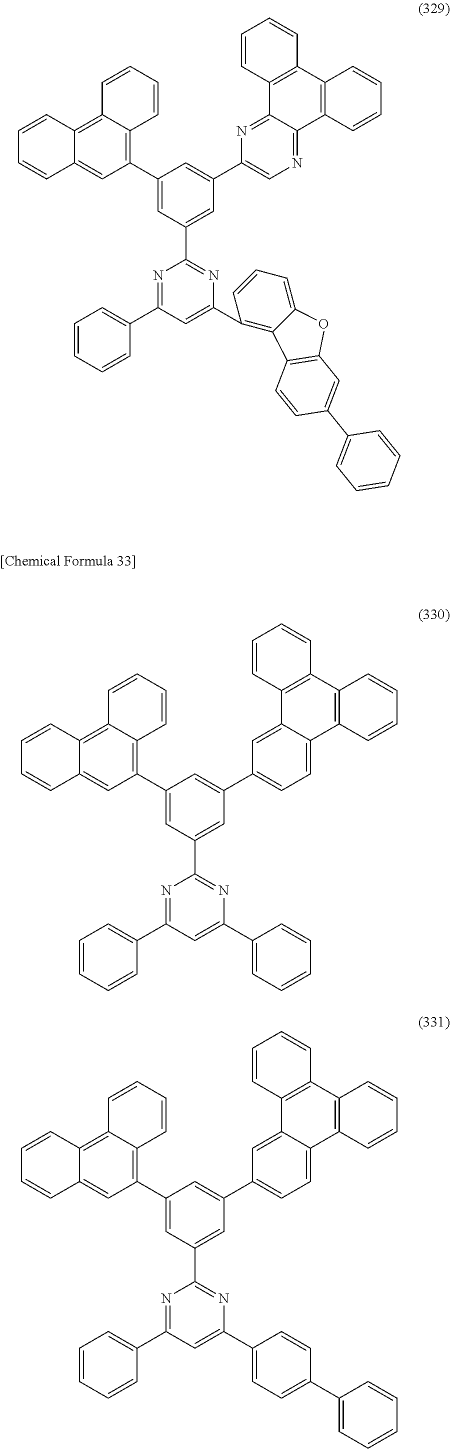

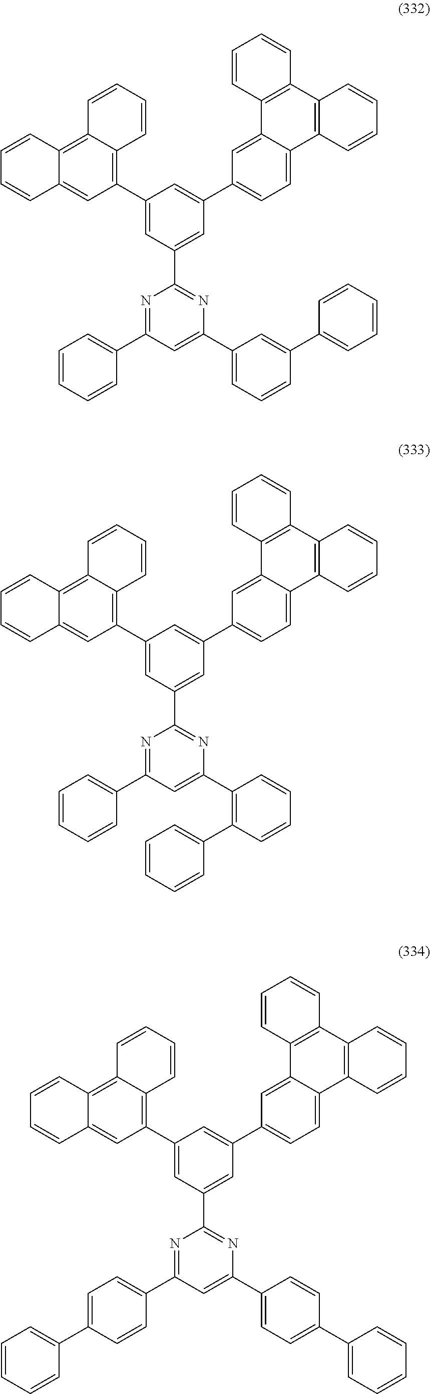

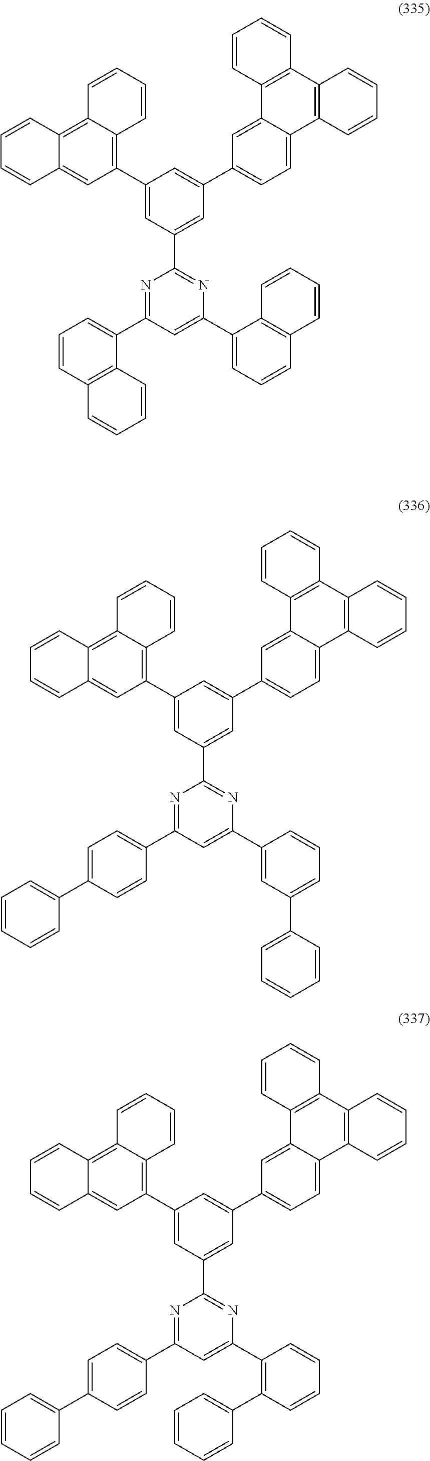

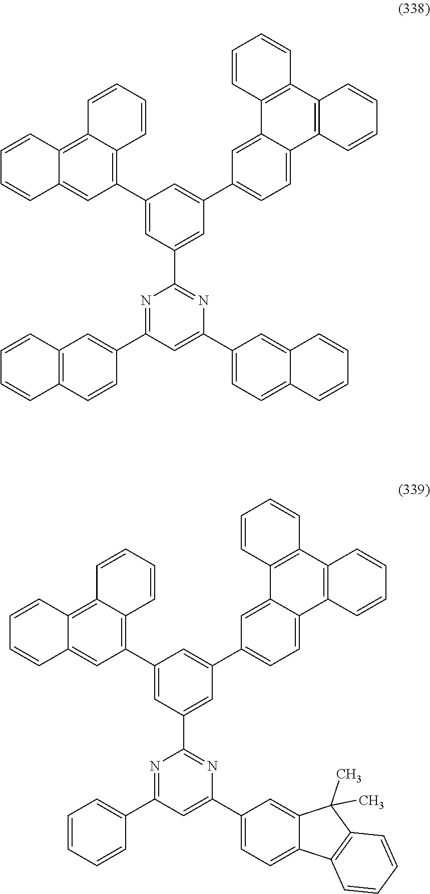

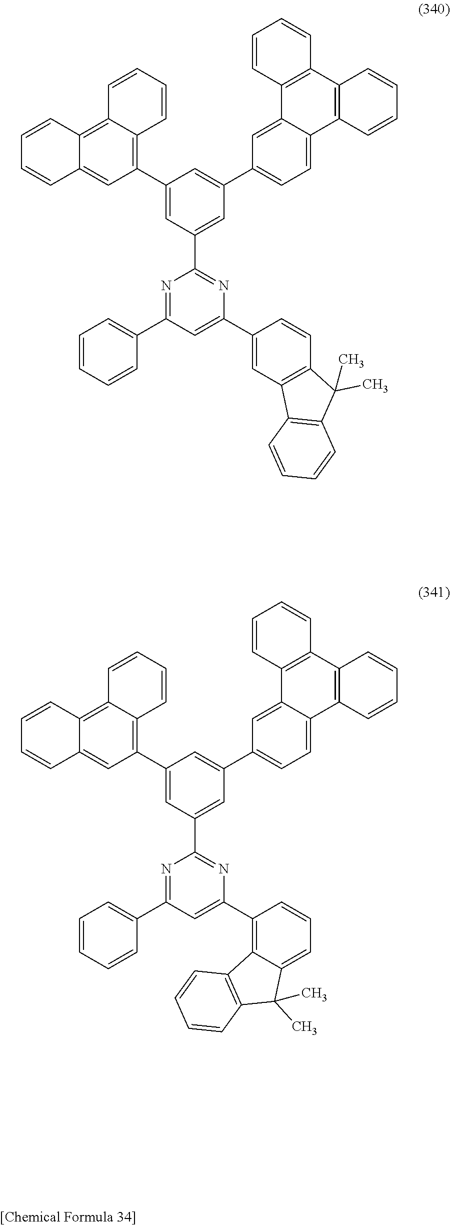

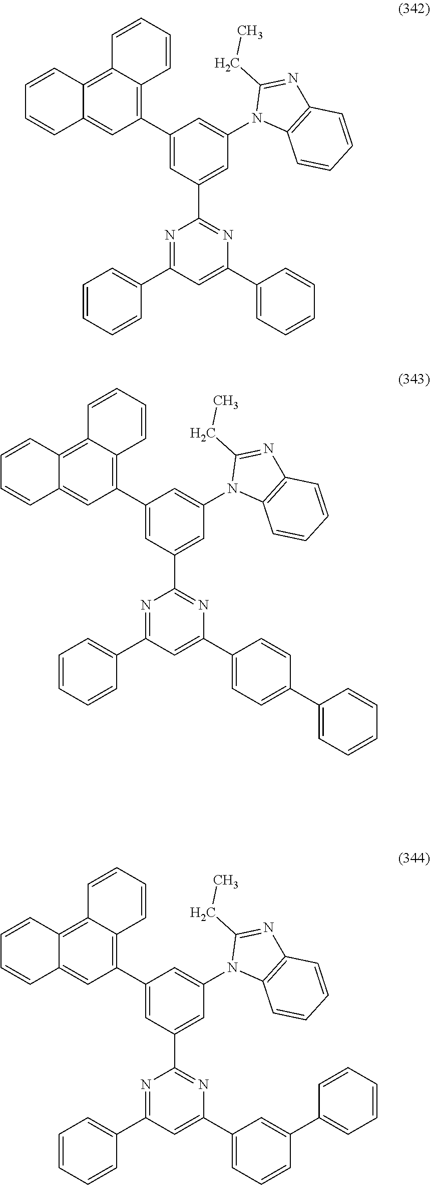

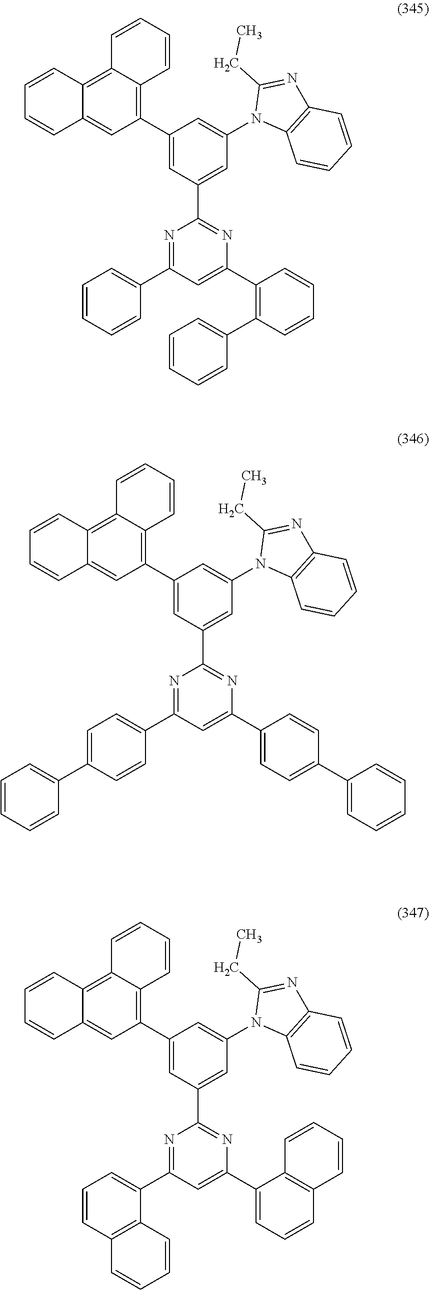

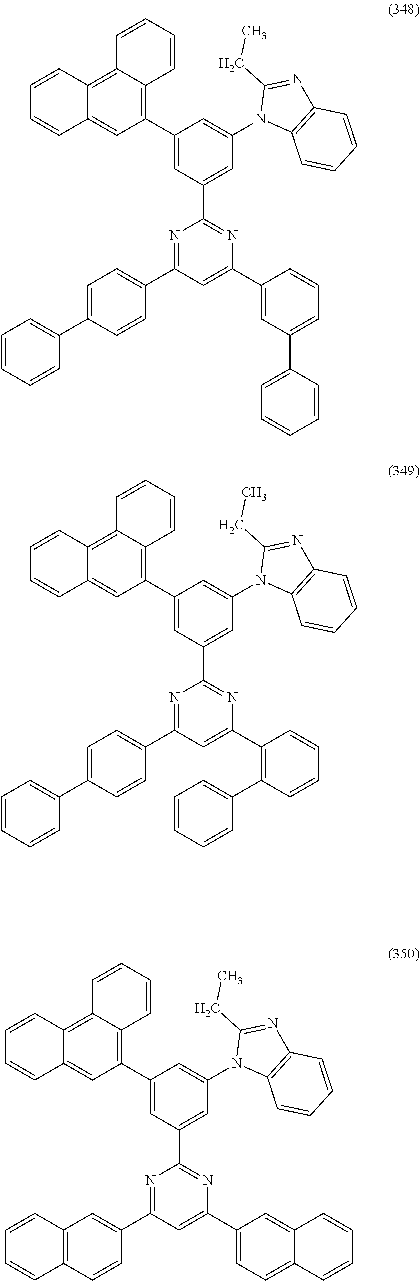

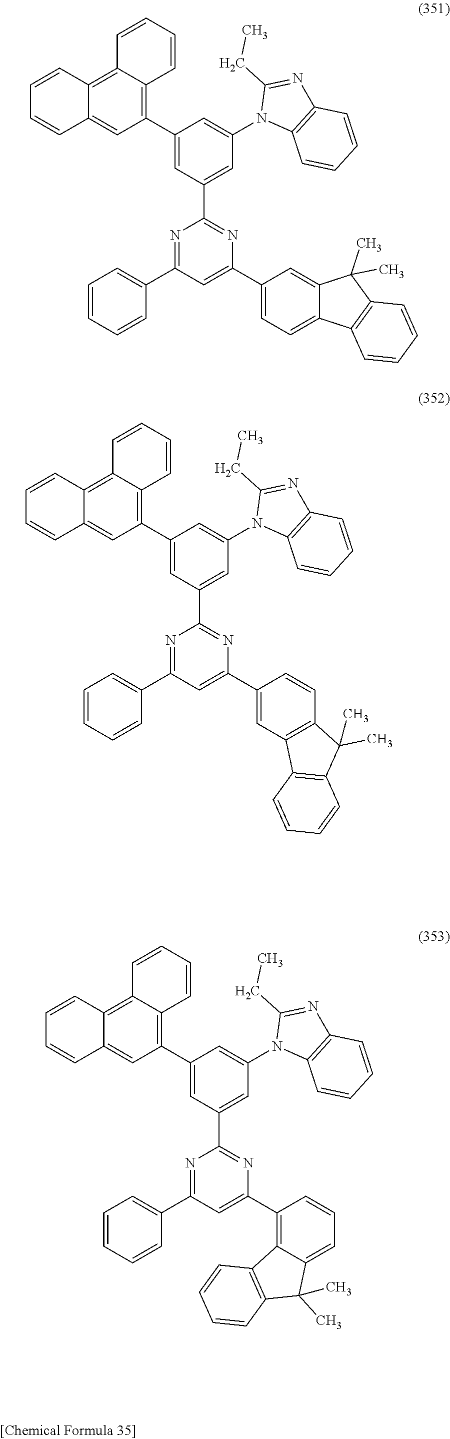

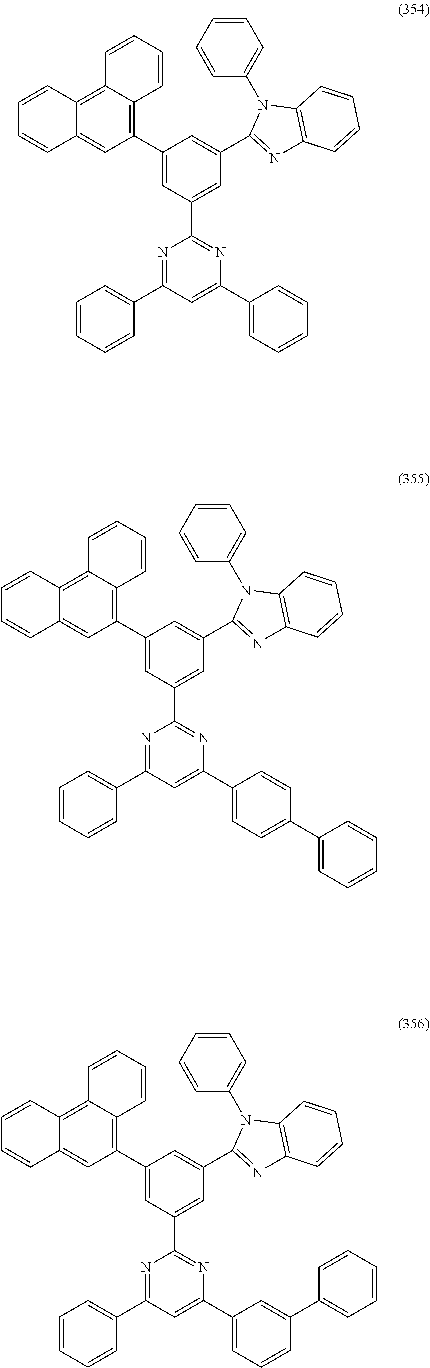

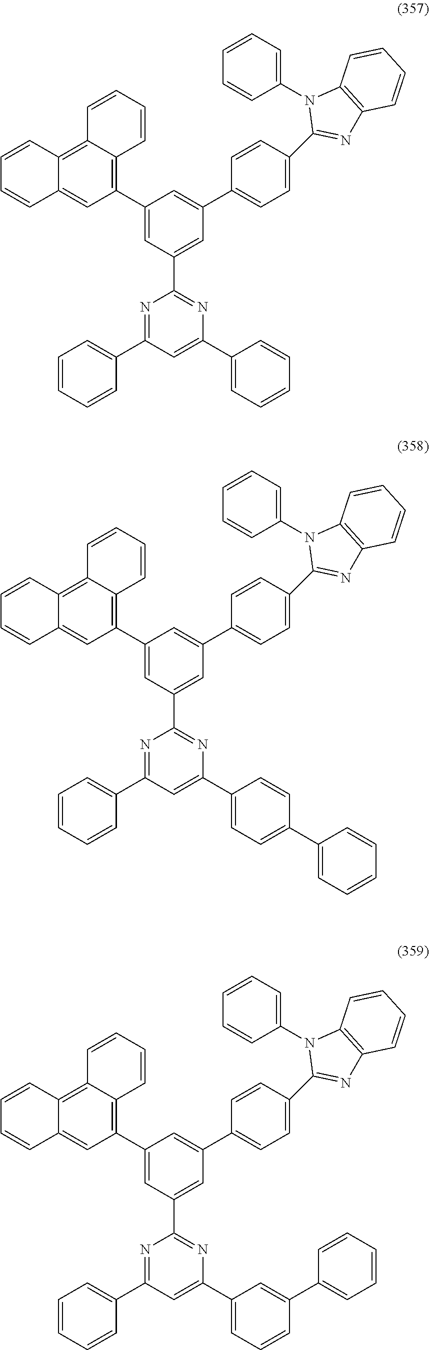

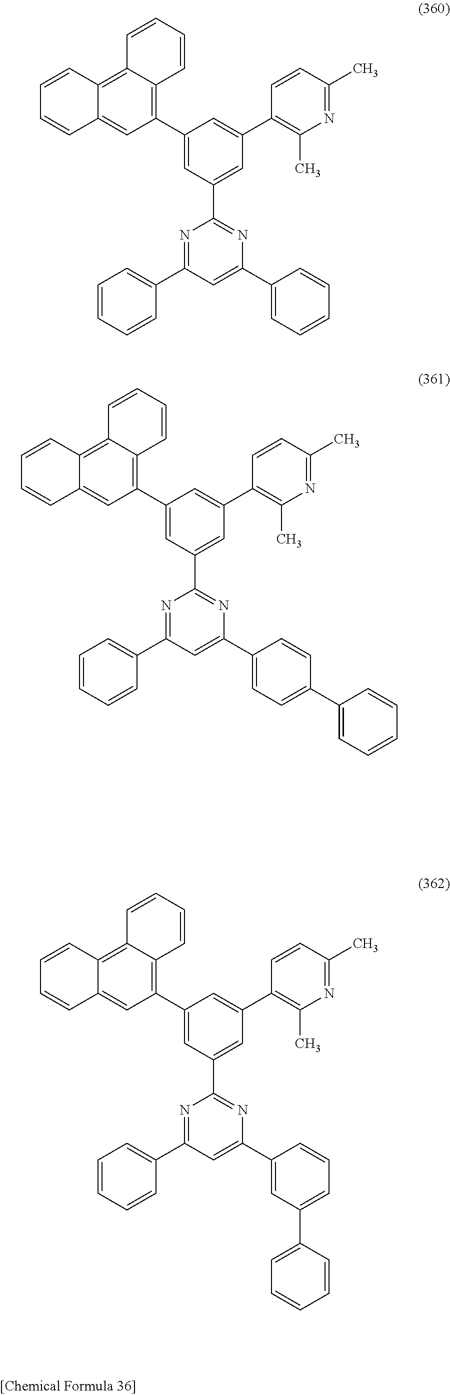

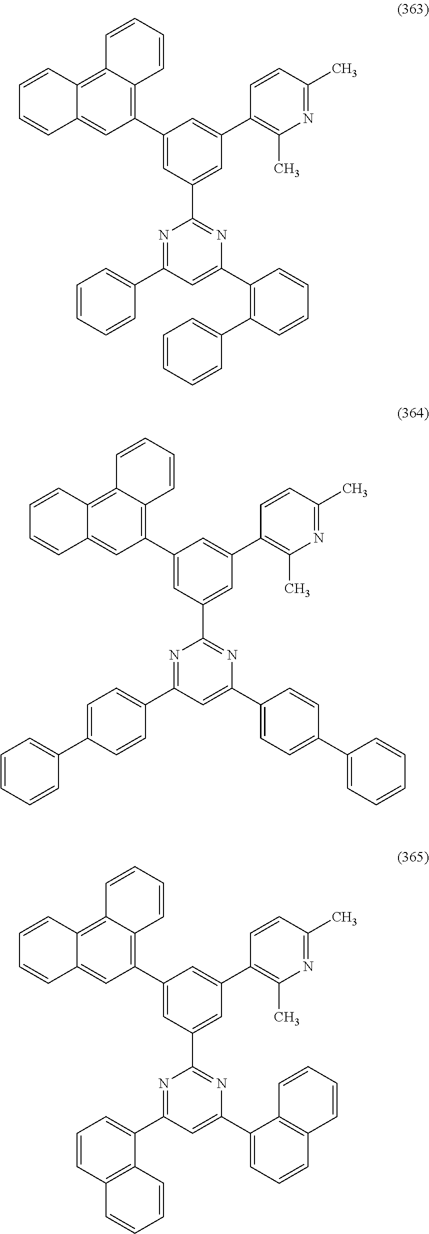

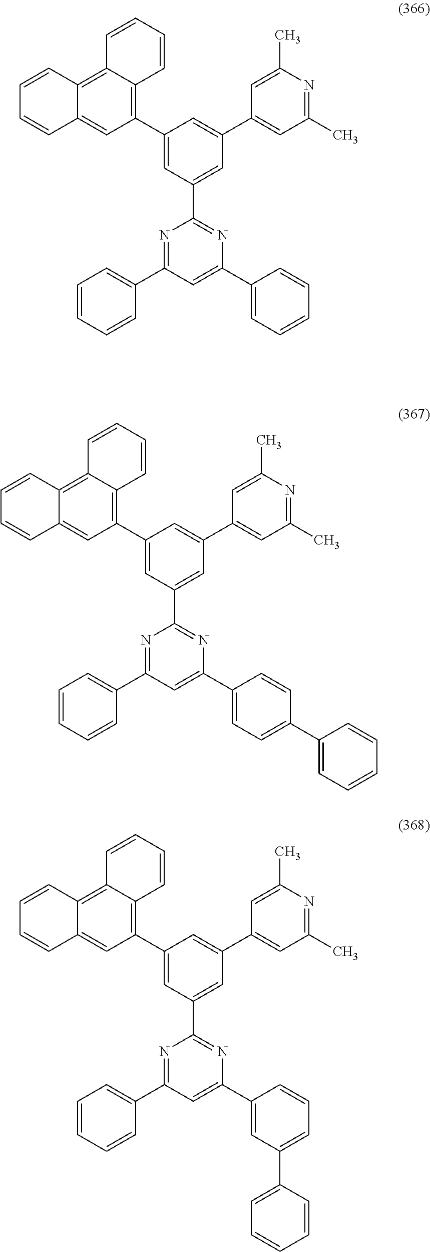

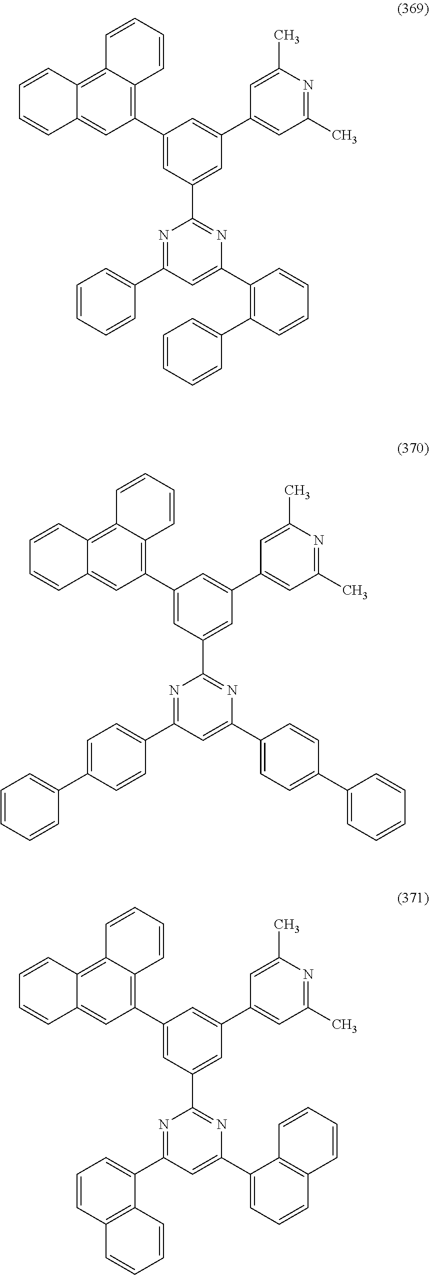

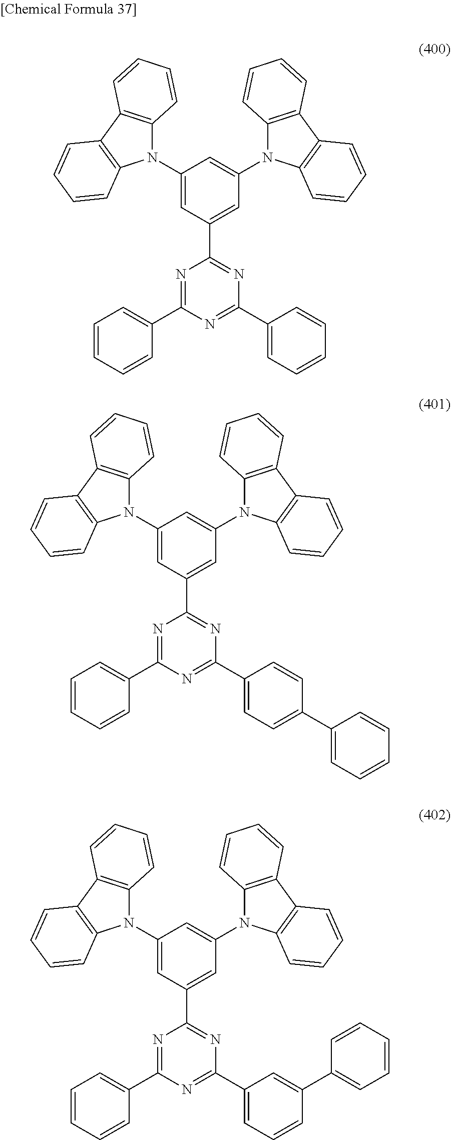

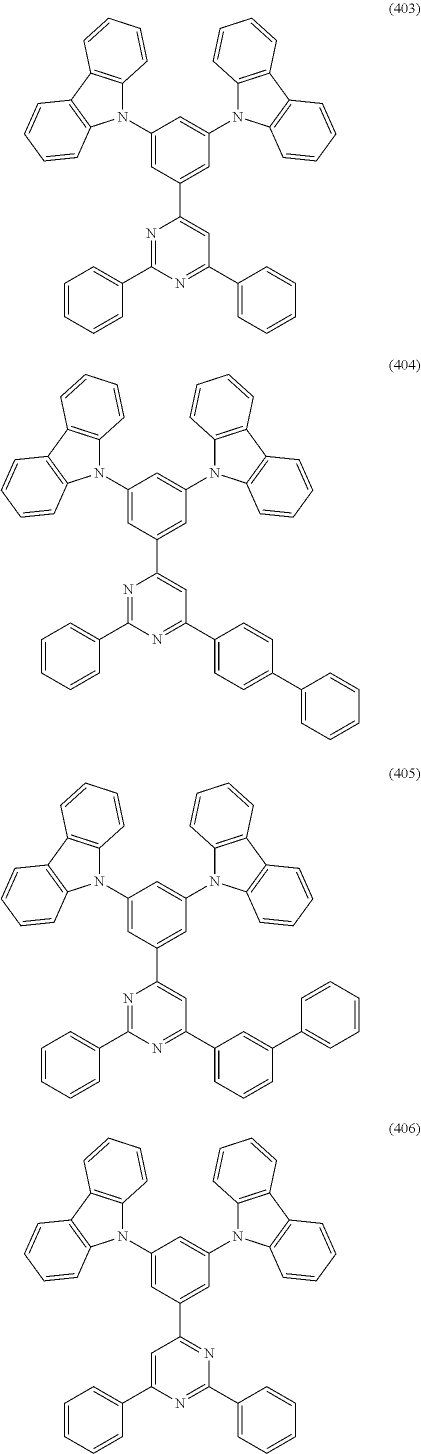

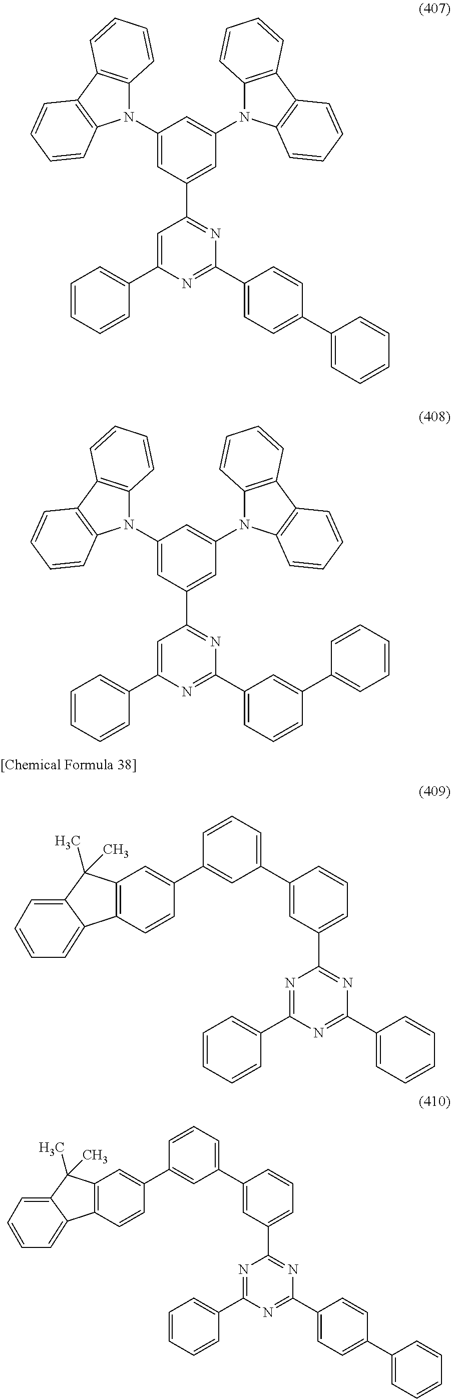

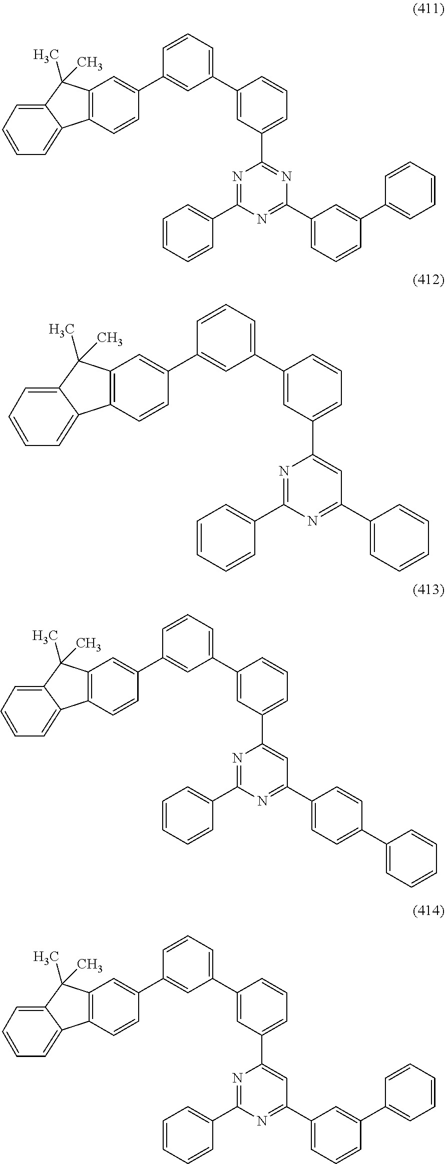

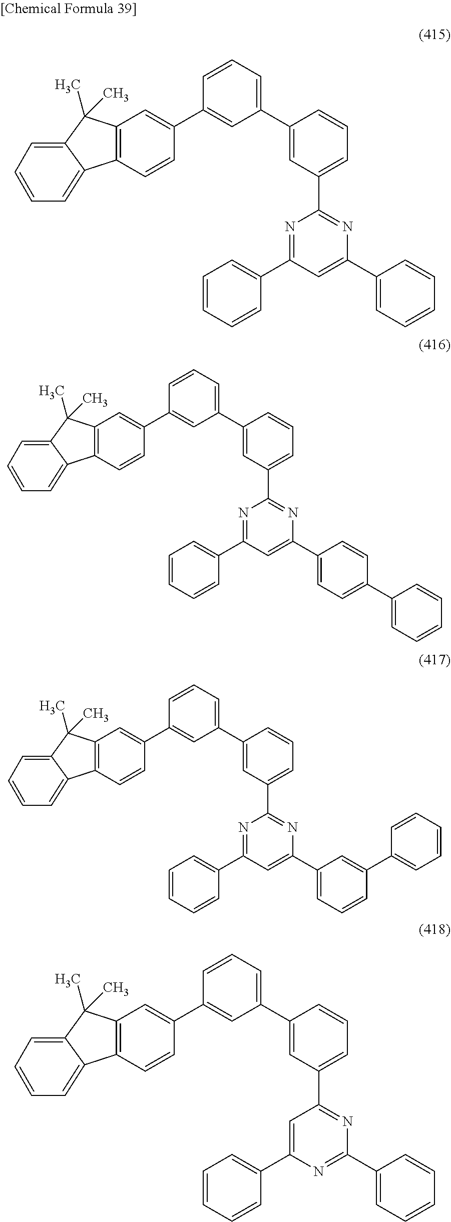

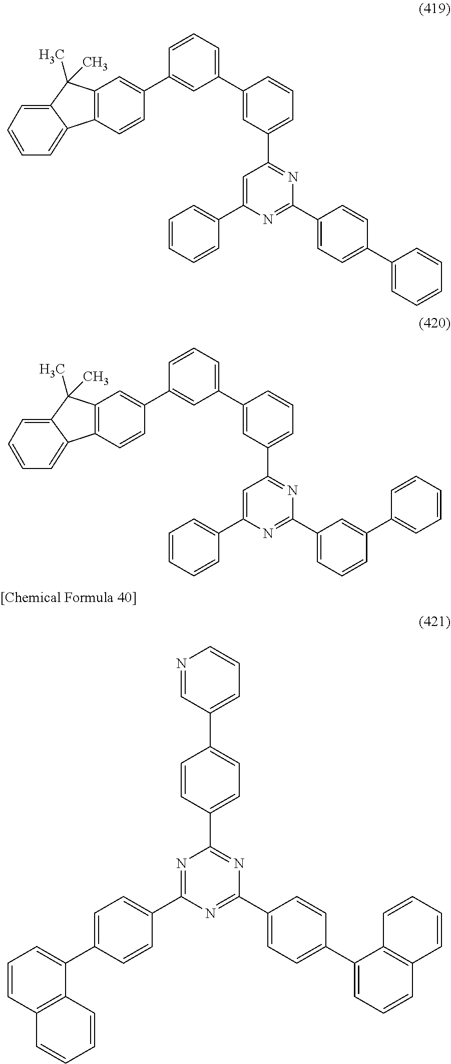

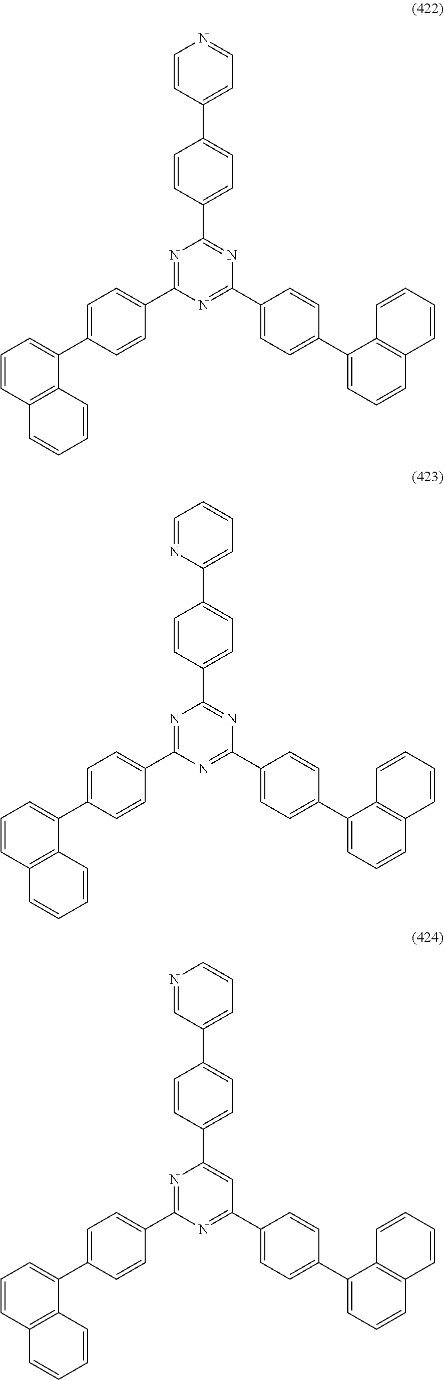

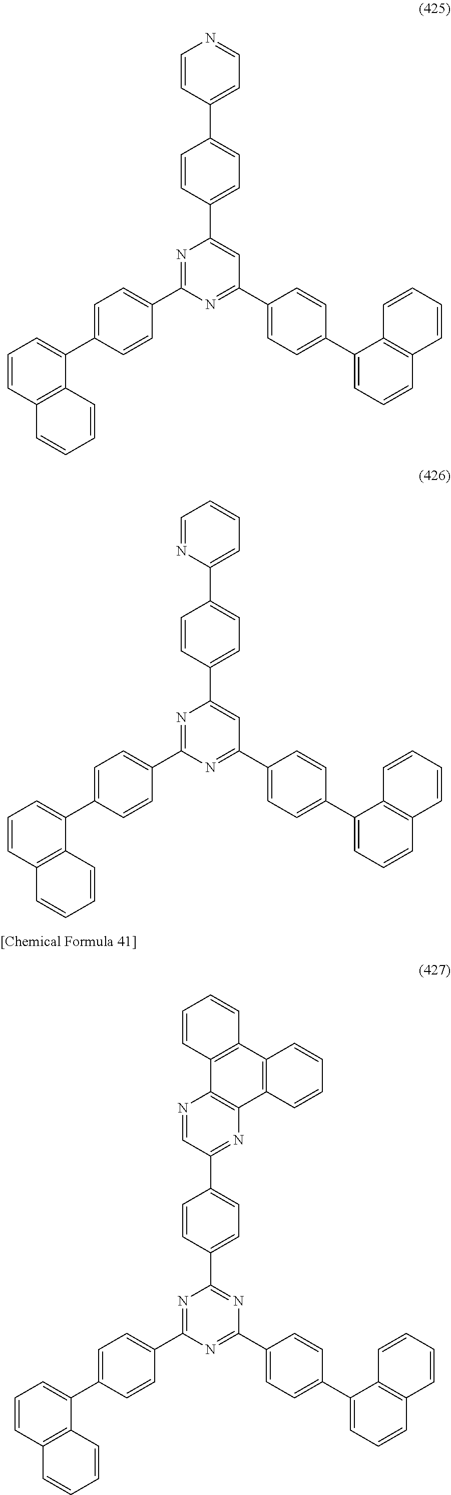

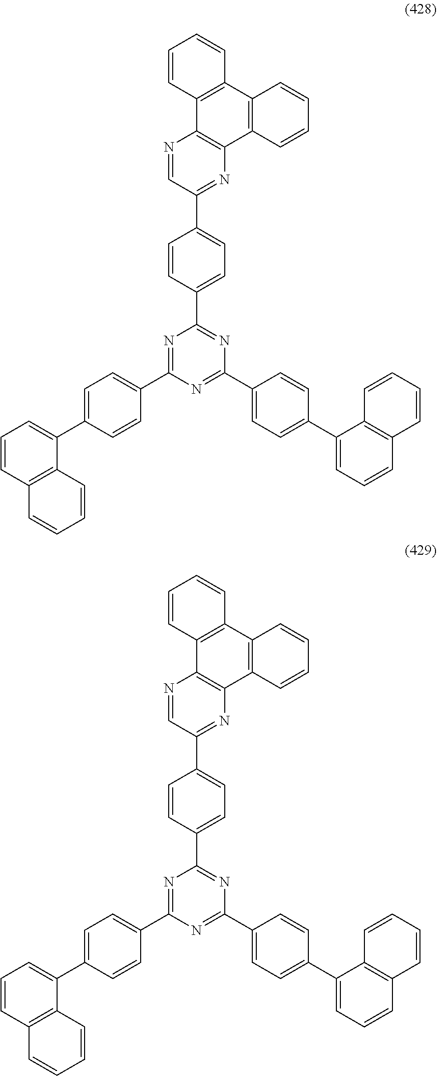

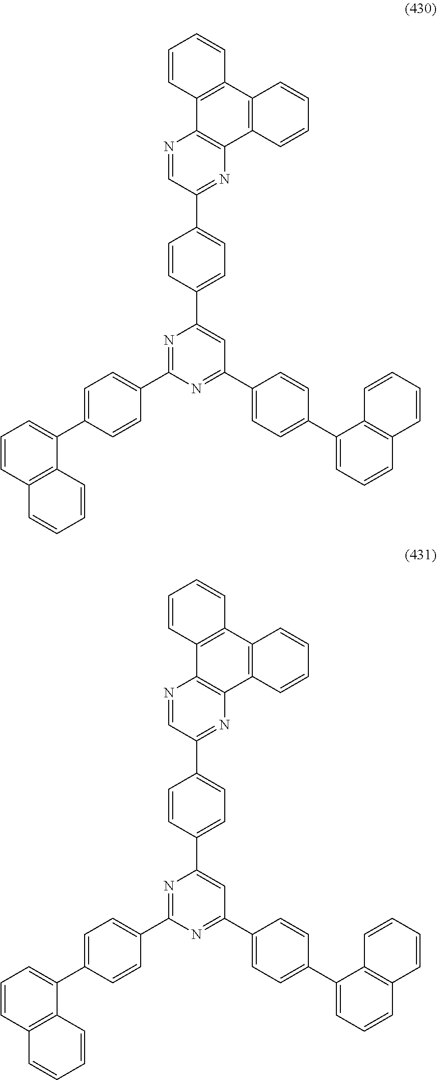

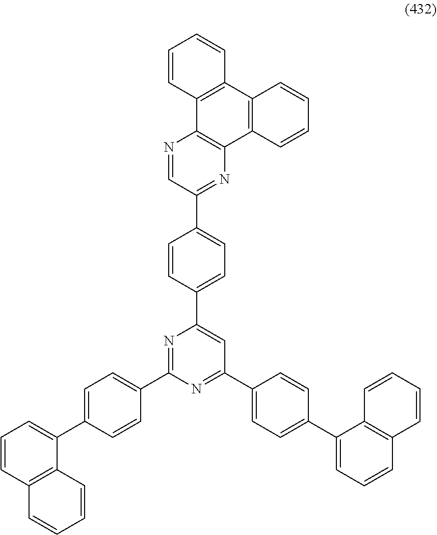

A variety of reactions can be employed as a method for synthesizing the compound of one embodiment of the present invention. For example, synthesis reactions described below enable the synthesis of the compound of one embodiment of the present invention represented by General Formula (G1). Note that the method for synthesizing the compound of one embodiment of the present invention is not limited to the synthesis methods below.

### <<Method for Synthesizing Compound Represented by General Formula (G1)>>

This embodiment describes a method for synthesizing the organic compound of one embodiment of the present invention represented by General Formula (G1) below.

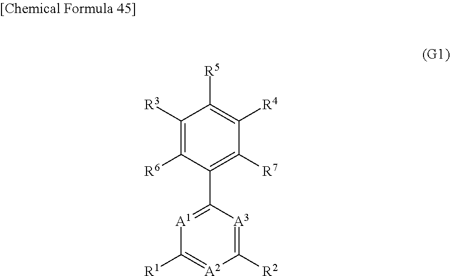

In General Formula (G1), any of the above-described elements or substituents can be used as A¹ to A³ or R¹ to R⁷.

The organic compound represented by General Formula (G1) can be synthesized as shown in Synthesis Schemes (a-1) to (a-5) below.

First, according to Reaction Formula (a-1), a compound 1 including an azine skeleton and a compound 2 including R¹ are coupled with each other, whereby a compound 3 can be obtained. Next, according to Reaction Formula (a-2), the compound 3 and a compound 4 including R² are coupled with each other, whereby a compound 5 can be obtained. Then, according to Reaction Formula (a-3), the compound 5 and a compound 6 are coupled with each other, whereby a compound 7 can be obtained. Next, according to Reaction Formula (a-4), the compound 7 and a compound 8 including R³ are coupled with each other, whereby a compound 9 can be obtained. Then, according to Reaction Formula (a-5), the compound 9 and a compound 10 including R⁴ are coupled with each other, whereby the compound represented by General Formula (G1) can be obtained.

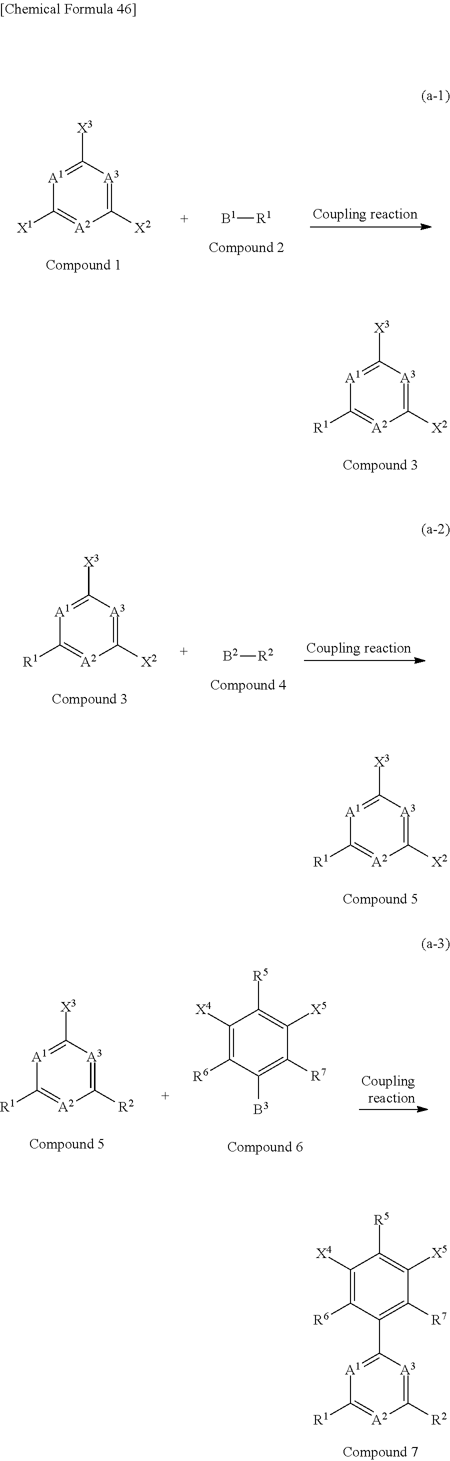

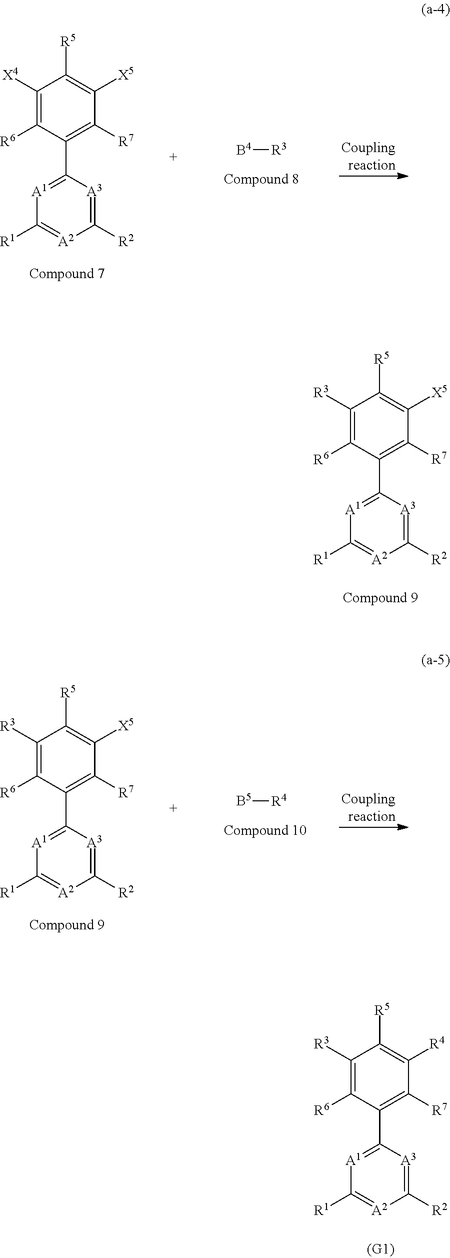

In Synthesis Schemes (a-1) to (a-5), any of the above-described elements or substituents can be used as A¹ to A³ or R¹ to R⁷.

In Synthesis Schemes (a-1) to (a-5), each of X¹ to X³ represents a halogen group (including chlorine, for example). In addition, each of B¹ to B⁵ represents a boronic acid. Note that without limitation to these examples, each of X¹ to X³ and B¹ to B⁵ may independently be chlorine, bromine, iodine, a triflate group, an organoboron group, a boronic acid, organoaluminum, organozirconium, organozinc, an organotin group, or the like. Note that in the case where R³ and R⁴ have different substituent structures in General Formula (G1), it is preferable that one of X⁴ and X⁵ be chlorine and the other be bromine or iodine. When one of X⁴ and X⁵ is chlorine and the other is bromine or iodine, the coupling reaction proceeds preferentially in X⁴ or X⁵ which is bromine or iodine, so that General Formula (G1) can be obtained with a high yield and high purity. In the case where R³ and R⁴ have the same substituent structure in General Formula (G1), it is preferable that X⁴ and X⁵ be the same halogen or each of X⁴ and X⁵ be independently bromine or iodine; in this case, by making two equivalents of the compound 8 react with the compound 7, General Formula (G1) can be obtained from the compound 7 only in one step.

Synthesis Schemes (a-1) to (a-5) can be performed by the Suzuki-Miyaura coupling reaction, for example. Examples of a palladium (Pd) catalyst in this case include palladium(II) acetate, tetrakis(triphenylphosphine)palladium(0), and bis(triphenylphosphine)palladium(II) dichloride. Examples of a ligand of the palladium catalyst include tri (ortho-tolyl)phosphine, triphenylphosphine, and tricyclohexylphosphine.

Examples of a base that can be used in the reactions in Synthesis Schemes (a-1) to (a-5) include organic bases such as sodium-tert-butoxide and inorganic bases such as potassium carbonate, sodium carbonate, potassium phosphate, and potassium acetate.

Examples of a solvent that can be used in the reactions in Synthesis Schemes (a-1) to (a-5) include a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; and a mixed solvent of water and an ether such as diethylene glycol dimethyl ether. Note that a mixed solvent of toluene and water, a mixed solvent of toluene, ethanol, and water, or a mixed solvent of water and an ether such as diethylene glycol dimethyl ether is further preferable.

Note that any palladium catalyst, ligand, base, and solvent other than the above examples may be used.

Alternatively, R¹ to R⁷ may each be deuterium. Substituents of A¹ to A³ or substituents of R¹ to R⁷ may each be deuterium. In that case, the coupling reaction is performed using a compound obtained by deuteration of the compound 1, the compound 2, the compound 4, the compound 6, the compound 8, or the compound 10. Examples of a solvent that can be used in a reaction performing deuteration include benzene-d6, toluene-d8, xylene-d10, and heavy water. Examples of a catalyst that can be used include molybdenum(V) chloride, tungsten(VI) chloride, niobium(V) chloride, tantalum(V) chloride, aluminum(III) chloride, titanium(IV) chloride, and tin (IV) chloride. However, the solvent and the catalyst are not limited to the above.

For example, in the case of manufacturing a compound of General Formula (G1) where R³ is an aryl group and R⁴ to R⁷ are each hydrogen, a compound 11 where X⁶ is halogen (including chlorine, for example) and X⁷ to X¹⁰ are each hydrogen is coupled with a compound 12. Similarly, in the case of manufacturing a compound where R³ and R⁴ are each an aryl group and R⁵ to R⁷ are each hydrogen, the compound 11 where X⁶ and X⁷ are each halogen and X⁸ to X¹⁰ are each hydrogen, the compound 12, and a compound 13 are coupled sequentially. Note that the compound 11 can be synthesized in a manner similar to that of the compound 7 using Reaction Formulae (a-1) to (a-3) in the synthesis method 1.

Each of X⁶ to X¹⁰ and B⁶ to B¹⁰ independently represents hydrogen, chlorine, bromine, iodine, a triflate group, an organoboron group, a boronic acid, organoaluminum, organozirconium, organozinc, an organotin group, or the like.

As the conditions (a palladium catalyst, a solvent, and the like) used in the coupling reactions, the conditions in the method shown in Reaction Formulae (a-1) to (a-5) can be used.

Note that a compound represented by General Formula (G2) or General Formula (G3) can also be synthesized by a similar synthesis method.

Some of the compounds represented by Structural Formulae (100) to (450) which are manufactured by the above synthesis method will be described later as examples. Needless to say, the compounds that are not described as examples can be manufactured by coupling any of the above substituents (R-1) to (R-112) with R¹ to R⁷ in Reaction Formulae (a-1) to (a-5) or R¹ to R⁷ in (b-1) as appropriate.

Note that the above synthesis method is an example and the compound of the present application can be manufactured by another synthesis method.

### Embodiment 2

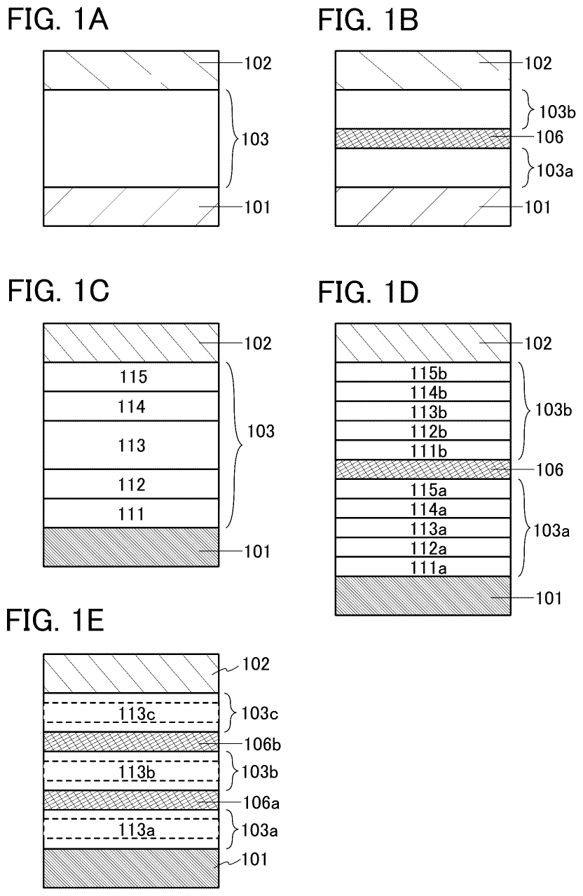

In this embodiment, structures of a light-emitting device using the organic compound described in Embodiment 1 will be described with reference to FIG. 1A to FIG. 1E.

### <<Basic Structure of Light-Emitting Device>>

A basic structure of a light-emitting device is described. FIG. 1A illustrates a light-emitting device in which an EL layer including a light-emitting layer is provided between a pair of electrodes. Specifically, the light-emitting device has a structure where an EL layer 103 is interposed between a first electrode 101 and a second electrode 102.

FIG. 1B illustrates a light-emitting device having a stacked-layer structure (a tandem structure) where a plurality of EL layers (two layers of 103a and 103b in FIG. 1B) are provided between a pair of electrodes and a charge-generation layer 106 is provided between the EL layers. A light-emitting device having a tandem structure can achieve a light-emitting apparatus having high efficiency without changing the amount of current.

The charge-generation layer 106 has a function of injecting electrons to one of the EL layers (103a or 103b) and injecting holes to the other of the EL layers (103b or 103a) when a potential difference is generated between the first electrode 101 and the second electrode 102. Thus, when voltage is applied such that the potential of the first electrode 101 is higher than that of the second electrode 102 in FIG. 1B, electrons are injected to the EL layer 103a and holes are injected to the EL layer 103b from the charge-generation layer 106.

Note that in terms of light extraction efficiency, the charge-generation layer 106 preferably has a light-transmitting property with respect to visible light (specifically, the charge-generation layer 106 preferably has a visible light transmittance of 40% or higher). The charge-generation layer 106 functions even when having lower conductivity than the first electrode 101 and the second electrode 102. The compound of the present application can also be used for a layer where electrons are injected from the charge-generation layer. In that case, it is preferable to use a mixed layer or a stacked-layer structure of a Li compound such as Li metal or lithium oxide and the compound of the present application.

FIG. 1C illustrates a stacked-layer structure of the EL layer 103 in the light-emitting device of one embodiment of the present invention. Note that in this case, the first electrode 101 functions as an anode and the second electrode 102 functions as a cathode. The EL layer 103 has a structure where a hole-injection layer 111, a hole-transport layer 112, a light-emitting layer 113, an electron-transport layer 114, and an electron-injection layer 115 are sequentially stacked over the first electrode 101. Note that the light-emitting layer 113 may have a stacked-layer structure of a plurality of light-emitting layers that emit light of different colors. For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. The structure where a plurality of light-emitting layers that emit light of the same color are stacked can achieve higher reliability than a single-layer structure in some cases. Even in the case where a plurality of EL layers are provided as in the tandem structure illustrated in FIG. 1B, the EL layers are sequentially stacked from the anode side as described above. When the first electrode 101 serves as a cathode and the second electrode 102 serves as an anode, the stacking order in the EL layer 103 is reversed. Specifically, the layer 111 over the first electrode 101 serving as the cathode denotes an electron-injection layer; the layer 112 denotes an electron-transport layer; the layer 113 denotes a light-emitting layer; the layer 114 denotes a hole-transport layer; and the layer 115 denotes a hole-injection layer.

The light-emitting layers 113 included in the EL layers (103, 103a, and 103b) each contain an appropriate combination of a light-emitting substance and a plurality of substances, so that fluorescent light or phosphorescent light of a desired emission color can be obtained. Furthermore, the light-emitting layer 113 may have a stacked-layer structure having different emission colors. In that case, light-emitting substances and other substances are different between the stacked light-emitting layers. Furthermore, a structure where different emission colors can be obtained from the plurality of EL layers (103a and 103b) illustrated in FIG. 1B may be employed. Also in that case, the light-emitting substances and other substances are different between the light-emitting layers.

In addition, the light-emitting device of one embodiment of the present invention can have an optical micro resonator (microcavity) structure with the first electrode 101 being a reflective electrode and the second electrode 102 being a semi-transmissive and semi-reflective electrode illustrated in FIG. 1C, for example, and light emission obtained from the light-emitting layer 113 included in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (a transparent conductive film), optical adjustment can be performed by adjusting the thickness of the transparent conductive film. Specifically, when the wavelength 2 of light obtained from the light-emitting layer 113 is 1, the optical path length between the first electrode 101 and the second electrode 102 (the product of the thickness and the refractive index) is preferably adjusted to be mλ/2 (note that m is an integer greater than or equal to 1) or a neighborhood thereof.

To amplify desired light (wavelength: 2) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (note that m′ is an integer greater than or equal to 1) or a neighborhood thereof. Note that the light-emitting region here refers to a region where holes and electrons are recombined in the light-emitting layer 113.

By performing such optical adjustment, the spectrum of specific monochromatic light obtained from the light-emitting layer 113 can be narrowed and light emission with high color purity can be obtained.

Note that in the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer from which the desired light is obtained is, to be exact, the optical path length between the reflective region in the first electrode 101 and the light-emitting region in the light-emitting layer from which the desired light is obtained. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

The light-emitting device illustrated in FIG. 1D is a light-emitting device having a tandem structure. Owing to a microcavity structure of the light-emitting device, light (monochromatic light) with different wavelengths from the EL layers (103a and 103b) can be extracted. Thus, side-by-side patterning for obtaining different emission colors (e.g., RGB) is not necessary. Accordingly, higher resolution can be easily achieved. In addition, a combination with coloring layers (color filters) is also possible. Furthermore, the emission intensity of light with a specific wavelength in the front direction can be increased, whereby power consumption can be reduced.

A light-emitting device illustrated in FIG. 1E is an example of the light-emitting device having the tandem structure illustrated in FIG. 1B, and includes three EL layers (103a, 103b, and 103c) stacked with charge-generation layers (106a and 106b) therebetween, as illustrated in the drawing. Note that the three EL layers (103a, 103b, and 103c) include respective light-emitting layers (113a, 113b, and 113c) and the emission colors of the light-emitting layers can be combined freely. For example, the light-emitting layer 113a can emit blue light, the light-emitting layer 113b can emit red, green, or yellow light, and the light-emitting layer 113c can emit blue light; for another example, the light-emitting layer 113a can emit red light, the light-emitting layer 113b can emit blue, green, or yellow light, and the light-emitting layer 113c can emit red light. The light-emitting layer 113a, the light-emitting layer 113b, and the light-emitting layer 113c may be fluorescent light-emitting layers or phosphorescent light-emitting layers. Each of the light-emitting layer 113a, the light-emitting layer 113b, and the light-emitting layer 113c may be independently one light-emitting layer or two or more stacked light-emitting layers. For example, in the case where the light-emitting layer 113a, the light-emitting layer 113b, and the light-emitting layer 113c are each two or more stacked light-emitting layers, the light-emitting layer 113a may be a blue-light-emitting layer, the light-emitting layer 113b may be a three-layer stack of red-, yellow-, and green-light-emitting layers, and the light-emitting layer 113c may be a blue-light-emitting layer. For example, in the case where the light-emitting layer 113a, the light-emitting layer 113b, and the light-emitting layer 113c are each two or more stacked light-emitting layers, the light-emitting layer 113a may be a blue-light-emitting layer, the light-emitting layer 113b may be a two-layer stack of red- and yellow-light-emitting layers, and the light-emitting layer 113c may be a blue-light-emitting layer. For example, in the case where the light-emitting layer 113a, the light-emitting layer 113b, and the light-emitting layer 113c are each two or more stacked light-emitting layers, the light-emitting layer 113a may be a blue-light-emitting layer, the light-emitting layer 113b may be a four-layer stack of red-, yellow-, yellow-, and green-light-emitting layers, and the light-emitting layer 113c may be a blue-light-emitting layer. When different colors are stacked by stacking light-emitting layers in this manner, light emission with a high color rendering property can be obtained. Using the compound of the present application for a plurality of EL layers is effective.

In the above light-emitting device of one embodiment of the present invention, at least one of the first electrode 101 and the second electrode 102 is an electrode having a light-transmitting property (a transparent electrode, a semi-transmissive and semi-reflective electrode, or the like). In the case where the electrode having a light-transmitting property is a transparent electrode, the visible light transmittance of the transparent electrode is higher than or equal to 40%. In the case where the electrode having a light-transmitting property is a semi-transmissive and semi-reflective electrode, the visible light reflectance of the semi-transmissive and semi-reflective electrode is higher than or equal to 20% and lower than or equal to 80%, preferably higher than or equal to 40% and lower than or equal to 70%. The resistivity of these electrodes is preferably lower than or equal to 1×10⁻² Ωcm.

In the case where one of the first electrode 101 and the second electrode 102 is an electrode having a reflecting property (a reflective electrode) in the above light-emitting device of one embodiment of the present invention, the visible light reflectance of the electrode having a reflecting property is higher than or equal to 40% and lower than or equal to 100%, preferably higher than or equal to 70% and lower than or equal to 100%. The resistivity of this electrode is preferably lower than or equal to 1×10⁻² Ωcm.

Although FIG. 1 illustrates the example of the light-emitting device having a tandem structure, the compound of one embodiment of the present application can be used for a monochromatic tandem light-emitting device that does not require separate coloring and a light-emitting device that requires separate coloring. The monochromatic tandem light-emitting device can be used for a full-color display when combined with a color filter or a color conversion layer.

### <<Specific Structure of Light-Emitting Device>>

Next, a specific structure of the light-emitting device of one embodiment of the present invention will be described. Here, description is made with reference to FIG. 1D illustrating the tandem structure. Note that the structure of the EL layer applies also to the light-emitting devices having a single structure in FIG. 1A and FIG. 1C. In the case where the light-emitting device illustrated in FIG. 1D has a microcavity structure, the first electrode 101 is formed as a reflective electrode and the second electrode 102 is formed as a semi-transmissive and semi-reflective electrode. Thus, a single-layer structure or a stacked-layer structure can be formed using one or more kinds of desired electrode materials. Note that the second electrode 102 is formed after formation of the EL layer 103b, with the use of a material selected as appropriate.

### <First Electrode and Second Electrode>

As materials for forming the first electrode 101 and the second electrode 102, any of the following materials can be used in an appropriate combination as long as the functions of the both electrodes described above can be fulfilled. For example, a metal, an alloy, an electrically conductive compound, and a mixture of these can be used as appropriate. Specifically, an In—Sn oxide (also referred to as ITO), an In—Si—Sn oxide (also referred to as ITSO), an In—Zn oxide, and an In—W—Zn oxide are given. In addition, it is also possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use an element belonging to Group 1 or Group 2 in the periodic table, which is not listed above as an example (for example, lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device illustrated in FIG. 1D, when the first electrode 101 is an anode, a hole-injection layer 111a and a hole-transport layer 112a of the EL layer 103a are sequentially stacked over the first electrode 101 by a vacuum evaporation method. After the EL layer 103a and the charge-generation layer 106 are formed, a hole-injection layer 111b and a hole-transport layer 112b of the EL layer 103b are sequentially stacked over the charge-generation layer 106 in a similar manner.

### <Hole-Injection Layer>

The hole-injection layers (111, 111a, and 111b) are layers which inject holes from the first electrode 101 serving as the anode and the charge-generation layers (106, 106a, and 106b) to the EL layers (103, 103a, and 103b) and contain an organic acceptor material and a material having a high hole-injection property.

The organic acceptor material in one organic compound allows holes to be generated in another organic compound whose HOMO level is close to the LUMO (lowest unoccupied molecular orbital) level of the organic acceptor material when charge separation is caused between the organic acceptor material and the organic compound. Thus, as the organic acceptor material, a compound having an electron-withdrawing group (a halogen group or a cyano group), such as a quinodimethane derivative, a chloranil derivative, or a hexaazatriphenylene derivative, can be used. For example, it is possible to use 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylen-1,3,4,5,6,8,9,10-octafluoro-7H-pyren-2-ylidene) malononitrile. Note that among organic acceptor materials, a compound in which electron-withdrawing groups are bonded to condensed aromatic rings each having a plurality of heteroatoms, such as HAT-CN, is particularly preferred because it has a high acceptor property and stable film quality against heat. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 in the periodic table (e.g., a transition metal oxide such as a molybdenum oxide, a vanadium oxide, a ruthenium oxide, a tungsten oxide, or a manganese oxide) can be used. As specific examples, a molybdenum oxide, a vanadium oxide, a niobium oxide, a tantalum oxide, a chromium oxide, a tungsten oxide, a manganese oxide, and a rhenium oxide are given. In particular, a molybdenum oxide is preferable because it is stable in the air, has a low hygroscopic property, and is easily handled. It is also possible to use phthalocyanine (abbreviation: H₂Pc), a phthalocyanine-based compound such as copper phthalocyanine (abbreviation: CuPc), or the like.

In addition to the above materials, it is also possible to use an aromatic amine compound, which is a low molecular compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N′-bis {4-[bis(3-methylphenyl)amino]phenyl}-N,N′-diphenylbiphenyl)-4,4′-diamine (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), or 3-[N-(1-naphthyl)-N-(9-phenylcarbazol-3-yl)amino]-9-phenylcarbazole (abbreviation: PCzPCN1).

It is also possible to use a high molecular compound (an oligomer, a dendrimer, a polymer, or the like) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl) methacrylamide] (abbreviation: PTPDMA), or poly[N,N′-bis(4-butylphenyl)-N,N′-bis(phenyl)benzidine] (abbreviation: Poly-TPD). It is also possible to use a high molecular compound to which acid such as poly(3,4-ethylenedioxythiophene)/polystyrenesulfonic acid (abbreviation: PEDOT/PSS) or polyaniline/polystyrenesulfonic acid (abbreviation: PAni/PSS) is added.

As the material having a high hole-injection property, a mixed material containing a hole-transport material and the above-described organic acceptor material (an electron-accepting material) can be used. In that case, the organic acceptor material extracts electrons from the hole-transport material, so that holes are generated in the hole-injection layer 111 and the holes are injected into the light-emitting layer 113 through the hole-transport layer 112. Note that the hole-injection layer 111 may be formed as a single layer of a mixed material containing the hole-transport material and the organic acceptor material (an electron-accepting material), or may be formed by stacking a layer containing the hole-transport material and a layer containing the organic acceptor material (the electron-accepting material).

The hole-transport material is preferably a substance having a hole mobility higher than or equal to 1×10⁻⁶ cm²/Vs in the case where the square root of the electric field strength [V/cm] is 600. Note that any other substance can be used as long as the substance has a hole-transport property higher than an electron-transport property.

As the hole-transport material, a material having a high hole-transport property such as a compound having a π-electron rich heteroaromatic ring (e.g., a carbazole derivative, a furan derivative, or a thiophene derivative) or an aromatic amine (an organic compound having an aromatic amine skeleton), is preferable. The compound in Embodiment 1 has a hole-transport property and thus can be used as a hole-transport material.

Examples of the above carbazole derivative (an organic compound having a carbazole ring) include a bicarbazole derivative (e.g., a 3,3′-bicarbazole derivative) and an aromatic amine having a carbazolyl group.

Specific examples of the above bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: BNCCP).

Specific examples of the aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]-bis(9,9-dimethyl-9H-fluoren-2-yl)amine (abbreviation: PCBFF), N-(biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(biphenyl-4-yl)-N-[4-(9-phenyl-9H-carbazol-3-yl)phenyl]-9,9-diphenyl-9H-fluoren-2-amine, N-(biphenyl-4-yl)-N-[4-(9-phenyl-9H-carbazol-3-yl)phenyl]-9,9-diphenyl-9H-fluoren-4-amine, N-(biphenyl-4-yl)-N-[4-(9-phenyl-9H-carbazol-3-yl)phenyl]-9,9′-spirobi (9H-fluoren)-2-amine, N-(biphenyl-4-yl)-N-[4-(9-phenyl-9H-carbazol-3-yl)phenyl]-9,9′-spirobi (9H-fluoren)-4-amine, N-[4-(9-phenyl-9H-carbazol-3-yl)phenyl]-N-[1,1′: 3′,1″-terphenyl-4-yl]-9,9-dimethyl-9H-fluoren-2-amine, N-[4-(9-phenyl-9H-carbazol-3-yl)phenyl]-N-[1,1′: 4′,1″-terphenyl-4-yl]-9,9-dimethyl-9H-fluoren-2-amine, N-[4-(9-phenyl-9H-carbazol-3-yl)phenyl]-N-[1,1′: 3′,1″-terphenyl-4-yl]-9,9-dimethyl-9H-fluoren-4-amine, N-[4-(9-phenyl-9H-carbazol-3-yl)phenyl]-N-[1,1′: 4′,1″-terphenyl-4-yl]-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N′-bis(9-phenylcarbazol-3-yl)-N,N′-diphenylbenzene-1,3-diamine (abbreviation: PCA2B), N,N′,N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]spiro-9,9′-bifluoren-2-amine (abbreviation: PCBASF), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N′-bis[4-(carbazol-9-yl)phenyl]-N,N′-diphenyl-9,9-dimethylfluorene-2,7-diamine (abbreviation: YGA2F), and 4,4′,4″-tris(carbazol-9-yl)triphenylamine (abbreviation: TCTA).

In addition to the above, other examples of the carbazole derivative include 3-[4-(9-phenanthryl)-phenyl]-9-phenyl-9H-carbazole (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the furan derivative (an organic compound having a furan ring) include 4,4′,4″-(benzene-1,3,5-triyl)tri (dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (the organic compound having a thiophene ring) include 4,4′,4″-(benzene-1,3,5-triyl)tri (dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N′-bis(3-methylphenyl)-N,N′-diphenyl-biphenyl-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]spiro-9,9′-bifluorene (abbreviation: DPASF), 2,7-bis[N-(4-diphenylaminophenyl)-N-phenylamino]-spiro-9,9′-bifluorene (abbreviation: DPA2SF), 4,4′,4″-tris[N-(1-naphthyl)-N-phenylamino]triphenylamine (abbreviation: 1′-TNATA), 4,4′,4″-tris(N,N-diphenylamino)triphenylamine (abbreviation: TDATA), 4,4′,4″-tris[N-(3-methylphenyl)-N-phenylamino]triphenylamine (abbreviation: m-MTDATA), N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), DNTPD, 1,3,5-tris[N-(4-diphenylaminophenyl)-N-phenylamino]benzene (abbreviation: DPA3B), N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNBNB), 4,4′-diphenyl-4″-(7; l′-binaphthyl-2-yl)triphenylamine (abbreviation: BBAαNBNB-03), 4,4′-diphenyl-4″-(7-phenyl) naphthyl-2-yltriphenylamine (abbreviation: BBAPβNB-03), 4,4′-diphenyl-4″-(6;2′-binaphthyl-2-yl)triphenylamine BBA(βN2)B), (abbreviation: 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-(abbreviation: BBAβNαNB-02), 4-(4-biphenylyl)-4′-(2-naphthyl)-4″-yl)triphenylamine phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)-triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF (4)), N-(biphenyl-2-yl)-N-(9,9-dimethyl-9H-fluoren-2-yl)-9,9′-spirobi[9H-fluoren]-4-amine (abbreviation: oFBiSF), N-(4-biphenyl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N, N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Alternatively, it is also possible to use, as the hole-transport material, a high molecular compound (e.g., an oligomer, a dendrimer, or a polymer) such as poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl) methacrylamide] (abbreviation: PTPDMA), poly[N,N′-bis(4-butylphenyl)-N,N′-bis(phenyl)benzidine] (abbreviation: Poly-TPD), or the like. It is also possible to use a high molecular compound to which acid such as poly(3,4-ethylenedioxythiophene)/polystyrenesulfonic acid (abbreviation: PEDOT/PSS) or polyaniline/polystyrenesulfonic acid (abbreviation: PAni/PSS) is added.

Note that the hole-transport material is not limited to the above, and one of or a combination of various known materials may be used as the hole-transport material.

Note that the hole-injection layers (111, 111a, and 111b) can be formed by any of various known film formation methods, and can be formed by a vacuum evaporation method, for example.

### <Hole-Transport Layer>

The hole-transport layers (112, 112a, and 112b) are layers transporting the holes, which are injected from the first electrode 101 by the hole-injection layers (111, 111a, and 111b), to the light-emitting layers (113, 113a, and 113b). Note that the hole-transport layers (112, 112a, and 112b) are each a layer containing a hole-transport material. Thus, for the hole-transport layers (112, 112a, and 112b), a hole-transport material that can be used for the hole-injection layers (111, 111a, and 111b) can be used.

Note that in the light-emitting device of one embodiment of the present invention, the organic compound used for the hole-transport layers (112, 112a, and 112b) can also be used for the light-emitting layers (113, 113a, 113b, and 113c). The use of the same organic compound for the hole-transport layers (112, 112a, and 112b) and the light-emitting layers (113, 113a, 113b, and 113c) is preferable, in which case holes can be efficiently transported from the hole-transport layers (112, 112a, and 112b) to the light-emitting layers (113, 113a, 113b, and 113c).

### <Light-Emitting Layer>

The light-emitting layers (113, 113a, 113b, and 113c) are each a layer containing a light-emitting substance. Note that as a light-emitting substance that can be used for the light-emitting layers (113, 113a, 113b, and 113c), a substance whose emission color is blue, violet, bluish violet, green, yellowish green, yellow, orange, red, or the like can be used as appropriate. In the case where a plurality of light-emitting layers are provided, different light-emitting substances are used for the light-emitting layers; thus, different emission colors can be exhibited (for example, complementary emission colors are combined to obtain white light emission). Furthermore, one light-emitting layer may have a stacked-layer structure containing different light-emitting substances.

The light-emitting layers (113, 113a, 113b, and 113c) may each contain one or more kinds of organic compounds (e.g., a host material) in addition to a light-emitting substance (a guest material).

In the case where a plurality of host materials are used in the light-emitting layers (113, 113a, 113b, and 113c), a second host material that is additionally used is preferably a substance having a larger energy gap than a known guest material and a first host material. Preferably, the lowest singlet excitation energy level (S1 level) of the second host material is higher than the S1 level of the first host material, and the lowest triplet excitation energy level (T1 level) of the second host material is higher than the T1 level of the guest material. Furthermore, the lowest triplet excitation energy level (T1 level) of the second host material is preferably higher than the T1 level of the first host material. With such a structure, an exciplex can be formed by the two kinds of host materials. To form an exciplex efficiently, it is particularly preferable to combine a compound that easily accepts holes (a hole-transport material) and a compound that easily accepts electrons (an electron-transport material). With this structure, high efficiency, low voltage, and a long lifetime can be achieved at the same time.

As an organic compound used as the above host material (including the first host material and the second host material), organic compounds such as the hole-transport materials that can be used for the hole-transport layers (112, 112a, and 112b) described above and electron-transport materials that can be used for electron-transport layers (114, 114a, and 114b) described later can be used as long as they satisfy requirements for the host material used in the light-emitting layer.

Another example is an exciplex formed by a plurality of kinds of organic compounds (the first host material and the second host material). An exciplex (also referred to as Exciplex) whose excited state is formed by a plurality of kinds of organic compounds has an extremely small difference between the S1 level and the T1 level and functions as a TADF material capable of converting triplet excitation energy into singlet excitation energy. As a combination of the plurality of kinds of organic compounds forming an exciplex, for example, it is preferable that one have a π-electron deficient heteroaromatic ring and the other have a π-electron rich heteroaromatic ring. A phosphorescent substance such as an iridium-, rhodium-, or platinum-based organometallic complex or a metal complex may be used as one of the combination forming an exciplex. The organic compound described in Embodiment 1 has an electron-transport property and thus can be efficiently used as the first host material. Furthermore, the organic compound has a hole-transport property, and thus can be used as the second host material.

There is no particular limitation on the light-emitting substances that can be used for the light-emitting layers (113, 113a, 113b, and 113c), and a light-emitting substance that converts singlet excitation energy into light in the visible light range or a light-emitting substance that converts triplet excitation energy into light in the visible light range can be used.

<<Light-Emitting Substance that Converts Singlet Excitation Energy into Light>>

The following substances emitting fluorescent light (fluorescent substances) are given as the light-emitting substance that can be used for the light-emitting layers (113, 113a, 113b, and 113c) and convert singlet excitation energy into light emission. The examples include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative. A pyrene derivative is particularly preferable because it has a high emission quantum yield. Specific examples of the pyrene derivative include N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-diphenyl-N,N′-bis[4-(9-phenyl-9H-fluoren-9-yl)phenyl]pyrene-1,6-diamine (abbreviation: 1,6FLPAPrn), N,N′-bis(dibenzofuran-2-yl)-N,N′-diphenylpyrene-1,6-diamine (abbreviation: 1,6FrAPrn), N,N′-bis(dibenzothiophen-2-yl)-N,N′-diphenylpyrene-1,6-diamine (abbreviation: 1,6ThAPrn), N,N′-(pyrene-1,6-diyl)bis[(N-phenylbenzo[b]naphtho[1,2-d]furan)-6-amine] (abbreviation: 1,6BnfAPrn), N,N′-(pyrene-1,6-diyl)bis[(N-phenylbenzo[b]naphtho[1,2-d]furan)-8-amine] (abbreviation: 1,6BnfAPrn-02), and N,N′-(pyrene-1,6-diyl)bis[(6,N-diphenylbenzo[b]naphtho[1,2-d]furan)-8-amine] (abbreviation: 1,6BnfAPrn-03).

In addition, it is possible to use 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), or the like.

It is also possible to use, for example, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene) propanedinitrile (abbreviation: DCM1), 2-{2-methyl-6-[2-(2,3,6,7-tetrahydro-1H,5H-benzo[ij]quinolizin-9-yl) ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl) ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl) ethenyl]-4H-pyran-4-ylidene}propanedinitrile (abbreviation: DCJTB), 2-(2,6-bis {2-[4-(dimethylamino)phenyl]ethenyl}-4H-pyran-4-ylidene) propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl) ethenyl]-4H-pyran-4-ylidene}propanedinitrile (abbreviation: BisDCJTM), 1,6BnfAPrn-03, 3,10-bis[N-(9-phenyl-9H-carbazol-2-yl)-N-phenylamino]naphtho[2,3-b;6,7-b′]bisbenzofuran (abbreviation: 3,10PCA2Nbf (IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). In particular, pyrenediamine compounds such as 1,6FLPAPrn, 1,6mMemFLPAPrn, and 1,6BnfAPrn-03 can be used, for example.

<Light-Emitting Substance that Converts Triplet Excitation Energy into Light Emission>>

Next, as examples of the light-emitting substance that converts triplet excitation energy into light emission and can be used for the light-emitting layer 113, a substance that emits phosphorescent light (a phosphorescent substance) and a thermally activated delayed fluorescent (TADF) material that exhibits thermally activated delayed fluorescence are given.

A phosphorescent substance refers to a compound that exhibits phosphorescence but does not exhibit fluorescence at a temperature higher than or equal to low temperatures (e.g., 77 K) and lower than or equal to room temperature (i.e., higher than or equal to 77 K and lower than or equal to 313 K). The phosphorescent substance preferably contains a metal element with large spin-orbit interaction, and can be an organometallic complex, a metal complex (a platinum complex), a rare earth metal complex, or the like. Specifically, a transition metal element is preferable and it is particularly preferable that a platinum group element (ruthenium (Ru), rhodium (Rh), palladium (Pd), osmium (Os), iridium (Ir), or platinum (Pt)), especially iridium, be contained, in which case the transition probability relating to direct transition between the singlet ground state and the triplet excited state can be increased.

<<Phosphorescent Substance (from 450 nm to 570 nm: Blue or Green)>>

As a phosphorescent substance that exhibits blue or green and whose emission spectrum has a peak wavelength at greater than or equal to 450 nm and less than or equal to 570 nm, the following substances are given.

The examples include: organometallic complexes having a 4H-triazole ring, such as tris {2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and organometallic complexes in which a ligand is a phenylpyridine derivative having an electron-withdrawing group such as bis[2-(4′,6′-difluorophenyl)pyridinato-N,C²′]iridium(III) tetrakis(1-pyrazolyl) borate (abbreviation: FIr6), bis[2-(4′,6′-difluorophenyl)pyridinato-N,C²′]iridium(III) picolinate (abbreviation: FIrpic), bis {2-[3′,5′-bis(trifluoromethyl)phenyl]pyridinato-N,C²}iridium(III) picolinate (abbreviation: [Ir(CF₃ppy)₂(pic)]), and bis[2-(4′,6′-difluorophenyl)pyridinato-N,C²′]iridium(III) acetylacetonate (abbreviation: FIr(acac)).

<<Phosphorescent Substance (from 495 nm to 590 nm: Green or Yellow)>>

As a phosphorescent substance that exhibits green or yellow and whose emission spectrum has a peak wavelength at greater than or equal to 495 nm and less than or equal to 590 nm, the following substances are given.

Examples include organometallic iridium complexes including a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis {4,6-dimethyl-2-[6-(2,6-dimethylphenyl)-4-pyrimidinyl-κN3]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes including a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes including a pyridine ring, such as tris(2-phenylpyridinato-N,C²′)iridium(III) (abbreviation: [Ir(ppy)₃]), bis(2-phenylpyridinato-N,C²′)iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C²′)iridium(III) (abbreviation: [Ir(pq)₃]), bis(2-phenylquinolinato-N,C²′)iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d3-methyl-8-(2-pyridinyl-κN)benzofuro[2,3-b]pyridine-κC]bis[2-(5-d3-methyl-2-pyridinyl-κN²)phenyl-κC]iridium(III) (abbreviation: Ir(5mppy-d3)₂(mbfpypy-d3)), [2-(methyl-d3)-8-[4-(1-methylethyl-1-d)-2-pyridinyl-κN]benzofuro[2,3-b]pyridin-7-yl-κC]bis[5-(methyl-d3)-2-[5-(methyl-d3)-2-pyridinyl-κN]phenyl-κC]iridium(III) (abbreviation: Ir(5mtpy-d₆)₂(mbfpypy-iPr-d₄)), [2-d3-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d3)), and [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)); organometallic complexes such as bis(2,4-diphenyl-1,3-oxazolato-N,C²′)iridium(III) acetylacetonate (abbreviation: [Ir(dpo)₂(acac)]), bis {2-[4′-(perfluorophenyl)phenyl]pyridinato-N,C²′}iridium(III) acetylacetonate (abbreviation: [Ir(p-PF-ph)₂(acac)]), and bis(2-phenylbenzothiazolato-N,C²′)iridium(III) acetylacetonate (abbreviation: [Ir(bt)₂(acac)]); and a rare earth metal complex such as tris(acetylacetonato) (monophenanthroline) terbium(III) (abbreviation: [Tb(acac)₃(Phen)]).

<<Phosphorescent Substance (from 570 nm to 750 nm: Yellow or Red)>>

As a phosphorescent material that exhibits yellow or red and whose emission spectrum has a peak wavelength at greater than or equal to 570 nm and less than or equal to 750 nm, the following substances are given.

The examples include organometallic complexes including a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes including a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis {4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis {4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis[2-(5-(2,6-dimethylphenyl)-3-(3,5-dimethylphenyl)-2-pyrazinyl-κN)-4,6-dimethylphenyl-κC](2,2′,6,6′-tetramethyl-3,5-heptadionato-κ²O,O′)iridium(III) (abbreviation: [Ir(dmdppr-dmp)₂(dpm)]), (acetylacetonato)bis[2-methyl-3-phenylquinoxalinato-N,C²′]iridium(III) (abbreviation: [Ir(mpq)₂(acac)]), (acetylacetonato)bis(2,3-diphenylquinoxalinato-N,C²′)iridium(III) (abbreviation: [Ir(dpq)₂(acac)]), and (acetylacetonato)bis[2,3-bis(4-fluorophenyl) quinoxalinato]iridium(III) (abbreviation: [Ir(Fdpq)₂(acac)]); organometallic complexes including a pyridine ring, such as tris(1-phenylisoquinolinato-N,C²′)iridium(III) (abbreviation: [Ir(piq)₃]), bis(1-phenylisoquinolinato-N,C²)iridium(III) acetylacetonate (abbreviation: [Ir(piq)₂(acac)]), and bis[4,6-dimethyl-2-(2-quinolinyl-κN)phenyl-κC](2,4-pentanedionato-κ²O,O′)iridium(III) (abbreviation: [Ir(dmpqn)₂(acac)]); platinum complexes such as 2,3,7,8,12,13,17,18-octaethyl-21H,23H-porphyrinplatinum(II) (abbreviation: [PtOEP]); and rare earth metal complexes such as tris(1,3-diphenyl-1,3-propanedionato) (monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]).

### <<TADF Material>>

Any of materials shown below can be used as the TADF material. The TADF material refers to a material that has a small difference (preferably, less than or equal to 0.2 eV) between the S1 level and the T1 level, can up-convert a triplet excited state into a singlet excited state (reverse intersystem crossing) using a little thermal energy, and efficiently exhibits light emission (fluorescence) from the singlet excited state. The thermally activated delayed fluorescence is efficiently obtained under the condition where the difference in energy between the triplet excited energy level and the singlet excited energy level is greater than or equal to 0 eV and less than or equal to 0.2 eV, preferably greater than or equal to 0 eV and less than or equal to 0.1 eV. Delayed fluorescence by the TADF material refers to light emission having a spectrum similar to that of normal fluorescence and an extremely long lifetime. The lifetime is longer than or equal to 1×10⁻⁶ seconds, preferably longer than or equal to 1×10⁻³ seconds. The organic compounds described in Embodiment 1 can also be used.

Note that the TADF material can be also used as an electron-transport material, a hole-transport material, or a host material.

Examples of the TADF material include fullerene, a derivative thereof, an acridine derivative such as proflavine, and eosin. Other examples include a metal-containing porphyrin such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (abbreviation: SnF₂(Proto IX)), a mesoporphyrin-tin fluoride complex (abbreviation: SnF₂(Meso IX)), a hematoporphyrin-tin fluoride complex (abbreviation: SnF₂(Hemato IX)), a coproporphyrin tetramethyl ester-tin fluoride complex (abbreviation: SnF₂(Copro III-4Me)), an octaethylporphyrin-tin fluoride complex (abbreviation: SnF₂(OEP)), an etioporphyrin-tin fluoride complex (abbreviation: SnF₂(Etio I)), and an octaethylporphyrin-platinum chloride complex (abbreviation: PtCl₂OEP).

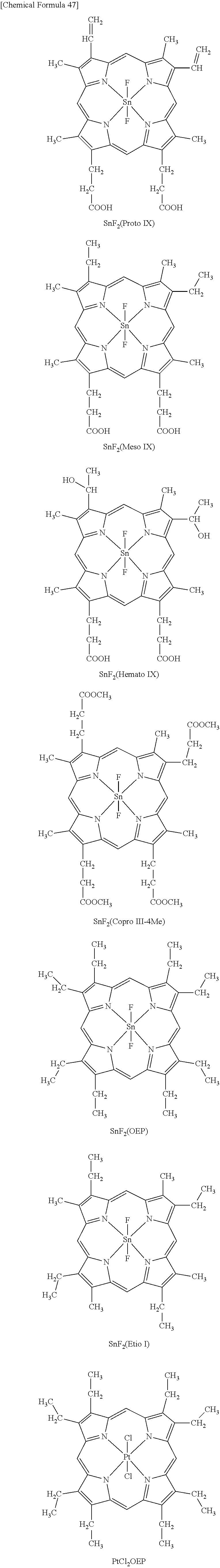

Alternatively, a heteroaromatic compound having a π-electron rich heteroaromatic compound and a π-electron deficient heteroaromatic compound, such as 2-(biphenyl-4-yl)-4,6-bis(12-phenylindolo[2,3-a]carbazol-11-yl)-1,3,5-triazine (abbreviation: PIC-TRZ), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), 10-phenyl-10H,10′H-spiro[acridin-9,9′-anthracen]-10′-one (abbreviation: ACRSA), 4-(9′-phenyl-3,3′-bi-9H-carbazol-9-yl)benzofuro[3,2-d]pyrimidine (abbreviation: 4PCCzBfpm), 4-[4-(9′-phenyl-3,3′-bi-9H-carbazol-9-yl)phenyl]benzofuro[3,2-d]pyrimidine (abbreviation: 4PCCzPBfpm), and 9-[3-(4,6-diphenyl-1,3,5-triazin-2-yl)phenyl]-9′-phenyl-2,3′-bi-9H-carbazole (abbreviation: mPCCzPTzn-02), may be used.

Note that a substance in which a π-electron rich heteroaromatic compound is directly bonded to a π-electron deficient heteroaromatic compound is particularly preferable because both the donor property of the π-electron rich heteroaromatic compound and the acceptor property of the π-electron deficient heteroaromatic compound are improved and the energy difference between the singlet excited state and the triplet excited state becomes small. As the TADF material, a TADF material (TADF 100) in which a singlet excited state and a triplet excited state are in a thermal equilibrium state may be used. Since such a TADF material enables a short emission lifetime (excitation lifetime), an efficiency decrease of a light-emitting device in a high-luminance region can be inhibited.

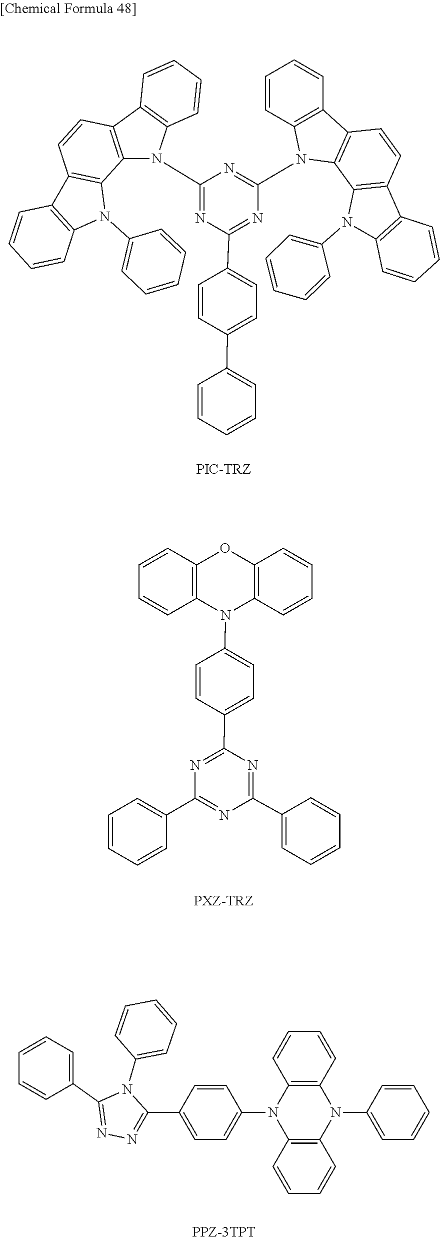 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1]

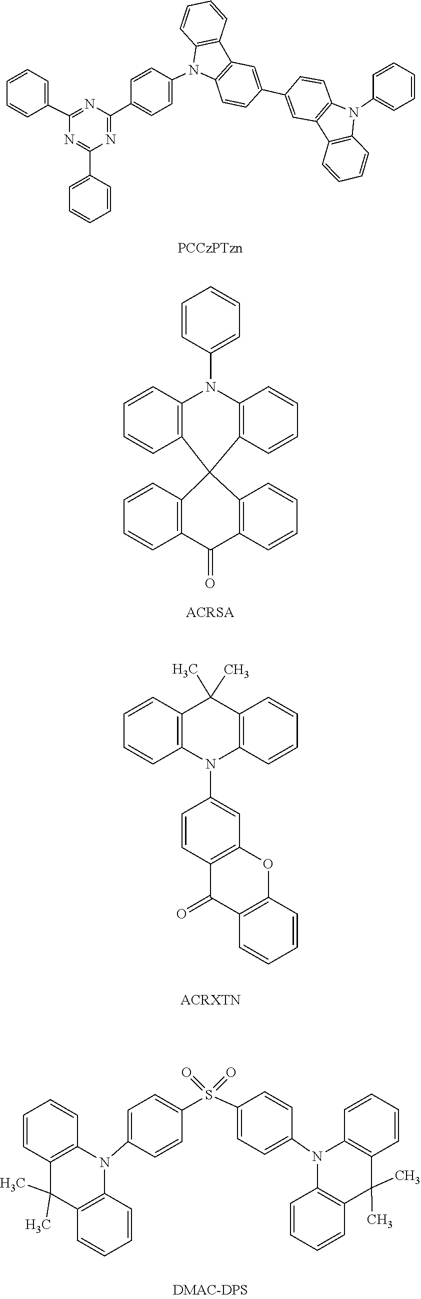

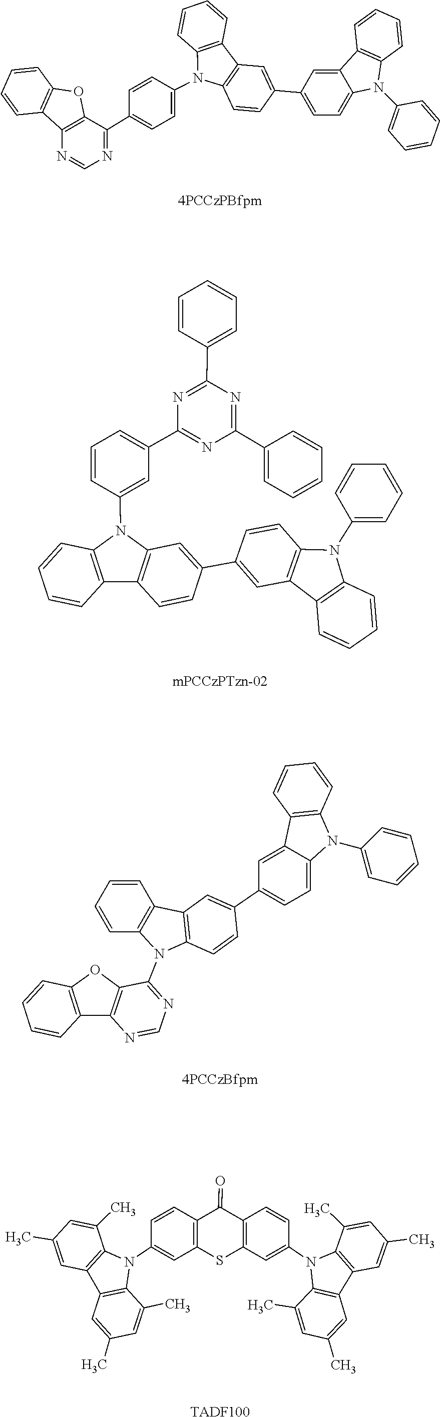

As the material having a function of converting triplet excitation energy into light emission, a nanostructure of a transition metal compound having a perovskite structure is also given in addition to the above. In particular, a nanostructure of a metal-halide perovskite material is preferable. The nanostructure is preferably a nanoparticle or a nanorod.

As the organic compounds (the host material and the like) used in combination with the above-described light-emitting substance (the guest material) in the light-emitting layers (113, 113a, 113b, and 113c), one or more kinds of substances having a larger energy gap than the light-emitting substance (the guest material) may be selected to be used.

### <<Host Material for Fluorescent Light Emission>>

In the case where the light-emitting substance used in the light-emitting layers (113, 113a, 113b, and 113c) is a fluorescent substance, an organic compound (a host material) used in combination with the light-emitting substance is preferably an organic compound having a high energy level in a singlet excited state and a low energy level in a triplet excited state or an organic compound having a high fluorescence quantum yield. Therefore, the hole-transport material (described above) or the electron-transport material (described below) shown in this embodiment, for example, can be used as long as they are organic compounds that satisfy such a condition. The organic compounds described in Embodiment 1 can also be used.

In terms of a preferable combination with the light-emitting substance (the fluorescent substance), examples of the organic compound (the host material) include condensed polycyclic aromatic compounds such as an anthracene derivative, a tetracene derivative, a phenanthrene derivative, a pyrene derivative, a chrysene derivative, and a dibenzo[g,p]chrysene derivative, although some of them overlap with the above specific examples.

Note that specific examples of the organic compound (the host material) preferably used in combination with the fluorescent substance include 9-phenyl-3-[4-(10-phenyl-9-anthryl)phenyl]-9H-carbazole (abbreviation: PCzPA), 3,6-diphenyl-9-[4-(10-phenyl-9-anthryl)phenyl]-9H-carbazole (abbreviation: DPCzPA), 3-[4-(1-naphthyl)-phenyl]-9-phenyl-9H-carbazole (abbreviation: PCPN), 9,10-diphenylanthracene (abbreviation: DPAnth), N,N-diphenyl-9-[4-(10-phenyl-9-anthryl)phenyl]-9H-carbazol-3-amine (abbreviation: CzA1PA), 4-(10-phenyl-9-anthryl)triphenylamine (abbreviation: DPhPA), YGAPA, PCAPA, N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 6,12-dimethoxy-5,11-diphenylchrysene, N,N,N′,N′,N″,N″,N′″,N′″-octaphenyldibenzo[g,p]chrysene-2,7,10,15-tetraamine (abbreviation: DBC1), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)-biphenyl-4′-yl}-anthracene (abbreviation: FLPPA), 9,10-bis(3,5-diphenylphenyl) anthracene (abbreviation: DPPA), 9,10-di(2-naphthyl) anthracene (abbreviation: DNA), 2-tert-butyl-9,10-di(2-naphthyl) anthracene (abbreviation: t-BuDNA), 9-(1-naphthyl)-10-(2-naphthyl) anthracene (abbreviation: α,β-ADN), 2-(10-phenylanthracen-9-yl)dibenzofuran, 2-(10-phenyl-9-anthracenyl)-benzo[b]naphtho[2,3-d]furan (abbreviation: Bnf(II)PhA), 9-(1-naphthyl)-10-[4-(2-naphthyl)phenyl]anthracene (abbreviation: αN-βNP Anth), 2,9-di(1-naphthyl)-10-phenylanthracene (abbreviation: 2αN-αNPhA), 9-(1-naphthyl)-10-[3-(1-naphthyl)phenyl]anthracene (abbreviation: αN-mαNPAnth), 9-(2-naphthyl)-10-[3-(1-naphthyl)phenyl]anthracene (abbreviation: βN-mαNPAnth), 9-(1-naphthyl)-10-[4-(1-naphthyl)phenyl]anthracene (abbreviation: αN-αNPAnth), 9-(2-naphthyl)-10-[4-(2-naphthyl)phenyl]anthracene (abbreviation: βN-βNPAnth), 2-(1-naphthyl)-9-(2-naphthyl)-(10-phanylanthracene (abbreviation: 2αN-βNPhA), 9-(2-naphthyl)-10-[3-(2-naphthyl)phenyl]anthracene (abbreviation: βN-mβNPAnth), 1-[4-(10-(biphenyl-4-yl)-9-anthracenyl)phenyl]-2-ethyl-1H-benzimidazole (abbreviation: EtBImPBPhA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 1,3,5-tri (1-pyrenyl)benzene (abbreviation: TPB3), 5,12-diphenyltetracene, and 5,12-bis(biphenyl-2-yl)tetracene.

### <<Host Material for Phosphorescent Light Emission>>

In the case where the light-emitting substance used for the light-emitting layers (113, 113a, 113b, and 113c) is a phosphorescent substance, an organic compound having triplet excitation energy (energy difference between a ground state and a triplet excited state) which is higher than triplet excitation energy of the light-emitting substance is preferably selected as the organic compound (the host material) used in combination with the light-emitting substance. Note that in the case where a plurality of organic compounds (e.g., the first host material and the second host material (or an assist material)) are used in combination with a light-emitting substance in order to form an exciplex, the plurality of organic compounds are preferably mixed with a phosphorescent substance. The organic compounds described in Embodiment 1 can also be used.

Such a structure makes it possible to efficiently obtain light emission utilizing ExTET (Exciplex-Triplet Energy Transfer), which is energy transfer from an exciplex to a light-emitting substance. Note that a combination of the plurality of organic compounds that easily forms an exciplex is preferably employed, and it is particularly preferable to combine a compound that easily accepts holes (a hole-transport material) and a compound that easily accepts electrons (an electron-transport material).

In terms of a preferable combination with the light-emitting substance (phosphorescent substance), examples of the organic compounds (the host material and the assist material) include an aromatic amine (an organic compound having an aromatic amine skeleton), a carbazole derivative (an organic compound having a carbazole ring), a dibenzothiophene derivative (an organic compound having a dibenzothiophene ring), a dibenzofuran derivative (an organic compound having a dibenzofuran ring), an oxadiazole derivative (an organic compound having an oxadiazole ring), a triazole derivative (an organic compound having a triazole ring), a benzimidazole derivative (an organic compound having a benzimidazole ring), a quinoxaline derivative (an organic compound having a quinoxaline ring), a dibenzoquinoxaline derivative (an organic compound having a dibenzoquinoxaline ring), a pyrimidine derivative (an organic compound having a pyrimidine ring), a triazine derivative (an organic compound having a triazine ring), a pyridine derivative (an organic compound having a pyridine ring), a bipyridine derivative (an organic compound having a bipyridine ring), a phenanthroline derivative (an organic compound having a phenanthroline ring), a furodiazine derivative (an organic compound having a furodiazine ring), and zinc- and aluminum-based metal complexes, although some of them overlap with the above specific examples.

Among the above organic compounds, specific examples of the aromatic amine and the carbazole derivative, which are organic compounds having a high hole-transport property, are the same as the specific examples of the hole-transport materials described above. Any of these is preferable as the host material.

Among the above organic compounds, specific examples of the dibenzothiophene derivative and the dibenzofuran derivative, which are organic compounds having a high hole-transport property, include 4-{3-[3-(9-phenyl-9H-fluoren-9-yl)phenyl]phenyl}dibenzofuran (abbreviation: mmDBFFLBi-II), 4,4′,4″-(benzene-1,3,5-triyl)tri (dibenzofuran) (abbreviation: DBF3P-II), DBT3P-II, 2,8-diphenyl-4-[4-(9-phenyl-9H-fluoren-9-yl)phenyl]dibenzothiophene (abbreviation: DBTFLP-III), 4-[4-(9-phenyl-9H-fluoren-9-yl)phenyl]-6-phenyldibenzothiophene (abbreviation: DBTFLP-IV), and 4-[3-(triphenylen-2-yl)phenyl]dibenzothiophene (abbreviation: mDBTPTp-II). Any of these is preferable as the host material.

In addition, a metal complex having an oxazole-based or thiazole-based ligand, such as bis[2-(2-benzoxazolyl)phenolato]zinc(II) (abbreviation: ZnPBO) or bis[2-(2-benzothiazolyl)phenolato]zinc(II) (abbreviation: ZnBTZ), or the like is given as a preferable example of the host material.

Among the above organic compounds, specific examples of the oxadiazole derivative, the triazole derivative, the benzimidazole derivative, the quinoxaline derivative, the dibenzoquinoxaline derivative, the quinazoline derivative, the phenanthroline derivative, and the like, which are organic compounds having a high electron-transport property, include an organic compound including a heteroaromatic ring having a polyazole ring, such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl) stilbene (abbreviation: BzOs); an organic compound including a heteroaromatic ring having a pyridine ring, such as bathophenanthroline (abbreviation: Bphen), bathocuproine (abbreviation: BCP), 2,9-di(naphthalen-2-yl)-4,7-diphenyl-1,10-phenanthroline (abbreviation: NBphen), 2,2′-(1,3-phenylene)bis(9-phenyl-1,10-phenanthroline) (abbreviation: mPPhen2P), and 2,2′-(1,1′-biphenyl)-4,4′-diylbis(9-phenyl-1,10-phenanthroline) (abbreviation: PPhen2BP); 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 2-{4-[9,10-di(2-naphthyl)-2-anthryl]phenyl}-1-phenyl-1H-benzimidazole (abbreviation: ZADN), and 2-[4′-(9-phenyl-9H-carbazol-3-yl)-3,1′-biphenyl-1-yl]dibenzo[f,h]quinoxaline (abbreviation: 2mpPCBPDBq). Any of these is preferable as the host material.

Among the above organic compounds, specific examples of the pyridine derivative, the diazine derivative (including the pyrimidine derivative, the pyrazine derivative, and the pyridazine derivative), the triazine derivative, and the furodiazine derivative, which are organic compounds having a high electron-transport property, include organic compounds including a heteroaromatic ring having a diazine ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 3,5-bis[3-(9H-carbazol-9-yl)phenyl]pyridine (abbreviation: 35DCzPPy), 1,3,5-tri[3-(3-pyridyl)phenyl]benzene (abbreviation: TmPyPB), 9,9′-[pyrimidine-4,6-diylbis(biphenyl-3,3′-diyl)]bis(9H-carbazole) (abbreviation: 4,6mCzBP2Pm), 2-[3′-(9,9-dimethyl-9H-fluoren-2-yl)biphenyl-3-yl]-4,6-diphenyl-1,3,5-triazine (abbreviation: mFBPTzn), 8-(biphenyl-4-yl)-4-[3-(dibenzothiophen-4-yl)phenyl]-[1]benzofuro[3,2-d]pyrimidine (abbreviation: 8BP-4mDBtPBfpm), 9-[3′-(dibenzothiophen-4-yl)biphenyl-3-yl]naphtho[1′,2′: 4,5]furo[2,3-b]pyrazine (abbreviation: 9mDBtBPNfpr), 9-[3′-(dibenzothiophen-4-yl)biphenyl-4-yl]naphtho[1′,2′: 4,5]furo[2,3-b]pyrazine (abbreviation: 9pmDBtBPNfpr), 11-[3′-dibenzothiophen-4-yl)bipheny-3-yl]phenanthro[9′,10′: 4,5]furo[2,3-b]pyrazine (abbreviation: 11mDBtBPPnfpr), 11-[3′-(dibenzothiophen-4-yl)biphenyl-4-yl]phenanthro[9′,10′: 4,5]furo[2,3-b]pyrazine, 11-[(3′-(9H-carbazol-9-yl)biphenyl-3-yl]phenanthro[9′,10′: 4,5]furo[2,3-b]pyrazine, 12-(9′-phenyl-3,3′-bi-9H-carbazol-9-yl) phenanthro[9′,10′: 4,5]furo[2,3-b]pyrazine (abbreviation: 12PCCzPnfpr), 9-[(3′-9-phenyl-9H-carbazol-3-yl)biphenyl-4-yl]naphtho[1′,2′: 4,5]furo[2,3-b]pyrazine (abbreviation: 9pmPCBPNfpr), 9-(9′-phenyl-3,3′-bi-9H-carbazol-9-yl) naphtho[1′,2′: 4,5]furo[2,3-b]pyrazine (abbreviation: 9PCCzNfpr), 10-(9′-phenyl-3,3′-bi-9H-carbazol-9-yl) naphtho[1′,2′: 4,5]furo[2,3-b]pyrazine (abbreviation: 10PCCzNfpr), 9-[3′-(6-phenylbenzo[b]naphtho[1,2-d]furan-8-yl)biphenyl-3-yl]naphtho[1′,2′: 4,5]furo[2,3-b]pyrazine (abbreviation: 9mBnfBPNfpr), 9-{3-[6-(9,9-dimethylfluoren-2-yl)dibenzothiophen-4-yl]phenyl}naphtho[1′,2′: 4,5]furo[2,3-b]pyrazine (abbreviation: 9mFDBtPNfpr), 9-[3′-(6-phenyldibenzothiophen-4-yl)biphenyl-3-yl]naphtho[1′,2′: 4,5]furo[2,3-b]pyrazine (abbreviation: 9mDBtBPNfpr-02), 9-[3-(9′-phenyl-3,3′-bi-9H-carbazol-9-yl)phenyl]naphtho[1′,2′: 4,5]furo[2,3-b]pyrazine (abbreviation: 9mPCCzPNfpr), 9-{(3′-[2,8-diphenyldibenzothiophen-4-yl)biphenyl-3-yl}naphtho[1′,2′: 4,5]furo[2,3-b]pyrazine, 11-{(3′-[2,8-diphenyldibenzothiophen-4-yl)biphenyl-3-yl]phenanthro[9′,10′: 4,5]furo[2,3-b]pyrazine, 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 2-(biphenyl-4-yl)-4-phenyl-6-[9,9′-spirobi (9H-fluoren)-2-yl]-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 9-[4-(4,6-diphenyl-1,3,5-triazin-2-yl)-2-dibenzothienyl]-2-phenyl-9H-calbazole (abbreviation: PCzDBfTzn), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-(8-[(1, l′: 4′,1″-terphenyl)-4-yl-1-dibenzofuranyl]-1,3,5-triazine (abbreviation: mBP-TPDBfTzn), 6-(biphenyl-3-yl)-4-[3,5-bis(9H-carbazol-9-yl)phenyl]-2-phenylpyrimidine (abbreviation: 6mBP-4Cz2PPm), and 4-[3,5-bis(9H-carbazol-9-yl)phenyl]-2-phenyl-6-(biphenyl-4-yl)pyrimidine (abbreviation: 6BP-4Cz2PPm). Any of these are preferable as the host material.

Among the above organic compounds, specific examples of metal complexes that are organic compounds having a high electron-transport property include zinc- and aluminum-based metal complexes, such as tris(8-quinolinolato)aluminum(III) (abbreviation: Alq), tris(4-methyl-8-quinolinolato)aluminum(III) (abbreviation: Almq₃), bis(10-hydroxybenzo[h]quinolinato) beryllium(II) (abbreviation: BeBq₂), bis(2-methyl-8-quinolinolato)(4-phenylphenolato)aluminum(III) (abbreviation: BAlq), and bis(8-quinolinolato)zinc(II) (abbreviation: Znq), and metal complexes having a quinoline ring or a benzoquinoline ring. Any of these are preferable as the host material.

In addition, a high molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy) is also preferable as the host material.

It is also possible to use an organic compound having a bipolar property, i.e., both a high hole-transport property and a high electron-transport property, and including a diazine ring, such as 9-phenyl-9′-(4-phenyl-2-quinazolinyl)-3,3′-bi-9H-carbazole (abbreviation: PCCzQz), 2-[4′-(9-phenyl-9H-carbazol-3-yl)-3,1′-biphenyl-1-yl]dibenzo[f,h]quinoxaline (abbreviation: 2mpPCBPDBq), 5-[3-(4,6-diphenyl-1,3,5-triazin-2yl)phenyl]-7,7-dimethyl-5H,7H-indeno[2,1-b]carbazole (abbreviation: mINc(II)PTzn), 11-[4-(biphenyl-4-yl)-6-phenyl-1,3,5-triazin-2-yl]-11,12-dihydro-12-phenyl-indolo[2,3-a]carbazole (abbreviation: BP-Icz(II)Tzn), or 7-[4-(9-phenyl-9H-carbazol-2-yl) quinazolin-2-yl]-7H-dibenzo[c,g]carbazole (abbreviation: PC-cgDBCzQz) can be used as the host material.

### <Electron-Transport Layer>

The electron-transport layers (114, 114a, and 114b) are layers transporting the electrons, which are injected from the second electrode 102 and the charge-generation layers (106, 106a, and 106b) by electron-injection layers (115, 115a, and 115b) described later, to the light-emitting layers (113, 113a, 113b, and 113c). The light-emitting device of one embodiment of the present invention can have improved heat resistance when the electron-transport layer has a stacked-layer structure. The electron-transport materials used for the electron-transport layers (114, 114a, and 114b) are preferably substances with an electron mobility higher than or equal to 1×10⁻⁶ cm²/Vs in the case where the square root of the electric field strength [V/cm] is 600. Note that any other substance can be used as long as the substance has an electron-transport property higher than a hole-transport property. Each of the electron-transport layers (114, 114a, and 114b) functions even in the form of a single layer but may have a stacked-layer structure of two or more layers. Note that since the above-described mixed material has heat resistance, performing a photolithography step over the electron-transport layer including such a material can inhibit the influence of a thermal process on the device characteristics.

### <<Electron-Transport Material>>

As the electron-transport material that can be used for the electron-transport layers (114, 114a, and 114b), an organic compound with a high electron-transport property can be used; for example, a heteroaromatic compound can be used. Note that the heteroaromatic compound refers to a cyclic compound containing at least two different kinds of elements in a ring. Examples of a cyclic structure include a three-membered ring, a four-membered ring, a five-membered ring, a six-membered ring, and the like, among which a five-membered ring and a six-membered ring are particularly preferable; the elements contained in the heteroaromatic compound are preferably one or more of nitrogen, oxygen, sulfur, and the like, as well as carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used. The compound in Embodiment 1 has an electron-transport property and thus can be used as an electron-transport material. It is further preferable that the compound of the present application be used for both the electron-transport layer 114a and the electron-transport layer 114b.

Note that a material different from a material used for the light-emitting layer can be used as the electron-transport material. Not all excitons formed by recombination of carriers in the light-emitting layer can contribute to light emission and some excitons are diffused into a layer in contact with or in the vicinity of the light-emitting layer. In order to avoid this phenomenon, the energy level (the lowest singlet excitation energy level or the lowest triplet excitation energy level) of a material used for the layer in contact with or in the vicinity of the light-emitting layer is preferably higher than that of a material used for the light-emitting layer. Therefore, when a material different from a material used for the light-emitting layer is used as the electron-transport material, a light-emitting device with high efficiency can be obtained.

The heteroaromatic compound is an organic compound including at least one heteroaromatic ring.

Note that the heteroaromatic ring has any one of a pyridine ring, a diazine ring, a triazine ring, a polyazole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having a polyazole ring includes a heteroaromatic ring having an imidazole ring, a triazole ring, or an oxadiazole ring.

The heteroaromatic ring includes a condensed heteroaromatic ring having a fused ring structure. Examples of the condensed heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of a heteroaromatic compound containing carbon and one or more of nitrogen, oxygen, sulfur, and the like and having a five-membered ring structure include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of a heteroaromatic compound containing carbon and one or more of nitrogen, oxygen, sulfur, and the like and having a six-membered ring structure include a heteroaromatic compound having a heteroaromatic ring such as a pyridine ring, a diazine ring (including a pyrimidine ring, a pyrazine ring, a pyridazine ring, or the like), a triazine ring, or a polyazole ring.

Other examples include a heteroaromatic compound having a bipyridine structure and a heteroaromatic compound having a terpyridine structure, which are included in heteroaromatic compounds in which pyridine rings are connected.

Examples of the heteroaromatic compound with a fused ring structure which partly has the above six-membered ring structure include a heteroaromatic compound having a condensed heteroaromatic ring such as a quinoline ring, a benzoquinoline ring, a quinoxaline ring, a dibenzoquinoxaline ring, a phenanthroline ring, a furodiazine ring (including a structure where an aromatic ring is fused to the furan ring of a furodiazine ring), or a benzimidazole ring.

Specific examples of the above-described heteroaromatic compound having a five-membered ring structure (e.g., a polyazole ring (including an imidazole ring, a triazole ring, or an oxadiazole ring), an oxazole ring, a thiazole ring, or a benzimidazole ring) include 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 3-(4-biphenylyl)-4-phenyl-5-(4-tert-butylphenyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), and 4,4′-bis(5-methylbenzoxazol-2-yl) stilbene (abbreviation: BzOs).

Specific examples of the above-described heteroaromatic compound having a six-membered ring structure (including a heteroaromatic ring having a pyridine ring, a diazine ring, a triazine ring, or the like) include a heteroaromatic compound including a heteroaromatic ring having a pyridine ring, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB), a heteroaromatic compound including a heteroaromatic ring having a triazine ring, such as 2-{4-[3-(N-phenyl-9H-carbazol-3-yl)-9H-carbazol-9-yl]phenyl}-4,6-diphenyl-1,3,5-triazine (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)-1,1′-biphenyl-3-yl]-4,6-diphenyl-1,3,5-triazine (abbreviation: mTpBPTzn), 2-[(1,1′-biphenyl)-4-yl]-4-phenyl-6-[9,9′-spirobi (9H-fluoren)-2-yl]-1,3,5-triazine (abbreviation: BP-SFTzn), 2,6-bis(4-naphthalen-1-ylphenyl)-4-[4-(3-pyridyl)phenyl]pyrimidine (abbreviation: 2,4NP-6PyPPm), 9-[4-(4,6-diphenyl-1,3,5-triazin-2-yl)-2-dibenzothienyl]-2-phenyl-9H-carbazole (abbreviation: PCzDBfTzn), 3-[9-(4,6-diphenyl-1,3,5-triazin-2-yl)-2-dibenzofuranyl]-9-phenyl-9H-carbazole (abbreviation: PCDBfTzn), 2-(biphenyl-3-yl)-4-phenyl-6-(8-[1,1′: 4′,1″-terphenyl]-4-yl-1-dibenzofuranyl)-1,3,5-triazine (abbreviation: mBP-TPDBfTzn), or 2-[3′-(9,9-dimethyl-9H-fluoren-2-yl)biphenyl-3-yl]-4,6-diphenyl-1,3,5-triazine (abbreviation: mFBPTzn), and a heteroaromatic compound including a heteroaromatic ring having a diazine (pyrimidine) ring, such as 4,6-bis[3-(phenanthren-9-yl)phenyl]pyrimidine (abbreviation: 4,6mPnP2Pm), 4,6-bis[3-(4-dibenzothienyl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 4,6mCzBP2Pm, 6-(biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(1,1′-biphenyl-4-yl)pyrimidine (abbreviation: 6BP-4-[3-(dibenzothiophen-4-yl)phenyl]-8-(naphthalen-2-yl)-[1]benzofuro[3,2-4Cz2PPm), d]pyrimidine (abbreviation: 8βN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)(biphenyl-3-yl)]naphtho[1′,2′: 4,5]furo[3,2-d]pyrimidine (abbreviation: 8mDBtBPNfpm), or 8-[(2,2′-binaphthalen)-6-yl]-4-[3-(dibenzothiophen-4-yl)phenyl]-[1]benzofuro[3,2-d]pyrimidine (abbreviation: 8(βN2)-4mDBtPBfpm). Note that the above aromatic compounds including a heteroaromatic ring include a heteroaromatic compound having a condensed heteroaromatic ring.

Other examples include a heteroaromatic compound including a heteroaromatic ring having a diazine (pyrimidine) ring, such as 2,2′-(pyridine-2,6-diyl)bis(4-phenylbenzo[h]quinazoline) (abbreviation: 2,6(P-Bqn)2Py), 2,2′-(2,2′-bipyridine-6,6′-diyl)bis(4-phenylbenzo[h]quinazoline) (abbreviation: 6,6′(P-Bqn)2BPy), 2,2′-(pyridine-2,6-diyl)bis {4-[4-(2-naphthyl)phenyl]-6-phenylpyrimidine} (abbreviation: 2,6(NP-PPm)2Py), or 6-(biphenyl-3-yl)-4-[3,5-bis(9H-carbazol-9-yl)phenyl]-2-phenylpyrimidine (abbreviation: 6mBP-4Cz2PPm), and a heteroaromatic compound including a heteroaromatic ring having a triazine ring, such as 2,4,6-tris(3′-(pyridin-3-yl)biphenyl-3-yl)-1,3,5-triazine (abbreviation: TmPPPyTz), 2,4,6-tris(2-pyridyl)-1,3,5-triazine (abbreviation: 2Py3Tz), or 2-[3-(2,6-dimethyl-3-pyridyl)-5-(9-phenanthrenyl)phenyl]-4,6-diphenyl-1,3,5-triazine (abbreviation: mPn-mDMePyPTzn).

Specific examples of the above-described heteroaromatic compound with a fused ring structure which partly has the six-membered ring structure (a heteroaromatic compound having a fused ring structure) include a heteroaromatic compound having a quinoxaline ring, such as bathophenanthroline (abbreviation: BPhen), bathocuproine (abbreviation: BCP), 2,9-di(naphthalen-2-yl)-4,7-diphenyl-1,10-phenanthroline (abbreviation: NBphen), 2,2′-(1,3-phenylene)bis(9-phenyl-1,10-phenanthroline) (abbreviation: mPPhen2P), 2,2′-(1,1′-biphenyl)-4,4′-diylbis(9-phenyl-1,10-phenanthroline) (abbreviation: PPhen2BP), 2,2′-(pyridine-2,6-diyl)bis(4-phenylbenzo[h]quinazoline) (abbreviation: 2,6(P-Bqn)2Py), 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), or 2mpPCBPDBq.

For the electron-transport layers (114, 114a, and 114b), any of the metal complexes given below as well as the heteroaromatic compounds given above can be used. Examples of the metal complexes include a metal complex including a quinoline ring or a benzoquinoline ring, such as tris(8-quinolinolato)aluminum(III) (abbreviation: Alq₃), Almq₃, 8-quinolinolatolithium(I) (abbreviation: Liq), BeBq₂, bis(2-methyl-8-quinolinolato)(4-phenylphenolato)aluminum(III) (abbreviation: BAlq), or bis(8-quinolinolato)zinc(II) (abbreviation: Znq), and a metal complex including an oxazole ring or a thiazole ring, such as bis[2-(2-benzoxazolyl)phenolato]zinc(II) (abbreviation: ZnPBO) or bis[2-(2-benzothiazolyl)phenolato]zinc(II) (abbreviation: ZnBTZ).

A high molecular compound such as poly(2,5-pyridinediyl) (abbreviation: PPy), poly[(9,9-dihexylfluorene-2,7-diyl)-co-(pyridine-3,5-diyl)] (abbreviation: PF-Py), or poly[(9,9-dioctylfluorene-2,7-diyl)-co-(2,2′-bipyridine-6,6′-diyl)] (abbreviation: PF-BPy) can also be used as an electron-transport material.

Each of the electron-transport layers (114, 114a, and 114b) is not limited to a single layer, and may be a stack of two or more layers each made of any of the above substances.

### <Electron-Injection Layer>

The electron-injection layers (115, 115a, and 115b) are each a layer containing a substance having a high electron-injection property. The electron-injection layers (115, 115a, and 115b) are each a layer for increasing the efficiency of electron injection from the second electrode 102 and are each preferably formed using a material whose LUMO level value has a small difference (0.5 eV or less) from the work function value of the material used for the second electrode 102. Thus, the electron-injection layer 115 can be formed using an alkali metal, an alkaline earth metal, or a compound thereof, such as lithium, cesium, lithium fluoride (LiF), cesium fluoride (CsF), calcium fluoride (CaF₂), Liq, 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), lithium oxide (LiOₓ), or cesium carbonate. A rare earth metal or a rare earth metal compound, such as erbium fluoride (ErF₃) or ytterbium (Yb), can also be used. Note that to form the electron-injection layers (115, 115a, and 115b), a plurality of kinds of the above-described materials may be mixed or a plurality of kinds of the above-described materials may be stacked. Electride may also be used for the electron-injection layers (115, 115a, and 115b). Examples of the electride include a substance in which electrons are added at high concentration to calcium oxide-aluminum oxide. Note that any of the above-described substances contained in the electron-transport layers (114, 114a, and 114b) can also be used. The compound of the present application has an excellent electron-transport property, and thus is suitably used for an electron-injection layer. It is further preferable that the compound of the present application be used for both the electron-injection layer 115a and the electron-injection layer 115b.

A mixed material in which an organic compound and an electron donor (donor) are mixed may also be used for the electron-injection layers (115, 115a, and 115b). Such a mixed material is excellent in an electron-injection property and an electron-transport property because electrons are generated in the organic compound by the electron donor. In this case, the organic compound is preferably a material excellent in transporting the generated electrons; specifically, for example, the above-described electron-transport materials (e.g., metal complexes and heteroaromatic compounds) used in the electron-transport layers (114, 114a, and 114b) can be used. Any substance showing an electron-donating property with respect to the organic compound can serve as an electron donor. Specifically, an alkali metal, an alkaline earth metal, and a rare earth metal are preferable, and lithium, cesium, magnesium, calcium, erbium, ytterbium, and the like are given as examples. In addition, an alkali metal oxide and an alkaline earth metal oxide are preferable, and lithium oxide, calcium oxide, barium oxide, and the like are given as examples. A Lewis base such as magnesium oxide can also be used. An organic compound such as tetrathiafulvalene (abbreviation: TTF) can also be used. Alternatively, a stack of these materials may be used.

Moreover, a mixed material in which an organic compound and a metal are mixed may also be used for the electron-injection layers (115, 115a, and 115b). The organic compound used here preferably has a LUMO level higher than or equal to −3.6 eV and lower than or equal to −2.3 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, and the like), a triazine ring, a bipyridine ring, or a terpyridine ring), and a heteroaromatic compound with a fused ring structure which partly has a six-membered ring structure (e.g., a quinoline ring, a benzoquinoline ring, a quinoxaline ring, a dibenzoquinoxaline ring, or a phenanthroline ring). Since the materials are specifically described above, description thereof is omitted here.

As the metal used for the above mixed material, a transition metal belonging to Group 5, Group 7, Group 9, or Group 11 in the periodic table or a material belonging to Group 13 is preferably used, and Ag, Cu, Al, In, and the like can be given as examples. Here, the organic compound forms a singly occupied molecular orbital (SOMO) with the transition metal.

To amplify light obtained from the light-emitting layer 113b, for example, the optical path length between the second electrode 102 and the light-emitting layer 113b is preferably less than one-fourth of the wavelength λ of light emitted from the light-emitting layer 113b. In that case, the optical path length can be adjusted by changing the thickness of the electron-transport layer 114b or the electron-injection layer 115b.

When the charge-generation layer 106 is provided between two EL layers (103a and 103b) as in the light-emitting device illustrated in FIG. 1D, a structure where a plurality of EL layers are stacked between the pair of electrodes (also referred to as a tandem structure) can be employed.

### <Charge-Generation Layer>

The charge-generation layer 106 has a function of injecting electrons into the EL layer 103a and injecting holes into the EL layer 103b when a voltage is applied between the first electrode (anode) 101 and the second electrode (cathode) 102. The charge-generation layer 106 may have either a structure where an electron acceptor (acceptor) is added to a hole-transport material (also referred to as a P-type layer) or a structure where an electron donor (donor) is added to an electron-transport material (also referred to as an electron-injection buffer layer). Alternatively, both of these structures may be stacked. Furthermore, an electron-relay layer may be provided between the P-type layer and the electron-injection buffer layer. Note that forming the charge-generation layer 106 with the use of any of the above materials can inhibit an increase in driving voltage caused by the stack of the EL layers. The compound of the present application has an excellent transport property, and thus is suitably used for the charge-generation layer 106.

In the case where the charge-generation layer 106 has a structure where an electron acceptor is added to a hole-transport material that is an organic compound (a P-type layer), any of the materials described in this embodiment can be used as the hole-transport material. As examples of the electron acceptor, 7,7,8,8-tetracyano-2,3,5,6-tetrafluoroquinodimethane (abbreviation: F₄-TCNQ), chloranil, and the like can be given. Other examples include oxides of metals belonging to Group 4 to Group 8 in the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of the P-type layer or a stack of single films containing the respective materials may be used.

In the case where the charge-generation layer 106 has a structure where an electron donor is added to an electron-transport material (an electron-injection buffer layer), any of the materials described in this embodiment can be used as the electron-transport material. As the electron donor, it is possible to use an alkali metal, an alkaline earth metal, a rare earth metal, a metal belonging to Group 2 or Group 13 in the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An organic compound such as tetrathianaphthacene may be used as the electron donor.

When an electron-relay layer is provided between a P-type layer and an electron-injection buffer layer in the charge-generation layer 106, the electron-relay layer contains at least a substance having an electron-transport property and has a function of preventing an interaction between the electron-injection buffer layer and the P-type layer and transferring electrons smoothly. The LUMO level of the substance having an electron-transport property in the electron-relay layer is preferably between the LUMO level of the acceptor substance in the P-type layer and the LUMO level of the substance having an electron-transport property in the electron-transport layer in contact with the charge-generation layer 106. A specific energy level of the LUMO level of the substance having an electron-transport property which is used for the electron-relay layer is preferably higher than or equal to −5.0 eV, further preferably higher than or equal to −5.0 eV and lower than or equal to −3.0 eV. Note that as the substance having an electron-transport property in the electron-relay layer, a phthalocyanine-based material or a metal complex having a metal-oxygen bond and an aromatic ligand is preferably used.

Although FIG. 1D illustrates the structure where two EL layers 103 are stacked, three or more EL layers may be stacked with a charge-generation layer provided between different EL layers.

### <Cap Layer>

Although not illustrated in FIG. 1A to FIG. 1E, a cap layer may be provided over the second electrode 102 of the light-emitting device. For example, a material with a high refractive index can be used for the cap layer. When the cap layer is provided over the second electrode 102, extraction efficiency of light emitted from the second electrode 102 can be improved. The refractive index of a cap layer material is preferably higher than the refractive index of an electron-transport layer material. Furthermore, the refractive index of the cap layer material is preferably higher than the refractive index of the electron-transport layer material, higher than the refractive index of a host material in a light-emitting layer, and higher than the refractive index of a hole-transport layer material. The cap layer may have a stacked-layer structure. In that case, a stack of a layer including the compound of the present application and a layer including a material with a higher refractive index than the compound of the present application thereover is preferable.

In a bendable device (also referred to as flexible device) that uses a flexible substrate as a substrate, a bent portion might be decreased in light extraction efficiency or might be decreased in emission efficiency due to influence of external light or the like. Thus, a material with a high refractive index is preferably used for a cap layer in the bent portion. More specifically, a cap layer, a cathode, a layer including the compound of the present application, and a light-emitting layer are preferably placed to overlap with each other in the bent portion. In that case, the refractive index of the compound of the present application is preferably lower than the refractive index of the cap layer material and the refractive index of the host material in the light-emitting layer. In a device including a plurality of bent portions or in a wind-up device, many bent portions are included; thus, a cap layer, a layer including the compound of the present application, and a light-emitting layer are preferably provided to overlap with each other in each of the bent portions.

The LUMO level of the electron-transport layer material is lower than the LUMO level of the cap layer material preferably by 0.3 eV or more, further preferably by 0.5 eV or more. The LUMO level of the host material in the light-emitting layer is higher than the LUMO level of the cap layer material preferably by 0.1 eV or more, further preferably by 0.3 eV or more. It is particularly preferable that the LUMO levels satisfy the relation of the cap layer material >the host material in the light-emitting layer >the electron-transport layer material. In the case where a plurality of light-emitting devices (e.g., a red device, a blue device, and a green device) are included, each of the light-emitting devices preferably satisfies the relation of the LUMO levels.

Specific examples of a material that can be used for the cap layer include 5,5′-diphenyl-2,2′-di-5H-[1]benzothieno[3,2-c]carbazole (abbreviation: BisBTc) and 4,4′,4″-(benzene-1,3,5-triyl)tri (dibenzothiophene) (abbreviation: DBT3P-II). A compound having a triarylamine skeleton is preferable because of its stability. In view of the stability, it is effective to use a compound having a triarylamine skeleton for both the cap layer and the hole-injection layer. It is also preferable to use a compound having a triarylamine skeleton for all of the cap layer, the hole-injection layer, and the hole-transport layer. The organic compounds described in Embodiment 1 can also be used.

### <Substrate>

The light-emitting device described in this embodiment can be formed over a variety of substrates. Note that the type of substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, paper including a fibrous material, and a base material film including a fibrous material.

Examples of the glass substrate include barium borosilicate glass, aluminoborosilicate glass, and soda lime glass. Examples of the flexible substrate, the attachment film, and the base material film include plastics typified by polyethylene terephthalate (PET), polyethylene naphthalate (PEN), and polyether sulfone (PES), a synthetic resin such as acrylic resin, polypropylene, polyester, polyvinyl fluoride, polyvinyl chloride, polyamide, polyimide, aramid, epoxy resin, an inorganic vapor deposition film, and paper.

For fabrication of the light-emitting device described in this embodiment, a vapor phase method such as an evaporation method or a liquid phase method such as a spin coating method and an ink-jet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, layers having a variety of functions (the hole-injection layer 111, the hole-transport layer 112, the light-emitting layer 113, the electron-transport layer 114, and the electron-injection layer 115) included in the EL layer of the light-emitting device can be formed by an evaporation method (e.g., a vacuum evaporation method), a coating method (e.g., a dip coating method, a die coating method, a bar coating method, a spin coating method, or a spray coating method), a printing method (e.g., an ink-jet method, a screen printing (stencil) method, an offset printing (planography) method, a flexography (relief printing) method, a gravure printing method, or a micro-contact printing method), or the like.

In the case where a film formation method such as the coating method or the printing method is employed, a high molecular compound (e.g., an oligomer, a dendrimer, or a polymer), a middle molecular compound (a compound between a low molecular compound and a high molecular compound with a molecular weight of greater than or equal to 400 and less than or equal to 4000), an inorganic compound (e.g., a quantum dot material), or the like can be used. As the quantum dot material, a colloidal quantum dot material, an alloyed quantum dot material, a core-shell quantum dot material, a core quantum dot material, or the like can be used.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

Note that in this specification and the like, the term “layer” and the term “film” can be interchanged with each other as appropriate.

The compound of the present application can be used as a hole-transport material, a host material, an electron-transport material, a cap layer, or the like.

The structures described in this embodiment can be used in an appropriate combination with any of the structures described in the other embodiments.

### Embodiment 3

This embodiment will describe a light-emitting and light-receiving apparatus 700 as a specific structure example of a light-emitting and light-receiving apparatus of one embodiment of the present invention, and an example of the manufacturing method thereof. The light-emitting and light-receiving apparatus 700 includes a light-emitting device and thus can be regarded as a light-emitting apparatus; includes a light-receiving device and thus can be regarded as a light-receiving apparatus; and can be used in a display portion in an electronic device and thus can be regarded as a display panel or a display apparatus.

### <Structure Example of Light-Emitting and Light-Receiving Apparatus 700>

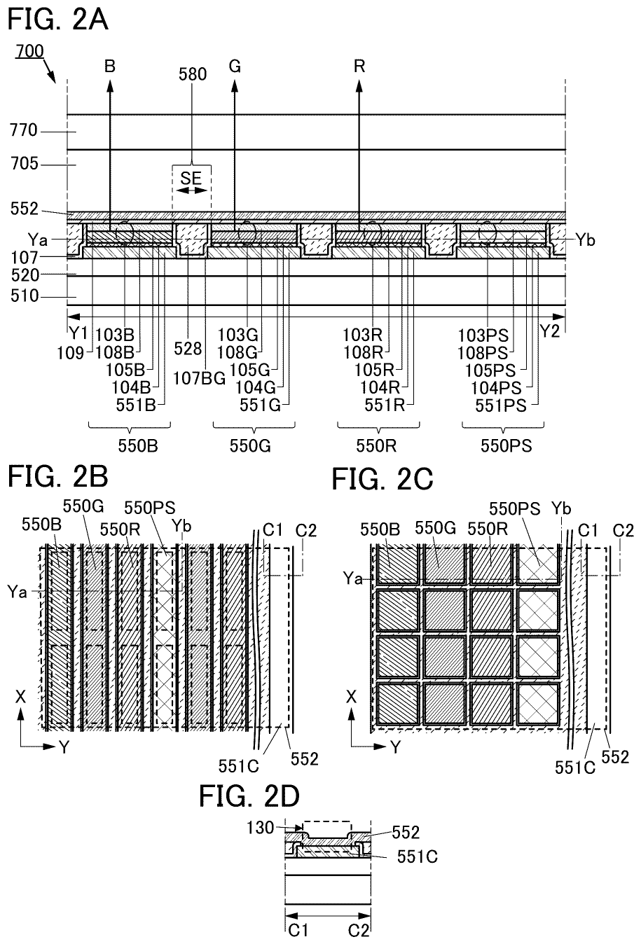

The light-emitting and light-receiving apparatus 700 illustrated in FIG. 2A includes a light-emitting device 550B, a light-emitting device 550G, a light-emitting device 550R, and a light-receiving device 550PS. The light-emitting device 550B, the light-emitting device 550G, the light-emitting device 550R, and the light-receiving device 550PS are formed over a functional layer 520 provided over a first substrate 510. The functional layer 520 includes, for example, driver circuits composed of a plurality of transistors, such as a gate driver and a source driver, and wirings that electrically connect these circuits. Note that these driver circuits are electrically connected to the light-emitting device 550B, the light-emitting device 550G, the light-emitting device 550R, and the light-receiving device 550PS, for example, and can drive them. The light-emitting and light-receiving apparatus 700 includes an insulating layer 705 over the functional layer 520 and the devices (the light-emitting devices and the light-receiving device), and the insulating layer 705 has a function of bonding a second substrate 770 and the functional layer 520.

The light-emitting device 550B, the light-emitting device 550G, and the light-emitting device 550R have the device structure described in Embodiment 2, and the light-receiving device 550PS has a device structure described later in Embodiment 8. Although this embodiment describes the case where the devices (a plurality of light-emitting devices and a light-receiving device) are formed separately, part of an EL layer of the light-emitting device (a hole-injection layer, a hole-transport layer, or an electron-transport layer) and part of an active layer of the light-receiving device (a first transport layer or a second transport layer) may be formed using the same material at the same time in the manufacturing process. The detailed description will be made in Embodiment 8.

Note that in this specification and the like, a structure where light-emitting layers in light-emitting devices of different colors (e.g., blue (B), green (G), and red (R)) and a light-receiving layer in a light-receiving device are separately formed or separately patterned may be referred to as an SBS (Side By Side) structure. Note that in the light-emitting and light-receiving apparatus 700 illustrated in FIG. 2A, the light-emitting device 550B, the light-emitting device 550G, the light-emitting device 550R, and the light-receiving device 550PS are arranged in this order; however, one embodiment of the present invention is not limited thereto. For example, in the light-emitting and light-receiving apparatus 700, these devices may be arranged in the order of the light-emitting device 550R, the light-emitting device 550G, the light-emitting device 550B, and the light-receiving device 550PS.

In FIG. 2A, the light-emitting device 550B includes an electrode 551B, an electrode 552, and an EL layer 103B. The light-emitting device 550G includes an electrode 551G, the electrode 552, and an EL layer 103G. The light-emitting device 550R includes an electrode 551R, the electrode 552, and an EL layer 103R. The light-receiving device 550PS includes an electrode 551PS, the electrode 552, and a light-receiving layer 103PS. Note that a specific structure of each layer of the light-receiving device is as described in Embodiment 8. A specific structure of each layer of the light-emitting device is as described in Embodiment 2. The EL layer 103B, the

EL layer 103G, and the EL layer 103R each have a stacked-layer structure of layers having different functions including light-emitting layers (105B, 105G, and 105R). The light-receiving layer 103PS has a stacked-layer structure of layers having different functions including an active layer 105PS. FIG. 2A illustrates a case where the EL layer 103B includes a hole-injection/transport layer 104B, the light-emitting layer 105B, an electron-transport layer 108B, and an electron-injection layer 109; the EL layer 103G includes a hole-injection/transport layer 104G, the light-emitting layer 105G, an electron-transport layer 108G, and the electron-injection layer 109; the EL layer 103R includes a hole-injection/transport layer 104R, the light-emitting layer 105R, an electron-transport layer 108R, and the electron-injection layer 109; and the light-receiving layer 103PS includes a first transport layer 104PS, the active layer 105PS, a second transport layer 108PS, and the electron-injection layer 109. However, the present invention is not limited thereto. Note that each of the hole-injection/transport layers (104B, 104G, and 104R) represents the layer having the functions of the hole-injection layer and the hole-transport layer described in Embodiment 2 and may have a stacked-layer structure.

Note that the electron-transport layers (108B, 108G, and 108R) and the second transport layer 108PS may have a function of blocking holes moving from the anode side to the cathode side through the light-emitting layers (103B, 103G, and 103R) and the light-receiving layer 103PS of the light-receiving device. The electron-injection layer 109 may have a stacked-layer structure where some or all of layers are formed using different materials.

As illustrated in FIG. 2A, an insulating layer 107 may be formed on side surfaces (or end portions) of the hole-injection/transport layers (104B, 104G, and 104R), the light-emitting layers (105B, 105G, and 105R), and the electron-transport layers (108B, 108G, and 108R) included in the EL layers (103B, 103G, and 103R), and side surfaces (or end portions) of the first transport layer 104PS, the active layer 105PS, and the second transport layer 108PS included in the light-receiving layer 103PS. The insulating layer 107 is formed in contact with the side surfaces (or the end portions) of the EL layers (103B, 103G, and 103R) and the light-receiving layer 103PS. This can inhibit entry of oxygen, moisture, or constituent elements thereof into the inside through the side surfaces of the EL layers (103B, 103G, and 103R) and the light-receiving layer 103PS. For the insulating layer 107, aluminum oxide, magnesium oxide, hafnium oxide, gallium oxide, indium gallium zinc oxide, silicon nitride, silicon nitride oxide, or the like can be used, for example. Some of the above-described materials may be stacked to form the insulating layer 107. The insulating layer 107 can be formed by a sputtering method, a CVD method, an MBE method, a

PLD method, an ALD method, or the like and is formed preferably by an ALD method, which enables favorable coverage. Note that the insulating layer 107 continuously covers the side surfaces (or the end portions) of parts of the EL layers (103B, 103G, and 103R) and part of the light-receiving layer 103PS of adjacent devices. For example, in FIG. 2A, the side surfaces of part of the EL layer 103B of the light-emitting device 550B and part of the EL layer 103G of the light-emitting device 550G are covered with the insulating layer 107. In regions covered with the insulating layer 107, partition walls 528 formed using an insulating material are preferably formed, as illustrated in FIG. 2A.

In addition, the electron-injection layer 109 is formed over the electron-transport layers (108B, 108G, and 108R) that are parts of the EL layers (103B, 103G, and 103R), the second transport layer 108PS that is part of the light-receiving layer 103PS, and the insulating layer 107. Note that the electron-injection layer 109 may have a stacked-layer structure of two or more layers (for example, stacked layers having different electric resistances).

The electrode 552 is formed over the electron-injection layer 109. Note that the electrodes (551B, 551G, and 551R) and the electrode 552 have overlapping regions. The light-emitting layer 105B is provided between the electrode 551B and the electrode 552, the light-emitting layer 105G is provided between the electrode 551G and the electrode 552, the light-emitting layer 105R is provided between the electrode 551R and the electrode 552, and the light-receiving layer 103PS is provided between the electrode 551PS and the electrode 552.

The EL layers (103B, 103G, and 103R) illustrated in FIG. 2A each have a structure similar to that of the EL layer 103 described in Embodiment 2. The light-receiving layer 103PS has a structure similar to that of a light-receiving layer to be described in Embodiment 8. The light-emitting layer 105B can emit blue light, the light-emitting layer 105G can emit green light, and the light-emitting layer 105R can emit red light, for example.

The partition walls 528 are provided in regions surrounded by the electron-injection layer 109 and the insulating layer 107. As illustrated in FIG. 2A, the partition walls 528 are in contact with the side surfaces (or the end portions) of the electrodes (551B, 551G, 551R, and 551PS) and parts of the EL layers (103B, 103G, and 103R) of the light-emitting devices and part of the light-receiving layer 103PS with the insulating layer 107 therebetween.

In each of the EL layers and the light-receiving layer, particularly the hole-injection layer, which is included in a hole-transport region between the anode and the light-emitting layer and a hole-transport region between the anode and the active layer, often has high conductivity; thus, the hole-injection layer formed as a layer shared by adjacent light-emitting devices might cause crosstalk. Thus, as described in this structure example, the partition walls 528 formed using an insulating material are provided between the EL layers and between the EL layer and the light-receiving layer, which can inhibit occurrence of crosstalk between adjacent devices.

In the manufacturing method described in this embodiment, side surfaces (or end portions) of the EL layer and the light-receiving layer are exposed in the patterning step. This may promote deterioration of the EL layer and the light-receiving layer by allowing entry of oxygen, water, or the like through the side surfaces (or the end portions) of the EL layer and the light-receiving layer. Thus, providing the partition wall 528 can inhibit the deterioration of the EL layer and the light-receiving layer in the manufacturing process.

Furthermore, a depressed portion formed between adjacent devices can be flattened by provision of the partition wall 528. When the depressed portion is flattened, disconnection of the electrode 552 formed over the EL layers and the light-receiving layer can be inhibited. As an insulating material used for forming the partition wall 528, an organic material such as an acrylic resin, a polyimide resin, an epoxy resin, an imide resin, a polyamide resin, a polyimide-amide resin, a silicone resin, a siloxane resin, a benzocyclobutene-based resin, a phenol resin, or precursors of these resins can be used, for example. An organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin may be used. A photosensitive resin such as a photoresist can also be used. Note that as the photosensitive resin, a positive photosensitive material or a negative photosensitive material can be used.

With the use of the photosensitive resin, the partition wall 528 can be fabricated only by light exposure and development steps. The partition wall 528 may be formed using a negative photosensitive resin (e.g., a resist material). In the case where an insulating layer containing an organic material is used as the partition wall 528, a material absorbing visible light is suitably used. When a material absorbing visible light is used for the partition wall 528, light emitted from the EL layer can be absorbed by the partition wall 528, so that light that might leak to the adjacent EL layer or the adjacent light-receiving layer (stray light) can be inhibited. Thus, a display panel having high display quality can be provided.

The difference between the top-surface level of the partition wall 528 and the top-surface level of any of the EL layer 103B, the EL layer 103G, the EL layer 103R, and the light-receiving layer 103PS is preferably 0.5 times or less, further preferably 0.3 times or less the thickness of the partition wall 528, for example. The partition wall 528 may be provided such that the top-surface level of any of the EL layer 103B, the EL layer 103G, the EL layer 103R, and the light-receiving layer 103PS is higher than the top-surface level of the partition wall 528, for example. Alternatively, the partition wall 528 may be provided such that the top-surface level of the partition wall 528 is higher than the top-surface level of each of the EL layer 103B, the EL layer 103G, the EL layer 103R, and the light-receiving layer 103PS, for example.

When electrical continuity is established between the EL layer 103B, the EL layer 103G, the EL layer 103R, and the light-receiving layer 103PS in a light-emitting and light-receiving apparatus (display panel) with a high resolution exceeding 1000 ppi, a crosstalk phenomenon occurs, resulting in a narrower color gamut of the light-emitting and light-receiving apparatus. Providing the partition wall 528 in a high-resolution display panel with more than 1000 ppi, preferably more than 2000 ppi, or further preferably in an ultrahigh-resolution display panel with more than 5000 ppi allows the display panel to express vivid colors.

FIG. 2B and FIG. 2C are each a schematic top view of the light-emitting and light-receiving apparatus 700 taken along the dashed-dotted line Ya-Yb in the cross-sectional view of FIG. 2A. The light-emitting devices 550B, the light-emitting devices 550G, and the light-emitting devices 550R are arranged in a matrix. FIG. 2B illustrates what is called stripe arrangement, in which the light-emitting devices of the same color are arranged in X-direction. FIG. 2C illustrates a structure where the light-emitting devices of the same color are arranged in the X-direction and separated by patterning for each pixel. Note that the arrangement method of the light-emitting devices is not limited thereto; another arrangement method such as delta arrangement, zigzag arrangement, PenTile arrangement, or diamond arrangement may also be used.

The EL layers (103B, 103G, and 103R) and the light-receiving layer 103PS are processed to be separated from each other by patterning using a photolithography method; hence, a high resolution light-emitting and light-receiving apparatus (display panel) can be manufactured. End portions (side surfaces) of the layers of the EL layer processed by patterning using a photolithography method have substantially the same surface (or are positioned on substantially the same plane). In addition, the side surfaces (end portions) of the layers of the light-receiving layer processed by patterning using a photolithography method have substantially the same surface (or are positioned on substantially the same plane). In this case, the width (SE) of a space 580 between the EL layers and between the EL layer and the light-receiving layer is preferably less than or equal to 5 μm, further preferably less than or equal to 1 μm. Note that the width of the space between the EL layer and the light-receiving layer is preferably greater than the width of the space between the EL layers.

In the EL layer, particularly the hole-injection layer, which is included in the hole-transport region between the anode and the light-emitting layer, often has high conductivity; thus, the hole-injection layer formed as a layer shared by adjacent light-emitting devices might cause crosstalk. Therefore, processing the EL layers to be separated by patterning using a photolithography method as shown in this structure example can inhibit occurrence of crosstalk between adjacent light-emitting devices.

FIG. 2D is a schematic cross-sectional view taken along the dashed-dotted line C1-C2 in FIG. 2B and FIG. 2C. FIG. 2D illustrates a connection portion 130 where a connection electrode 551C and the electrode 552 are electrically connected. In the connection portion 130, the electrode 552 is provided over and in contact with the connection electrode 551C. In addition, the partition wall 528 is provided to cover the end portion of the connection electrode 551C.

### <Manufacturing Method Example of Light-Emitting and Light-Receiving Apparatus>

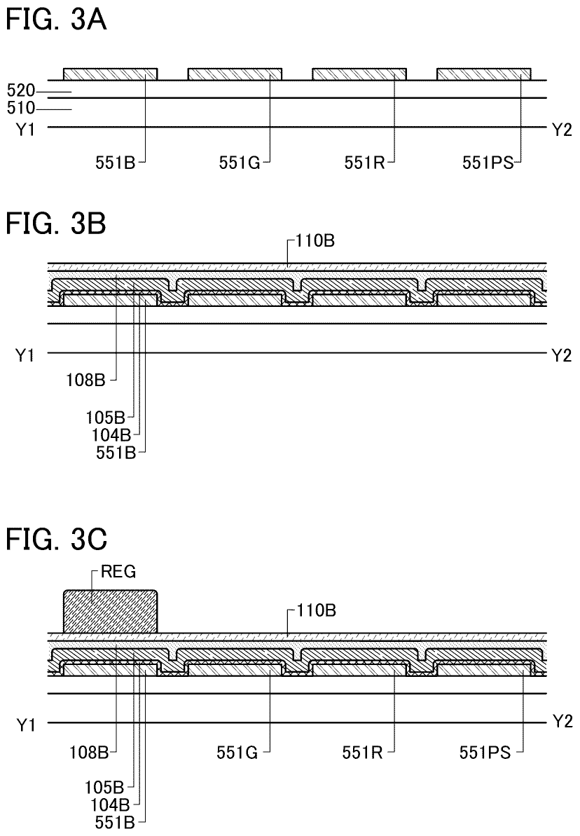

The electrode 551B, the electrode 551G, the electrode 551R, and the electrode 551PS are formed as illustrated in FIG. 3A. For example, a conductive film is formed over the functional layer 520 over the first substrate 510 and processed into predetermined shapes by a photolithography method.

The conductive film can be formed by a sputtering method, a chemical vapor deposition (CVD) method, a molecular beam epitaxy (MBE) method, a vacuum evaporation method, a pulsed laser deposition (PLD) method, an atomic layer deposition (ALD) method, or the like. Examples of the CVD method include a plasma-enhanced chemical vapor deposition (PECVD) method and a thermal CVD method. As an example of the thermal CVD method, a metal organic chemical vapor deposition (MOCVD: Metal Organic CVD) method can be given.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask.

There are the following two typical examples of a photolithography method. In one of the methods, a resist mask is formed over a thin film that is to be processed, the thin film is processed by etching or the like, and then the resist mask is removed. In the other method, a photosensitive thin film is formed and then processed into a desired shape by light exposure and development. The former method involves heat treatment steps such as heating after resist application (PAB: Pre Applied Bake) and heating after light exposure (PEB: Post Exposure Bake). In one embodiment of the present invention, a lithography method is used not only for processing of a conductive film but also for processing of a thin film used for formation of an EL layer (a film made of an organic compound or a film partly including an organic compound).

As the light used for light exposure in the photolithography method, for example, an i-line (with a wavelength of 365 nm), a g-line (with a wavelength of 436 nm), an h-line (with a wavelength of 405 nm), or combined light of any of them can be used. Alternatively, ultraviolet rays, KrF laser light, ArF laser light, or the like can be used. In addition, light exposure may be performed by liquid immersion exposure technique. As the light for light exposure, extreme ultraviolet (EUV) light or X-rays may also be used. Furthermore, instead of the light used for the light exposure, an electron beam can also be used. It is preferable to use extreme ultraviolet light, X-rays, or an electron beam because they can perform extremely fine processing. Note that a photomask is not needed when light exposure is performed by scanning with a beam such as an electron beam.

For etching of a thin film using a resist mask, a dry etching method, a wet etching method, a sandblast method, or the like can be used.

Subsequently, as illustrated in FIG. 3B, the hole-injection/transport layer 104B, the light-emitting layer 105B, and the electron-transport layer 108B are formed over the electrode 551B, the electrode 551G, the electrode 551R, and the electrode 551PS. Note that the hole-injection/transport layer 104B, the light-emitting layer 105B, and the electron-transport layer 108B can be formed by a vacuum evaporation method, for example. Furthermore, a sacrificial layer 110B is formed over the electron-transport layer 108B. As materials for forming the hole-injection/transport layer 104B, the light-emitting layer 105B, and the electron-transport layer 108B, any of the materials described in Embodiment 2 can be used.

For the sacrificial layer 110B, it is preferable to use a film highly resistant to etching treatment performed on the hole-injection/transport layer 104B, the light-emitting layer 105B, and the electron-transport layer 108B, i.e., a film having high etching selectivity. The sacrificial layer 110B preferably has a stacked-layer structure of a first sacrificial layer and a second sacrificial layer which have different etching selectivities. Moreover, for the sacrificial layer 110B, it is possible to use a film that can be removed by a wet etching method less likely to cause damage to the EL layer 103B. In wet etching, oxalic acid or the like can be used as an etching material.

The sacrificial layer 110B can be formed using an inorganic film such as a metal film, an alloy film, a metal oxide film, a semiconductor film, or an inorganic insulating film, for example. The sacrificial layer 110B can be formed by any of a variety of film formation methods such as a sputtering method, an evaporation method, a CVD method, and an ALD method.

For the sacrificial layer 110B, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used. It is particularly preferable to use a low-melting-point material such as aluminum or silver.

The sacrificial layer 110B can be formed using a metal oxide such as indium gallium zinc oxide (In—Ga—Zn oxide, also referred to as IGZO). It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon or the like can also be used.

Note that an element M (M is one or more kinds selected from aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) can be employed instead of gallium. In particular, M is preferably one or more kinds selected from gallium, aluminum, and yttrium.

For the sacrificial layer 110B, an inorganic insulating material such as aluminum oxide, hafnium oxide, or silicon oxide can be used.

The sacrificial layer 110B is preferably formed using a material that can be dissolved in a solvent chemically stable with respect to the electron-transport layer 108B, which is the uppermost layer. In particular, a material that will be dissolved in water or alcohol can be suitably used for the sacrificial layer 110B. In formation of the sacrificial layer 110B, it is preferable that application of such a material dissolved in a solvent such as water or alcohol be performed by a wet film formation method and followed by heat treatment for evaporating the solvent. At this time, the heat treatment is preferably performed in a reduced-pressure atmosphere, in which case the solvent can be removed at a low temperature in a short time and thermal damage to the hole-injection/transport layer 104B, the light-emitting layer 105B, and the electron-transport layer 108B can be reduced accordingly.

In the case where the sacrificial layer 110B having a stacked-layer structure is formed, the stacked-layer structure can include the first sacrificial layer formed using any of the above-described materials and the second sacrificial layer formed thereover.

The second sacrificial layer in that case is a film used as a hard mask for etching of the first sacrificial layer. In processing the second sacrificial layer, the first sacrificial layer is exposed. Thus, a combination of films having high etching selectivity therebetween is selected for the first sacrificial layer and the second sacrificial layer. Thus, a film that can be used for the second sacrificial layer can be selected in accordance with the etching conditions of the first sacrificial layer and the etching conditions of the second sacrificial layer.

For example, in the case where dry etching using a gas containing fluorine (also referred to as a fluorine-based gas) is performed for the etching of the second sacrificial layer, silicon, silicon nitride, silicon oxide, tungsten, titanium, molybdenum, tantalum, tantalum nitride, an alloy containing molybdenum and niobium, an alloy containing molybdenum and tungsten, or the like can be used for the second sacrificial layer. Here, a metal oxide film of IGZO, ITO, or the like is given as an example of a film having high etching selectivity (that is, enabling low etching rate) in dry etching using the fluorine-based gas, and such a film can be used as the first sacrificial layer.

Note that the material for the second sacrificial layer is not limited to the above and can be selected from a variety of materials in accordance with the etching conditions of the first sacrificial layer and the etching conditions of the second sacrificial layer. For example, any of the films that can be used for the first sacrificial layer can be selected.

As the second sacrificial layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used as the second sacrificial layer. Typically, a film of an oxide or an oxynitride such as silicon oxide, silicon oxynitride, aluminum oxide, aluminum oxynitride, hafnium oxide, or hafnium oxynitride can be used.

Next, as illustrated in FIG. 3C, a resist is applied onto the sacrificial layer 110B, and the resist having a desired shape (a resist mask REG) is formed by a photolithography method. Such a method involves heat treatment steps such as heating after resist application (PAB: Pre Applied Bake) and heating after light exposure (PEB: Post Exposure Bake). The PAB temperature reaches approximately 100° C. and the PEB temperature reaches approximately 120° C., for example. Therefore, the light-emitting device needs to be resistant to such treatment temperatures.

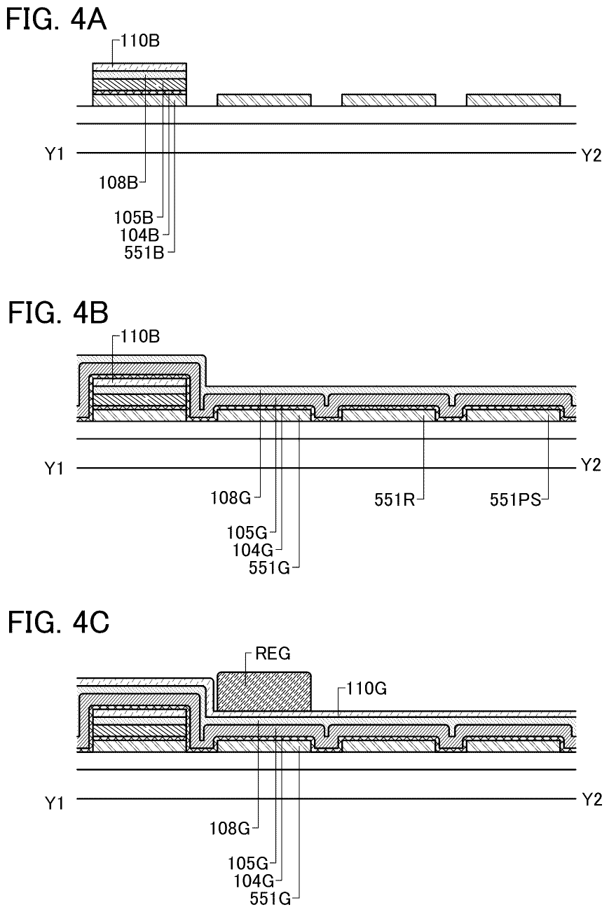

Next, part of the sacrificial layer 110B which is not covered with the resist mask REG is removed by etching using the obtained resist mask REG, the resist mask REG is removed, and then parts of the hole-injection/transport layer 104B, the light-emitting layer 105B, and the electron-transport layer 108B which are not covered with the sacrificial layer 110B are removed by etching, so that the hole-injection/transport layer 104B, the light-emitting layer 105B, and the electron-transport layer 108B are processed to have side surfaces (or have their side surfaces exposed) over the electrode 551B or have belt-like shapes extending in the direction intersecting the sheet. Note that dry etching is preferably employed for the etching. In the case where the sacrificial layer 110B has the aforementioned stacked-layer structure of the first sacrificial layer and the second sacrificial layer, the hole-injection/transport layer 104B, the light-emitting layer 105B, and the electron-transport layer 108B may be processed into desired shapes in the following manner: part of the second sacrificial layer is etched using the resist mask REG, the resist mask REG is then removed, and part of the first sacrificial layer is etched using the second sacrificial layer as a mask. The shape in FIG. 4A is obtained through the etching treatment.

Subsequently, as illustrated in FIG. 4B, the hole-injection/transport layer 104G, the light-emitting layer 105G, and the electron-transport layer 108G are formed over the sacrificial layer 110B, the electrode 551G, the electrode 551R, and the electrode 551PS. As materials for forming the hole-injection/transport layer 104G, the light-emitting layer 105G, and the electron-transport layer 108G, any of the materials described in Embodiment 2 can be used. Note that the hole-injection/transport layer 104G, the light-emitting layer 105G, and the electron-transport layer 108G can be formed by a vacuum evaporation method, for example.

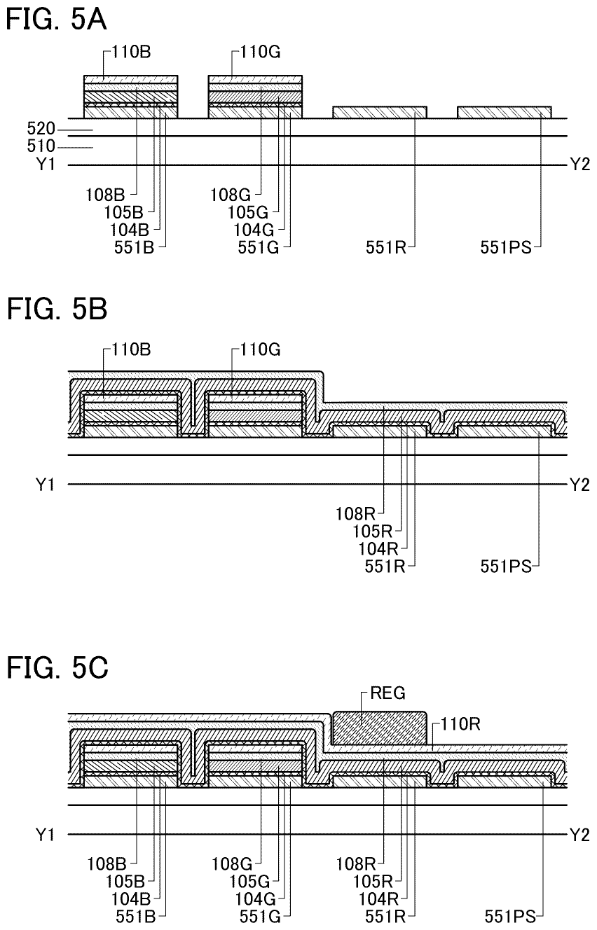

Next, as illustrated in FIG. 4C, the sacrificial layer 110G is formed over the electron-transport layer 108G, a resist is applied onto the sacrificial layer 110G, and the resist having a desired shape (the resist mask REG) is formed by a lithography method. Part of the sacrificial layer 110G which is not covered with the obtained resist mask REG is removed by etching, the resist mask REG is removed, and then parts of the hole-injection/transport layer 104G, the light-emitting layer 105G, and the electron-transport layer 108G which are not covered with the sacrificial layer 110G are removed by etching. Thus, the hole-injection/transport layer 104G, the light-emitting layer 105G, and the electron-transport layer 108G are processed to have side surfaces (or have their side surfaces exposed) over the electrode 551G or have belt-like shapes extending in the direction intersecting the sheet. Note that dry etching is preferably employed for the etching. The sacrificial layer 110G can be formed using a material similar to that for the sacrificial layer 110B. In the case where the sacrificial layer 110G has the above-mentioned stacked-layer structure of the first sacrificial layer and the second sacrificial layer, the hole-injection/transport layer 104G, the light-emitting layer 105G, and the electron-transport layer 108G may be processed into desired shapes in the following manner: part of the second sacrificial layer is etched using the resist mask REG, the resist mask REG is then removed, and part of the first sacrificial layer is etched using the second sacrificial layer as a mask. The shape in FIG. 5A is obtained through the etching treatment.

Next, as illustrated in FIG. 5B, the hole-injection/transport layer 104R, the light-emitting layer 105R, and the electron-transport layer 108R are formed over the sacrificial layer 110B, the sacrificial layer 110G, the electrode 551R, and the electrode 551PS. As materials for forming the hole-injection/transport layer 104R, the light-emitting layer 105R, and the electron-transport layer 108R, any of the materials described in Embodiment 2 can be used. Note that the hole-injection/transport layer 104R, the light-emitting layer 105R, and the electron-transport layer 108R can be formed by a vacuum evaporation method, for example.

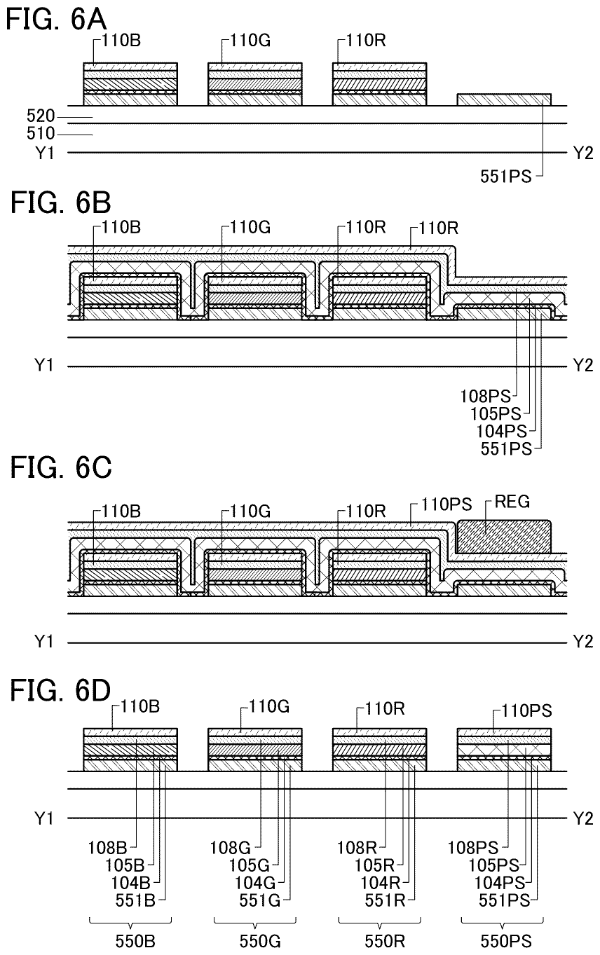

Next, as illustrated in FIG. 5C, the sacrificial layer 110R is formed over the electron-transport layer 108R, a resist is applied onto the sacrificial layer 110R, and the resist having a desired shape (the resist mask REG) is formed by a photolithography method. Part of the sacrificial layer 110R which is not covered with the obtained resist mask REG is removed by etching, the resist mask REG is removed, and then parts of the hole-injection/transport layer 104R, the light-emitting layer 105R, and the electron-transport layer 108R which are not covered with the sacrificial layer 110R are removed by etching. Thus, the hole-injection/transport layer 104R, the light-emitting layer 105R, and the electron-transport layer 108R are processed to have side surfaces (or have their side surfaces exposed) over the electrode 551R or have belt-like shapes extending in the direction intersecting the sheet. Note that dry etching is preferably employed for the etching. The sacrificial layer 110R can be formed using a material similar to that for the sacrificial layer 110B. In the case where the sacrificial layer 110R has the above-mentioned stacked-layer structure of the first sacrificial layer and the second sacrificial layer, the hole-injection/transport layer 104R, the light-emitting layer 105R, and the electron-transport layer 108R may be processed into desired shapes in the following manner: part of the second sacrificial layer is etched using the resist mask REG, the resist mask REG is then removed, and part of the first sacrificial layer is etched using the second sacrificial layer as a mask. The shape in FIG. 6A is obtained through the etching treatment.

Next, as illustrated in FIG. 6B, the first transport layer 104PS, the active layer 105PS, and the second transport layer 108PS are formed over the sacrificial layer 110B, the sacrificial layer 110G, the sacrificial layer 110R, and the electrode 551PS. As a material for forming the first transport layer 104PS, for example, the material for the hole-injection layer and the hole-transport layer described in Embodiment 2 can be used. As a material for the active layer 105PS, a material described in Embodiment 8 can be used. Furthermore, as a material for forming the second transport layer 108PS, for example, the material for the electron-transport layer and the electron-injection layer described in Embodiment 2 can be used. Note that the first transport layer 104PS, the active layer 105PS, and the second transport layer 108PS can be formed by a vacuum evaporation method, for example.

Next, as illustrated in FIG. 6C, the sacrificial layer 110PS is formed over the second transport layer 108PS, a resist is applied onto the sacrificial layer 110PS, and the resist having a desired shape (the resist mask REG) is formed by a photolithography method. Part of the sacrificial layer 110PS that is not covered with the obtained resist mask REG is removed by etching, the resist mask REG is removed, and then parts of the first transport layer 104PS, the active layer 105PS, and the second transport layer 108PS which are not covered with the sacrificial layer 110PS are removed by etching. Thus, the first transport layer 104PS, the active layer 105PS, and the second transport layer 108PS are processed to have side surfaces (or have their side surfaces exposed) over the electrode 551PS or have belt-like shapes extending in the direction intersecting the sheet. Note that dry etching is preferably employed for the etching. The sacrificial layer 110PS can be formed using a material similar to that for the sacrificial layer 110B. In the case where the sacrificial layer 110PS has the above-described stacked-layer structure of the first sacrificial layer and the second sacrificial layer, the first transport layer 104PS, the active layer 105PS, and the second transport layer 108PS may be processed into desired shapes in the following manner: part of the second sacrificial layer is etched using the resist mask REG, the resist mask REG is then removed, and part of the first sacrificial layer is etched using the second sacrificial layer as a mask. The shape in FIG. 6D is obtained through the etching treatment.

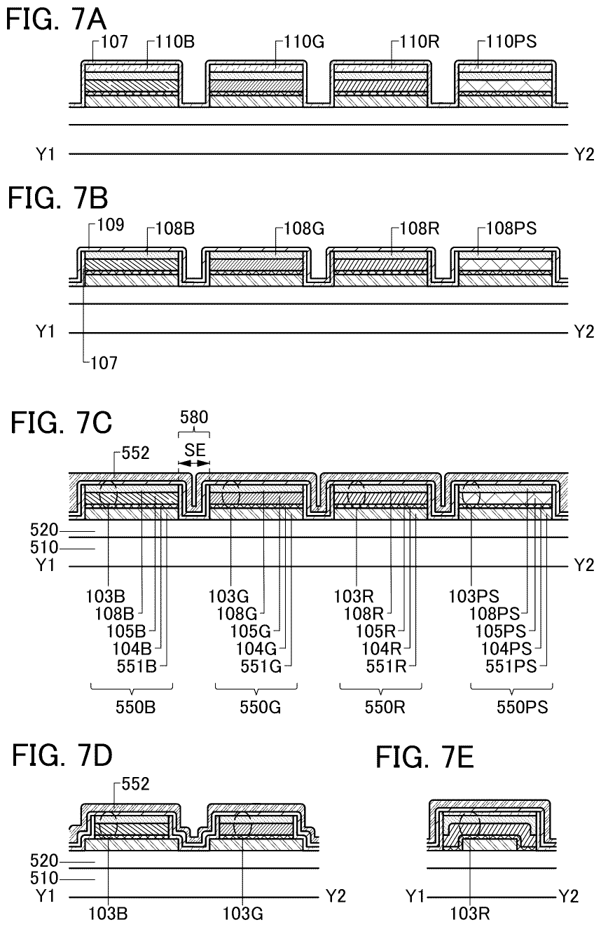

Next, as illustrated in FIG. 7A, the insulating layer 107 is formed over the sacrificial layer 110B, the sacrificial layer 110G, the sacrificial layer 110R, and the sacrificial layer 110PS.

For formation of the insulating layer 107, an ALD method can be used, for example. In this case, as illustrated in FIG. 7A, the insulating layer 107 is formed to be in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device. This can inhibit entry of oxygen, moisture, or constituent elements thereof into the inside through the side surfaces. As a material used for the insulating layer 107, aluminum oxide, magnesium oxide, hafnium oxide, gallium oxide, indium gallium zinc oxide, silicon nitride, silicon nitride oxide, or the like can be used, for example.

Then, as illustrated in FIG. 7B, after part of the insulating layer 107 and the sacrificial layers (110B, 110G, 110R, and 110PS) are removed, the electron-injection layer 109 is formed over the insulating layer 107, the electron-transport layers (108B, 108G, and 108R), and the second transport layer 108PS. For forming the electron-injection layer 109, any of the materials described in Embodiment 2 can be used. The electron-injection layer 109 is formed by a vacuum evaporation method, for example. Note that the electron-injection layer 109 is in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device with the insulating layer 107 therebetween.

Next, as illustrated in FIG. 7C, the electrode 552 is formed. The electrode 552 is formed by a vacuum evaporation method, for example. The electrode 552 is formed over the electron-injection layer 109. Note that the electrode 552 is in contact with the side surfaces (end portions) of the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device with the electron-injection layer 109 and the insulating layer 107 therebetween. This can prevent electrical short circuits between the electrode 552 and each of the following layers: the hole-injection/transport layers (104B, 104G, and 104R), the light-emitting layers (105B, 105G, and 105R), and the electron-transport layers (108B, 108G, and 108R) of the light-emitting devices and the first transport layer 104PS, the active layer 105PS, and the second transport layer 108PS of the light-receiving device.

Through the above steps, the EL layer 103B, the EL layer 103G, the EL layer 103R, and the light-receiving layer 103PS in the light-emitting device 550B, the light-emitting device 550G, the light-emitting device 550R, and the light-receiving layer 550PS can be processed to be separated from each other.

The EL layers (103B, 103G, and 103R) and the light-receiving layer 103PS are processed to be separated by patterning using a photolithography method; hence, a light-emitting and light-receiving apparatus (display panel) with a high resolution can be manufactured. End portions (side surfaces) of the layers of the EL layer processed by patterning using a photolithography method have substantially the same surface (or are positioned on substantially the same plane). In addition, the side surfaces (end portions) of the layers of the light-receiving layer processed by patterning using a photolithography method have substantially the same surface (or are positioned on substantially the same plane).

The hole-injection/transport layers (104B, 104G, and 104R) of the EL layers and the first transport layer 104PS of the light-receiving layer often have high conductivity, and thus might cause crosstalk when formed as layers shared by adjacent devices. Therefore, processing the layers to be separated from each other by patterning using a photolithography method as described in this structure example can inhibit occurrence of crosstalk between adjacent devices.

In this structure example, the hole-injection/transport layers (104B, 104G, and 104R), the light-emitting layers (105B, 105G, and 105R), and the electron-transport layers (108B, 108G, and 108R) of the EL layers (103B, 103G, and 103R) included in the light-emitting devices and the first transport layer 104PS, the active layer 105PS, and the second transport layer 108PS of the light-receiving layer 103PS included in the light-receiving device are processed to be separated by patterning using a photolithography method; thus, the end portions (side surfaces) of the processed layers of the EL layer have substantially the same surface (or are positioned on substantially the same plane). In addition, the side surfaces (end portions) of the layers of the light-receiving layer processed by patterning using a photolithography method have substantially the same surface (or are positioned on substantially the same plane).

In addition, the hole-injection/transport layers (104B, 104G, and 104R), the light-emitting layers (105B, 105G, and 105R), and the electron-transport layers (108B, 108G, and 108R) of the EL layers (103B, 103G, and 103R) included in the light-emitting devices and the first transport layer 104PS, the active layer 105PS, and the second transport layer 108PS of the light-receiving layer 103PS included in the light-receiving device are processed to be separated by patterning using a photolithography method. Thus, the space 580 is provided between the processed end portions (side surfaces) of adjacent light-emitting devices. In FIG. 7C, when the space 580 is denoted by a distance SE between the EL layers in the adjacent light-emitting devices, the aperture ratio can be increased and resolution can be increased as the distance SE decreases. By contrast, as the distance SE increases, the effect of the difference in the fabrication process between the adjacent light-emitting devices becomes permissible, which leads to an increase in manufacturing yield. Since the light-emitting device and the light-receiving device manufactured according to this specification are suitable for a miniaturization process, the distance SE between the EL layers or between the EL layer and the light-receiving layer of adjacent devices can be longer than or equal to 0.5 μm and shorter than or equal to 5 μm, preferably longer than or equal to 1 μm and shorter than or equal to 3 μm, further preferably longer than or equal to 1 μm and shorter than or equal to 2.5 μm, and still further preferably longer than or equal to 1 μm and shorter than or equal to 2 μm. Typically, the distance SE is preferably longer than or equal to 1 μm and shorter than or equal to 2 μm (e.g., 1.5 μm or a neighborhood thereof).

In this specification and the like, a device manufactured using a metal mask or an FMM (a fine metal mask, a high-resolution metal mask) is sometimes referred to as a device having an MM (metal mask) structure. In this specification and the like, a device manufactured without using a metal mask or an FMM is sometimes referred to as a device having an MML (metal maskless) structure. A light-emitting apparatus having an MML structure is formed without using a metal mask and thus has higher flexibility in designing the pixel arrangement, the pixel shape, and the like than a light-emitting apparatus having an FMM structure or an MM structure.

Note that an island-shaped EL layer of a light-emitting and light-receiving apparatus having an MML structure is formed not by patterning using a metal mask but by processing after formation of an EL layer. Accordingly, a light-emitting and light-receiving apparatus with a higher resolution or a higher aperture ratio than a conventional one can be achieved. Moreover, EL layers can be formed separately for the respective colors, enabling the light-emitting and light-receiving apparatus to perform extremely clear display with high contrast and high display quality. Moreover, providing the sacrificial layer over the EL layer can reduce damage to the EL layer in the manufacturing process, resulting in an increase in the reliability of the light-emitting device.

The compound including an azine skeleton of one embodiment of the present invention (also referred to as the compound of the present application), which is described in Embodiment 1 or the like, has a feature of high heat resistance or the like; thus, the use of the compound for an electron-transport/injection layer, an electron-transport layer, a light-emitting layer, a hole-transport layer, a hole-injection layer, an intermediate layer, or a cap layer can enhance the effect of reducing damage during the fabrication process of the MML structure. In particular, the use of the compound of the present application for a layer in an upper position, such as a cap layer, an electron-injection layer, or an electron-transport layer, can reduce damage to layers in a lower position. For example, in the case where the compound of the present application is used for the electron-transport layer, a material with a higher Tg than the host material in the light-emitting layer is preferably used as the compound. Similarly, in the case where the compound of the present application is used for the electron-transport layer, a material with a higher Tg than the hole-transport layer material is preferably used as the compound. As the compound including an azine skeleton, any of the compounds represented by General Formulae (G1) to (G4) described in Embodiment 1 is preferably used; however, the present invention is not limited thereto.

In the case where the compound of the present application is used for a common layer such as the electron-injection layer 109, the electron-injection layer 109 and the partition wall 528 are preferably placed to overlap with each other. The compound of the present application has a high Tg and thus can reduce damage to the partition wall 528. The transport layer (the electron-transport layer 108B, the electron-transport layer 108G, the electron-transport layer 108R, or the second transport layer 108PS) may be formed over the partition wall 528. In that case, it is preferable to use the compound of the present application for the transport layer and place the transport layer and the partition wall to overlap with each other. It is also effective to place a layer including the compound of the present application to overlap with the partition wall between the light-receiving device 550PS and the light-emitting device. Note that the electron-transport layer 108B, the electron-transport layer 108G, the electron-transport layer 108R, and the second transport layer 108PS may be provided not as separate layers but as a continuous layer.

It is effective to employ the compound of the present application with high resistance (heat resistance, stability, or the like) for a bendable device (also referred to as flexible device) that uses a flexible substrate as a substrate. For example, it is effective to provide a layer including the compound of the present application to overlap with a bent portion on which load is likely to be applied. The compound of the present application may be placed to overlap with a space between the partition wall 528 and the bent portion. In a device including a plurality of bent portions or in a wind-up device, many bent portions are included; thus, a cap layer and a layer including the compound of the present application are preferably provided to overlap with each other in each of the bent portions. It is also preferable to place the cap layer, the layer including the compound of the present invention, and the partition wall to overlap with each other in each of the bent portions.

In FIG. 2A and FIG. 7C, the widths of the EL layers (103B, 103G, and 103R) are substantially equal to the widths of the electrodes (551B, 551G, and 551R) in the light-emitting device 550B, the light-emitting device 550G, and the light-emitting device 550R, and the width of the light-receiving layer 103PS is substantially equal to the width of the electrode 551PS in the light-receiving device 550PS; however, one embodiment of the present invention is not limited thereto.

In the light-emitting device 550B, the light-emitting device 550G, and the light-emitting device 550R, the widths of the EL layers (103B, 103G, and 103R) may be smaller than the widths of the electrodes (551B, 551G, and 551R). In the light-receiving device 550PS, the width of the light-receiving layer 103PS may be smaller than the width of the electrode 551PS. FIG. 7D illustrates an example in which the widths of the EL layers (103B and 103G) are smaller than the widths of the electrodes (551B and 551G) in the light-emitting device 550B and the light-emitting device 550G.

In the light-emitting device 550B, the light-emitting device 550G, and the light-emitting device 550R, the widths of the EL layers (103B, 103G, and 103R) may be larger than the widths of the electrodes (551B, 551G, and 551R). In the light-receiving device 550PS, the width of the light-receiving layer 103PS may be larger than the width of the electrode 551PS. FIG. 7E illustrates an example in which the width of the EL layer 103R is larger than the width of the electrode 551R in the light-emitting device 550R.

The structures described in this embodiment can be used in an appropriate combination with any of the structures described in the other embodiments.

### Embodiment 4

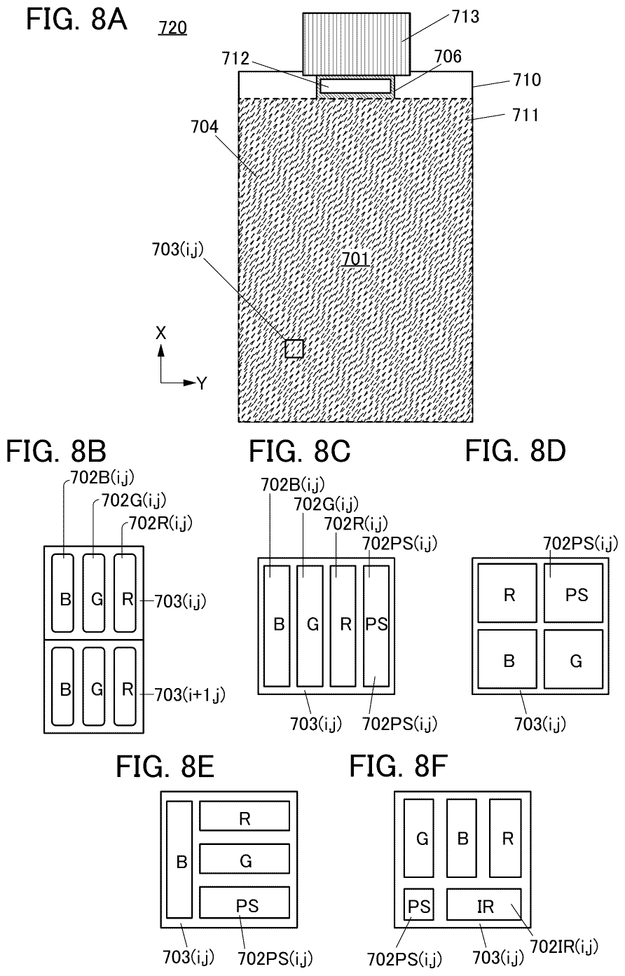

In this embodiment, an apparatus 720 will be described with reference to FIG. 8 to FIG. 10. The apparatus 720 illustrated in FIG. 8 to FIG. 10 includes any of the light-emitting devices described in Embodiment 2 and thus is a light-emitting apparatus. Furthermore, the apparatus 720 described in this embodiment can be used in a display portion of an electronic appliance or the like and thus can also be referred to as a display panel or a display apparatus. Moreover, when the apparatus includes the light-emitting device as a light source and a light-receiving device that can receive light from the light-emitting device, the apparatus can also be referred to as a light-emitting and light-receiving apparatus. Note that the light-emitting apparatus, the display panel, the display apparatus, and the light-emitting and light-receiving apparatus each include at least a light-emitting device.

Furthermore, the light-emitting apparatus, the display panel, the display apparatus, and the light-emitting and light-receiving apparatus of this embodiment can each have a high definition or a large size. Accordingly, the light-emitting apparatus, the display panel, the display apparatus, and the light-emitting and light-receiving apparatus can be used for display portions of electronic appliances such as a digital camera, a digital video camera, a digital photo frame, a mobile phone, a portable game console, a smartphone, a wristwatch terminal, a tablet terminal, a portable information terminal, and an audio reproducing device, in addition to display portions of electronic appliances with a relatively large screen, such as a television device, a desktop or notebook personal computer, a monitor of a computer or the like, digital signage, and a large game machine such as a pachinko machine.

FIG. 8A is a top view of the apparatus 720 (including the light-emitting apparatus, the display panel, the display apparatus, and the light-emitting and light-receiving apparatus).

In FIG. 8A, the apparatus 720 has a structure where a substrate 710 and a substrate 711 are bonded to each other. In addition, the apparatus 720 includes a display region 701, a circuit 704, a wiring 706, and the like. Note that the display region 701 includes a plurality of pixels. As illustrated in FIG. 8B, a pixel 703(i, j) illustrated in FIG. 8A has a pixel 703(i+1, j) adjacent to the pixel 703(i, j).

Furthermore, as illustrated in the example of FIG. 8A, the substrate 710 is provided with an IC (integrated circuit) 712 by a COG (Chip On Glass) method, a COF (Chip On Film) method, or the like in the apparatus 720. As the IC 712, an IC including a scan line driver circuit, a signal line driver circuit, or the like can be used, for example. FIG. 8A illustrates a structure where an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through an FPC (Flexible Printed Circuit) 713 or to the wiring 706 from the IC 712. Note that the apparatus 720 is not necessarily provided with the IC. The IC may be mounted on the FPC by a COF method or the like.

FIG. 8B illustrates the pixel 703(i, j) and the pixel 703(i+1, j) of the display region 701. The pixel 703(i, j) can have a plurality of kinds of subpixels including light-emitting devices that emit light of different colors. In addition to the above, a plurality of subpixels including light-emitting devices that emit light of the same color may be included. In the case where a plurality of kinds of subpixels including light-emitting devices that emit light of different colors are included in the pixel, three kinds of subpixels can be included, for example. As the three subpixels, subpixels of three colors of red (R), green (G), and blue (B), subpixels of three colors of yellow (Y), cyan (C), and magenta (M), and the like can be given. Alternatively, the pixel can include four kinds of subpixels. As the four subpixels, subpixels of four colors of R, G, B, and white (W), subpixels of four colors of R, G, B, and Y, and the like can be given. Specifically, the pixel 703(i, j) can be composed of a subpixel 702B(i, j) displaying blue, a subpixel 702G(i, j) displaying green, and a subpixel 702R(i, j) displaying red.

The apparatus 720 includes not only a subpixel including a light-emitting device, but also a subpixel including a light-receiving device.

FIG. 8C to FIG. 8E illustrate various layout examples of the pixel 703(i, j) including a subpixel 702PS(i, j) including a light-receiving device. The pixel arrangement illustrated in FIG. 8C is stripe arrangement, and the pixel arrangement illustrated in FIG. 8D is matrix arrangement. In the pixel arrangement illustrated in FIG. 8E, three subpixels (the subpixel R, the subpixel G, and the subpixel PS) are vertically arranged next to one subpixel (the subpixel B).

Furthermore, as illustrated in FIG. 8F, a subpixel 702IR(i, j) that emits infrared rays may be added to any of the above-described sets of subpixels to form the pixel 703(i, j). In the pixel arrangement illustrated in FIG. 8F, the three vertically long subpixel G, subpixel B, and subpixel R are arranged laterally, and the subpixel PS and the horizontally long subpixel IR are arranged laterally below the three subpixels. Specifically, the subpixel 702IR(i, j) that emits light including light with a wavelength greater than or equal to 650 nm and less than or equal to 1000 nm may be used in the pixel 703(i, j). Note that the wavelength of light detected by the subpixel 702PS(i, j) is not particularly limited; however, the light-receiving device included in the subpixel 702PS(i, j) preferably has sensitivity to light emitted from the light-emitting device included in the subpixel 702R(i, j), the subpixel 702G(i, j), the subpixel 702B(i, j), or the subpixel 702IR(i, j). The light-receiving device preferably detects one or more of light in blue, violet, bluish violet, green, yellowish green, yellow, orange, red, and infrared wavelength ranges, for example.

Note that the arrangement of subpixels is not limited to the structures illustrated in FIG. 8B to FIG. 8F and a variety of arrangement methods can be employed. The arrangement of subpixels may be stripe arrangement, S stripe arrangement, matrix arrangement, delta arrangement, Bayer arrangement, or PenTile arrangement, for example.

Examples of a top surface shape of the subpixel include polygons such as a triangle, a tetragon (including a rectangle and a square), and a pentagon; polygons with rounded corners; an ellipse; and a circle. Here, the top surface shape of the subpixel corresponds to a top surface shape of a light-emitting region of the light-emitting device.

In the case where a pixel includes a light-receiving device in addition to a light-emitting device, the pixel has a light-receiving function; thus, a touch or an approach of an object can be detected while an image is being displayed. For example, all the subpixels included in the light-emitting apparatus can display an image; alternatively, some of the subpixels can emit light as a light source, and the rest of the subpixels can display an image.

Note that the light-receiving area of the subpixel 702PS(i, j) is preferably smaller than the light-emitting areas of the other subpixels. A smaller light-receiving area leads to a narrower image-capturing range, inhibits a blur in a capturing result, and improves the definition. Thus, by using the subpixel 702PS(i, j), high-resolution or high-definition image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i, j).

Moreover, the subpixel 702PS(i, j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably detects infrared light. Thus, a touch can be detected even in a dark place.

Here, the touch sensor or the near touch sensor can detect the approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the display apparatus is preferably capable of detecting an object positioned in the range of 0.1 mm to 300 mm inclusive, further preferably 3 mm to 50 mm inclusive from the light-emitting and light-receiving apparatus. This structure enables the light-emitting and light-receiving apparatus to be operated without direct contact of an object, that is, enables the light-emitting and light-receiving apparatus to be operated in a contactless (touchless) manner. With the above-described structure, the light-emitting and light-receiving apparatus can be operated with a reduced risk of making the light-emitting and light-receiving apparatus dirty or damaging the light-emitting and light-receiving apparatus or without the object directly touching a dirt (e.g., dust, bacteria, or a virus) attached to the light-emitting and light-receiving apparatus.

For high-resolution image capturing, the subpixels 702PS(i, j) are preferably provided in all pixels included in the light-emitting and light-receiving apparatus. Meanwhile, in the case where the subpixel 702PS(i, j) is used in a touch sensor, a near touch sensor, or the like, high accuracy is not required as compared to the case of capturing an image of a fingerprint or the like; accordingly, the subpixel 702PS(i, j) may be provided in some pixels in the light-emitting and light-receiving apparatus. When the number of the subpixels 702PS(i, j) included in the light-emitting and light-receiving apparatus is smaller than the number of the subpixels 702R(i, j) or the like, higher detection speed can be achieved.

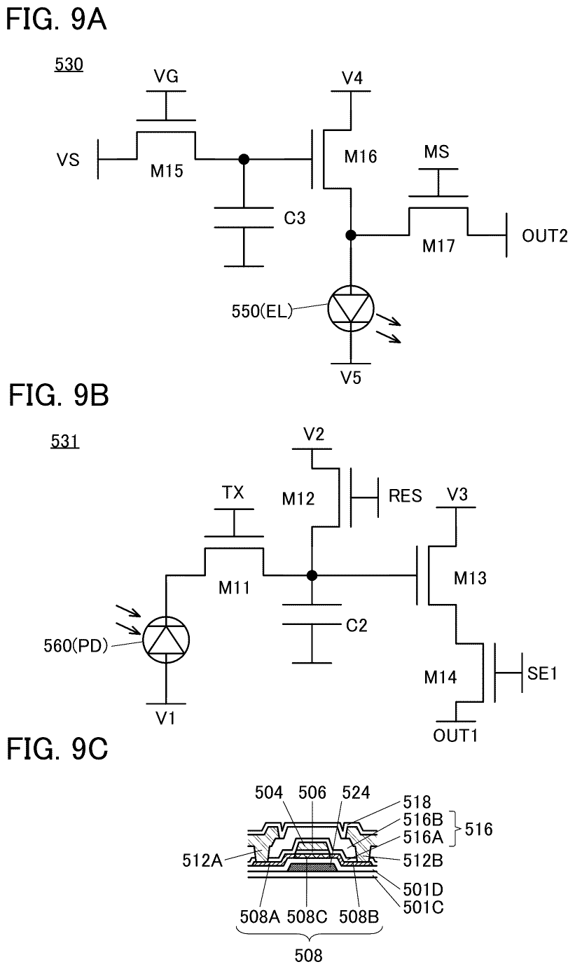

Next, an example of a pixel circuit of a subpixel including the light-emitting device is described with reference to FIG. 9A. A pixel circuit 530 illustrated in FIG. 9A includes a light-emitting device (EL) 550, a transistor M15, a transistor M16, a transistor M17, and a capacitor C3. Note that a light-emitting diode can be used as the light-emitting device 550. In particular, any of the light-emitting devices described in Embodiment 2 is preferably used as the light-emitting device 550.

In the transistor M15 illustrated in FIG. 9A, a gate is electrically connected to a wiring VG, one of a source and a drain is electrically connected to a wiring VS, and the other of the source and the drain is electrically connected to one electrode of the capacitor C3 and a gate of the transistor M16. One of a source and a drain of the transistor M16 is electrically connected to a wiring V4, and the other of the source and the drain of the transistor M16 is electrically connected to an anode of the light-emitting device 550 and one of a source and a drain of the transistor M17. A gate of the transistor M17 is electrically connected to a wiring MS, and the other of the source and the drain of the transistor M17 is electrically connected to a wiring OUT2. A cathode of the light-emitting device 550 is electrically connected to a wiring V5.

A constant potential is supplied to the wiring V4 and the wiring V5. In the light-emitting device 550, the anode side can have a high potential and the cathode side can have a lower potential than the anode side. The transistor M15 is controlled by a signal supplied to the wiring VG and functions as a selection transistor for controlling a selection state of the pixel circuit 530. The transistor M16 functions as a driving transistor that controls a current flowing through the light-emitting device 550 in accordance with a potential supplied to the gate of the transistor M16. When the transistor M15 is in a conduction state, a potential supplied to the wiring VS is supplied to the gate of the transistor M16, and the emission luminance of the light-emitting device 550 can be controlled in accordance with the potential. The transistor M17 is controlled by a signal supplied to the wiring MS and has a function of outputting a potential between the transistor M16 and the light-emitting device 550 to the outside through the wiring OUT2.

Here, transistors in which a metal oxide (an oxide semiconductor) is used in a semiconductor layer where a channel is formed are preferably used as the transistor M15, the transistor M16, and the transistor M17 included in the pixel circuit 530 illustrated in FIG. 9A, and a transistor M11, a transistor M12, a transistor M13, and a transistor M14 included in the pixel circuit 530 illustrated in FIG. 9B.

A transistor using a metal oxide having a wider band gap and a lower carrier density than silicon achieves an extremely low off-state current. Therefore, owing to the low off-state current, charge accumulated in a capacitor that is connected in series to the transistor can be retained for a long time. Accordingly, it is particularly preferable to use transistors containing an oxide semiconductor as the transistor M11, the transistor M12, and the transistor M15 each of which is connected in series to a capacitor C2 or the capacitor C3. When transistors using an oxide semiconductor are used as the other transistors, the manufacturing cost can be reduced.

Alternatively, transistors using silicon for a semiconductor where a channel is formed can be used as the transistor M11 to the transistor M17. It is particularly preferable to use silicon with high crystallinity, such as single crystal silicon or polycrystalline silicon, because high field-effect mobility can be achieved and higher-speed operation can be performed.

Alternatively, a transistor using an oxide semiconductor may be used as one or more of the transistor M11 to the transistor M17, and transistors using silicon may be used as the other transistors.

Next, an example of a pixel circuit of a subpixel including a light-receiving device is described with reference to FIG. 9B. A pixel circuit 531 illustrated in FIG. 9B includes a light-receiving device (PD) 560, the transistor M11, the transistor M12, the transistor M13, the transistor M14, and the capacitor C2. Here, an example in which a photodiode is used as the light-receiving device (PD) 560 is illustrated.

In the light-receiving device (PD) 560 illustrated in FIG. 9B, an anode is electrically connected to a wiring V1, and a cathode is electrically connected to one of a source and a drain of the transistor M11. A gate of the transistor M11 is electrically connected to a wiring TX, and the other of the source and the drain of the transistor M11 is electrically connected to one electrode of the capacitor C2, one of a source and a drain of the transistor M12, and a gate of the transistor M13. A gate of the transistor M12 is electrically connected to a wiring RES, and the other of the source and the drain of the transistor M12 is electrically connected to a wiring V2. One of a source and a drain of the transistor M13 is electrically connected to a wiring V3, and the other of the source and the drain of the transistor M13 is electrically connected to one of a source and a drain of the transistor M14. A gate of the transistor M14 is electrically connected to a wiring SE1, and the other of the source and the drain of the transistor M14 is electrically connected to a wiring OUT1.

A constant potential is supplied to each of the wiring V1, the wiring V2, and the wiring V3. When the light-receiving device (PD) 560 is driven with a reverse bias, the wiring V2 is supplied with a potential higher than the potential of the wiring V1. The transistor M12 is controlled by a signal supplied to the wiring RES and has a function of resetting the potential of a node connected to the gate of the transistor M13 to a potential supplied to the wiring V2. The transistor M11 is controlled by a signal supplied to the wiring TX and has a function of controlling the timing at which the potential of the node changes, in accordance with a current flowing through the light-receiving device (PD) 560. The transistor M13 functions as an amplifier transistor for performing output corresponding to the potential of the node. The transistor M14 is controlled by a signal supplied to the wiring SE1 and functions as a selection transistor for making an external circuit connected to the wiring OUT1 read the output corresponding to the potential of the node. Although n-channel transistors are shown as the transistors in FIG. 9A and FIG. 9B, p-channel transistors can alternatively be used.

The transistors included in the pixel circuit 530 and the transistors included in the pixel circuit 531 are preferably formed to be arranged over the same substrate. It is particularly preferable that the transistors included in the pixel circuit 530 and the transistors included in the pixel circuit 531 be periodically arranged in one region.

One or more layers including the transistor and/or the capacitor are preferably provided to overlap with the light-receiving device (PD) 560 or the light-emitting device (EL) 550. Thus, the effective area occupied by each pixel circuit can be reduced, and a high-resolution light-receiving portion or display portion can be achieved.

FIG. 9C illustrates an example of a specific structure of a transistor that can be used in the pixel circuit described with reference to FIG. 9A and FIG. 9B. As the transistor, a bottom-gate transistor, a top-gate transistor, or the like can be used as appropriate.

The transistor illustrated in FIG. 9C includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region interposed between the semiconductor film 508 and the conductive film 504. The insulating film 506 has a function of a first gate insulating film.

The conductive film 512A has one of a function of a source electrode and a function of a drain electrode, and the conductive film 512B has the other of the function of the source electrode and the function of the drain electrode.

A conductive film 524 can be used in the transistor. The conductive film 524 includes a region where the semiconductor film 508 is interposed between the conductive film 504 and the conductive film 524. The conductive film 524 has a function of a second gate electrode. An insulating film 501D is interposed between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For example, a material having a function of inhibiting diffusion of oxygen, hydrogen, water, an alkali metal, an alkaline earth metal, and the like is preferably used for the insulating film 518. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film with the same composition as the semiconductor film used in the transistor of the pixel circuit can be used in the driver circuit, for example.

The semiconductor film 508 preferably contains indium, M (M is one or more kinds selected from gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more kinds selected from aluminum, gallium, yttrium, and tin.

It is particularly preferable that an oxide containing indium (In), gallium (Ga), and zinc (Zn) (also referred to as IGZO) be used for the semiconductor film 508. Alternatively, it is preferable to use an oxide containing indium, tin, and zinc. Further alternatively, it is preferable to use an oxide containing indium, gallium, tin, and zinc. Alternatively, it is preferable to use an oxide containing indium (In), aluminum (Al), and zinc (Zn) (also referred to as IAZO). Further alternatively, it is preferable to use an oxide containing indium (In), aluminum (Al), gallium (Ga), and zinc (Zn) (also referred to as IAGZO).

In the case where the semiconductor film is an In-M-Zn oxide, the atomic proportion of In is preferably greater than or equal to the atomic proportion of M in the In-M-Zn oxide.

Examples of the atomic ratio of the metal elements in such an In-M-Zn oxide include In:M:Zn=1:1:1 or a composition in the neighborhood thereof, In:M:Zn=1:1:1.2 or a composition in the neighborhood thereof, In:M:Zn=1:3:2 or a composition in the neighborhood thereof, In:M:Zn=1:3:4 or a composition in the neighborhood thereof, In:M:Zn=2:1:3 or a composition in the neighborhood thereof, In:M:Zn=3:1:2 or a composition in the neighborhood thereof, In:M:Zn=4:2:3 or a composition in the neighborhood thereof, In:M:Zn=4:2:4.1 or a composition in the neighborhood thereof, In:M:Zn=5:1:3 or a composition in the neighborhood thereof, In:M:Zn=5:1:6 or a composition in the neighborhood thereof, In:M:Zn=5:1:7 or a composition in the neighborhood thereof, In:M:Zn=5:1:8 or a composition in the neighborhood thereof, In:M:Zn=6:1:6 or a composition in the neighborhood thereof, and In:M:Zn=5:2:5 or a composition in the neighborhood thereof. Note that a composition in the neighborhood includes the range of +30% of an intended atomic ratio.

For example, when the atomic ratio is described as In:Ga:Zn=4:2:3 or a composition in the neighborhood thereof, the case is included where the atomic ratio of Ga is greater than or equal to 1 and less than or equal to 3 and the atomic ratio of Zn is greater than or equal to 2 and less than or equal to 4 with the atomic ratio of In being 4. When the atomic ratio is described as In:Ga:Zn=5:1:6 or a composition in the neighborhood thereof, the case is included where the atomic ratio of Ga is greater than 0.1 and less than or equal to 2 and the atomic ratio of Zn is greater than or equal to 5 and less than or equal to 7 with the atomic ratio of In being 5. When the atomic ratio is described as In:Ga:Zn=1:1:1 or a composition in the neighborhood thereof, the case is included where the atomic ratio of Ga is greater than 0.1 and less than or equal to 2 and the atomic ratio of Zn is greater than 0.1 and less than or equal to 2 with the atomic ratio of In being 1.

There is no particular limitation on the crystallinity of a semiconductor material used for the transistors, and any of an amorphous semiconductor and a semiconductor having crystallinity (a microcrystalline semiconductor, a polycrystalline semiconductor, a single crystal semiconductor, or a semiconductor partly including crystal regions) may be used. A semiconductor having crystallinity is preferably used, in which case degradation of the transistor characteristics can be inhibited.

The semiconductor layer of the transistor preferably includes a metal oxide (also referred to as an oxide semiconductor). As the oxide semiconductor having crystallinity, a CAAC (c-axis aligned crystalline)-OS, an nc (nanocrystalline)-OS, and the like are given.

Alternatively, a transistor using silicon in a channel formation region (a Si transistor) may be used. Examples of silicon include single crystal silicon (single crystal Si), polycrystalline silicon, and amorphous silicon. In particular, a transistor containing low-temperature polysilicon (LTPS) in its semiconductor layer (hereinafter also referred to as an LTPS transistor) can be used. The LTPS transistor has high field-effect mobility and favorable frequency characteristics.

With the use of a Si transistor such as an LTPS transistor, a circuit required to be driven at a high frequency (e.g., a source driver circuit) can be formed on the same substrate as the display portion. This allows simplification of an external circuit mounted on the light-emitting apparatus and a reduction in component cost and mounting cost.

An OS transistor has much higher field-effect mobility than a transistor using amorphous silicon. In addition, an OS transistor has an extremely low leakage current between a source and a drain in an off state (hereinafter also referred to as off-state current), and charge accumulated in a capacitor that is connected in series to the transistor can be retained for a long period. Furthermore, the power consumption of the light-emitting apparatus can be reduced with the OS transistor.

The off-state current value per micrometer of channel width of the OS transistor at room temperature can be lower than or equal to 1 aA (1×10⁻¹⁸ A), lower than or equal to 1 zA (1×10⁻²¹ A), or lower than or equal to 1 yA (1×10⁻²⁴ A). Note that the off-state current value per micrometer of channel width of a Si transistor at room temperature is higher than or equal to 1 fA (1×10⁻¹⁵ A) and lower than or equal to 1 pA (1×10⁻¹² A). In other words, the off-state current of an OS transistor is lower than that of a Si transistor by approximately ten orders of magnitude.

To increase the emission luminance of the light-emitting device included in a pixel circuit, it is necessary to increase the amount of current flowing through the light-emitting device. For that purpose, the source-drain voltage of the driving transistor included in the pixel circuit needs to be increased. Since an OS transistor has a higher withstand voltage between the source and the drain than a Si transistor, a high voltage can be applied between the source and the drain of the OS transistor. Thus, with use of an OS transistor as a driving transistor included in the pixel circuit, the amount of current flowing through the light-emitting device can be increased, resulting in an increase in emission luminance of the light-emitting device.

When a transistor operates in a saturation region, a change in source-drain current relative to a change in gate-source voltage can be smaller in an OS transistor than in a Si transistor. Accordingly, when an OS transistor is used as the driving transistor included in the pixel circuit, current flowing between the source and the drain can be set minutely by a change in gate-source voltage; hence, the amount of current flowing through the light-emitting device can be controlled. Accordingly, the number of gray levels in the pixel circuit can be increased.

Regarding saturation characteristics of current flowing when a transistor operates in a saturation region, even in the case where the source-drain voltage of an OS transistor increases gradually, more stable current (saturation current) can be made flow through an OS transistor than through a Si transistor. Thus, with use of an OS transistor as a driving transistor, current can be made flow stably through the light-emitting device, for example, even when a variation in current-voltage characteristics of the light-emitting device occurs. In other words, when the OS transistor operates in the saturation region, the source-drain current hardly changes with an increase in the source-drain voltage; hence, the emission luminance of the light-emitting device can be stable.

As described above, with use of an OS transistor as the driving transistor included in the pixel circuit, it is possible to achieve “inhibition of black floating”, “increase in emission luminance”, “increase in the number of gray levels”, “inhibition of variation in light-emitting devices”, and the like.

The semiconductor film used in the transistor of the driver circuit can be formed in the same step as the semiconductor film used in the transistor of the pixel circuit. The driver circuit can be formed over a substrate where the pixel circuit is formed. The number of components of an electronic appliance can be reduced.

Silicon may be used for the semiconductor film 508. Examples of silicon include single crystal silicon, polycrystalline silicon, and amorphous silicon. In particular, a transistor containing low-temperature polysilicon (LTPS) in its semiconductor layer (hereinafter also referred to as an LTPS transistor) is preferably used. The LTPS transistor has high field-effect mobility and favorable frequency characteristics.

With the use of a transistor containing silicon, such as an LTPS transistor, a circuit required to be driven at a high frequency (e.g., a source driver circuit) can be formed on the same substrate as the display portion. This allows simplification of an external circuit mounted on the light-emitting apparatus and a reduction in component cost and mounting cost.

It is preferable to use a transistor containing a metal oxide (hereinafter also referred to as an oxide semiconductor) in its semiconductor where a channel is formed (hereinafter also referred to as an OS transistor) as at least one of the transistors included in the pixel circuit. An OS transistor has much higher field-effect mobility than a transistor using amorphous silicon. In addition, an OS transistor has an extremely low leakage current between a source and a drain in an off state (hereinafter also referred to as off-state current), and charge accumulated in a capacitor that is connected in series to the transistor can be retained for a long period. Furthermore, the power consumption of the light-emitting apparatus can be reduced with the OS transistor.

When LTPS transistors are used as some of the transistors included in the pixel circuit and OS transistors are used as the rest, the light-emitting apparatus can have low power consumption and high driving capability. As a favorable example, it is preferable that an OS transistor be used as a transistor functioning as a switch for controlling electrical continuity between wirings and an LTPS transistor be used as a transistor for controlling current. Note that a structure where an LTPS transistor and an OS transistor are combined is referred to as LTPO in some cases. LTPO enables the display panel to have low power consumption and high driving capability.

For example, one transistor provided in the pixel circuit functions as a transistor for controlling current flowing through the light-emitting device and can also be referred to as a driving transistor. One of a source and a drain of the driving transistor is electrically connected to the pixel electrode of the light-emitting device. An LTPS transistor is preferably used as the driving transistor. Thus, current flowing through the light-emitting device in the pixel circuit can be increased.

In contrast, another transistor provided in the pixel circuit functions as a switch for controlling selection and non-selection of a pixel and can also be referred to as a selection transistor. A gate of the selection transistor is electrically connected to a gate line, and one of a source and a drain thereof is electrically connected to a source line (signal line). An OS transistor is preferably used as the selection transistor. Accordingly, the gray level of the pixel can be maintained even with an extremely low frame frequency (e.g., 1 fps or less); thus, power consumption can be reduced by stopping the driver in displaying a still image.

In the case of using an oxide semiconductor in a semiconductor film, the apparatus 720 includes a light-emitting device using an oxide semiconductor in its semiconductor film and having an MML (metal maskless) structure. With this structure, the leakage current that might flow through the transistor and the leakage current that might flow between adjacent light-emitting devices (also referred to as lateral leakage current, side leakage current, or the like) can be extremely low. With the structure, a viewer can notice any one or more of the image crispness, the image sharpness, a high chroma, and a high contrast ratio in an image displayed on the display apparatus. When the leakage current that might flow through the transistor and the lateral leakage current that might flow between light-emitting devices are extremely low, display with little leakage of light at the time of black display (what is called black floating) (such display is also referred to as deep black display) can be achieved.

In particular, in the case where a light-emitting device having an MML structure employs the above-described SBS structure, a layer provided between light-emitting devices (for example, also referred to as an organic layer or a common layer which is shared by the light-emitting devices) is divided; accordingly, display with no or extremely small lateral leakage can be achieved.

The structure of transistors used in a display panel may be selected as appropriate depending on the screen size of the display panel. For example, single crystal Si transistors can be used in the display panel with a screen diagonal greater than or equal to 0.1 inches and less than or equal to 3 inches. In addition, LTPS transistors can be used in the display panel with a screen diagonal greater than or equal to 0.1 inches and less than or equal to 30 inches, preferably greater than or equal to 1 inch and less than or equal to 30 inches. In addition, an LTPO structure (where an LTPS transistor and an OS transistor are used in combination) can be used in the display panel with a screen diagonal greater than or equal to 0.1 inches and less than or equal to 50 inches, preferably greater than or equal to 1 inch and less than or equal to 50 inches. In addition, OS transistors can be used in the display panel with a screen diagonal greater than or equal to 0.1 inches and less than or equal to 200 inches, preferably greater than or equal to 50 inches and less than or equal to 100 inches.

Note that with use of single crystal Si transistors, an increase in screen size is extremely difficult because of the size of a single crystal Si substrate. Furthermore, since a laser crystallization apparatus is used in the manufacturing process, LTPS transistors are unlikely to respond to an increase in screen size (typically to a screen diagonal greater than 30 inches). By contrast, since the manufacturing process does not necessarily require a laser crystallization apparatus or the like or can be performed at a relatively low process temperature (typically, lower than or equal to 450° C.), OS transistors can be used for a display panel with a relatively large area (typically, a screen diagonal greater than or equal to 50 inches and less than or equal to 100 inches). In addition, LTPO is applicable to a display panel with a size midway between the case of using LTPS transistors and the case of using OS transistors (typically, a diagonal size greater than or equal to 1 inch and less than or equal to 50 inches).

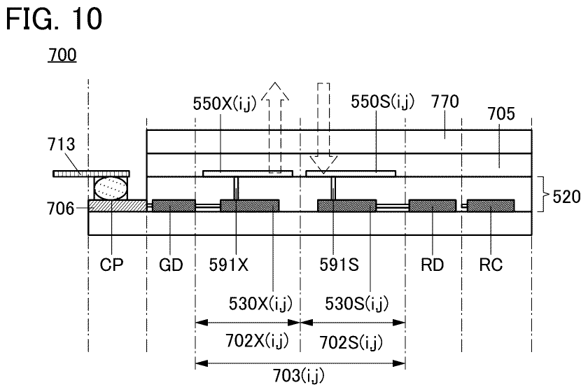

Next, a cross-sectional view of the light-emitting and light-receiving apparatus is shown. FIG. 10 is a cross-sectional view of the light-emitting and light-receiving apparatus illustrated in FIG. 8A.

FIG. 10 is a cross-sectional view of part of a region including the FPC 713 and the wiring 706, and part of the display region 701 including the pixel 703(i, j).

In FIG. 10, the light-emitting and light-receiving apparatus 700 includes the functional layer 520 between the first substrate 510 and the second substrate 770. The functional layer 520 includes, as well as the transistors (M11, M12, M13, M14, M15, M16, and M17), the capacitor (C2 and C3), and the like described with reference to FIG. 9, the wirings (VS, VG, V1, V2, V3, V4, and V5) electrically connecting these components, for example. The structure of the functional layer 520 illustrated in FIG. 10 includes a pixel circuit 530X(i, j), a pixel circuit 530S(i, j), and the driver circuit GD; however, it is not limited thereto.

Each pixel circuit (e.g., the pixel circuit 530X(i, j) and the pixel circuit 530S(i, j) in FIG. 10) included in the functional layer 520 is electrically connected to light-emitting devices and light receiving devices (e.g., a light-emitting device 550X(i, j) and a light-receiving device 550S(i, j) in FIG. 10) formed over the functional layer 520. Specifically, the light-emitting device 550X(i, j) is electrically connected to the pixel circuit 530X(i, j) through a wiring 591X, and the light-receiving device 550S(i, j) is electrically connected to the pixel circuit 530S(i, j) through a wiring 591S. The insulating layer 705 is provided over the functional layer 520, the light-emitting devices, and the light-receiving devices and the insulating layer 705 has a function of bonding the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light receiving apparatus of one embodiment of the present invention can be used as a touch panel.

Note that the structure described in this embodiment can be used in an appropriate combination with any of the structures described in the other embodiments.

### Embodiment 5

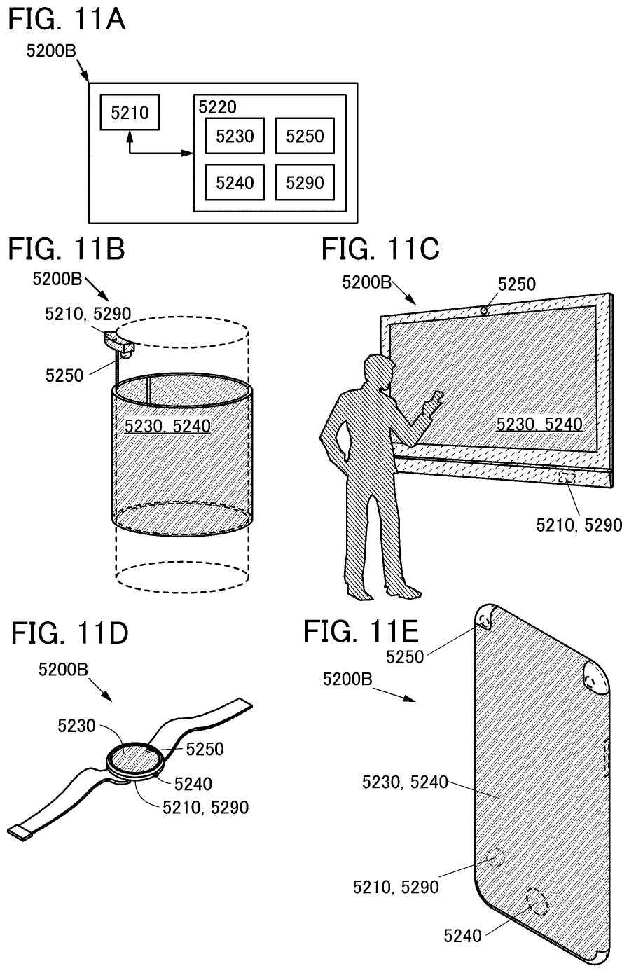

In this embodiment, electronic appliances of embodiments of the present invention will be described with reference to FIG. 11A to FIG. 13B.

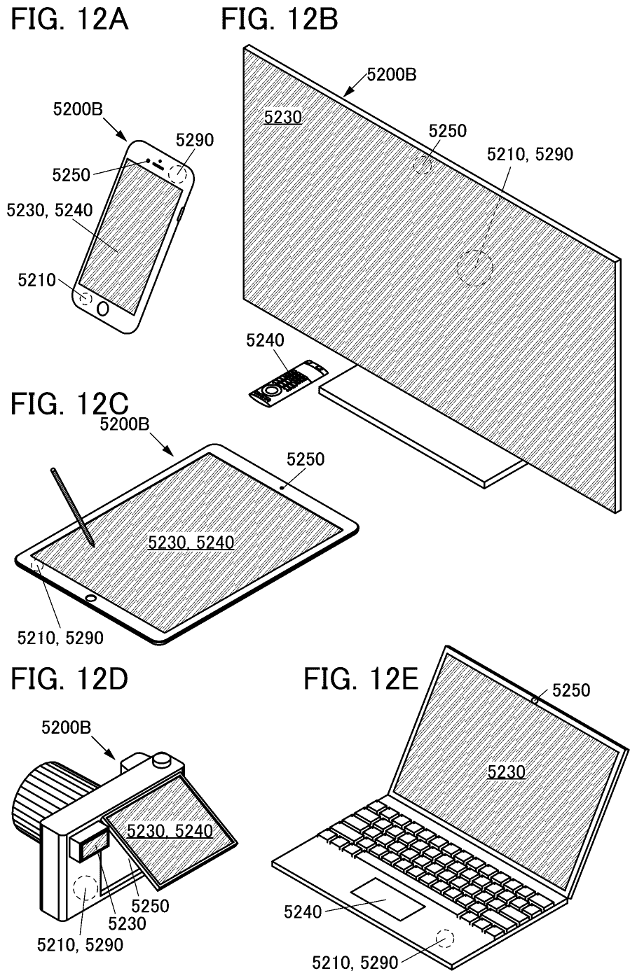

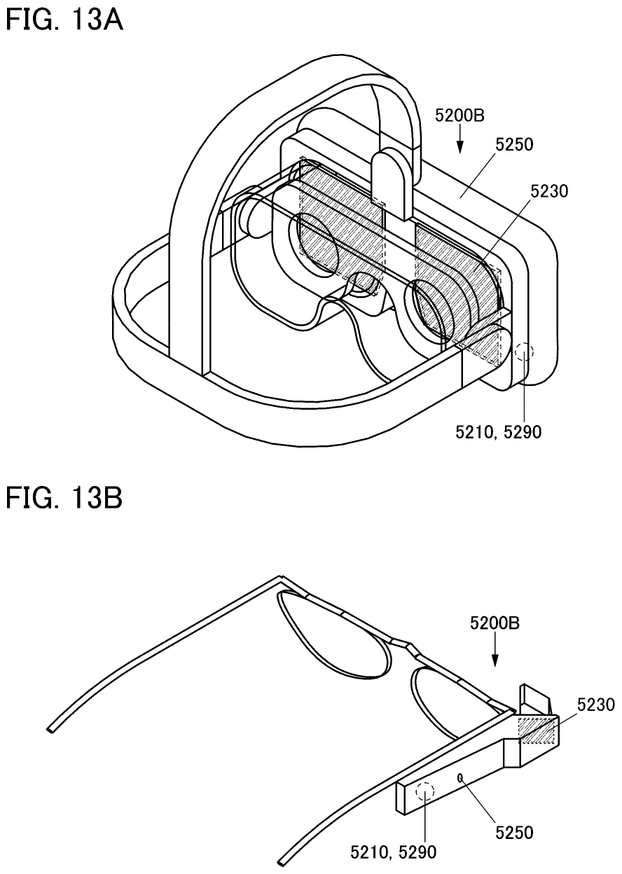

FIG. 11A to FIG. 13B are diagrams illustrating structures of electronic appliances of embodiments of the present invention. FIG. 11A is a block diagram of the electronic appliance and FIG. 11B to FIG. 11E are perspective views illustrating structures of the electronic appliances. FIG. 12A to FIG. 12E are perspective views illustrating structures of electronic appliances. FIG. 13A and FIG. 13B are perspective views illustrating structures of electronic appliances.

An electronic appliance 5200B described in this embodiment includes an arithmetic device 5210 and an input/output device 5220 (see FIG. 11A).

The arithmetic device 5210 has a function of being supplied with operation data and has a function of supplying image data on the basis of the operation data.

The input/output device 5220 includes a display portion 5230, an input portion 5240, a detecting portion 5250, and a communication portion 5290 and has a function of supplying operation data and a function of being supplied with image data. The input/output device 5220 also has a function of supplying detection data, a function of supplying communication data, and a function of being supplied with communication data.

The input portion 5240 has a function of supplying operation data. For example, the input portion 5240 supplies operation data on the basis of operation by a user of the electronic appliance 5200B.

Specifically, a keyboard, a hardware button, a pointing device, a touch sensor, an illuminance sensor, an imaging device, an audio input device, an eye-gaze input device, an attitude detection device, or the like can be used as the input portion 5240.

The display portion 5230 includes a display panel and has a function of displaying image data. For example, the display panel described in Embodiment 3 can be used for the display portion 5230.

The detecting portion 5250 has a function of supplying detection data. For example, the detecting portion 5250 has a function of detecting a surrounding environment where the electronic appliance is used and supplying detection data.

Specifically, an illuminance sensor, an imaging device, an attitude detection device, a pressure sensor, a human motion sensor, or the like can be used as the detecting portion 5250.

The communication portion 5290 has a function of being supplied with communication data and a function of supplying communication data. For example, the communication portion 5290 has a function of being connected to another electronic appliance or a communication network through wireless communication or wired communication. Specifically, the communication portion 5290 has a function of wireless local area network communication, telephone communication, near field communication, or the like.

FIG. 11B illustrates an electronic appliance having an outer shape along a cylindrical column or the like. An example of such an electronic appliance is digital signage. The display panel of one embodiment of the present invention can be used for the display portion 5230. Note that the electronic appliance has a function of changing its display method in accordance with the illuminance of a usage environment. Furthermore, the electronic appliance has a function of changing displayed content in response to detected existence of a person. Thus, for example, the electronic appliance can be provided on a column of a building. The electronic appliance can display advertising, guidance, or the like.

FIG. 11C illustrates an electronic appliance having a function of generating image data on the basis of the path of a pointer used by the user. Examples of such an electronic appliance include an electronic blackboard, an electronic bulletin board, and digital signage. Specifically, the display panel with a diagonal size of 20 inches or longer, preferably 40 inches or longer, and further preferably 55 inches or longer can be used. Alternatively, a plurality of display panels can be arranged and used as one display region. Alternatively, a plurality of display panels can be arranged and used as a multiscreen.

FIG. 11D illustrates an electronic appliance that is capable of receiving data from another device and displaying the data on the display portion 5230. An example of such an electronic appliance is a wearable electronic appliance. Specifically, the electronic appliance can display several options, or allow a user to choose some from the options and send a reply to the data transmitter. Alternatively, for example, the electronic appliance has a function of changing its display method in accordance with the illuminance of a usage environment. Thus, the power consumption of the wearable electronic appliance can be reduced, for example. Alternatively, an image can be displayed on the wearable electronic appliance so that the wearable electronic appliance can be suitably used even in an environment under strong external light, e.g., outdoors in fine weather, for example.

FIG. 11E illustrates an electronic appliance including the display portion 5230 having a surface gently curved along a side surface of a housing. An example of such an electronic appliance is a mobile phone. The display portion 5230 includes a display panel, and the display panel has a function of performing display on the front surface, the side surfaces, the top surface, and the rear surface, for example. Thus, for example, a mobile phone can display data not only on the front surface but also on the side surfaces, the top surface, and the rear surface.

FIG. 12A illustrates an electronic appliance that is capable of receiving data via the Internet and displaying the data on the display portion 5230. An example of such an electronic appliance is a smartphone. For example, a created message can be checked on the display portion 5230. The created message can be sent to another device. The electronic appliance has a function of changing its display method in accordance with the illuminance of a usage environment, for example. Thus, the power consumption of a smartphone can be reduced. A smartphone can display an image so that the smartphone can be suitably used even in an environment under strong external light, e.g., outdoors in fine weather, for example.

FIG. 12B illustrates an electronic appliance that can use a remote controller as the input portion 5240. An example of such an electronic appliance is a television system. For example, the electronic appliance that is capable of receiving data from a broadcast station or via the Internet and performing display on the display portion 5230. An image of a user can be taken using the detecting portion 5250. The image of the user can be transmitted. The electronic appliance can acquire a viewing history of the user and provide it to a cloud service. The electronic appliance can acquire recommendation data from a cloud service and display the data on the display portion 5230. A program or a moving image can be displayed on the basis of the recommendation data. The electronic appliance has a function of changing its display method in accordance with the illuminance of a usage environment, for example. Accordingly, for example, the television system can display an image so that the television system can be suitably used even when irradiated with strong external light that enters a room in fine weather.

FIG. 12C illustrates an electronic appliance that is capable of receiving educational materials via the Internet and displaying them on the display portion 5230. An example of such an electronic appliance is a tablet computer. An assignment can be input with the input portion 5240 and sent via the Internet. A corrected assignment or the evaluation of the assignment can be obtained from a cloud service and displayed on the display portion 5230. Suitable educational materials can be selected on the basis of the evaluation and displayed.

For example, the display can be performed on the display portion 5230 using an image signal received from another electronic appliance. When the electronic appliance is placed on a stand or the like, the display portion 5230 can be used as a sub-display. Thus, for example, a tablet computer can display an image so that the tablet computer can be suitably used even in an environment under strong external light, e.g., outdoors in fine weather.

FIG. 12D illustrates an electronic appliance including a plurality of display portions 5230. An example of such an electronic appliance is a digital camera. For example, the display portion 5230 can display an image that the detecting portion 5250 is capturing. A captured image can be displayed on the detecting portion. A captured image can be decorated using the input portion 5240. A message can be attached to a captured image. A captured image can be transmitted via the Internet. The electronic appliance has a function of changing its shooting conditions in accordance with the illuminance of a usage environment. Accordingly, for example, the digital camera can display an object so that an image is favorably viewed even in an environment under strong external light, e.g., outdoors in fine weather.

FIG. 12E illustrates an electronic appliance in which the electronic appliance of this embodiment is used as a master to control another electronic appliance used as a slave. An example of such an electronic appliance is a portable personal computer. As an example, part of image data can be displayed on the display portion 5230 and another part of image data can be displayed on a display portion of another electronic appliance. Image signals can be supplied to another electronic appliance. With the communication portion 5290, data to be written can be obtained from an input portion of another electronic appliance. Thus, a large display region can be utilized by using the portable personal computer, for example.

FIG. 13A illustrates an electronic appliance including the detecting portion 5250 that detects an acceleration or a direction. An example of such an electronic appliance is a goggles-type electronic appliance. The detecting portion 5250 can supply data on the position of the user or the direction in which the user faces. The electronic appliance can generate image data for the right eye and image data for the left eye in accordance with the position of the user or the direction in which the user faces. The display portion 5230 includes a display region for the right eye and a display region for the left eye. Thus, a virtual reality image that gives the user a sense of immersion can be displayed on the goggles-type electronic appliance, for example.

FIG. 13B illustrates an electronic appliance including the detecting portion 5250 that detects an acceleration or a direction. An example of such an electronic appliance is a glasses-type electronic appliance. The detecting portion 5250 can supply data on the position of the user or the direction in which the user faces. The electronic appliance can generate image data in accordance with the position of the user or the direction in which the user faces. Accordingly, the data can be shown together with a real-world scene, for example. An augmented reality image can be displayed on the glasses-type electronic appliance.

Note that this embodiment can be combined with any of the other embodiments described in this specification as appropriate.

### Embodiment 6

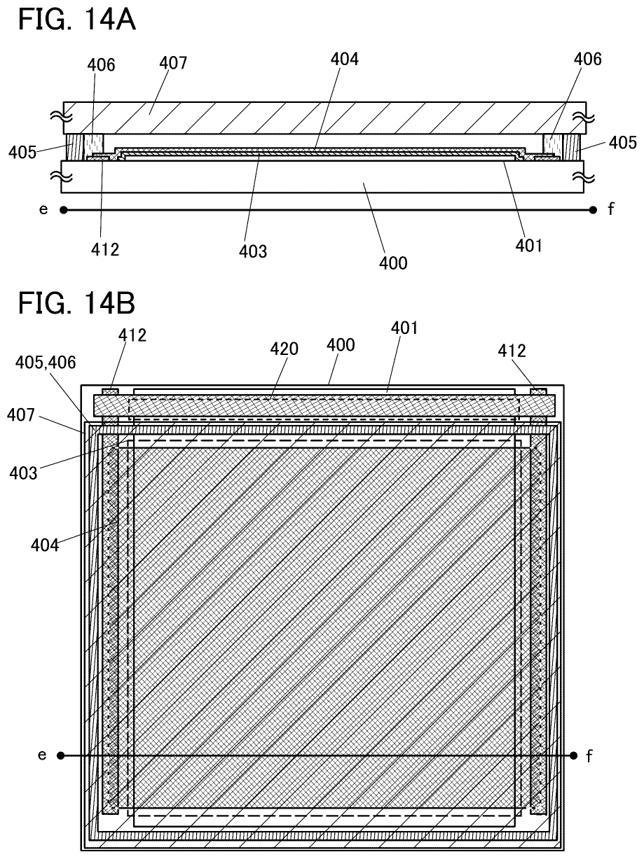

In this embodiment, a structure where the light-emitting device described in Embodiment 2 is used for a lighting device will be described with reference to FIG. 14. FIG. 14A is a cross-sectional view taken along the line e-f in FIG. 14B which is a top view of a lighting device.

In the lighting device in this embodiment, a first electrode 401 is formed over a substrate 400 which is a support and has a light-transmitting property. The first electrode 401 corresponds to the first electrode 101 in Embodiment 2. In the case where light emission is extracted from the first electrode 401 side, the first electrode 401 is formed using a material having a light-transmitting property.

A pad 412 for supplying a voltage to a second electrode 404 is formed over the substrate 400.

An EL layer 403 is formed over the first electrode 401. The structure of the EL layer 403 corresponds to, for example, the structure of the EL layer 103 in Embodiment 2. Note that for these structures, the corresponding description can be referred to.

The second electrode 404 is formed to cover the EL layer 403. The second electrode 404 corresponds to the second electrode 102 in Embodiment 2. In the case where light-emission is extracted from the first electrode 401 side, the second electrode 404 is formed using a material having high reflectivity. The second electrode 404 is supplied with a voltage when connected to the pad 412.

As described above, the lighting device described in this embodiment includes a light-emitting device including the first electrode 401, the EL layer 403, and the second electrode 404. Since the light-emitting device is a light-emitting device with a high emission efficiency, the lighting device in this embodiment can be a lighting device with low power consumption.

The substrate 400 over which the light-emitting device having the above structure is formed is fixed to a sealing substrate 407 with sealants (405 and 406) and sealing is performed, whereby the lighting device is completed. It is possible to use only either the sealant 405 or the sealant 406. In addition, the inner sealant 406 (not illustrated in FIG. 14B) can be mixed with a desiccant, which enables moisture to be adsorbed, resulting in improved reliability.

When parts of the pad 412 and the first electrode 401 are provided to extend to the outside of the sealants 405 and 406, those can serve as external input terminals. An IC chip 420 mounted with a converter or the like, for example, may be provided over the external input terminals.

### Embodiment 7

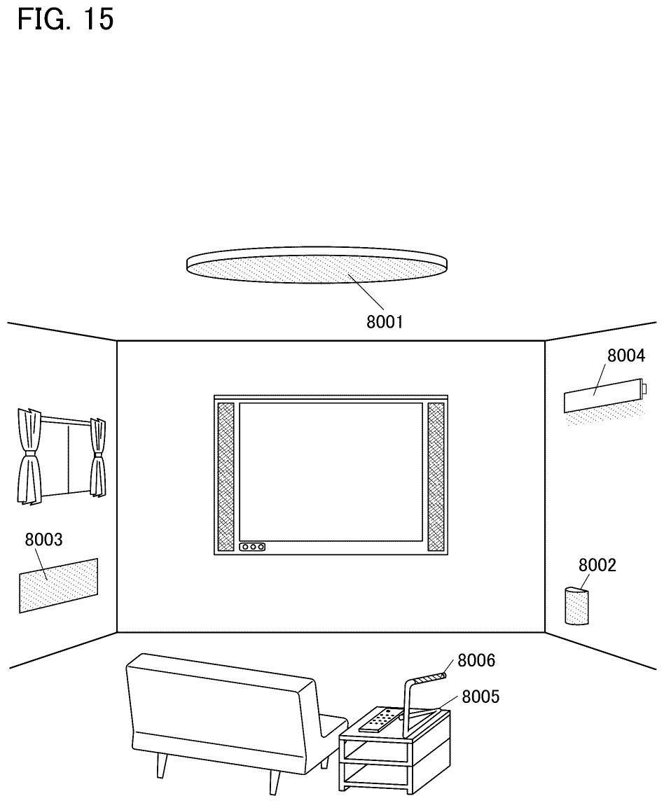

In this embodiment, application examples of lighting devices fabricated using the light-emitting apparatus of one embodiment of the present invention or the light-emitting device, which is part of the light-emitting apparatus, will be described with reference to FIG. 15.

A ceiling light 8001 can be used as an indoor lighting device. Examples of the ceiling light 8001 include a direct-mount light and an embedded light. Such lighting devices are fabricated using the light-emitting apparatus and a housing or a cover in combination. Other than that, application to a cord pendant light (light that is suspended from the ceiling by a cord) is also possible.

A foot light 8002 lights the floor so that safety on the floor can be improved. It can be effectively used in a bedroom, on a staircase, or in a passage, for example. In that case, the size or shape of the foot light can be changed in accordance with the area or structure of a room. The foot light can be a stationary lighting device made from the combination of the light-emitting apparatus and a support.

A sheet-like lighting 8003 is a thin sheet-like lighting device. The sheet-like lighting, which is attached to a wall when used, is space-saving and thus can be used for a wide variety of applications. Furthermore, the area of the sheet-like lighting can be easily increased. The sheet-like lighting can also be used on a wall or housing having a curved surface, for example.

In addition, a lighting device 8004 in which the light from a light source is controlled to be only in a desired direction can be used.

A desk lamp 8005 includes a light source 8006. As the light source 8006, the light-emitting apparatus of one embodiment of the present invention or the light-emitting device, which is part of the light-emitting apparatus, can be used.

In addition to the above examples, when the light-emitting apparatus of one embodiment of the present invention or the light-emitting device, which is part of the light-emitting apparatus, is used as a part of furniture in a room, a lighting device with functions of furniture can be obtained.

As described above, a variety of lighting devices that include the light-emitting apparatus can be obtained. Note that these lighting devices are also embodiments of the present invention.

The structures described in this embodiment can be used in an appropriate combination with any of the structures described in the other embodiments.

### Embodiment 8

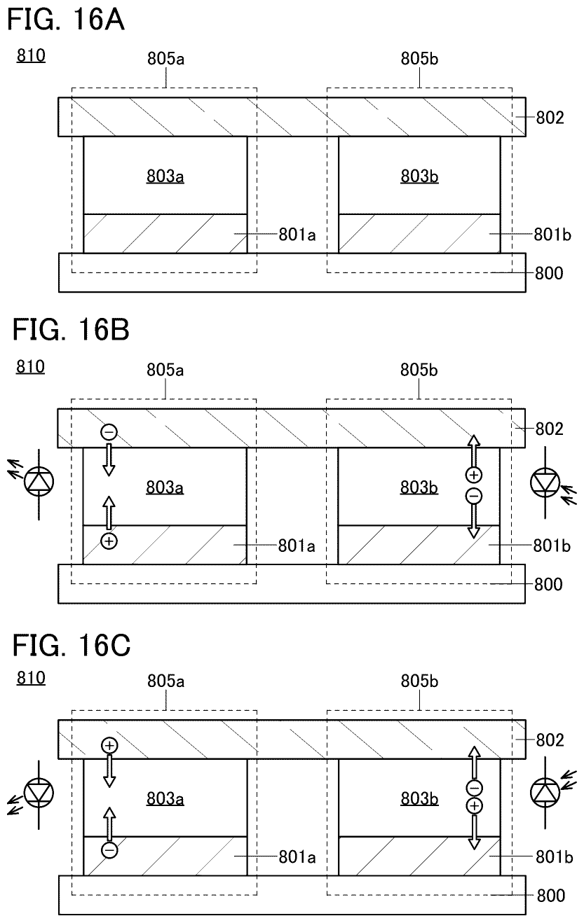

This embodiment will describe a light-emitting and light-receiving apparatus 810 that is illustrated in FIG. 16 and shows examples of a light-emitting device and a light-receiving device usable in a light-emitting apparatus of one embodiment of the present invention. The light-emitting and light-receiving apparatus 810 includes a light-emitting device and thus can be regarded as a light-emitting apparatus, includes a light-receiving device and thus can be regarded as a light-receiving apparatus, and can be used in a display portion in an electronic appliance and thus can be regarded as a display panel or a display apparatus.

FIG. 16A is a schematic cross-sectional view illustrating a light-emitting device 805a and a light-receiving device 805b included in the light-emitting and light-receiving apparatus 810 of one embodiment of the present invention.

The light-emitting device 805a has a function of emitting light (hereinafter also referred to as a light-emitting function). The light-emitting device 805a includes an electrode 801a, an EL layer 803a, and an electrode 802. The light-emitting device 805a is preferably a light-emitting device utilizing organic EL (an organic EL device) described in Embodiment 2. The EL layer 803a interposed between the electrode 801a and the electrode 802 includes at least a light-emitting layer. The light-emitting layer contains a light-emitting substance. The EL layer 803a emits light when voltage is applied between the electrode 801a and the electrode 802. The EL layer 803a may include any of a variety of layers such as a hole-injection layer, a hole-transport layer, an electron-transport layer, an electron-injection layer, a carrier-blocking (hole-blocking or electron-blocking) layer, and a charge-generation layer, in addition to the light-emitting layer.

The light-receiving device 805b has a function of detecting light (hereinafter also referred to as a light-receiving function). For example, a pn or pin photodiode can be used as the light-receiving device 805b. The light-receiving device 805b includes an electrode 801b, a light-receiving layer 803b, and the electrode 802. The light-receiving layer 803b interposed between the electrode 801b and the electrode 802 includes at least an active layer. Note that for the light-receiving layer 803b, any of materials that are used for the variety of layers (e.g., the hole-injection layer, the hole-transport layer, the light-emitting layer, the electron-transport layer, the electron-injection layer, the carrier-blocking (hole-blocking or electron-blocking) layer, and the charge-generation layer) included in the above-described EL layer 803a can be used. The light-receiving device 805b functions as a photoelectric conversion device and generates charge on the basis of incident light on the light-receiving layer 803b, and the charge can be extracted as a current. At this time, voltage may be applied between the electrode 801b and the electrode 802. The amount of generated charge is determined depending on the amount of light incident on the light-receiving layer 803b.

The light-receiving device 805b has a function of detecting visible light. The light-receiving device 805b has sensitivity to visible light. The light-receiving device 805b further preferably has a function of detecting visible light and infrared light. The light-receiving device 805b preferably has sensitivity to visible light and infrared light.

In this specification and the like, a blue (B) wavelength range is greater than or equal to 400 nm and less than 490 nm, and blue (B) light has at least one emission spectrum peak in the wavelength range. A green (G) wavelength range is greater than or equal to 490 nm and less than 580 nm, and green (G) light has at least one emission spectrum peak in the wavelength range. A red (R) wavelength range is greater than or equal to 580 nm and less than 700 nm, and red (R) light has at least one emission spectrum peak in the wavelength range. In this specification and the like, a visible light wavelength is greater than or equal to 400 nm and less than 700 nm, and visible light has at least one emission spectrum peak in the wavelength range. An infrared (IR) wavelength range is greater than or equal to 700 nm and less than 900 nm, and infrared (IR) light has at least one emission spectrum peak in the wavelength range.

The active layer of the light-receiving device 805b contains a semiconductor. Examples of the semiconductor include an inorganic semiconductor such as silicon and an organic semiconductor including an organic compound. As the light-receiving device 805b, an organic semiconductor device (or an organic photodiode) including an organic semiconductor in the active layer is preferably used. An organic photodiode, which is easily made thin, lightweight, and large in area and has high flexibility in shape and design, can be employed for a variety of display apparatuses. With use of an organic semiconductor, the EL layer 803a included in the light-emitting device 805a and the light-receiving layer 803b included in the light-receiving device 805b can be formed by the same method (e.g., a vacuum evaporation method) with the same manufacturing apparatus, which is preferable. Note that the organic compound of one embodiment of the present invention can be used for the light-receiving layer 803b in the light-receiving device 805b.

In the display apparatus of one embodiment of the present invention, an organic EL device can be suitably used as the light-emitting device 805a and an organic photodiode can be suitably used as the light-receiving device 805b. The organic EL device and the organic photodiode can be formed over the same substrate. Thus, the organic photodiode can be incorporated in the display apparatus using the organic EL device. The display apparatus of one embodiment of the present invention has one or both of an image capturing function and a sensing function in addition to an image displaying function.

The electrode 801a and the electrode 801b are provided on the same plane. In FIG. 16A, the electrode 801a and the electrode 801b are provided over a substrate 800. The electrode 801a and the electrode 801b can be formed by processing a conductive film formed over the substrate 800 into island-like shapes, for example. In other words, the electrode 801a and the electrode 801b can be formed through the same process.

As the substrate 800, a substrate having heat resistance high enough to withstand the formation of the light-emitting device 805a and the light-receiving device 805b can be used. When an insulating substrate is used as the substrate 800, a glass substrate, a quartz substrate, a sapphire substrate, a ceramic substrate, an organic resin substrate, or the like can be used. Alternatively, a single crystal semiconductor substrate or a polycrystalline semiconductor substrate using silicon or silicon carbide as a material, a compound semiconductor substrate of silicon germanium or the like, or a semiconductor substrate such as an SOI substrate can be used.

As the substrate 800, it is particularly preferable to use the above-described insulating substrate or semiconductor substrate where a semiconductor circuit including a semiconductor element such as a transistor is formed. The semiconductor circuit preferably forms a pixel circuit, a gate line driver circuit (a gate driver), a source line driver circuit (a source driver), or the like. In addition to the above, an arithmetic circuit, a memory circuit, or the like may be formed.

The electrode 802 is formed of a layer shared by the light-emitting device 805a and the light-receiving device 805b. A conductive film transmitting visible light and infrared light is used as the electrode through which light exits or enters among these electrodes. A conductive film reflecting visible light and infrared light is preferably used as the electrode through which light neither exits nor enters.

The electrode 802 in the display apparatus of one embodiment of the present invention functions as one of the electrodes in each of the light-emitting device 805a and the light-receiving device 805b.

In FIG. 16B, the electrode 801a of the light-emitting device 805a has a potential higher than that of the electrode 802. In this case, the electrode 801a functions as an anode and the electrode 802 functions as a cathode in the light-emitting device 805a. The electrode 801b of the light-receiving device 805b has a potential lower than that of the electrode 802. For easy understanding of the flow direction of current, a circuit symbol of a light-emitting diode is shown on the left of the light-emitting device 805a and a circuit symbol of a photodiode is shown on the right of the light-receiving device 805b in FIG. 16B. The flow directions of carriers (electrons and holes) are also schematically indicated in each device by arrows.

In the structure illustrated in FIG. 16B, when a first potential is supplied to the electrode 801a through a first wiring, a second potential is supplied to the electrode 802 through a second wiring, and a third potential is supplied to the electrode 801b through a third wiring, the following relationship is satisfied: the first potential >the second potential >the third potential.

FIG. 16C illustrates the case where the electrode 801a of the light-emitting device 805a has a potential lower than that of the electrode 802. In this case, the electrode 801a functions as a cathode and the electrode 802 function as an anode in the light-emitting device 805a. The electrode 801b of the light-receiving device 805b has a potential lower than that of the electrode 802 and a potential higher than that of the electrode 801a. For easy understanding of the flow direction of current, FIG. 16C illustrates a circuit symbol of a light-emitting diode on the left of the light-emitting device 805a and a circuit symbol of a photodiode on the right of the light-receiving device 805b. The flow directions of carriers (electrons and holes) are schematically indicated in each device by arrows.

In the structure illustrated in FIG. 16C, when the first potential is supplied to the electrode 801a through the first wiring, the second potential is supplied to the electrode 802 through the second wiring, and the third potential is supplied to the electrode 801b through the third wiring, the following relationship is satisfied: the second potential >the third potential >the first potential.

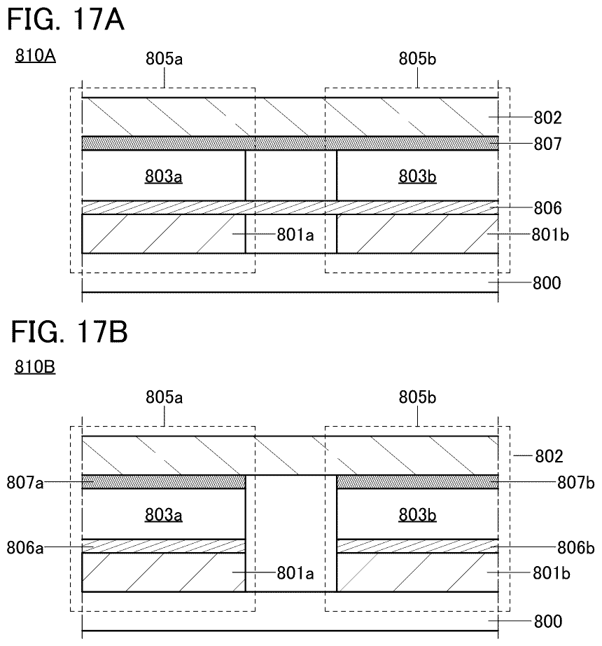

FIG. 17A illustrates a light-emitting and light-receiving apparatus 810A that is a variation example of the light-emitting and light-receiving apparatus 810. The light-emitting and light-receiving apparatus 810A is different from the light-emitting and light-receiving apparatus 810 in including a common layer 806 and a common layer 807. In the light-emitting device 805a, the common layer 806 and the common layer 807 function as part of the EL layer 803a. In the light-receiving device 805b, the common layer 806 and the common layer 807 function as part of the light-receiving layer 803b. The common layer 806 includes a hole-injection layer and a hole-transport layer, for example. The common layer 807 includes an electron-transport layer and an electron-injection layer, for example.

With the common layer 806 and the common layer 807, a light-receiving device can be incorporated without a significant increase in the number of times of separate formation of devices, whereby the light-emitting and light-receiving apparatus 810A can be manufactured with a high throughput.

FIG. 17B illustrates a light-emitting and light-receiving apparatus 810B that is a variation example of the light-emitting and light-receiving apparatus 810A. The light-emitting and light-receiving apparatus 810B is different from the light-emitting and light-receiving apparatus 810A in that the EL layer 803a includes a layer 806a and a layer 807a and the light-receiving layer 803b includes a layer 806b and a layer 807b. The layer 806a and the layer 806b are formed using different materials, and each include a hole-injection layer and a hole-transport layer, for example. Note that the layer 806a and the layer 806b may be formed using the same material. The layer 807a and the layer 807b are formed using different materials, and each include an electron-transport layer and an electron-injection layer, for example. Note that the layer 807a and the layer 807b may be formed using the same material.

An optimum material for forming the light-emitting device 805a is selected for the layer 806a and the layer 807a and an optimum material for forming the light-receiving device 805b is selected for the layer 806b and the layer 807b, whereby the light-emitting device 805a and the light-receiving device 805b can have higher performance in the light-emitting and light-receiving apparatus 810B.

Note that the light-receiving devices 805b described in this embodiment can be arranged at a resolution higher than or equal to 100 ppi, preferably higher than or equal to 200 ppi, more preferably higher than or equal to 300 ppi, further preferably higher than or equal to 400 ppi, still further preferably higher than or equal to 500 ppi and lower than or equal to 2000 ppi, lower than or equal to 1000 ppi, or lower than or equal to 600 ppi, for example. In particular, when the light-receiving devices 805b are arranged at a resolution higher than or equal to 200 ppi and lower than or equal to 600 ppi, preferably higher than or equal to 300 ppi and lower than or equal to 600 ppi, the light-receiving devices can be suitably used for image capturing of a fingerprint. In the case where fingerprint authentication is performed with the display apparatus of one embodiment of the present invention, the increased resolution of the light-receiving devices 805b enables, for example, highly accurate extraction of the minutiae of fingerprints; thus, the accuracy of the fingerprint authentication can be increased. The resolution is preferably higher than or equal to 500 ppi, in which case the authentication conforms to the standard by the National Institute of Standards and Technology (NIST) or the like. On the assumption that the resolution at which the light-receiving devices are arranged is 500 ppi, the size of each pixel is 50.8 μm, which indicates that the resolution is adequate for image capturing of a fingerprint ridge distance (typically, greater than or equal to 300 μm and less than or equal to 500 μm).

The structures described in this embodiment can be used in an appropriate combination with any of the structures described in the other embodiments.

### Example 1

### Synthesis Example 1

This synthesis example specifically describes a method for synthesizing 2-[3-(dibenzo[f,h]quinoxalin-2-yl)-5-(9-phenanthren-9-yl)phenyl]-4,6-diphenyl-1,3,5-triazine (abbreviation: Pn-mDBqPTzn) shown as Structural Formula (100) in Embodiment 1.

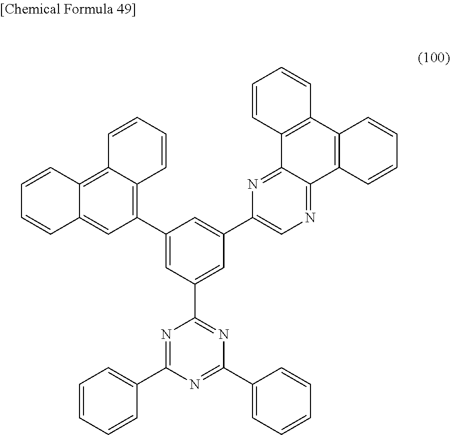 [SMILES: c1ccc(-c2nc(-c3ccccc3)nc(-c3cc(-c4cnc5c6ccccc6c6ccccc6c5n4)cc(-c4cc5ccccc5c5ccccc45)c3)n2)cc1]

### Step 1: Synthesis of 2-[3-chloro-5-(9-phenanthrenyl)phenyl]-4,6-diphenyl-1,3,5-trizazine

Step 1 was performed in the following manner. The synthesis scheme is shown in Formula (A-1).

First, into a 200-mL three-neck flask were put 5.1 g (12 mmol) of 2-(3-bromo-5-chlorophenyl)-4,6-diphenyl-1,3,5-triazine, 2.7 g (12 mmol) of 9-phenanthrene boronic acid, 4.0 g (29 mmol) of potassium carbonate (abbreviation: K₂CO₃), 70 mL of toluene, 10 mL of ethanol, and 10 mL of water. Then, the mixture was degassed by being stirred under reduced pressure, and the air in the flask was replaced with nitrogen. Next, the mixture was heated to 70° C. under a nitrogen stream, 0.42 g (0.60 mmol) of bis(triphenylphosphine)palladium(II) dichloride (abbreviation: Pd(PPh₃)₂Cl₂) was added, the temperature was increased to 90° C., and the mixture was stirred and refluxed for 4 hours. After the reaction, water was added to the mixture and suction filtration was performed, and the residue was washed with water and ethanol. Toluene was added to the obtained residue and heating was performed for dissolution, followed by filtration through Celite and alumina. The obtained filtrate was concentrated and dried to give 5.0 g of a white solid (in a yield of 79%).

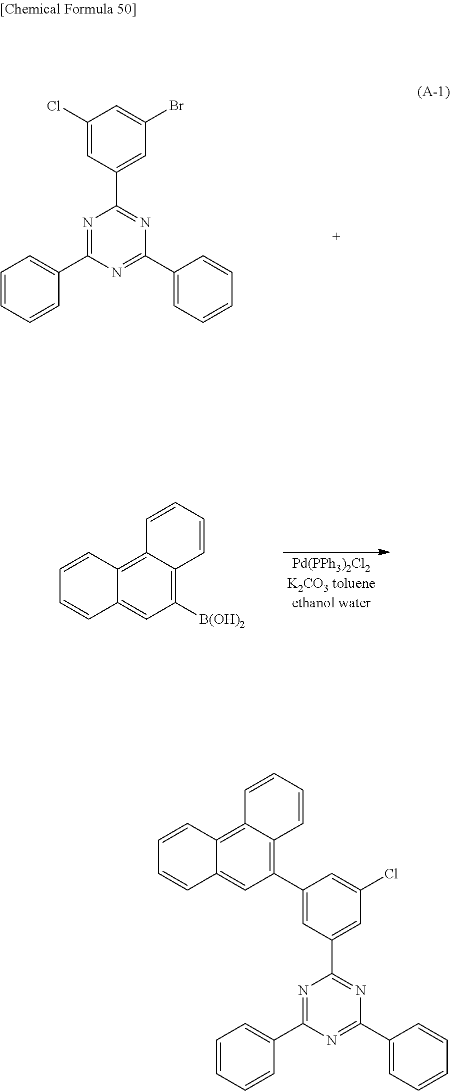 [SMILES: Clc1cc(Br)cc(-c2nc(-c3ccccc3)nc(-c3ccccc3)n2)c1 ; OB(O)c1cc2ccccc2c2ccccc12 ; Clc1cc(-c2nc(-c3ccccc3)nc(-c3ccccc3)n2)cc(-c2cc3ccccc3c3ccccc23)c1]

### Step 2: Synthesis of 2-[3-(9-phenanthrenyl)-5-(4,4,5,5-tetramethyl-1,3,2-dioxaborolan-2-yl)phenyl]-4,6-diphenyl-1,3,5-triazine

Step 2 was performed in the following manner. The synthesis scheme is shown in Formula (A-2).

First, into a 200-mL three-neck flask were put 2.6 g (4.9 mmol) of 2-[3-chloro-5-(9-phenanthrenyl)phenyl]-4,6-diphenyl-1,3,5-triazine obtained in Step 1, 1.9 g (7.4 mmol) of bis(pinacolato)diboron, 1.4 g (15 mmol) of potassium acetate (abbreviation: KOAc), 47 mg (10 μmol) of 2-dicyclohexylphosphino-2′,4′,6′-triisopropylbiphenyl (abbreviation; Xphos), and 50 mL of 1,4-dioxane. Next, the mixture was degassed by being stirred under reduced pressure, and the air in the flask was replaced with nitrogen. After that, the inside of the flask was heated to 70° C. under a nitrogen stream, 11 mg (50 μmol) of palladium acetate (abbreviation: Pd(OAc)₂) was added, the temperature was increased to 100° C., and the mixture was stirred and refluxed for 9 hours. After the reaction, the mixture was suction-filtered, and the obtained filtrate was subjected to extraction using ethyl acetate and washed with water. The obtained organic layer was washed with saturated saline, dried using magnesium sulfate, and then concentrated to give 2.4 g of a white solid (in a yield of 80%).

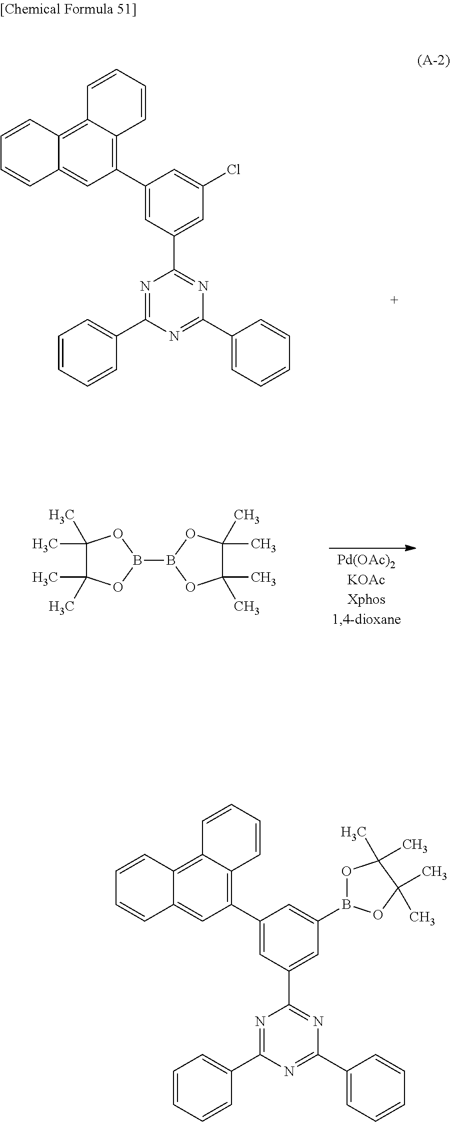

### Step 3: Synthesis of Pn-mDBqPTzn

Step 3 was performed in the following manner. The synthesis scheme is shown in Formula (A-2).

First, into a 200-mL three-neck flask were put 0.83 g (3.1 mmol) of 2-chloro-dibenzoquinoxaline, 1.9 g (3.1 mmol) of the compound obtained in Step 2, 1.0 g (7.2 mmol) of potassium carbonate, 0.10 g (0.33 mmol) of tris(2-methylphenyl)phosphine (abbreviation: P (o-tolyl)₃), 40 mL of toluene, 5 mL of ethanol, and 5 mL of water. Next, the mixture was degassed by being stirred under reduced pressure, and the air in the flask was replaced with nitrogen. After that, the mixture was heated to 70° C. under a nitrogen stream, 15 mg (70 μmol) of palladium acetate was added, the temperature was increased to 90° C., and the mixture was stirred and refluxed for 2.5 hours. After the reaction, the mixture was suction-filtered, and the obtained residue was washed with water and ethanol. Toluene was added to the obtained residue and heating was performed at 50° C., and then suction filtration was performed. The obtained residue was concentrated and dried to give 2.0 g of a white solid (in a yield of 90%).

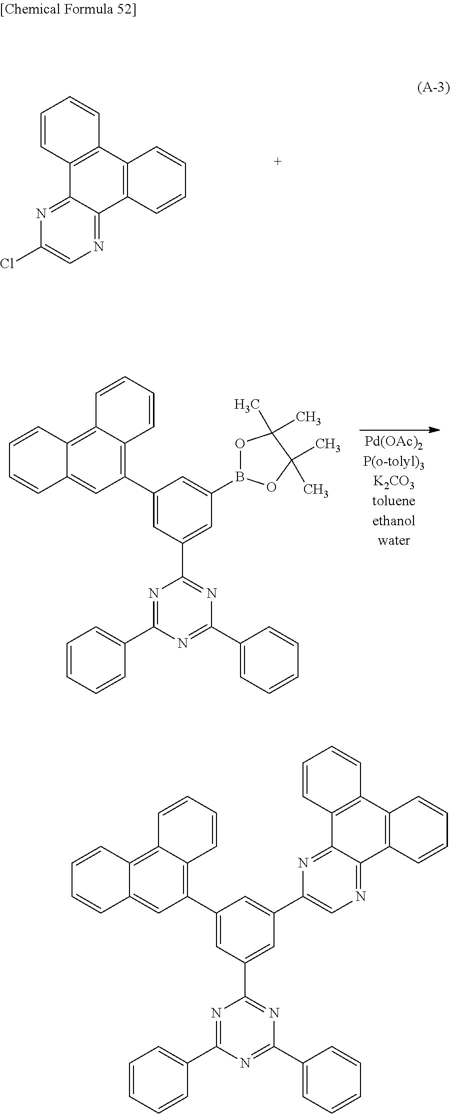 [SMILES: Clc1cnc2c3ccccc3c3ccccc3c2n1 ; CC1(C)OB(c2cc(-c3nc(-c4ccccc4)nc(-c4ccccc4)n3)cc(-c3cc4ccccc4c4ccccc34)c2)OC1(C)C ; c1ccc(-c2nc(-c3ccccc3)nc(-c3cc(-c4cnc5c6ccccc6c6ccccc6c5n4)cc(-c4cc5ccccc5c5ccccc45)c3)n2)cc1]

### <Purification by Sublimation>

Purification by a train sublimation method was performed by heating 2.0 g of the obtained white solid at 360° C. under a pressure of 2.8 Pa with an argon flow rate of 15 mL/min for 14 hours, so that 1.0 g of a white solid was obtained (in a collection rate of 50%).

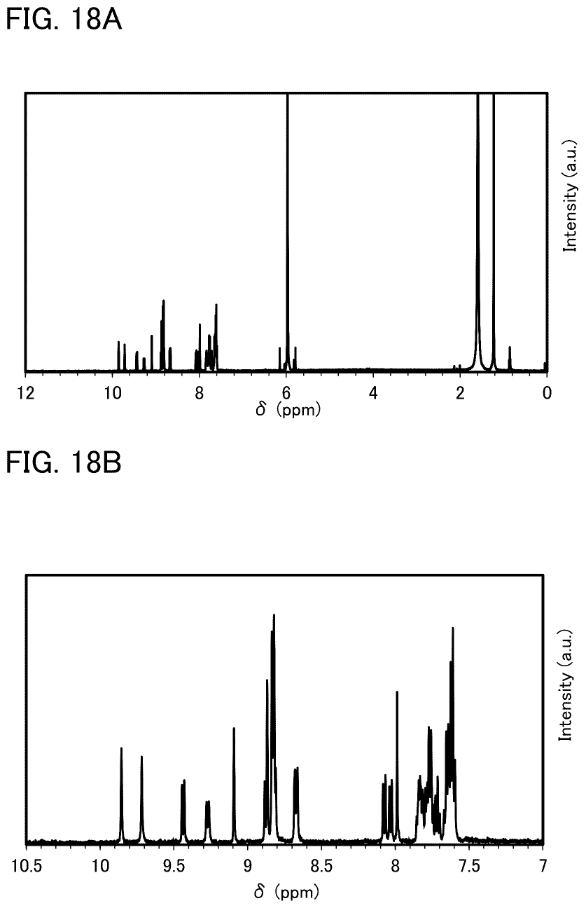

The results of analysis by nuclear magnetic resonance spectroscopy (¹H-NMR) performed on the obtained white solid in a deuterated 1,1,2,2-tetrachloroethane (abbreviation: TCE-d2) solution are shown below. FIG. 18 shows the ¹H-NMR charts (8=0 to 12 ppm in FIG. 18A, and δ=7.0 to 10.0 ppm in FIG. 18B (an enlarged chart)). These results show that Pn-mDBqPTzn, which is the organic compound of the present invention, was obtained in this synthesis example.

¹H-NMR (TCE-d2, 500 MHz): δ=7.60-7.86 (m, 14H), 7.99 (s, 1H), 8.05 (dd, 2H), 8.67 (d, 2H), 8.81-8.89 (m, 7H), 9.09 (s, 1H), 9.27 (d, 1H), 9.44 (d, 1H), 9.72 (s, 1H), 9.86 (s, 1H).

### <Measurement of Emission Spectrum and Absorption Spectrum of Pn-mDBqPTzn>

The absorption spectrum and emission spectrum of Pn-mDBqPTzn were measured. The absorption spectrum was measured with an ultraviolet-visible spectrophotometer (U-4100 produced by Hitachi, Ltd.). The emission spectrum was measured with a fluorescence spectrophotometer (FP-8600, produced by JASCO Corporation).

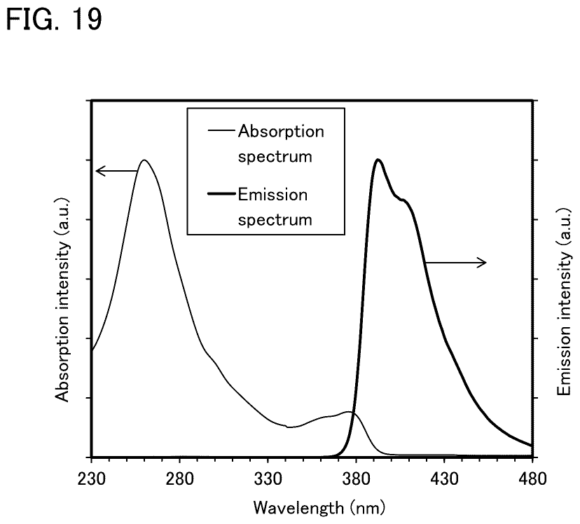

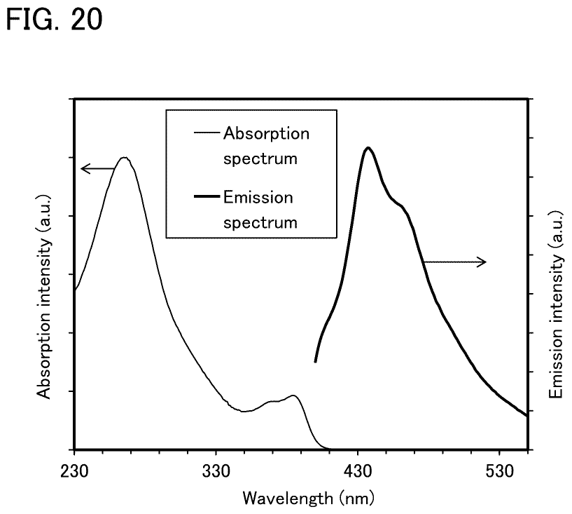

FIG. 19 shows the absorption spectrum and emission spectrum of Pn-mDBqPTzn in a dichloromethane solution. The absorption spectrum in a solution state was obtained by subtraction of a measured absorption spectrum of a solvent (dichloromethane) alone in a quartz cell from a measured absorption spectrum of the solution of Pn-mDBqPTzn in a quartz cell.

FIG. 20 shows an absorption spectrum and an emission spectrum of a thin film. The solid thin film was formed over a quartz substrate by a vacuum evaporation method and another quartz substrate as the counter substrate was used for sealing; thus, a measurement sample was fabricated. Note that the emission spectrum was obtained by measurement of the sealed sample, and the absorption spectrum was obtained by measurement of the sample from which the sealing was removed and the counter substrate was detached. The absorption spectrum of the thin film was obtained by subtraction of an absorption spectrum of a quartz substrate from an absorption spectrum of a film of Pn-mDBqPTzn formed over the quartz substrate. Even after the removal of the sealing at room temperature, no visible change in film quality was observed in the fabricated thin film and the stable amorphous film was found to be maintained. This reveals that the compound of the present application can provide a thin film with excellent stability, and an organic device fabricated using the compound of the present application exhibits excellent stability.

As shown in FIG. 19, in the case of Pn-mDBqPTzn in the dichloromethane solution, the absorption peaks were observed at around 375 nm, 364 nm, and 260 nm, and the emission peak was observed at around 392 nm (excitation wavelength: 260 nm). As shown in FIG. 20, in the case of the thin film of Pn-mDBqPTzn, the absorption peaks were observed at around 390 nm, 370 nm, and 269 nm, and the emission peak was observed at around 438 nm (excitation wavelength: 385 nm). These results indicate that Pn-mDBqPTzn can be effectively used as a light-emitting substance or a host material used in combination with a light-emitting substance in the visible region. It is also indicated that Pn-mDBqPTzn can be effectively used for a cap layer used over a cathode. When Pn-mDBqPTzn is used for these layers, the absorption peak is at desirably greater than or equal to 300 nm and less than or equal to 450 nm, further preferably greater than or equal to 320 nm and less than or equal to 380 nm. The emission peak is at desirably greater than or equal to 350 nm and less than or equal to 650 nm, further preferably greater than or equal to 380 nm and less than or equal to 500 nm.

### <Tg Measurement of Pn-mDBqPTzn>

The glass transition temperature (Tg) of Pn-mDBqPTzn was measured. The Tg was measured with a differential scanning calorimeter (PYRIS 1 DSC produced by PerkinElmer Japan Co., Ltd.) while a powder was put on an aluminum cell. As a result, the Tg of Pn-mDBqPTzn was 161° C. This reveals that the compound of the present invention exhibits an excellent thermal property and the thin film formed using such a compound is expected to have stable film quality. The use of the compound capable of forming a thin film with stable quality allows a highly heat-resistant organic device to be provided.

In an organic device such as a light-emitting device or a light-receiving device, a layer provided in a higher position preferably has higher resistance (heat resistance, film quality stability, or the like). This is because a layer provided in a higher position might be influenced more greatly by the formation process of the organic device. For example, when used for an electron-transport layer, the compound of the present application desirably has a Tg higher than that of a host material in a light-emitting layer in a lower position by 5° C. or more, further preferably 10° C. or more. It is also effective to make the Tg higher than that of a material for a hole-transport layer or a hole-injection layer that is in a lower position by 5° C. or more, preferably 10° C. or more. A layer (an electron-transport layer such as a hole-blocking layer) may be provided between the light-emitting layer and the electron-transport layer, and also in this case, the Tg of the electron-transport layer is preferably made higher than that of the hole-blocking layer.

### <Calculation of HOMO and LOMO of Pn-mDBqPTzn>

The HOMO level and the LUMO level of Pn-mDBqPTzn were calculated on the basis of cyclic voltammetry (CV) measurement. The calculation method is shown below.

An electrochemical analyzer (model number: ALS model 600A or 600C, produced by BAS Inc.) was used as a measurement apparatus. To prepare a solution for the CV measurement, dehydrated dimethylformamide (DMF) (produced by Sigma-Aldrich Inc., 99.8%, catalog No. 22705-6) was used as a solvent, tetra-n-butylammonium perchlorate (n-Bu₄NClO₄) (produced by Tokyo Chemical Industry Co., Ltd., catalog No. T0836) as a supporting electrolyte was dissolved at a concentration of 100 mmol/L, and the object to be measured was dissolved at a concentration of 2 mmol/L.

A platinum electrode (PTE platinum electrode, produced by BAS Inc.) was used as a working electrode, another platinum electrode (Pt counter electrode for VC-3 (5 cm), produced by BAS Inc.) was used as an auxiliary electrode, and an Ag/Ag⁺ electrode (RE7 reference electrode for a non-aqueous solvent, produced by BAS Inc.) was used as a reference electrode. Note that the measurement was performed at room temperature (higher than or equal to 20° C. and lower than or equal to 25° C.).

The scan speed in the CV measurement was fixed to 0.1 V/see, and an oxidation potential Ea [V] and a reduction potential Ec [V] with respect to the reference electrode were measured. Ea is an intermediate potential of an oxidation-reduction wave, and Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]=−4.94−Ea and LUMO level [eV]=−4.94−Ec.

Furthermore, CV measurement was repeated 100 times, and the oxidation-reduction wave in the hundredth cycle was compared with the oxidation-reduction wave in the first cycle to examine the electrical stability of the compound.

From the results, the oxidation potential Ea [V] of Pn-mDBqPTzn was found to be less than the lower measurement limit of the apparatus, and the HOMO level can be estimated to be lower than or equal to −6.4 eV. Meanwhile, from the measurement results of the reduction potential Ec [V], the LUMO level was found to be −3.04 eV.

According to the results of the LUMO level, it can be considered that electrons can be suitably donated and accepted, and Pn-mDBqPTzn can be suitably used for an electron-transport layer and a light-emitting layer of an organic device and a charge-generation layer of a tandem element.

In the case where Pn-mDBqPTzn is used for an electron-transport layer of an organic device, the LUMO level of an electron-transport layer material is preferably lower than the LUMO level of a host material in a light-emitting layer. In addition, the LUMO level of the electron-transport layer material is preferably lower than the LUMO level of a cap layer material. It is particularly preferable that the LUMO levels satisfy the relation of the cap layer material >the host material in the light-emitting layer >the electron-transport layer material. In the case where a plurality of light-emitting devices (e.g., a red device, a blue device, and a green device) are included, each of the light-emitting devices preferably satisfies the relation of the LUMO levels.

### <Measurement of Refractive Index of Pn-mDBqPTzn>

The refractive index of Pn-mDBqPTzn was measured with a spectroscopic ellipsometer (M-2000U produced by J. A. Woollam Japan Corp.). For the measurement, a film formed by depositing Pn-mDBqPTzn to a thickness of approximately 50 nm over a quartz substrate by a vacuum evaporation method was used. At a wavelength of 633 nm, the ordinary refractive index n Ordinary (no) was 1.85. The results show that Pn-mDBqPTzn can be effectively used as the host material, the hole-transport material, the electron-transport material, or the material for the cap layer provided over the cathode. In the case where is used as the material for the cap layer, the refractive index is preferably higher than or equal to 1.75 and lower than or equal to 2.50.

In the case where Pn-mDBqPTzn is used as a hole-transport material or an electron-transport material, lowering the refractive index can further increase the emission efficiency. An example of a method for lowering the refractive index is adding an alkyl group as a substituent to the compound of the present application, by which the refractive index can be adjusted to higher than or equal to 1.50 and lower than or equal to 1.85. For example, in the case of General Formulae (G1) to (G4), at least one of R¹ to R⁴ preferably includes an alkyl group.

When used as the host material, the hole-transport layer material, or the electron-transport layer material, the compound of the present application preferably has a refractive index lower than that of the cap layer material or the host material in the light-emitting layer. Specifically, the refractive index of the compound of the present application is lower than the refractive index of the cap layer preferably by 0.1 or more, further preferably by 0.2 or more. Furthermore, the refractive index of the compound of the present application is lower than the refractive index of the host material in the light-emitting layer preferably by 0.1 or more, further preferably by 0.2 or more. In particular, in the case where Pn-mDBqPTzn is used as the electron-transport layer material, making the refractive index lower than those of the cap layer and the light-emitting layer can increase the emission efficiency.

### Example 2

### Synthesis Example 2

This synthesis example describes a method for synthesizing 2-[3-(2,6-dimethyl-3-pyridinyl)-5-(9-phenanthrenyl)phenyl]-4,6-diphenyl-1,3,5-triazine (abbreviation: mPn-mDMePyPTzn), which is an organic compound usable as a material that can be contained in an organic device (e.g., an electron-transport material and a host material), and is represented by Structural Formula (160).

Note that mPn-mDMePyPTzn was manufactured in a manner similar to that of Synthesis example 1 except that 3-bromo-2,6-dimethylpyridine was used instead of 2-chloro-dibenzoquinoxaline in Step 3 of Synthesis example 1. The synthesis scheme is shown in Formula (B-1). Note that the heating time was 5 hours and the heating temperature was 65° C.

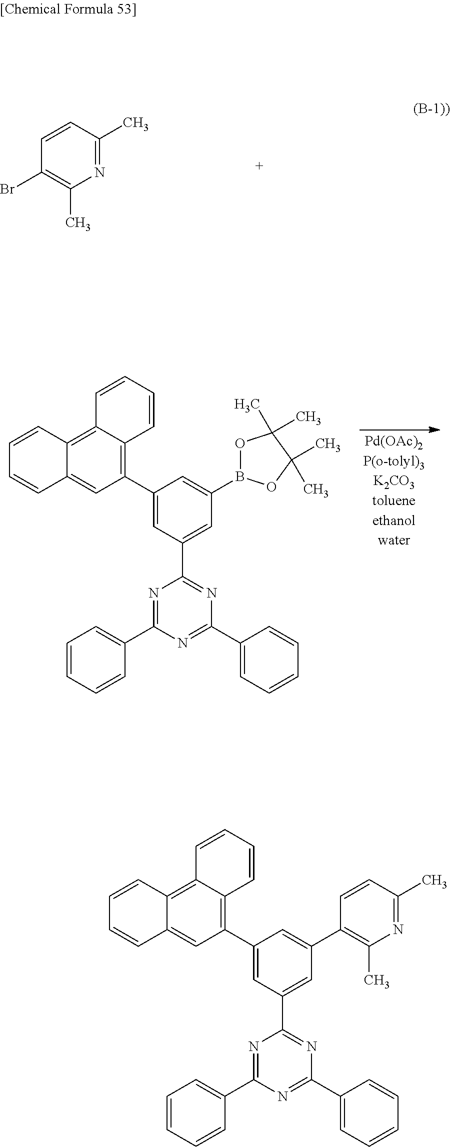

The absorption spectrum and emission spectrum of a light-emitting device containing mPn-mDMePyPTzn were measured in a manner similar to that of Example 1. In a solution state, the peak wavelengths of the absorption spectrum were 256 nm and 350 nm, and the peak wavelength of the emission spectrum was 436 nm. In a thin film state, the peak wavelength of the absorption spectrum was 336 nm, and the peak wavelength of the emission spectrum was 409 nm (excitation wavelength: 310 nm).

The Tg of mPn-mDMePyPTzn, which was measured in a manner similar to that of Example 1, was 121° C.

### Example 3

### Synthesis Example 3

This synthesis example describes a method for synthesizing 2-[(1,1′-biphenyl)-4-yl]-4-phenyl-6-[9,9′-spirobi (9H-fluoren)-2-yl]-1,3,5-triazine (abbreviation: BP-SFTzn), which is an organic compound usable as a material that can be contained in an organic device (e.g., an electron-transport material and a host material), and is represented by Structural Formula (440). The synthesis scheme is shown in Formula (C-1).

Into a 200-mL three-neck flask were put 1.7 g (4.8 mmol) of 2-(biphenyl-4-yl)-4-chloro-6-phenyl-1,3,5-triazine), 2.1 g (5.8 mmol) of 9,9′-spirobi[9H-fluorene]-2-boronic acid), 1.6 g (12 mmol) of potassium carbonate, 50 mL of toluene, 7 mL of ethanol, and 7 mL of water. Next, the mixture was degassed by being stirred under reduced pressure, and the air in the flask was replaced with nitrogen. After that, the inside of the flask was heated to 70° C. under a nitrogen stream, 0.20 g (0.29 mmol) of bis(triphenylphosphine)palladium(II) dichloride was added, the temperature was increased to 90° C., and the mixture was stirred and refluxed for 10 hours. Toluene and water were added to a reactant and an organic layer was extracted. The obtained organic layer was dried using magnesium sulfate and naturally filtered, and the obtained filtrate was concentrated to give a solid. The obtained solid was purified by silica gel column chromatography (toluene:hexane=1:1 in a developing solvent). The obtained solid was recrystallized with toluene and hexane, whereby 2.7 g of a white solid, which is a target compound BP-SFTzn, was obtained (in a yield of 90%).

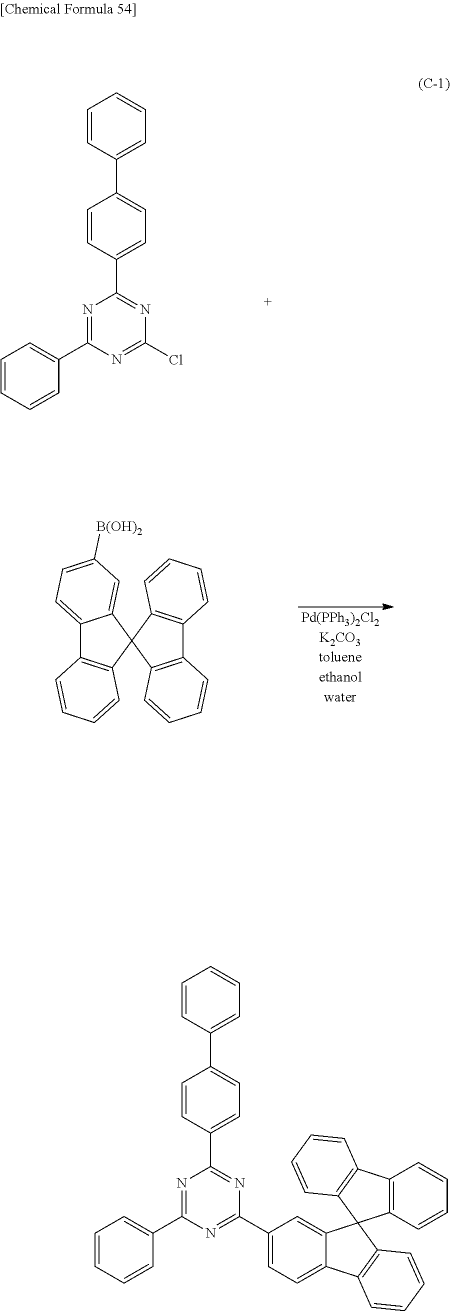 [SMILES: Clc1nc(-c2ccccc2)nc(-c2ccc(-c3ccccc3)cc2)n1 ; OB(O)c1ccc2c(c1)C1(c3ccccc3-c3ccccc31)c1ccccc1-2 ; c1ccc(-c2ccc(-c3nc(-c4ccccc4)nc(-c4ccc5c(c4)C4(c6ccccc6-c6ccccc64)c4ccccc4-5)n3)cc2)cc1]

The absorption spectrum and emission spectrum of BP-SFTzn were measured. In the measurement of a solution state, toluene was used as a solvent. Except for the above, the measurement was performed in a manner similar to that of Example 1. In the solution state, the peak wavelength of the absorption spectrum was 358 nm, and the peak wavelength of the emission spectrum was 388 nm. In a thin film state, the peak wavelength of the absorption spectrum was 352 nm, and the peak wavelength of the emission spectrum was 419 nm.

The Tg of BP-SFTzn, which was measured in a manner similar to that of Example 1, was 148° C.

### Example 4

### Synthesis Example 4

This synthesis example describes a method for synthesizing 2-[(1,1′-biphenyl)-4-yl]-4-phenyl-6-{(3′,5′-di-tert-butyl)-1,1′-biphenyl-3-yl}-1,3,5-triazine (abbreviation: BP-SF(4)Tzn), which is an organic compound usable as a material that can be contained in an organic device (e.g., an electron-transport material and a host material), and is represented by Structural Formula (434).

Note that BP-SF(4)Tzn was manufactured in a manner similar to that of Synthesis example 3 except that 2-(9,9′-spirobi[9H-fluoren]-4-yl)-4,4,5,5-tetramethyl-1,3,2-dioxaborolane) was used instead of 9,9′-spirobi[9H-fluorene]-2-boronic acid) in Synthesis example 3. The synthesis scheme is shown in Formula (D-1). Note that the heating time was 4.5 hours and the heating temperature was 90° C.

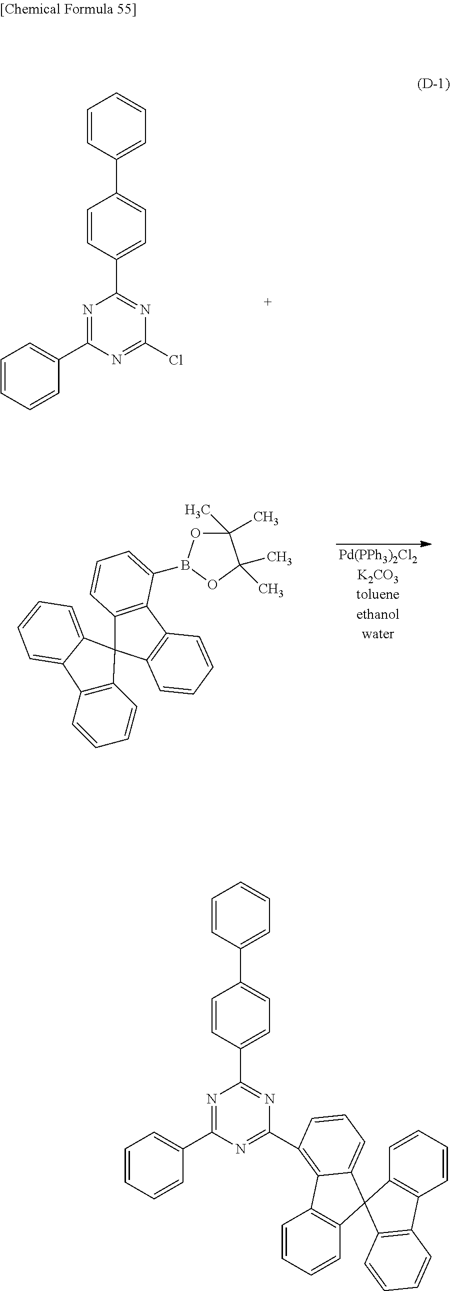 [SMILES: Clc1nc(-c2ccccc2)nc(-c2ccc(-c3ccccc3)cc2)n1 ; CC1(C)OB(c2cccc3c2-c2ccccc2C32c3ccccc3-c3ccccc32)OC1(C)C ; c1ccc(-c2ccc(-c3nc(-c4ccccc4)nc(-c4cccc5c4-c4ccccc4C54c5ccccc5-c5ccccc54)n3)cc2)cc1]

The absorption spectrum and emission spectrum of a light-emitting device containing BP-SF(4)Tzn were measured in a manner similar to that of Example 3. In a solution state, the peak wavelength of the absorption spectrum was 331 nm, and the peak wavelength of the emission spectrum was 423 nm. In a thin film state, the peak wavelength of the absorption spectrum was 357 nm, and the peak wavelength of the emission spectrum was 415 nm.

The Tg of BP-SF(4)Tzn, which was measured in a manner similar to that of Example 1, was 148° C.

### Example 5

### Synthesis Example 5

This synthesis example describes a method for synthesizing 2,4-bis[4-(1-naphthyl)phenyl]-6-[4-(3-pyridyl)phenyl]pyrimidine (abbreviation: 2,4NP-6PyPPm), which is an organic compound usable as a material that can be contained in an organic device (e.g., an electron-transport material and a host material), and is represented by Structural Formula (424).

Into a 200-mL three-neck flask were put 1.1 g (5.8 mmol) of 2,4,6-trichloropyrimidine, 1.2 g (5.8 mmol) of 4-(3-pyridyl)phenylboronic acid, 8.0 g (58 mmol) of potassium carbonate, 80 mL of 1,4-dioxane, and 30 mL of water, the mixture was degassed by being stirred under reduced pressure, and the air in the flask was replaced with nitrogen. Next, the inside of the flask was heated to 40 under a nitrogen stream, 0.20 g (0.29 mmol) of bis(triphenylphosphine)palladium(II) dichloride was added, and stirring was performed for 6.5 hours, so that a reactant containing 4-[4-(3-pyridinyl)phenyl]-2,6-dichloropyrimidine was obtained. Then, 3.3 g (13 mmol) of 4-(1-naphthyl)phenylboronic acid was added to the reactant containing 4-[4-(3-pyridinyl)phenyl]-2,6-dichloropyrimidine, the mixture was degassed by being stirred under reduced pressure, and the air in the flask was replaced with nitrogen. After that, the inside of the flask was heated to 80° C., 0.20 g (0.29 mmol) of bis(triphenylphosphine)palladium(II) dichloride was added, and stirring was performed for 6 hours. Next, ethyl acetate and water were added to a reactant, and an organic layer was extracted. The obtained organic layer was dried using magnesium sulfate and naturally filtered, and the obtained filtrate was concentrated to give a solid. The obtained solid was purified by silica gel column chromatography (toluene:ethyl acetate=2:1 in a developing solvent). The obtained compound was recrystallized with toluene, whereby 2.2 g of a pale yellow solid was obtained (in a yield of 60%). The synthesis scheme is shown in Formula (E-1).

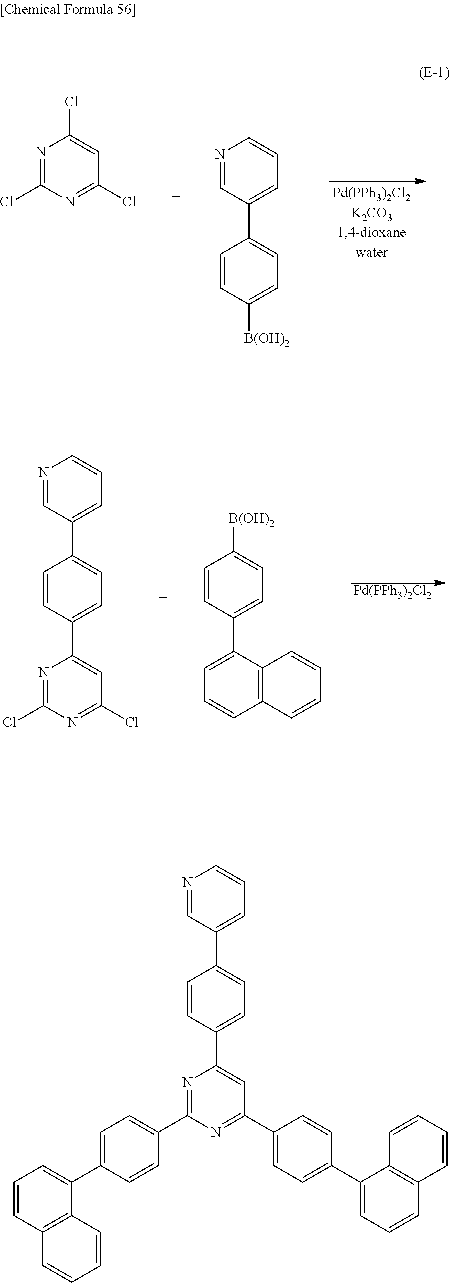 [SMILES: Clc1cc(Cl)nc(Cl)n1 ; OB(O)c1ccc(-c2cccnc2)cc1 ; Clc1cc(-c2ccc(-c3cccnc3)cc2)nc(Cl)n1 ; OB(O)c1ccc(-c2cccc3ccccc23)cc1 ; c1cncc(-c2ccc(-c3cc(-c4ccc(-c5cccc6ccccc56)cc4)nc(-c4ccc(-c5cccc6ccccc56)cc4)n3)cc2)c1 ; "Pd(PPh3)2Cl2"]

The synthesis was performed in one step in Formula (E-1), but can be performed in two steps. In that case, the reactant containing 4-[4-(3-pyridinyl)phenyl]-2,6-dichloropyrimidine is purified (e.g., filtered) to obtain the 4-[4-(3-pyridinyl)phenyl]-2,6-dichloropyrimidine, and then coupling reaction with 4-(1-naphthyl)phenylboronic acid is performed to obtain a target substance (2,4NP-6PyPPm).

The absorption spectrum and emission spectrum of 2,4NP-6PyPPm were measured in a manner similar to that of Example 3. In a solution state, the peak wavelength of the absorption spectrum was 320 nm, and the peak wavelength of the emission spectrum was 387 nm. In a thin film state, the peak wavelength of the absorption spectrum was 311 nm, and the peak wavelength of the emission spectrum was 414 nm.

The Tg of 2,4NP-6PyPPm, which was measured in a manner similar to that of Example 1, was 119° C.

### Example 6

### Synthesis Example 6

This synthesis example describes a method for synthesizing 4-[3,5-bis(9H-carbazol-9-yl)phenyl]-2-phenyl-6-(1,1′-biphenyl-4-yl)pyrimidine (abbreviation: 6BP-4Cz2PPm), which is an organic compound usable as a material that can be contained in an organic device (e.g., an electron-transport material and a host material), and is represented by Structural Formula (404).

Into a 200-mL three-neck flask were put 2.9 g (8.3 mmol) of 4-(biphenyl-4-yl)-6-chloro-2-phenylpyrimidine, 4.5 g (10 mmol) of 3,5-di-9H-carbazol-9-ylphenylboronic acid, 2.8 g (20 mmol) of potassium carbonate, 80 mL of toluene, 12 mL of ethanol, and 12 mL of water, the mixture was degassed by being stirred under reduced pressure, and the air in the flask was replaced with nitrogen. Next, the inside of the flask was heated to 70° C. under a nitrogen stream, 0.35 g (50 μmol) of bis(triphenylphosphine)palladium(II) dichloride was added, the temperature was increased to 90° C., and the mixture was stirred and refluxed for 6 hours. Water was added to a reactant, the obtained mixture was suction-filtered, and a residue was washed with ethanol and water. The obtained residue was dissolved in toluene by heating, the obtained solution was filtered through Celite and aluminum oxide, and the filtrate was concentrated to give a solid. The obtained solid was recrystallized with toluene, whereby 5.4 g of a white solid was obtained (in a yield of 90%). The synthesis scheme is shown in Formula (F-1).

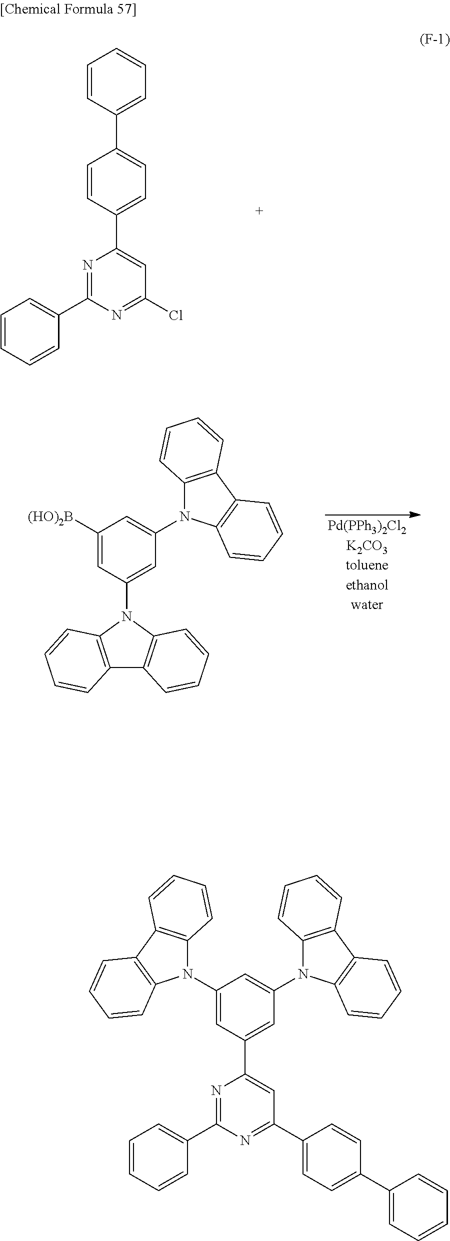 [SMILES: Clc1cc(-c2ccc(-c3ccccc3)cc2)nc(-c2ccccc2)n1 ; OB(O)c1cc(-n2c3ccccc3c3ccccc32)cc(-n2c3ccccc3c3ccccc32)c1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1]

The absorption spectrum and emission spectrum of 6BP-4Cz2PPm were measured in a manner similar to that of Example 1. In a solution state, the peak wavelength of the absorption spectrum was 296 nm, and the peak wavelength of the emission spectrum was 423 nm. In a thin film state, the peak wavelength of the absorption spectrum was 366 nm, and the peak wavelength of the emission spectrum was 425 nm.

### Example 7

### Synthesis Example 7

This synthesis example describes a method for synthesizing 6-(1,1′-biphenyl-3-yl)-4-[3,5-bis(9H-carbazol-9-yl)phenyl]-2-phenylpyrimidine (abbreviation: 6mBP-4Cz2PPm), which is an organic compound usable as a material that can be contained in an organic device (e.g., an electron-transport material and a host material), and is represented by Structural Formula (405).

Note that 6mBP-4Cz2PPm was manufactured in a manner similar to that of Synthesis example 6 except that 4-(biphenyl-3-yl)-6-chloro-2-phenylpyrimidine was used instead of 4-(biphenyl-4-yl)-6-chloro-2-phenylpyrimidine in Synthesis example 6. Note that the heating time was 11 hours and the heating temperature was 90° C. The synthesis scheme is shown in General Formula (G-1).

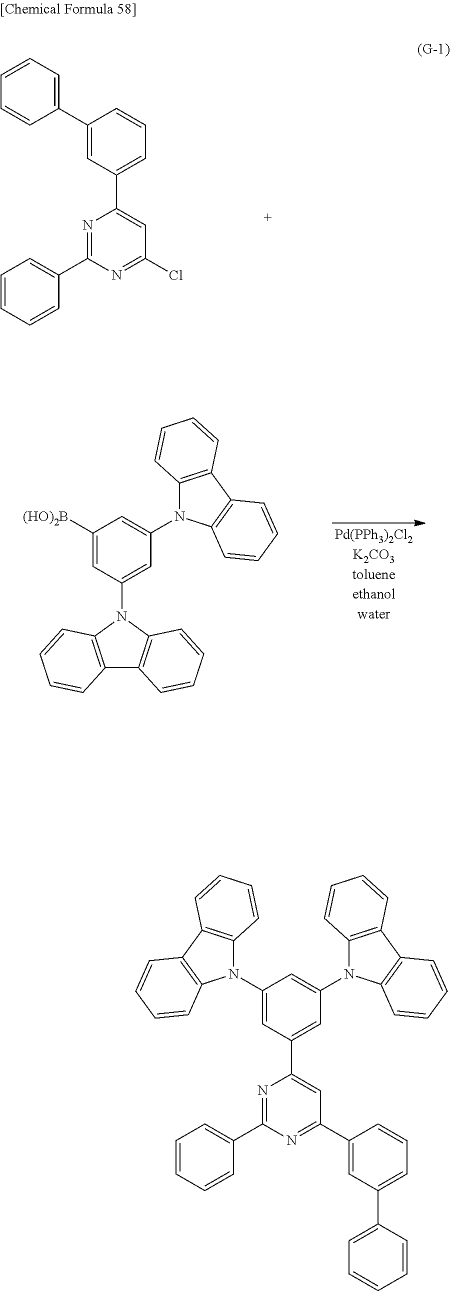

The absorption spectrum and emission spectrum of 6mBP-4Cz2PPm were measured in a manner similar to that of Example 1. In a solution state, the peak wavelength of the absorption spectrum was 338 nm, and the peak wavelength of the emission spectrum was 423 nm. In a thin film state, the peak wavelength of the absorption spectrum was 3356 nm, and the peak wavelength of the emission spectrum was 423 nm.

The Tg of 6mBP-4Cz2PPm, which was measured in a manner similar to that of Example 1, was 132° C.

### Example 8

### Synthesis Example 8

This synthesis example describes a method for synthesizing 2-[3′-(9,9-dimethyl-9H-fluoren-2-yl)biphenyl-3-yl]-4,6-diphenyl-1,3,5-triazine (abbreviation: mFBPTzn), which is an organic compound usable as a material that can be contained in an organic device (e.g., an electron-transport material and a host material), and is represented by Structural Formula (409).

Into a 1-L three-neck flask were put 15 g (36 mmol) of 2-(3′-chlorobiphenyl-3-yl)-4,6-diphenyl-1,3,5-triazine, 9.1 g (38 mmol) of 9,9-dimethyl-9Hfluorene-2-boronic acid, 0.77 mg (2.1 mmol) of di(1-adamantyl)-n-butylphosphine (another name: cataCXium (registered trademark) A) (abbreviation: cataCXium), 18 g (86 mmol) of tripotassium phosphate (abbreviation: K₃PO₄), and 185 mL of diethylene glycol dimethyl ether (abbreviation: diglyme). Next, this mixture was degassed by being stirred under reduced pressure, and the air in the flask was replaced with nitrogen. After that, the inside of the flask was heated to 80° C. under a nitrogen stream, 0.24 mg (1.1 mmol) of palladium acetate was added, the temperature was increased to 140° C., and the mixture was stirred and refluxed for 7 hours. Furthermore, 0.51 g (1.4 mmol) of cataCXium and 0.17 mg (0.75 mmol) of palladium acetate were added, and the mixture was stirred and refluxed at 150° C. for 8 hours. A reactant was dissolved in toluene, filtration through Celite and aluminum oxide was performed, and the obtained filtrate was concentrated to give a solid. The obtained solid was purified by silica gel column chromatography (toluene:hexane=2:3 in a developing solvent). The obtained solid was recrystallized with toluene and hexane, whereby 13 g of a white solid, which is a target compound mFBPTzn, was obtained (64%). The synthesis scheme is shown in Formula (H-1).

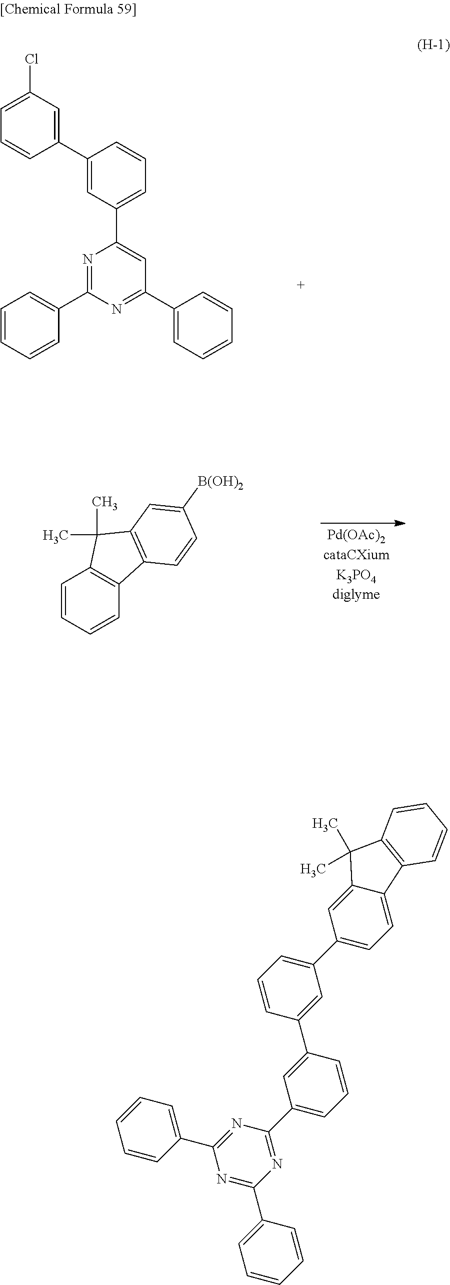 [SMILES: Clc1cccc(-c2cccc(-c3cc(-c4ccccc4)nc(-c4ccccc4)n3)c2)c1 ; CC1(C)c2ccccc2-c2ccc(B(O)O)cc21 ; CC1(C)c2ccccc2-c2ccc(-c3cccc(-c4cccc(-c5nc(-c6ccccc6)nc(-c6ccccc6)n5)c4)c3)cc21]

The absorption spectrum and emission spectrum of mFBPTzn were measured in a manner similar that of Example 1. In a solution state, the peak wavelength of the absorption spectrum was 315 nm, and the peak wavelength of the emission spectrum was 471 nm. In a thin film state, the peak wavelength of the absorption spectrum was 317 nm, and the peak wavelength of the emission spectrum was 410 nm.

The Tg of mFBPTzn, which was measured in a manner similar to that of Example 1, was 95° C.

### Example 9

### Synthesis Example 9

This synthesis example describes a method for synthesizing 3-[9-(4,6-diphenyl-1,3,5-triazin-2-yl)-2-dibenzofuranyl]-9-phenyl-9H-carbazole (abbreviation: PCDBfTzn), which is an organic compound usable as a material that can be contained in an organic device (e.g., an electron-transport material and a host material), and is represented by Structural Formula (445).

Into a three-neck flask were put 2.0 g (4.7 mmol) of 2-(8-chloro-1-dibenzofuranyl)-4,6-diphenyl-1,3,5-triazine, 1.6 g (5.6 mmol) of 9-phenyl-9H-carbazole-3-boronic acid, 85 mg (0.23 mmol) of di(1-adamantyl)-n-butylphosphine, and 3.0 g (14 mmol) of tripotassium phosphate. Next, the air in the flask was replaced with nitrogen, 30 mL of diethylene glycol dimethyl ether was added, and the mixture was degassed by being stirred under reduced pressure. After that, the inside of the flask was heated to 80° C., 18 mg (80 μmol) of palladium acetate was added, the temperature was increased to 120° C., and the mixture was stirred and refluxed for 14 hours. Furthermore, 50 mg (0.14 mmol) of di(1-adamantyl)-n-butylphosphine and 15 mg (70 μmol) of palladium acetate were added, and the mixture was stirred and refluxed at 140° C. for 12 hours. A reactant was suction-filtered, and the obtained residue was washed with toluene and water. The obtained residue was dissolved in toluene by heating, the obtained solution was filtered through Celite and aluminum oxide, and the obtained filtrate was concentrated to give a solid. The obtained solid was recrystallized with toluene, whereby 1.4 g of a yellow solid was obtained (in a yield of 45%). The synthesis method is shown in Formula (I-1).

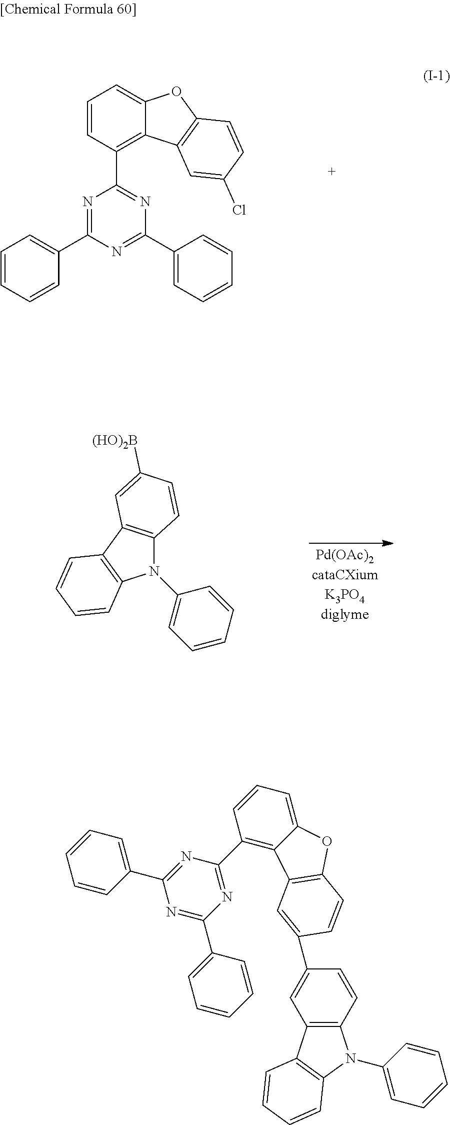 [SMILES: Clc1ccc2oc3cccc(-c4nc(-c5ccccc5)nc(-c5ccccc5)n4)c3c2c1 ; OB(O)c1ccc2c(c1)c1ccccc1n2-c1ccccc1 ; c1ccc(-c2nc(-c3ccccc3)nc(-c3cccc4oc5ccc(-c6ccc7c(c6)c6ccccc6n7-c6ccccc6)cc5c34)n2)cc1]

The absorption spectrum and emission spectrum of PCDBfTzn were measured in a manner similar to that of Example 1. In a thin film state, the peak wavelength of the absorption spectrum was 395 nm, and the peak wavelength of the emission spectrum was 500 nm.

The Tg of PCDBfTzn, which was measured in a manner similar to that of example 1, was 116° C.

Although the details are omitted, Structures (100) to (450) and the like described in Embodiment 1 can be manufactured in a manner similar to those of Examples 1 to 9. In this case, the structures can be manufactured by the method described in Synthesis Methods (a-1) to (a-5) or (b-1) with the use of a compound including any of the substituents (R-01) to (R-112) (a halogen, a boronic acid, or the like) described in Embodiment 1 as appropriate.

### Example 10

This example describes light-emitting devices of embodiments of the present invention described in the embodiments and a comparative light-emitting device. Structural formulae of organic compounds used in the light-emitting devices of embodiments of the present invention are shown below.

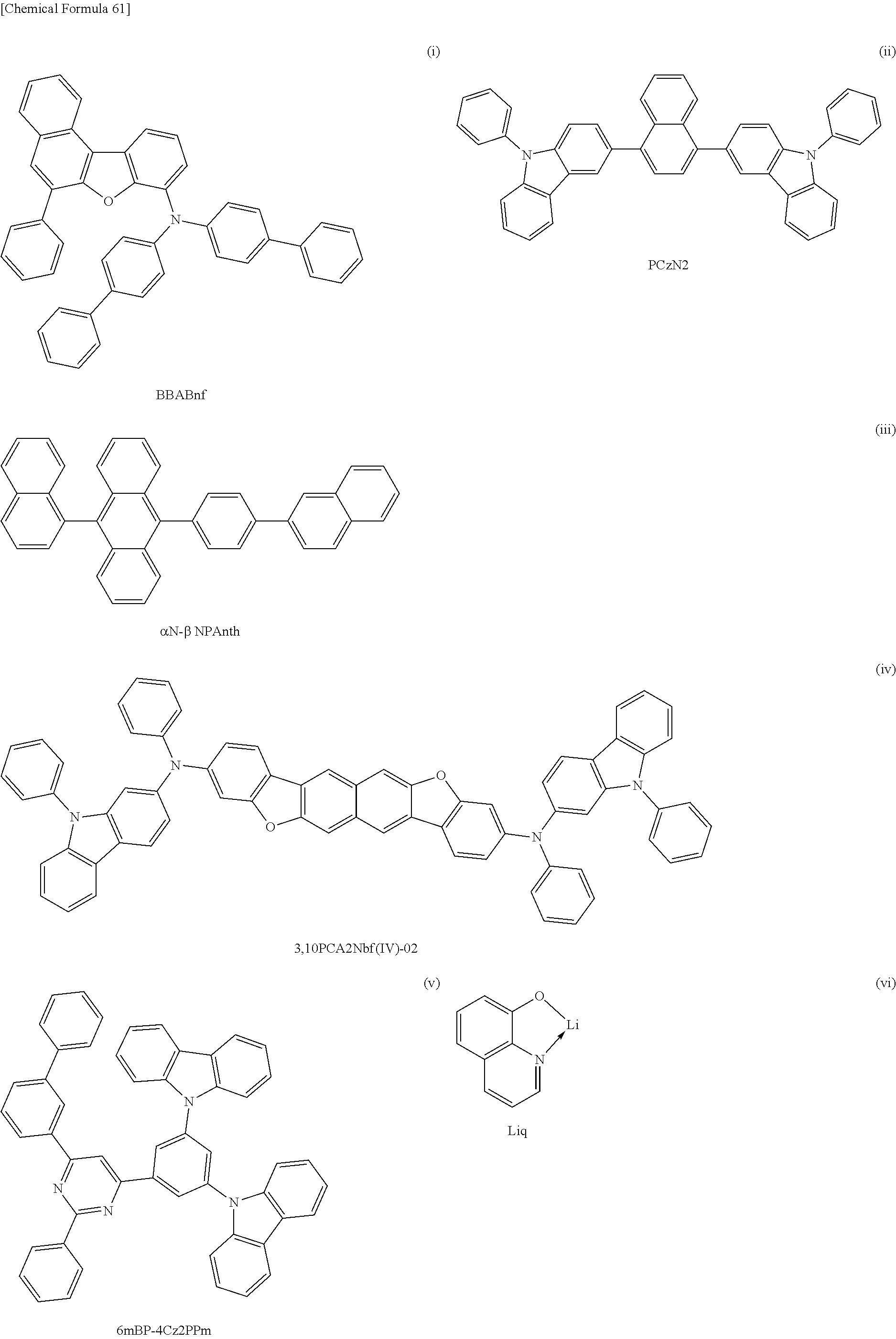

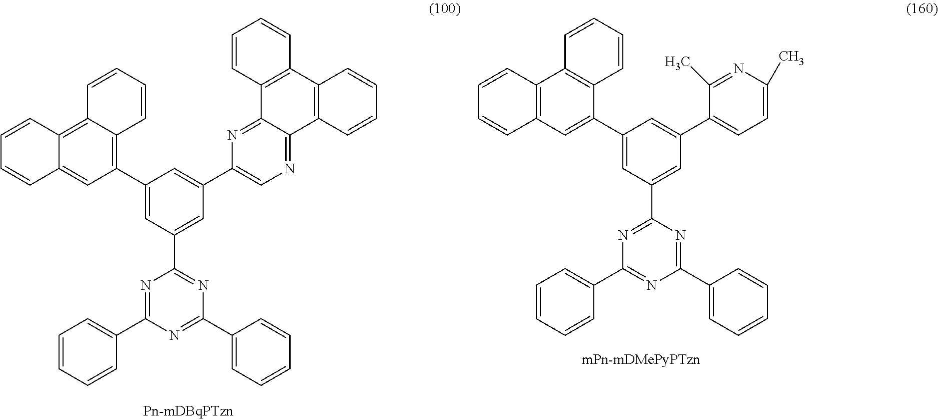 [SMILES: c1ccc(-c2nc(-c3ccccc3)nc(-c3cc(-c4cnc5c6ccccc6c6ccccc6c5n4)cc(-c4cc5ccccc5c5ccccc45)c3)n2)cc1 ; Cc1ccc(-c2cc(-c3nc(-c4ccccc4)nc(-c4ccccc4)n3)cc(-c3cc4ccccc4c4ccccc34)c2)c(C)n1]

### (Fabrication Method of Light-Emitting Device 1)

First, indium tin oxide containing silicon oxide (ITSO) was deposited over a glass substrate by a sputtering method, so that a first electrode was formed. Note that the thickness was 70 nm and the area of the electrode was 2 mm×2 mm.

Next, in pretreatment for forming the light-emitting device over the substrate, a surface of the substrate was washed with water and baking was performed at 200° C. for one hour, and then UV ozone treatment was performed for 370 seconds.

After that, the substrate was transferred into a vacuum evaporation apparatus in which the pressure was reduced to about 1×10⁻⁴ Pa, vacuum baking at 170° C. for 30 minutes was performed in a heating chamber in the vacuum evaporation apparatus, and then the substrate was naturally cooled down for about 30 minutes.

Next, the substrate on which the first electrode was formed was fixed to a substrate holder provided in the vacuum evaporation apparatus such that the surface on which the first electrode was formed faced downward, and BBABnf represented by Structural Formula (i) and OCHD-003 were co-evaporated over the first electrode in a weight ratio of 1:0.1(=BBABnf:OCHD-003) to a thickness of 10 nm by an evaporation method using resistance heating, so that a hole-injection layer was formed. Here, a substance containing halogen and having a mass number of 672 was used as OCHD-003 (also referred to as an electron-accepting substance).

Over the hole-injection layer, BBABnf represented by Structural Formula (i) was evaporated to a thickness of 20 nm, so that a first hole-transport layer was formed.

Over the first hole-transport layer, PCzN2 represented by Structural Formula (ii) was evaporated to a thickness of 10 nm, so that a second hole-transport layer was formed.

Over the second hole-transport layer, αN-βNPAnth (a material functioning as a host) represented by Structural Formula (iii) and 3,10PCA2Nbf(iv)-02 (a material functioning as a dopant) represented by Structural Formula (iv) were co-evaporated in a weight ratio of 1:0.015(=αN-βNPAnth: 3,10PCA2Nbf(iv)-02) to a thickness of 25 nm, so that a light-emitting layer was formed.

Over the light-emitting layer, 6mBP-4Cz2PPm represented by Structural Formula (v) was evaporated to a thickness of 10 nm, so that a first electron-transport layer was formed.

Over the first electron-transport layer, Pn-mDBqPTzn (Structural Formula (100)) of one embodiment of the present invention and Liq represented by Structural Formula (vi) were co-evaporated in a weight ratio of 1:1 to a thickness of 15 nm, so that a second electron-transport layer was formed.

Over the second electron-transport layer, Liq represented by Structural Formula (vi) was evaporated to a thickness of 1 nm, so that an electron-injection layer was formed.

After that, aluminum was evaporated over the electron-transport layer to a thickness of 200 nm to form a second electrode, whereby a light-emitting device 1 of this example was fabricated.

### (Fabrication Method of Light-Emitting Device 2)

In a light-emitting device 2, mPn-mDMePyPTzn (Structural Formula (160)) of one embodiment of the present invention was used instead of Pn-mDBqPTzn in the second electron-transport layer of the light-emitting device 1.

Table 1 shows the element structures of the light-emitting device 1 and the light-emitting device 2 fabricated in the above manner.

TABLE 1

| | | | First | Second | | First | Second | | |
|---|---|---|---|---|---|---|---|---|---|
| | | Hole- | hole- | hole- | Light- | electron- | electron- | Electron- | |
| | First | injection | transport | transport | emitting | transport | transport | injection | Second |
| | electrode | layer | layer | layer | layer | layer | layer | layer | electrode |
| Light- | ITSO | BBABnf: | BBABnf | PCzN2 | * | 6mBP-4Cz2PPm | Pn-mDBqPTzn: | Liq | Al |
| emitting | 70 nm | OCHD-003 | 20 nm | 10 nm | 25 nm | 10 nm | Lig | 1 nm | 200 nm |
| device 1 | | (=1:0.1) | | | | | (=1:1) | | |
| | | 10 nm | | | | | 15 nm | | |
| Light- | ITSO | BBABnf: | BBABnf | PCzN2 | * | 6mBP-4Cz2PPm | mPn-mDMePyPTzn: | Liq | Al |
| emitting | 70 nm | OCHD-003 | 20 nm | 10 mm | 25 nm | 10 nm | Liq | 1 nm | 200 nm |
| device 2 | | (=1:0.1) | | | | | (=1:1) | | |
| | | 10 nm | | | | | 15 nm | | |

*αN-βNPAnth: 3.10PCA2Nbf(IV)-02 (=1:0.015)

These light-emitting devices were subjected to sealing with a glass substrate (a sealant was applied to surround the devices, followed by UV treatment and one-hour heat treatment at 80° C. at the time of sealing) in a glove box containing a nitrogen atmosphere so that the light-emitting devices were not exposed to the air. Then, the initial characteristics were measured.

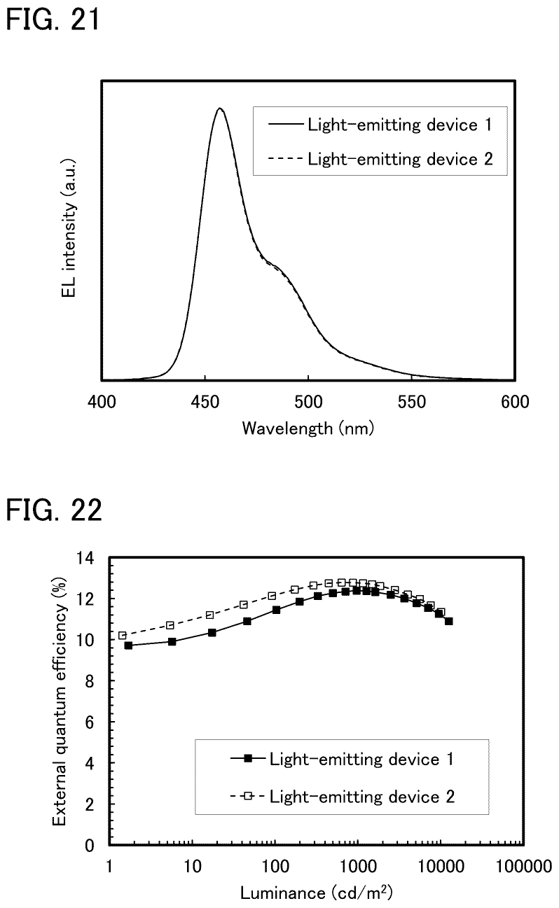

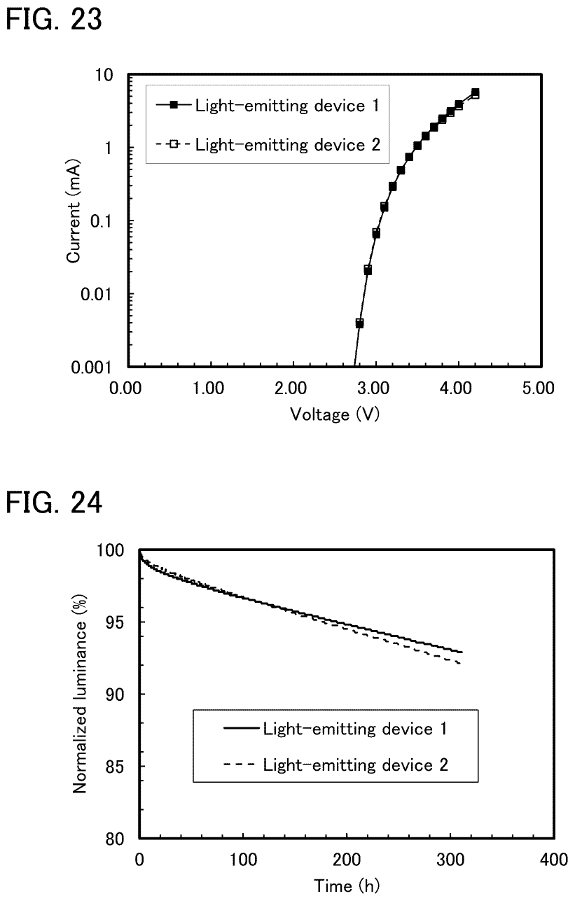

FIG. 21 shows the emission spectra of the light-emitting device 1 and the light-emitting device 2, FIG. 22 shows the external quantum efficiency-luminance characteristics thereof, and FIG. 23 shows the current-voltage characteristics thereof.

Table 2 shows the main characteristics of the light-emitting device 1 and the light-emitting device 2 at approximately 1000 cd/m². Luminance and CIE chromaticity were measured with a luminance colorimeter (BM-5A produced by TOPCON TECHNOHOUSE CORPORATION), and emission spectra were measured with a multi-channel spectrometer (PMA-11 produced by Hamamatsu Photonics K.K.). Note that the measurement of the light-emitting devices was performed at room temperature (in an atmosphere maintained at 23° C.).

TABLE 2

| | | Current | | | Current | External |
|---|---|---|---|---|---|---|
| | Voltage | density | | Luminance | efficiency | Quantum |
| | (V) | (mA/cm²) | Chromaticity x Chromaticity y | (cd/m²) | (cd/A) | efficiency (%) |
| Light-emitting | 3.8 | 8.6 | 0.137 0.103 | 968 | 11.3 | 12.4 |
| device 1 | | | | | | |
| Light-emitting | 3.7 | 7.6 | 0.137 0.102 | 873 | 11.5 | 12.8 |
| device 2 | | | | | | |

FIG. 21 shows that light emission derived from 3,10PCA2Nbf(iv)-02, which is a blue fluorescent dopant, is obtained in the light-emitting device 1 and the light-emitting device 2 that are embodiments of the present invention.

Furthermore, FIG. 22 shows that the light-emitting device 1 and the light-emitting device 2 that are embodiments of the present invention have favorable external quantum efficiency.

FIG. 23 shows that the light-emitting device 1 and the light-emitting device 2 that are embodiments of the present invention can be driven by sufficiently low driving voltage.

FIG. 24 shows the results of the reliability tests of the light-emitting device 1 and the light-emitting device 2. In the reliability test, luminance change over driving time of the elements under conditions of a constant current density of 50 mA/cm² and room temperature was measured. In FIG. 24, the vertical axis represents normalized luminance (%) with the initial luminance being regarded as 100%, and the horizontal axis represents driving time (h) of the element.

As shown in FIG. 24, the light-emitting device 1 and the light-emitting device 2, which are the light-emitting devices of embodiments of the present invention, are found to be light-emitting devices with a long lifetime.

The above element characteristics show that Pn-mDBqPTzn (Structural Formula (100)) and mPn-mDMePyPTzn (Structural Formula (160)) that are embodiments of the present invention are materials having excellent electron-transport properties and being particularly suitable as electron-transport layer materials of a light-emitting device.

### Example 11

This example describes reliability tests of the light-emitting device 1 and the light-emitting device 2 at high temperature.

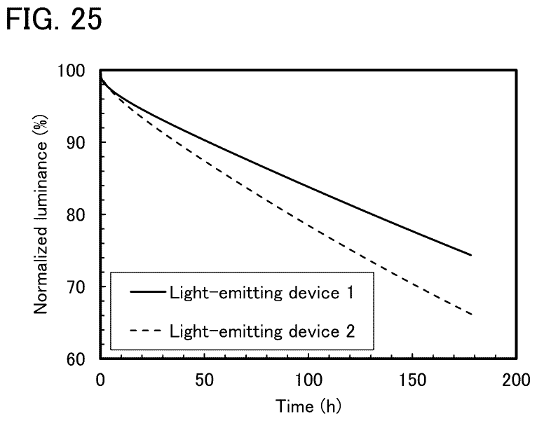

FIG. 25 shows results of the reliability tests of the light-emitting device 1 and light-emitting device 2 at 85° C. The conditions were the same as those of the reliability tests at room temperature (FIG. 24) except that the temperature was set to 85° C.

As shown in FIG. 25, in the reliability test at 85° C., the light-emitting device 1 was found to have a longer lifetime than the light-emitting device 2. That is, Pn-mDBqPTzn (Structural Formula (100)) used for the light-emitting device 1 can be regarded as being superior to mPn-mDMePyPTzn (Structural Formula (160)) used for the light-emitting device 2 in high-temperature resistance.

One factor of the excellent high-temperature resistance of Pn-mDBqPTzn (Structural Formula (100)) is the above-described high glass transition temperature Tg of 161° C. In contrast, the Tg of mPn-mDMePyPTzn (Structural Formula (160)) is 123° C.

Another possible factor of the excellent high-temperature resistance of Pn-mDBqPTzn (Structural Formula (100)) is that, when R³ has a molecular weight of 80 or more in General Formula (G1), which is a molecular structure of a compound of the present application, a thin film with more stable film quality can be obtained. Note that mPn-mDMePyPTzn used for the light-emitting device 2 is a pyridyl group having a molecular weight of 76 (excluding two methyl groups). In contrast, as to Pn-mDBqPTzn used for the light-emitting device 1, R³ in General Formula (G1) is dibenzo[f.h]quinoxaline, and the molecular weight of Pn-mDBqPTzn as a substituent is 229. These differences probably caused the difference in stability of film quality and driving lifetime at high temperature between the fabricated elements.

From the above, the glass transition temperature of a compound used for a light-emitting device is preferably higher than or equal to 130° C., further preferably higher than or equal to 140° C., still further preferably higher than or equal to 150° C., yet still further preferably higher than or equal to 160° C.

Another factor is the number of condensed rings in Pn-mDBqPTzn larger than that in mPn-mDMePyPTzn. Another factor is an increased molecular weight of the condensed rings. Another factor is an increased number of nitrogen atoms contained in the condensed rings.

### REFERENCE NUMERALS

GD: circuit, IR: subpixel, M11: transistor, M12: transistor, M13: transistor, M14: transistor, M15: transistor, M16: transistor, M17: transistor, MS: wiring, PS: subpixel, REG: resist mask, RES: wiring, SE1: wiring, SE: distance, Si: single crystal, TX: wiring, VG: wiring, VS: wiring, 101: first electrode, 102: second electrode, 103: EL layer, 103a: EL layer, 103b: EL layer, 103B: EL layer, 103G: EL layer, 103R: EL layer, 103PS: light-receiving layer, 104B: hole-injection/transport layer, 104G: hole-injection/transport layer, 104R: hole-injection/transport layer, 104PS: first transport layer, 105B: light-emitting layer, 105G: light-emitting layer, 105R: light-emitting layer, 105PS: active layer, 106: charge-generation layer, 106a: charge-generation layer, 106b: charge-generation layer, 107: insulating layer, 108B: electron-transport layer, 108G: electron-transport layer, 108R: electron-transport layer, 108PS: second transport layer, 109: electron-injection layer, 110B: sacrificial layer, 110G: sacrificial layer, 110R: sacrificial layer, 110PS: sacrificial layer, 111: hole-injection layer, 111a: hole-injection layer, 111b: hole-injection layer, 112: hole-transport layer, 112a: hole-transport layer, 112b: hole-transport layer, 113: light-emitting layer, 113a: light-emitting layer, 113b: light-emitting layer, 113c: light-emitting layer, 114: electron-transport layer, 114a: electron-transport layer, 114b: electron-transport layer, 115: electron-injection layer, 115a: electron-injection layer, 115b: electron-injection layer, 130: connection portion, 400: substrate, 401: first electrode, 403: EL layer, 404: second electrode, 405: sealant, 406: sealant, 407: sealing substrate, 412: pad, 420: IC chip, 501C: insulating film, 501D: insulating film, 504: conductive film, 506: insulating film, 508: semiconductor film, 508A: region, 508B: region, 508C: region, 510: first substrate, 512A: conductive film, 512B: conductive film, 516: insulating film, 516A: insulating film, 516B: insulating film, 518: insulating film, 520: functional layer, 524: conductive film, 528: partition wall, 530: pixel circuit, 531: pixel circuit, 550: light-emitting device, 550B: light-emitting device, 550G: light-emitting device, 550R: light-emitting device, 550PS: light-receiving device, 550X: light-emitting device, 550S: light-receiving device, 551B: electrode, 551C: connection electrode, 551G: electrode, 551R: electrode, 551PS: electrode, 552: electrode, 580: space, 591X: wiring, 591S: wiring, 700: light-emitting and light-receiving device, 701: display region, 702B: subpixel, 702G: subpixel, 702PS: subpixel, 702R: subpixel, 702IR: subpixel, 703: pixel, 704: circuit, 705: insulating layer, 706: wiring, 710: substrate, 711: substrate, 712: IC, 713: FPC, 720: apparatus, 770: substrate, 800: substrate, 801a: electrode, 801b: electrode, 802: electrode, 803a: EL layer, 803b: light-receiving layer, 805a: light-emitting device, 805b: light-receiving device, 810: light-emitting and light-receiving device, 810A: light-emitting and light-receiving device, 810B: light-emitting and light-receiving device, 5200B: electronic device, 5210: arithmetic device, 5220: input/output device, 5230: display portion, 5240: input portion, 5250: detecting portion, 5290: communication portion, 8001: ceiling light, 8002: foot light, 8003: sheet-like lighting, 8004: lighting device, 8005: desk lamp, 8006: light source

1-10. (canceled)

11. A compound represented by Formula (G1): 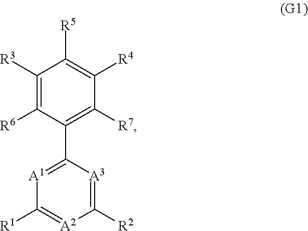
wherein each of R¹, R², R⁵, R⁶ and R⁷ independently represents any of hydrogen, an alkyl group comprising 1 to 10 carbon atoms, a substituted or unsubstituted aryl group comprising 1 to 60 carbon atoms, and a substituted or unsubstituted heteroaryl group comprising 1 to 60 carbon atoms,
wherein R³ represents a condensed ring composed of 4 to 10 rings comprising nitrogen as an element forming the ring,
wherein R⁴ represents any of a substituted or unsubstituted condensed ring comprising 1 to 60 carbon atoms, a substituted or unsubstituted aryl group with a molecular weight of 78 or more, and a substituted or unsubstituted heteroaryl group with a molecular weight of 80 or more,
wherein one of A¹ to A³ represents nitrogen and the others each represent nitrogen or carbon bonding a first unit, and
wherein each of the first units in A¹ to A³ independently represents any one of hydrogen, an alkyl group comprising 1 to 10 carbon atoms, a substituted or unsubstituted aryl group comprising 1 to 60 carbon atoms, and a substituted or unsubstituted heteroaryl group comprising 1 to 60 carbon atoms.

12. The compound according to claim 11, wherein the hydrogen atoms in R¹, R², R⁵, R⁶ and R⁷ include a deuterium atom.

13. The compound according to claim 11, wherein the hydrogen atoms in the units of A¹ to A³ include a deuterium atom.

14. The compound according to claim 11,
wherein R³ represents Formula (G1-1), 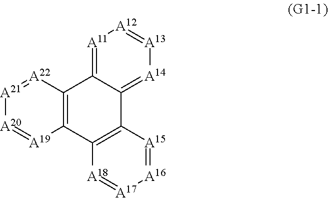
wherein R⁴ represents any one of Formulae (G1-2) to (G1-6), 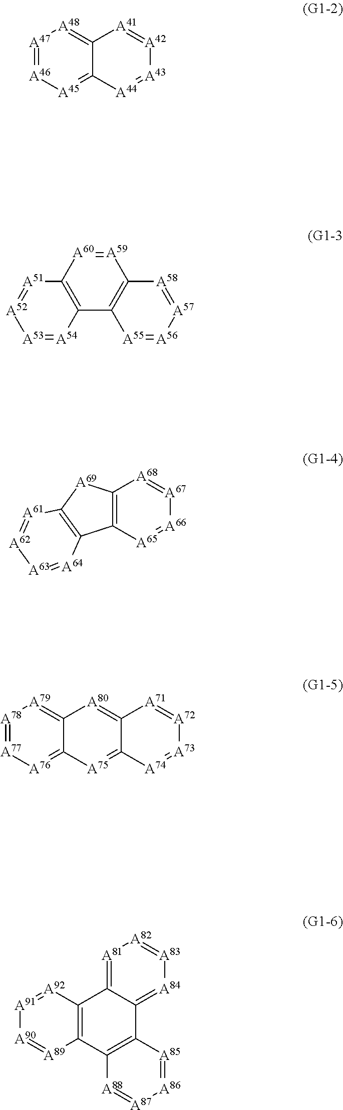
wherein any one of A¹¹ to A²² represents nitrogen, another represents carbon, and the others each independently represent nitrogen or carbon,
wherein one of the carbon atoms in A¹¹ to A²² is a bonding site of R³ in Formula (G1), and the others each independently represent carbon bonding a second unit,
wherein each of the second units in A¹¹ to A²² independently represents hydrogen, an alkyl group comprising 1 to 10 carbon atoms, a substituted or unsubstituted aryl group comprising 1 to 60 carbon atoms, and a substituted or unsubstituted heteroaryl group comprising 1 to 60 carbon atoms,
wherein one of A⁴¹ to A⁴⁸ represents carbon, and the others each independently represent nitrogen or carbon,
wherein one of the carbon atoms of A⁴¹ to A⁴⁸ is a bonding site of R⁴ in Formula (G1), and the others each independently represent carbon bonding a third unit,
wherein one of A⁵¹ to A⁶⁰ represents carbon, and the others each independently represent nitrogen or carbon,
wherein one of the carbon atoms of A⁵¹ to A⁶⁰ is a bonding site of R⁴ in Formula (G1), and the others each independently represent carbon bonding a third unit,
wherein one of A⁶¹ to A⁶⁸ represents carbon, and the others each independently represent nitrogen or carbon,
wherein one of the carbon atoms of A⁶¹ to A⁶⁸ is a bonding site of R⁴ in Formula (G1), and the others each independently represent carbon bonding a third unit,
wherein A⁶⁹ represents any one of nitrogen, carbon, sulfur and oxygen,
wherein one of A⁷¹ to A⁸⁰ represents carbon, and the others each independently represent nitrogen or carbon,
wherein one of the carbon atoms of A⁷¹ to A⁸⁰ is a bonding site of R⁴ in Formula (G1), and the others each independently represent carbon bonding a third unit,
wherein one of A⁸¹ to A⁹² represents carbon, and the others each independently represent nitrogen or carbon,
wherein one of the carbon atoms of A⁸¹ to A⁹² is a bonding site of R⁴ in Formula (G1), and the others each independently represent carbon bonding a third unit, and
wherein each of the third units independently represents any of hydrogen, an alkyl group comprising 1 to 10 carbon atoms, a substituted or unsubstituted aryl group comprising 1 to 60 carbon atoms, and a substituted or unsubstituted heteroaryl group comprising 1 to 60 carbon atoms.

15. The compound according to claim 14, wherein the hydrogen atoms in the second units of A¹¹ to A²² include a deuterium atom.

16. The compound according to claim 14, wherein adjacent two among substituents bonded to A¹¹ to A²² form a ring by bonding to each other.

17. The compound according to claim 14, wherein the hydrogen atoms in the third units of A⁴¹ to A⁴⁸, A⁵¹ to A⁶⁰, A⁷¹ to A⁸⁰ and A⁸¹ to A⁹² include a deuterium atom.

18. The compound according to claim 14, wherein adjacent two among substituents bonded to A⁴¹ to A⁴⁸, adjacent two among substituents bonded to A⁵¹ to A⁶⁰, adjacent two among substituents bonded to A⁷¹ to A⁸⁰ or adjacent two among substituents bonded to A⁸¹ to A⁹² form a ring by bonding to each other.

19. The compound according to claim 11, wherein each of R¹ and R² represents a substituted or unsubstituted phenyl group.

20. The compound according to claim 11, wherein the compound has a glass transition temperature higher than or equal to 150° C.

21. The compound according to claim 11, wherein each of R³ and R⁴ has a molecular weight of 150 or more.

22. The compound according to claim 11, wherein the compound is represented by Formula (100):

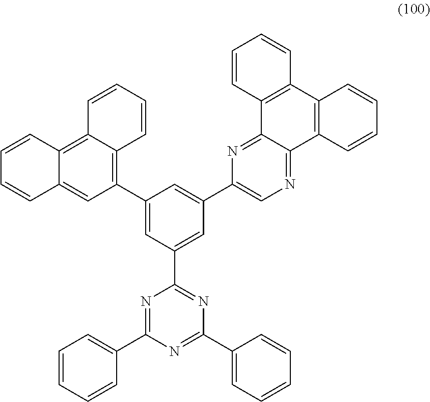 [SMILES: c1ccc(-c2nc(-c3ccccc3)nc(-c3cc(-c4cnc5c6ccccc6c6ccccc6c5n4)cc(-c4cc5ccccc5c5ccccc45)c3)n2)cc1]

23. An organic device comprising the compound according to claim 11.

24. A light-emitting apparatus comprising:
the organic device according to claim 23; and
one of a transistor and a substrate.

25. An electronic appliance comprising the compound according to claim 11.

26. An electronic appliance comprising:
the light-emitting apparatus according to claim 24; and
one of a sensor, an operation button, a speaker and a microphone.
